(19) United States
(12) Patent Application Publication
(10) Pub. No.: US 2008/0280855 A1
(43) Pub. Date: Nov. 13, 2008

(54) Process For the Production of Intermediates For the Preparation of Tricyclic Benzimidazoles

(75) Inventors: Maria Vittoria Chiesa, Konstanz (DE); Andreas Palmer, Singen (DE); Wilm Buhr, Konstanz (DE); Peter Jan Zimmermann, Ehningen (DE); Christof Brehm, Mannheim (DE); Wolfgang-Alexander Simon, Konstanz (DE); Stefan Postius, Konstanz (DE); Wolfgang Kromer, Konstanz (DE); Antonio Zanotti-Gerosa, Cambridge (GB)

Correspondence Address: NATH & ASSOCIATES PLLC, 112 South West Street, Alexandria, VA, 22314, US

(73) Assignee: NYCOMED GmbH, Konstanz (DE)

(21) Appl. No.: 11/922,213

(22) PCT Filed: Jun. 20, 2006
(86) PCT No.: PCT/EP2006/063350

§ 371 Date: Jan. 25, 2008

(30) Foreign Application Priority Data
Jun. 22, 2005 (EP) 05105566.3
Feb. 15, 2006 (EP) 06101699.4

(51) Int. Cl. A61K 31/695; C07F 7/08; C07D 403/02; A61K 31/4184; A61K 31/4188; C07D 491/052

(52) U.S. Cl. 514/63; 548/110; 514/210.21; 548/306.1; 548/302.1; 514/394

(57) ABSTRACT

The invention relates to a process for the synthesis of compounds of the formula 1-a and compounds of the formula 1-b.

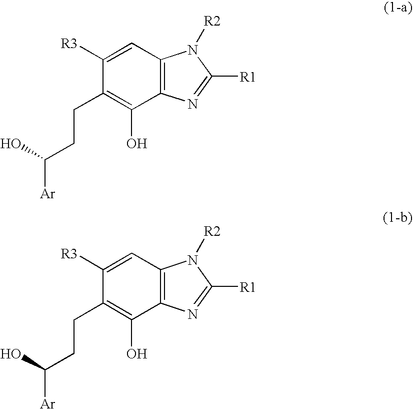

The compounds of the formula 1-a and the compounds of the formula 1-b, in which the substituents R1, R2, R3, and Ar have the meanings indicated in the description, are valuable intermediates for the preparation of pharmaceutically active compounds.

### TECHNICAL FIELD

The invention relates to a process for the production of intermediates, which are used in the pharmaceutical industry for the preparation of active compounds, to the use of certain catalysts in that process, to the intermediates prepared by that process and to active compounds which can be used in medicaments.

### BACKGROUND ART

### (a) Use of Benzimidazole Derivatives for the Treatment of Gastrointestinal Disorders:

In the European patent application EP 0266326 (which corresponds to U.S. Pat. No. 5,106,862), benzimidazole derivatives having a broad variety of substituents are disclosed which are said to be active as anti-ulcer agents. In the international patent application WO 97/47603 (which corresponds to the U.S. Pat. No. 6,465,505), benzimidazole derivatives having a very specific substitution pattern are disclosed, which are said to be suitable for inhibition of gastric acid secretion and thus can be used in the prevention and treatment of gastrointestinal inflammatory diseases.

In the International Patent Application WO 04/054984, benzimidazole derivatives with a variety of substituents are disclosed, which are said to be active as anti-ulcer agents.

The international patent application WO 04/087701 describes cyclic benzimidazoles which inhibit gastric acid secretion and possess excellent gastric and intestinal protective properties. Enantiopure pharmaceutically active compounds of that type are produced from enantiopure prescursors, which can be obtained by an asymmetric hydrogenation of prochiral starting materials using a chiral hydrogenation catalyst.

The international patent applications WO 05/058893, WO 05/103057, WO 05/121139, WO 06/037748 and WO 06/037759 describe tricyclic benzimidazole derivatives having different substitution patterns at the heterocyclic core structure, which compounds likewise inhibit gastric acid secretion and possess excellent gastric and intestinal protective properties.

The international patent application WO 05/058325 describes tricyclic imidazopyridine derivatives which inhibit gastric acid secretion and possess excellent gastric and intestinal protective properties.

Enantiopure compounds of that type are produced from enantiopure precursors, which can be obtained by an asymmetric hydrogenation of prochiral starting materials using a chiral hydrogenation catalyst.

The international patent application WO 05/058894 describes the synthesis of enantiopure hydroxyl intermediates which can be further transformed into pharmaceutically active imidazopyridine derivatives, for example those from described in WO 05/058325. The enantiopure hydroxyl intermediates are obtained from prochiral ketone precursors by an asymmetric catalytic hydrogenation reaction using chiral hydrogenation catalysts.

### (b) Asymmetric Reduction of Carbonyl Compounds to Alcohols in the Presence of Homogenous Hydrogenation Catalysts:

The European patent application EP 0718265 discloses a method for the reduction of carbonyl compounds to alcohols in the presence of a homogeneous hydrogenation catalyst, a base, and a nitrogen-containing organic compound. More specifically, a system consisting of a transition metal complex of a VIII-group metal (preferably Rh, Ru, Ir, Pd, Pt), a hydroxide of an alkali metal or an alkali earth metal or a quarternary ammonium salt, and an amine is employed for this transformation. The reduction of carbonyl compounds can be conducted in an asymmetric manner when optically active bis(diarylphosphane) and diamine ligands are used. Specific examples for suitable ligands comprise BINAP (2,2′-bis(diphenylphosphanyl)-1,1′-binaphthyl), TolBINAP (2,2′-bis(di-4-tolylphosphanyl)-1,1′-binaphthyl), H₈BINAP (2,2′-bis(diphenylphosphanyl)-5,6,7,8,5′,6′,7′,8′-octahydro-[1,1′]-binaphthyl), CHIRAPHOS (2,3-bis(diphenylphosphanyl)butane), DPEN (1,2-diphenylethylenediamine), 1,2-dicyclohexylethylenediamine, DAMEN (1,1-di(4-anisyl)-2-methyl-1,2-ethylenediamine), DAIBEN (1,1-di(4-anisyl)-2-isobutyl-1,2-ethylenediamine) and DAIPEN (1,1-di(4-anisyl)-2-isopropyl-1,2-ethylenediamine). In the following description, ligands belonging to the structural classes bis(diarylphosphane) and diamine are represented by the generic formula PP and NN, respectively.

In a typical experimental procedure, the carbonyl derivative is dissolved in isopropanol and hydrogenated (4-50 atm hydrogen pressure, 28° C., 1-16 hours) in the presence of potassium hydroxide and a homogenous hydrogenation catalyst, which might be formed in situ, for example from (S,S)-DPEN and RuCl₂[(S)-BINAP] (DMF)ₙ. The method is described in more detail in J. Am. Chem. Soc. 1995, 117, 2675-2676, J. Am. Chem. Soc. 1995, 117, 10417-10418, J. Am. Chem. Soc. 1998, 120, 1086-1087 and in the patent applications JP 10273456 and EP 901997.

In a modification of this procedure, the ternary system described above is replaced by a pure ruthenium complex of the generic formula RuXY[PP][NN], where X and Y represent anionic ligands, like e.g. halogen or hydride, and [PP]/[NN] stands for a bis(diarylphosphane)/diamine ligand. The complex RuCl₂[(S)-BINAP][(S,S)-DPEN] represents a specific example for a hydrogenation pre-catalyst. The use of preformed catalyst complexes offers several advantages, like increased reaction rates, higher productivity, and increased stability against air and moisture. The synthesis and the use of these complexes are described—inter alia—in Angew. Chem. 1998, 110, 1792-1796 and in the patent application JP 1189600.

The scope of the catalyst system RuXY[PP][NN] has been investigated thoroughly. In one aspect, these efforts resulted in the discovery of immobilized hydrogenation catalysts, which allow catalyst recycling and an easier work-up of the reaction (see e.g. WO 02/062809, WO 04/084834, US 2004192543). In another aspect, catalysts were found which permit an (asymmetric) reduction of carbonyl compounds in the absence of a base. These catalysts (X═H, Y═BH₄) can be prepared easily by reduction of the corresponding pre-catalysts (X═Y═Cl) with sodium borohydride and are suitable for the preparation of alcohols containing acid-labile groups, like e.g. ester functions. The synthesis and the use of these complexes are described in the patent applications U.S. Pat. No. 6,720,439, JP 2003104993, and JP 2004238306.

Hydrogenation catalysts of the structural class RuCl₂[PP][NN], where [PP] is an optically pure (substituted) BI NAP derivative and [NN] is an optically active 1,2-diamine have been used for the asymmetric reduction of ketones and mines bearing a large variety of functional groups. Nevertheless, considerable efforts have been devoted to identify hydrogenation catalysts with structurally different ligands [PP] and/or [NN] (for a representative list of ligands see e.g. Angew. Chem. 2001, 113, 40-75 and WO 05/007662). The synthesis of a family of ligands [PP], which has been found to be particularly suitable for the asymmetric reduction of carbonyl compounds has been disclosed in Tetrahedron Lett. 2002, 43, 1539-1543. The preparation of hydrogenation catalysts RuCl₂[PP][NN] containing these new ligands [P-Phos (2,2′,6,6′-tetramethoxy-4,4′-bis(diphenylphosphino)-3,3′-bipyridinyl), Tol-P-Phos (2,2′,6,6′-tetramethoxy-4,4′-bis[di(p-tolyl)phosphinol-3,3′-bipyridinyl), Xyl-P-Phos (2,2′,6,6′-tetramethoxy-4,4′-bis[di(3,5-dimethylphenyl)phosphino]-3,3′-bipyridinyl)] in combination with a 1,2-diamine is described in J. Org. Chem. 2002, 67, 7908-7910 and in Chem. Eur. J. 2003, 9, 2963-2968. Furthermore, it has been demonstrated that a wide variety of aromatic and heteroaromatic ketones can be hydrogenated with excellent enantioselectivities. Typically, these reactions are performed in isopropanol in the presence of potassium tert-butoxide using substrate to catalyst ratios (S/C-ratios) up to 100.000:1 and a hydrogen pressure of 1 bar to 400 psi. The new catalysts, like e.g. trans-RuCl₂[(R)-Xyl-P-Phos][(R,R)-DPEN], are said to possess favourable properties.

### DISCLOSURE OF INVENTION

### Technical Problem

The technical problem underlying the present invention is to provide a process for the preparation of intermediates useful for the preparation of enantiomers of tricyclic benzimidazole derivatives, which can be used in therapy.

### Technical Solution

It has now been found that (3R)-6-[3-aryl-3-hydroxypropyl]-7-hydroxy-3H-benzimidazole derivatives can be prepared by an asymmetric catalytic hydrogenation reaction from the corresponding prochiral ketones by using RuXY[(S)-Xyl-P-Phos][(S)-DAIPEN] or RuXY[(S)-Xyl-BINAP][(S)-DAIPEN] as hydrogenation catalyst.

Furthermore it has been found that (3S)-6-[3-aryl-3-hydroxypropyl]-7-hydroxy-3H-benzimidazole derivatives can be prepared by an asymmetric catalytic hydrogenation reaction from the corresponding prochiral ketones by using RuXY[(R)-Xyl-P-Phos][(R)-DAIPEN] or RuXY[(R)-Xyl-BINAP][(R)-DAIPEN] as hydrogenation catalyst.

The invention therefore relates in a first aspect (aspect a) to a process of preparing a compound of the formula 1-a comprising a catalytic hydrogenation of a compound of the formula 2 in the presence of a hydrogenation catalyst which is selected from the group consisting of RuXY[(S)-Xyl-P-Phos][(S)-DAIPEN] and RuXY[(S)-Xyl-BINAP][(S)-DAIPEN],

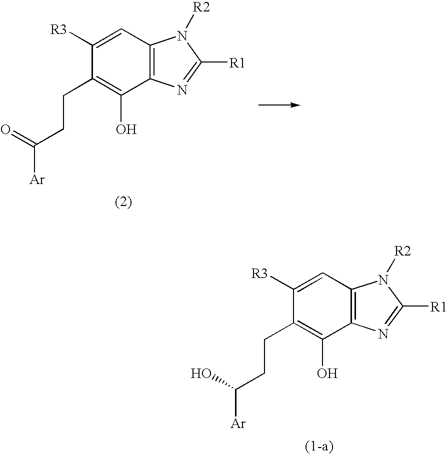

where
X and Y are the same or different substituents selected from the group consisting of hydrogen, halogen, BH₄ and carboxylate,
   and in which
R1 is hydrogen, halogen, hydroxyl, 1-4C-alkyl, 3-7C-cycloalkyl, 3-7C-cycloalkyl-1-4C-alkyl, 1-4C-alkoxy, 1-4C-alkoxy-1-4C-alkyl, 1-4C-alkoxycarbonyl, 2-4C-alkenyl, 2-4C-alkynyl, fluoro-1-4C-alkyl, hydroxy-1-4C-alkyl or mono- or di-1-4C-alkylamino,
R2 is hydrogen, 1-4C-alkyl, 1-4C-alkoxy, 1-4C-alkoxy-1-4C-alkyl, aryl, 3-7C-cycloalkyl, 3-7C-cycloalkyl-1-4C-alkyl, 1-4C-alkoxycarbonyl, mono- or di-1-4C-alkylamino-1-4C-alkylcarbonyl, hydroxy-1-4C-alkyl, fluoro-2-4C-alkyl, 1-4C-alkoxy-1-4C-alkoxy-1-4C-alkyl, silyl substituted 1-4C-alkoxy-1-4Calkyl, 1-4C-alkylcarbonyl, aryl-CH₂-oxycarbonyl
R3 is hydrogen, halogen, fluoro-1-4C-alkyl, carboxyl, 1-4C-alkoxycarbonyl, hydroxy-1-4C-alkyl, 1-4C-alkoxy-1-4C-alkyl, 1-4C-alkoxy-1-4C-alkoxy-1-4C-alkyl, fluoro-1-4C-alkoxy-1-4C-alkyl, 1-4C-alkoxy-1-4C-alkoxy, 1-4C-alkylcarbonylamino, 1-4C-alkylcarbonyl-N-1-4C-alkylamino, 1-4C-alkoxy-1-4C-alkylcarbonylamino or the group —CO—NR31R32,
   where
   R31 is hydrogen, hydroxyl, 1-7C-alkyl, 3-7C-cycloalkyl, hydroxy-1-4C-alkyl or 1-4C-alkoxy-1-4C-alkyl and
   R32 is hydrogen, 1-7C-alkyl, 3-7C-cycloalkyl, hydroxy-1-4C-alkyl or 1-4C-alkoxy-1-4C-alkyl,
   or where
   R31 and R32 together, including the nitrogen atom to which both are bonded, are a pyrrolidino, hydroxy-pyrrolidino, aziridino, azetidino, piperidino, piperazino, N-1-4C-alkylpiperazino or morpholino group,
Ar is a mono- or bicyclic aromatic residue, substituted by R4, R5, R6 and R7, which is selected from the group consisting of phenyl, naphthyl, pyrrolyl, pyrazolyl, imidazolyl, 1,2,3-triazolyl, indolyl, benzimidazolyl, furyl, benzofuryl, thienyl, benzothienyl, thiazolyl, isoxazolyl, pyridinyl, pyrimidinyl, chinolinyl and isochinolinyl,
   wherein
   R4 is hydrogen, 1-4C-alkyl, hydroxy-1-4C-alkyl, 1-4C-alkoxy, 2-4C-alkenyloxy, carboxy, 1-4C-alkoxycarbonyl, carboxy-1-4C-alkyl, 1-4C-alkoxycarbonyl-1-4C-alkyl, 1-4C-alkoxy-1-4C-alkyl, aryloxy-1-4C-alkyl, halogen, hydroxy, aryl, aryl-1-4C-alkyl, aryl-oxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
   R5 is hydrogen, 1-4C-alkyl, 1-4C-alkoxy, 1-4C-alkoxycarbonyl, halogen, trifluoromethyl or hydroxy,
   R6 is hydrogen, 1-4C-alkyl or halogen and
   R7 is hydrogen, 1-4C-alkyl or halogen,
   and wherein
   aryl is phenyl or substituted phenyl with one, two or three same or different substituents selected from the group consisting of 1-4C-alkyl, 1-4C-alkoxy, carboxy, 1-4C-alkoxycarbonyl, halogen, trifluoromethyl, nitro, trifluoromethoxy, hydroxy and cyano.

The invention also relates to a process according to aspect a), in which
R4 is hydrogen, 1-4C-alkyl, hydroxy-1-4C-alkyl, 1-4C-alkoxy, 2-4C-alkenyloxy, carboxy, 1-4C-alkoxycarbonyl, carboxy-1-4C-alkyl, 1-4C-alkoxycarbonyl-1-4C-alkyl, halogen, hydroxy, aryl, aryl-1-4C-alkyl, aryl-oxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
   and the other substituents are defined as outlined above.

The invention also relates to a process according to aspect a), in which
R4 is 1-4C-alkoxy-1-4C-alkyl or aryloxy-1-4C-alkyl
   and the other substituents are defined as outlined above.

The invention further relates in a second aspect (aspect b) to a process of preparing a compound of the formula 1-b comprising a catalytic hydrogenation of a compound of the formula 2 in the presence of a hydrogenation catalyst which is selected from the group consisting of RuXY[(R)-Xyl-P-Phos][(R)-DAIPEN] and RuXY[(R)-Xyl-BINAP][(R)-DAIPEN],

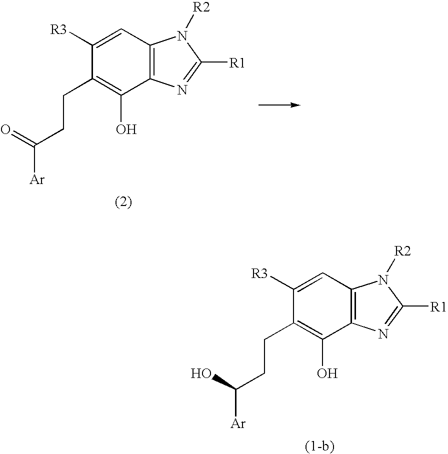

where
X and Y are the same or different substituents selected from the group consisting of hydrogen, halogen, BH₄ and carboxylate
   and in which
R1 is hydrogen, halogen, hydroxyl, 1-4C-alkyl, 3-7C-cycloalkyl, 3-7C-cycloalkyl-1-4C-alkyl, 1-4C-alkoxy, 1-4C-alkoxy-1-4C-alkyl, 1-4C-alkoxycarbonyl, 2-4C-alkenyl, 2-4C-alkynyl, fluoro-1-4C-alkyl, hydroxy-1-4C-alkyl or mono- or di-1-4C-alkylamino,
R2 is hydrogen, 1-4C-alkyl, 1-4C-alkoxy, 1-4C-alkoxy-1-4C-alkyl, aryl, 3-7C-cycloalkyl, 3-7C-cycloalkyl-1-4C-alkyl, 1-4C-alkoxycarbonyl, mono- or di-1-4C-alkylamino-1-4C-alkylcarbonyl, hydroxy-1-4C-alkyl, fluoro-2-4C-alkyl, 1-4C-alkoxy-1-4C-alkoxy-1-4C-alkyl, silyl substituted 1-4C-alkoxy-1-4Calkyl, 1-4C-alkylcarbonyl, aryl-CH₂-oxycarbonyl
R3 is hydrogen, halogen, fluoro-1-4C-alkyl, carboxyl, 1-4C-alkoxycarbonyl, hydroxy-1-4C-alkyl, 1-4C-alkoxy-1-4C-alkyl, 1-4C-alkoxy-1-4C-alkoxy-1-4C-alkyl, fluoro-1-4C-alkoxy-1-4C-alkyl, 1-4C-alkoxy-1-4C-alkoxy, 1-4C-alkylcarbonylamino, 1-4C-alkylcarbonyl-N-1-4C-alkylamino, 1-4C-alkoxy-1-4C-alkylcarbonylamino or the group —CO—NR31R32,
   where
   R31 is hydrogen, hydroxyl, 1-7C-alkyl, 3-7C-cycloalkyl, hydroxy-1-4C-alkyl or 1-4C-alkoxy-1-4C-alkyl and
   R32 is hydrogen, 1-7C-alkyl, 3-7C-cycloalkyl, hydroxy-1-4C-alkyl or 1-4C-alkoxy-1-4C-alkyl,
   or where
   R31 and R32 together, including the nitrogen atom to which both are bonded, are a pyrrolidino, hydroxy-pyrrolidino, aziridino, azetidino, piperidino, piperazino, N-1-4C-alkylpiperazino or morpholino group,
Ar is a mono- or bicyclic aromatic residue, substituted by R4, R5, R6 and R7, which is selected from the group consisting of phenyl, naphthyl, pyrrolyl, pyrazolyl, imidazolyl, 1,2,3-triazolyl, indolyl, benzimidazolyl, furyl, benzofuryl, thienyl, benzothienyl, thiazolyl, isoxazolyl, pyridinyl, pyrimidinyl, chinolinyl and isochinolinyl,
   wherein
   R4 is hydrogen, 1-4C-alkyl, hydroxy-1-4C-alkyl, 1-4C-alkoxy, 2-4C-alkenyloxy, carboxy, 1-4C-alkoxycarbonyl, carboxy-1-4C-alkyl, 1-4C-alkoxycarbonyl-1-4C-alkyl, 1-4C-alkoxy-1-4C-alkyl, aryloxy-1-4C-alkyl, halogen, hydroxy, aryl, aryl-1-4C-alkyl, aryl-oxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
   R5 is hydrogen, 1-4C-alkyl, 1-4C-alkoxy, 1-4C-alkoxycarbonyl, halogen, trifluoromethyl or hydroxy,
   R6 is hydrogen, 1-4C-alkyl or halogen and
   R7 is hydrogen, 1-4C-alkyl or halogen,
   and wherein
   aryl is phenyl or substituted phenyl with one, two or three same or different substituents selected from the group consisting of 1-4C-alkyl, 1-4C-alkoxy, carboxy, 1-4C-alkoxycarbonyl, halogen, trifluoromethyl, nitro, trifluoromethoxy, hydroxy and cyano.

The invention also relates to a process according to aspect b), in which
R4 is hydrogen, 1-4C-alkyl, hydroxy-1-4C-alkyl, 1-4C-alkoxy, 2-4C-alkenyloxy, carboxy, 1-4C-alkoxycarbonyl, carboxy-1-4C-alkyl, 1-4C-alkoxycarbonyl-1-4C-alkyl, halogen, hydroxy, aryl, aryl-1-4C-alkyl, aryl-oxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
   and the other substituents are defined as outlined above.

The invention also relates to a process according to aspect b), in which
R4 is 1-4C-alkoxy-1-4C-alkyl or aryloxy-1-4C-alkyl
   and the other substituents are defined as outlined above.

Halogen within the meaning of the invention is bromo, chloro and fluoro.

1-4C-Alkyl represents a straight-chain or branched alkyl group having 1 to 4 carbon atoms. Examples which may be mentioned are the butyl, isobutyl, sec-butyl, tert-butyl, propyl, isopropyl, ethyl and the methyl group.

3-7C-Cycloalkyl represents cyclopropyl, cyclobutyl, cyclopentyl, cyclohexyl and cycloheptyl, of which cyclopropyl, cyclobutyl and cyclopentyl are preferred.

3-7C-Cycloalkyl-1-4C-alkyl represents one of the aforementioned 1-4C-alkyl groups, which is substituted by one of the aforementioned 3-7C-cycloalkyl groups. Examples which may be mentioned are the cyclopropylmethyl, the cyclohexylmethyl and the cyclohexylethyl group.

1-4C-Alkoxy represents a group, which in addition to the oxygen atom contains one of the aforementioned 1-4C-alkyl groups. Examples which may be mentioned are the butoxy, isobutoxy, sec-butoxy, tert-butoxy, propoxy, isopropoxy and preferably the ethoxy and methoxy group.

1-4C-Alkoxy-1-4C-alkyl represents one of the aforementioned 1-4C-alkyl groups, which is substituted by one of the aforementioned 1-4C-alkoxy groups. Examples which may be mentioned are the methoxymethyl, the methoxyethyl group and the butoxyethyl group.

1-4C-Alkoxycarbonyl (1-4C-alkoxy-CO—) represents a carbonyl group, to which one of the aforementioned 1-4C-alkoxy groups is bonded. Examples which may be mentioned are the methoxycarbonyl (CH₃O—C(O)—), ethoxycarbonyl group (CH₃CH₂O—C(O)—) and the tert-butoxycarbonyl group.

2-4C-Alkenyl represents a straight-chain or branched alkenyl group having 2 to 4 carbon atoms. Examples which may be mentioned are the 2-butenyl, 3-butenyl, 1-propenyl and the 2-propenyl group (allyl group).

2-4C-Alkynyl represents a straight-chain or branched alkynyl group having 2 to 4 carbon atoms. Examples which may be mentioned are the 2-butynyl, 3-butynyl, and preferably the 2-propynyl, group (propargyl group).

Fluoro-1-4C-alkyl represents one of the aforementioned 1-4C-alkyl groups, which is substituted by one or more fluorine atoms. An example which may be mentioned are the trifluoromethyl group, the difluoromethyl, the 2-fluoroethyl, the 2,2-difluoroethyl or the 2,2,2-trifluoroethyl group.

Hydroxy-1-4C-alkyl represents one of the aforementioned 1-4C-alkyl groups, which is substituted by a hydroxy group. Examples which may be mentioned are the hydroxymethyl, the 2-hydroxyethyl and the 3-hydroxypropyl group. Hydroxy-1-4C-alkyl within the scope of the invention is understood to include 1-4C-alkyl groups with two or more hydroxy groups. Examples which may be mentioned are the 3,4-di-hydroxybutyl and in particular the 2,3-dihydroxypropyl group.

Mono- or di-1-4C-alkylamino represents an amino group, which is substituted by one or by two—identical or different—groups from the aforementioned 1-4C-alkyl groups. Examples which may be mentioned are the dimethylamino, the diethylamino and the diisopropylamino group.

Mono- or di-1-4C-alkylamino-1-4C-alkylcarbonyl represents a 1-4C-alkylcarbonyl group, which is substituted by a mono- or di-1-4C-alkylamino groups. Examples, which may be mentioned, are the dimethylamino-methylcarbonyl and the dimethylamino-ethylcarbonyl group.

Fluoro-2-4C-alkyl represents a 2-4C-alkyl group, which is substituted by one or more fluorine atoms. An example which may be mentioned is the 2,2,2-trifluoroethyl group.

Silyl substituted 1-4C-alkoxy-1-4Calkyl represents an 1-4C-alkoxy-1-4C-alkyl group which is substituted by a silyl group. A silyl group in this regard is a Si atom to which are attached three identical or different substituents selected from 1-4C-alkyl or aryl groups. Examples which may be mentioned are the 2-(trimethylsilyl)-ethoxymethyl, the (phenyldimethylsilyl)methoxymethyl or the 1-[2-(trimethylsilyl)ethoxy]ethyl groups.

Aryl-CH₂-oxycarbonyl represents an CH₂-oxycarbonyl group (CH₂—O—C(O)) which is substituted by an above mentioned aryl group. An example which may be mentioned is the benzyloxycarbonyl group.

1-4C-Alkoxy-1-4C-alkoxy represents one of the aforementioned 1-4C-alkoxy groups, which is substituted by a further 1-4C-alkoxy group. Examples which may be mentioned are the groups 2-(methoxy)-ethoxy (CH₃—O—CH₂—CH₂—O—) and 2-(ethoxy)ethoxy (CH₃—CH₂—O—CH₂—CH₂—O—).

1-4C-Alkoxy-1-4C-alkoxy-1-4C-alkyl represents one of the aforementioned 1-4C-alkoxy-1-4C-alkyl groups, which is substituted by one of the aforementioned 1-4C-alkoxy groups. An example which may be mentioned is the group 2-(methoxy)ethoxymethyl (CH₃—O—CH₂—CH₂—O—CH₂—).

Fluoro-1-4C-alkoxy-1-4C-alkyl represents one of the aforementioned 1-4C-alkyl groups, which is substituted by a fluoro-1-4C-alkoxy group. Fluoro-1-4C-alkoxy in this case represents one of the aforementioned 1-4C-alkoxy groups, which substituted by one or more fluorine atoms. Examples of fluoro-substituted 1-4C-alkoxy groups which may be mentioned are the 2-fluoro-ethoxy, 1,1,1,3,3,3-hexafluoro-2-propoxy, the 2-trifluoromethyl-2-propoxy, the 1,1,1-trifluoro-2-propoxy, the perfluoro-tert-butoxy, the 2,2,3,3,4,4,4-heptafluoro-1-butoxy, the 4,4,4-trifluoro-1-butoxy, the 2,2,3,3,3-pentafluoropropoxy, the perfluoroethoxy, the 1,2,2-trifluoroethoxy, in particular the 1,1,2,2-tetrafluoroethoxy, the 2,2,2-trifluoroethoxy, the trifluoromethoxy and preferably the difluoromethoxy group. Examples of fluoro-1-4C-alkoxy-1-4C-alkyl radicals which may be mentioned are, 1,1,2,2-tetrafluoroethoxymethyl, the 2,2,2-trifluoroethoxymethyl, the trifluoromethoxymethyl, 2-fluoroethoxyethyl, the 1,1,2,2-tetrafluoroethoxyethyl, the 2,2,2-trifluoroethoxyethyl, the trifluoromethoxyethyl and preferably the difluoromethoxymethyl and the difluoromethoxyethyl radicals.

1-4C-Alkylcarbonyl-N-1-4C-alkylamino represents an 1-4C-alkylamino group to which a 1-4C-alkylcarbonyl group is bonded. Examples which may be mentioned are the propionyl-N-methylamino (C₃H₇C(O)NCH₃—) and the acetyl-N-methylamino group (CH₃C(O)NCH₃—).

1-4C-Alkoxy-1-4C-alkylcarbonylamino represents a 1-4C-alkylcarbonylamino group to which a 1-4C-alkoxy group is bonded. Examples which may be mentioned are the methoxy-propionylamino (CH₃O—C₃H₆C(O)NH—) and the methoxy-acetylamino group (CH₃O—CH₂C(O)NH—).

1-7C-Alkyl represents a straight-chain or branched alkyl group having 1 to 7 carbon atoms. Examples which may be mentioned are the heptyl, isoheptyl (5-methylhexyl), hexyl, isohexyl (4-methylpentyl), neohexyl (3,3-dimethylbutyl), pentyl, isopentyl (3-methylbutyl), neopentyl (2,2-dimethylpropyl), butyl, isobutyl, sec-butyl, tert-butyl, propyl, isopropyl, ethyl and the methyl group.

Groups Ar which may be mentioned are, for example, the following substituents: 4-acetoxyphenyl, 4-acetamidophenyl, 2-methoxyphenyl, 3-methoxyphenyl, 4-methoxyphenyl, 3-benzyloxyphenyl, 4-benzyloxyphenyl, 3-benzyloxy-4-methoxyphenyl, 4-benzyloxy-3-methoxyphenyl, 3,5-bis(trifluoromethyl)phenyl, 4-butoxyphenyl, 2-chlorophenyl, 3-chlorophenyl, 4-chlorophenyl, 2-chloro-6-fluorophenyl, 3-chloro-4-fluorophenyl, 2-chloro-5-nitrophenyl, 4-chloro-3-nitrophenyl, 3-(4-chlorophenoxy)phenyl, 2,4-dichlorophenyl, 3,4-difluorophenyl, 2,4-dihydroxyphenyl, 2,6-dimethoxyphenyl, 3,4-dimethoxy-5-hydroxyphenyl, 2,5-dimethylphenyl, 3-ethoxy-4-hydroxyphenyl, 2-fluorophenyl, 4-fluorophenyl, 4-hydroxyphenyl, 2-hydroxy-5-nitrophenyl, 3-methoxy-2-nitrophenyl, 3-nitrophenyl, 2,3,5-trichlorophenyl, 2,4,6-trihydroxyphenyl, 2,3,4-trimethoxyphenyl, 2-hydroxy-1-naphthyl, 2-methoxy-1-naphthyl, 4-methoxy-1-naphthyl, 1-methyl-2-pyrrolyl, 2-pyrrolyl, 3-methyl-2-pyrrolyl, 3,4-dimethyl-2-pyrrolyl, 4-(2-methoxycarbonylethyl)-3-methyl-2-pyrrolyl, 5-ethoxycarbonyl-2,4-dimethyl-3-pyrrolyl, 3,4-dibromo-5-methyl-2-pyrrolyl, 2,5-dimethyl-1-phenyl-3-pyrrolyl, 5-carboxy-3-ethyl-4-methyl-2-pyrrolyl, 3,5-dimethyl-2-pyrrolyl, 2,5-dimethyl-1-(4-trifluoromethylphenyl)-3-pyrrolyl, 1-(2,6-dichloro-4-trifluoromethylphenyl)-2-pyrrolyl, 1-(2-nitrobenzyl)-2-pyrrolyl, 1-(2-fluorophenyl)-2-pyrrolyl, 1-(4-trifluoromethoxyphenyl)-2-pyrrolyl, 1-(2-nitrobenzyl)-2-pyrrolyl, 1-(4-ethoxycarbonyl)-2,5-dimethyl-3-pyrrolyl, 5-chloro-1,3-dimethyl-4-pyrazolyl, 5-chloro-1-methyl-3-trifluoromethyl-4-pyrazolyl, 1-(4-chlorobenzyl)-5-pyrazolyl, 1,3-dimethyl-5-(4-chlorphenoxy)-4-pyrazolyl, 1-methyl-3-trifluromethyl-5-(3-trifluoro methylphenoxy)-4-pyrazolyl, 4-methoxycarbonyl-1-(2,6-dichlorophenyl)-5-pyrazolyl, 5-allyl-oxy-1-methyl-3-trifluoromethyl-4-pyrazolyl, 5-chloro-1-phenyl-3-trifluoromethyl-4-pyrazolyl, 3,5-di-methyl-1-phenyl-4-imidazolyl, 4-bromo-1-methyl-5-imidazolyl, 2-butylimidazolyl, 1-phenyl-1,2,3-triazol-4-yl, 3-indolyl, 4-indolyl, 7-indolyl, 5-methoxy-3-indolyl, 5-benzyloxy-3-indolyl, 1-benzyl-3-indolyl, 2-(4-chlorophenyl)-3-indolyl, 7-benzyloxy-3-indolyl, 6-benzyloxy-3-indolyl, 2-methyl-5-nitro-3-indolyl, 4,5,6,7-tetrafluoro-3-indolyl, 1-(3,5-difluorobenzyl)-3-indolyl, 1-methyl-2-(4-trifluorophenoxy)-3-indolyl, 1-methyl-2-benzimidazolyl, 5-nitro-2-furyl, 5-hydroxymethyl-2-furyl, 2-furyl, 3-furyl, 5-(2-nitro-4-trifluoromethylphenyl)-2-furyl, 4-ethoxycarbonyl-5-methyl-2-furyl, 5-(2-trifluoromethoxyphenyl)-2-furyl, 5-(4-methoxy-2-nitrophenyl)-2-furyl, 4-bromo-2-furyl, 5-dimethylamino-2-furyl, 5-bromo-2-furyl, 5-sulfo-2-furyl, 2-benzofuryl, 2-thienyl, 3-thienyl, 3-methyl-2-thienyl, 4-bromo-2-thienyl, 5-bromo-2-thienyl, 5-nitro-2-thienyl, 5-methyl-2-thienyl, 5-(4-methoxyphenyl)-2-thienyl, 4-methyl-2-thienyl, 3-phenoxy-2-thienyl, 5-carboxy-2-thienyl, 2,5-dichloro-3-thienyl, 3-methoxy-2-thienyl, 2-benzothienyl, 3-methyl-2-benzothienyl, 2-bromo-5-chloro-3-benzothienyl, 2-thiazolyl, 2-amino-4-chloro-5-thiazolyl, 2,4-dichloro-5-thiazolyl, 2-diethylamino-5-thiazolyl, 3-methyl-4-nitro-5-isoxazolyl, 2-pyridyl, 3-pyridyl, 4-pyridyl, 6-methyl-2-pyridyl, 3-hydroxy-5-hydroxymethyl-2-methyl-4-pyridyl, 2,6-dichloro-4-pyridyl, 3-chloro-5-trifluoromethyl-2-pyridyl, 4,6-dimethyl-2-pyridyl, 4-(4-chlorophenyl)-3-pyridyl, 2-chloro-5-methoxy-carbonyl-6-methyl-4-phenyl-3-pyridyl, 2-chloro-3-pyridyl, 6-(3-trifluoromethylphenoxy)-3-pyridyl, 2-(4-chlorophenoxy)-3-pyridyl, 2,4-dimethoxy-5-pyrimidinyl, 2-quinolinyl, 3-quinolinyl, 4-quinolinyl, 2-chloro-3-quinolinyl, 2-chloro-6-methoxy-3-quinolinyl, 8-hydroxy-2-quinolinyl and 4-isoquinolinyl.

2-4C-Alkenyloxy represents a group, which in addition to the oxygen atom contains one of the above-mentioned 2-4C-alkenyl groups. Examples, which may be mentioned, are the 2-butenyloxy, 3-butenyl-oxy, 1-propenyloxy and the 2-propenyloxy group (allyloxy group).

1-4C-Alkylcarbonyl represents a group, which in addition to the carbonyl group contains one of the abovementioned 1-4C-alkyl groups. Examples which may be mentioned are the acetyl and the pivaloyl group.

Carboxy-1-4C-alkyl represents a 1-4C-alkyl group which is substituted by a carboxyl group. Examples, which may be mentioned, are the carboxymethyl and the 2-carboxyethyl group.

1-4C-Alkoxycarbonyl-1-4C-alkyl represents a 1-4C-alkyl group, which is substituted by one of the abovementioned 1-4C-alkoxycarbonyl groups. Examples, which may be mentioned, are the Methoxy-carbonylmethyl and the ethoxycarbonylmethyl group.

Aryl-1-4C-alkyl represents one of the aforementioned 1-4C-alkyl groups, which is substituted by one of the abovementioned aryl groups. An exemplary preferred aryl-1-4C-alkyl group is the benzyl group.

Aryl-1-4C-alkoxy represents one of the aforementioned 1-4C-alkoxy groups, which is substituted by one of the abovementioned aryl groups. An exemplary preferred aryl-1-4C-alkoxy group is the benzyloxy group.

1-4C-Alkylcarbonylamino represents an amino group to which a 1-4C-alkylcarbonyl group is bonded. Examples which may be mentioned are the propionylamino (C₃H₇C(O)NH—) and the acetylamino group (acetamido group) (CH₃C(O)NH—).

1-4C-Alkoxycarbonylamino represents an amino group, which is substituted by one of the aforementioned 1-4C-alkoxycarbonyl groups. Examples, which may be mentioned, are the ethoxycarbonylamino and the methoxycarbonylamino group.

1-4C-Alkoxy-1-4C-alkoxycarbonyl represents a carbonyl group, to which one of the aforementioned 1-4C-alkoxy-1-4C-alkoxy groups is bonded. Examples which may be mentioned are the 2-(methoxy)-ethoxycarbonyl (CH₃—O—CH₂CH₂—O—CO—) and the 2-(ethoxy)ethoxycarbonyl group (CH₃CH₂—O—CH₂CH₂—O—CO—).

1-4C-Alkoxy-1-4C-alkoxycarbonylamino represents an amino group, which is substituted by one of the aforementioned 1-4C-alkoxy-1-4C-alkoxycarbonyl groups. Examples which may be mentioned are the 2-(methoxy)ethoxycarbonylamino and the 2-(ethoxy)ethoxycarbonylamino group.

Aryloxy represents a group, which in addition to the oxygen atom contains one of the abovementioned aryl groups. An example which may be mentioned is the benzyloxy group.

Aryloxy-1-4C-alkyl represents an 1-4C-alkyl group which is substituted by one of the above mentioned aryloxy groups. An example which may be mentioned is the benzyloxy-methyl group.

Preference is given to aspect a according to the present invention.

In a first embodiment (embodiment 1) of the invention RuXY[(S)-Xyl-P-Phos][(S)-DAIPEN] is used as the hydrogenation catalyst for the synthesis of (3R)-6-[3-aryl-3-hydroxypropyl]-7-hydroxy-3H-benzimidazole derivatives.

In a second embodiment (embodiment 2) of the invention RuXY[(S)-Xyl-BINAP][(S)-DAIPEN] is used as the hydrogenation catalyst for the synthesis of (3R)-6-[3-aryl-3-hydroxypropyl]-7-hydroxy-3H-benzimidazole derivatives.

In a third embodiment (embodiment 3) of the invention RuXY[(R)-Xyl-P-Phos][(R)-DAIPEN] is used as the hydrogenation catalyst for the synthesis of (3S)-6-[3-aryl-3-hydroxypropyl]-7-hydroxy-3H-benzimidazole derivatives.

In a forth embodiment (embodiment 4) of the invention RuXY[(R)-Xyl-BINAP][(R)-DAIPEN] is used as the hydrogenation catalyst for the synthesis of (3S)-6-[3-aryl-3-hydroxypropyl]-7-hydroxy-3H-benzimidazole derivatives

Particular emphasis is given to embodiment 1 according to the invention.

Preferred is a process for the preparation of compounds of the formula 1-a according to aspect a or of compounds of the formula 1-b according to aspect b from compounds of the formula 2,

where
X and Y are the same or different substituents selected from the group consisting of hydrogen, halogen, BH₄ and carboxylate
and in which
R1 is hydrogen, 1-4C-alkyl, 3-7C-cycloalkyl, 3-7C-cycloalkyl-1-4C-alkyl, 1-4C-alkoxy-1-4C-alkyl or hydroxy-1-4C-alkyl,
R2 is hydrogen, 1-4C-alkyl, 1-4C-alkoxy-1-4C-alkyl, 3-7C-cycloalkyl, 3-7C-cycloalkyl-1-4C-alkyl, hydroxy-1-4C-alkyl, fluoro-2-4C-alkyl, 1-4C-alkoxy-1-4C-alkoxy-1-4C-alkyl, silyl substituted 1-4C-alkoxy-1-4Calkyl, 1-4C-alkylcarbonyl, aryl-CH₂-oxycarbonyl
R3 is hydrogen, halogen, fluoro-1-4C-alkyl, carboxyl, 1-4C-alkoxycarbonyl, hydroxy-1-4C-alkyl, 1-4C-alkoxy-1-4C-alkyl, 1-4C-alkoxy-1-4C-alkoxy-1-4C-alkyl, fluoro-1-4C-alkoxy-1-4C-alkyl, 1-4C-alkoxy-1-4C-alkoxy, 1-4C-alkylcarbonylamino, 1-4C-alkylcarbonyl-N-1-4C-alkylamino, 1-4C-alkoxy-1-4C-alkylcarbonylamino or the group —CO—NR31R32,
   where
   R31 is hydrogen, hydroxyl, 1-7C-alkyl, 3-7C-cycloalkyl, hydroxy-1-4C-alkyl or 1-4C-alkoxy-1-4C-alkyl and
   R32 is hydrogen, 1-7C-alkyl, 3-7C-cycloalkyl, hydroxy-1-4C-alkyl or 1-4C-alkoxy-1-4C-alkyl,
   or where
   R31 and R32 together, including the nitrogen atom to which both are bonded, are a pyrrolidino, hydroxy-pyrrolidino, aziridino, azetidino, piperidino, piperazino, N-1-4C-alkylpiperazino or morpholino group,
Ar is a phenyl, naphthyl, pyrrolyl, thienyl, benzothienyl or pyridinyl substituted by R4, R5, R6 and R7,
   wherein
   R4 is hydrogen, 1-4C-alkyl, hydroxy-1-4C-alkyl, 1-4C-alkoxy, 2-4C-alkenyloxy, carboxy, 1-4C-alkoxycarbonyl, carboxy-1-4C-alkyl, 1-4C-alkoxycarbonyl-1-4C-alkyl, 1-4C-alkoxy-1-4C-alkyl, aryloxy-1-4C-alkyl halogen, hydroxy, aryl, aryl-1-4C-alkyl, aryl-oxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
   R5 is hydrogen, 1-4C-alkyl, 1-4C-alkoxy, 1-4C-alkoxycarbonyl, halogen, trifluoromethyl or hydroxy,
   R6 is hydrogen, 1-4C-alkyl or halogen and
   R7 is hydrogen, 1-4C-alkyl or halogen,
   and wherein
   aryl is phenyl or substituted phenyl with one, two or three same or different substituents selected from the group consisting of 1-4C-alkyl, 1-4C-alkoxy, carboxy, 1-4C-alkoxycarbonyl, halogen, trifluoromethyl, nitro, trifluoromethoxy, hydroxy and cyano.

Particularly preferred is a process for the preparation of compounds of the formula 1-a according to aspect a or of compounds of the formula 1-b according to aspect b from compounds of the formula 2,

where
X and Y are the same or different substituents selected from the group consisting of hydrogen, halogen, BH₄ and carboxylate
and in which
R1 is hydrogen, 1-4C-alkyl, 3-7C-cycloalkyl or hydroxy-1-4C-alkyl,
R2 is hydrogen, 1-4C-alkyl, hydroxy-1-4C-alkyl, 1-4C-alkoxy-1-4C-alkoxy-1-4C-alkyl, silyl substituted 1-4C-alkoxy-1-4Calkyl, 1-4C-alkylcarbonyl or aryl-CH₂-oxycarbonyl
R3 is carboxyl, 1-4C-alkoxycarbonyl, hydroxy-1-4C-alkyl, 1-4C-alkoxy-1-4C-alkyl, 1-4C-alkoxy-1-4C-alkoxy-1-4C-alkyl, or the group —CO—NR31R32,
   where
   R31 is hydrogen, hydroxyl, 1-7C-alkyl, 3-7C-cycloalkyl, hydroxy-1-4C-alkyl or 1-4C-alkoxy-1-4C-alkyl and
   R32 is hydrogen, 1-7C-alkyl, 3-7C-cycloalkyl, hydroxy-1-4C-alkyl or 1-4C-alkoxy-1-4C-alkyl,
   or where
   R31 and R32 together, including the nitrogen atom to which both are bonded, are a pyrrolidino, hydroxy-pyrrolidino, aziridino, azetidino, piperidino, piperazino, N-1-4C-alkylpiperazino or morpholino group,
Ar is a phenyl, naphthyl, pyrrolyl, thienyl, benzothienyl or pyridinyl substituted by R4, R5, R6 and R7,
   wherein
   R4 is hydrogen, 1-4C-alkyl, 1-4C-alkoxy, halogen, hydroxy-1-4C-alkyl, 1-4C-alkoxy-1-4C-alkyl or aryloxy-1-4C-alkyl,
   R5 is hydrogen, 1-4C-alkyl, or halogen,
   R6 is hydrogen, 1-4C-alkyl or halogen and
   R7 is hydrogen, 1-4C-alkyl or halogen.

Emphasis is a given to a process for the preparation of compounds of the formula 1-a according to aspect a or of compounds of the formula 1-b according to aspect b from compounds of the formula 2,

where
X and Y are the same or different substituents selected from the group consisting of hydrogen, halogen, BH₄ and carboxylate
and in which

### R1 is 1-4C-alkyl,

R2 is hydrogen, 1-4C-alkyl or silyl substituted 1-4C-alkoxy-1-4Calkyl,
R3 is 1-4C-alkoxy-1-4C-alkyl or the group —CO—NR31R32,
where
R31 is hydrogen, 1-7C-alkyl or 3-7C-cycloalkyl and
R32 is hydrogen or 1-7C-alkyl,
or where
R31 and R32 together, including the nitrogen atom to which both are bonded, are a pyrrolidino, or azetidino group,
Ar is a phenyl, naphthyl, thienyl or benzothienyl substituted by R4, R5, R6 and R7,
   wherein
   R4 is hydrogen, 1-4C-alkyl, halogen, 1-4C-alkoxy-1-4C-alkyl, aryloxy-1-4C-alkyl or trifluoromethyl,
   R5 is hydrogen or halogen,
   R6 is hydrogen and
   R7 is hydrogen.

Emphasis is also given to a process for the preparation of compounds of the formula 1-a according to aspect a or of compounds of the formula 1-b according to aspect b from compounds of the formula 2,

where
X and Y are the same or different substituents selected from the group consisting of hydrogen, halogen, BH₄ and carboxylate
and in which

### R1 is 1-4C-alkyl,

### R2 is 1-4C-alkyl,

R3 is the group —CO—NR31R32,
where
R31 is hydrogen, 1-7C-alkyl or 3-7C-cycloalkyl and
R32 is hydrogen or 1-7C-alkyl,
or where
R31 and R32 together, including the nitrogen atom to which both are bonded, are a pyrrolidino or a azetidino group,
Ar is a phenyl, naphthyl, pyrrolyl, thienyl, benzothienyl or pyridinyl substituted by R4, R5, R6 and R7,
   wherein
   R4 is hydrogen, 1-4C-alkyl, 1-4C-alkoxy, halogen, hydroxy-1-4C-alkyl or 1-4C-alkoxy-1-4C-alkyl,
   R5 is hydrogen, 1-4C-alkyl, or halogen,
   R6 is hydrogen, 1-4C-alkyl or halogen and
   R7 is hydrogen, 1-4C-alkyl or halogen.

Particular emphasis is given to a process for the preparation of compounds of the 1-a according to aspect a from compounds of the formula 2, using RuXY[(S)-Xyl-P-Phos][(S)-DAIPEN] as hydrogenation catalyst

where
X and Y are each a chlorine radical,
and in which

### R1 is 1-4C-alkyl.

R2 is hydrogen, 1-4C-alkyl or silyl substituted 1-4C-alkoxy-1-4Calkyl,
R3 is 1-4C-alkoxy-1-4C-alkyl or the group —CO—NR31R32,
where
R31 is hydrogen, 1-7C-alkyl or 3-7C-cycloalkyl and
R32 is hydrogen or 1-7C-alkyl,
or where
R31 and R32 together, including the nitrogen atom to which both are bonded, are a pyrrolidino, or azetidino group,
Ar is a phenyl, naphthyl, thienyl or benzothienyl substituted by R4, R5, R6 and R7,
   wherein
   R4 is hydrogen, 1-4C-alkyl, halogen, 1-4C-alkoxy-1-4C-alkyl, aryloxy-1-4C-alkyl, or trifluoromethyl,
   R5 is hydrogen or halogen,
   R6 is hydrogen and
   R7 is hydrogen.

Particular emphasis is also given to a process for the preparation of compounds of the 1-a according to aspect a from compounds of the formula 2, using RuXY[(S)-Xyl-P-Phos][(S)-DAIPEN] as hydrogenation catalyst

where X and Y are each a chlorine radical,
and in which

### R1 is 1-4C-alkyl,

R2 is 1-4C-alkyl or silyl substituted 1-4C-alkoxy-1-4Calkyl,
R3 is the group —CO—NR31R32,
where
R31 is hydrogen or 1-7C-alkyl,
R32 is hydrogen or 1-7C-alkyl,
or where
R31 and R32 together, including the nitrogen atom to which both are bonded, are a azetidino group,
   Ar is a phenyl substituted by R4
wherein
R4 is hydrogen, 1-4C-alkyl or halogen.

Hydrogenation catalysts which are to be emphasized in connection with the present invention are those hydrogenation catalysts mentioned above in which X and Y are each a chlorine radical, that is the hydrogenation catalysts RuCl₂[(S)-Xyl-P-Phos][(S)-DAIPEN], RuCl₂[(S)-Xyl-BINAP][(S)-DAIPEN], RuCl₂[(R)-Xyl-P-Phos][(R)-DAIPEN] and RuCl₂[(R)-Xyl-BINAP][(R)-DAIPEN].

A hydrogenation catalyst which is to be particularly emphasized in connection with the present invention is the hydrogenation catalyst RuCl₂[(S)-Xyl-P-Phos][(S)-DAIPEN].

The compounds according to the invention can be synthesized from corresponding starting compounds, for example according to the reaction schemes given below. The synthesis is carried out in a manner known to the expert, for example as described in more detail in the examples, which follow the schemes.

The compounds of the formula 1-a and 1-b are prepared as outlined in the following scheme 1.

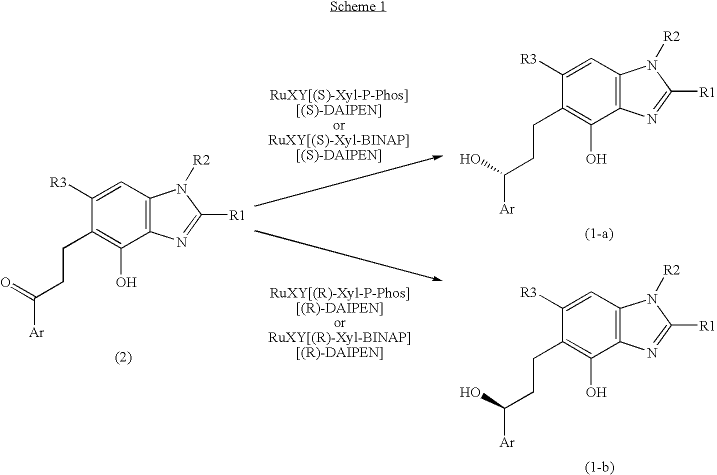

Prochiral ketones of the formula 2 are reduced to optically pure diols of the formula 1-a by homogenous catalytic hydrogenation using RuXY[(S)-Xyl-P-Phos][(S)-DAIPEN] or RuXY[(S)-Xyl-BINAP][(S)-DAIPEN].

Prochiral ketones of the formula 2 are reduced to optically pure diols of the formula 1-b by homogenous catalytic hydrogenation using RuXY[(R)-Xyl-P-Phos][(R)-DAIPEN] or RuXY[(R)-Xyl-BINAP][(R)-DAIPEN].

Compared to the processes known from the prior art for the synthesis of related compounds of the formula 1-a or 1-b, for example those processes mentioned in the International Patent Application WO 04/087701, the process according to the present invention is particularly distinguished inter alia by numerous advantages:
1. Increased isolated yield and/or enantiomeric excess.
2. Easier purification of the product and easier removal of metal residues due to full conversion even at low catalyst:substrate ratio.
3. Volume-efficacy due to the tolerance of water in the solvent system which allows the use of high substrate concentrations.
4. Tolerance of a wide variety of substituents, especially in ortho-position of the aromatic ring Ar

The processes according to the present invention are performed in a manner known to the expert (see e.g. the documents mentioned in the outset of the present application and the studies described in J. Am. Chem. Soc. 2003, 125, 13490-13503). More specifically, the conditions discussed below or in the experimental section are preferably applied. However, it has to be emphasized that the asymmetric reduction of ketones of the formula 2 according to the present invention is not limited to these conditions. Due to his expert knowledge, a person skilled in the art is able to identify reaction conditions suitable for optimal performance of the asymmetric catalytic hydrogenation reaction described in the present invention.

The asymmetric catalytic hydrogenation reaction according to the present invention is advantageously carried out in a suitable organic solvent. Solvents which are to be mentioned are inter alia aliphatic alcohols like for example methanol, ethanol or preferably isopropanol or tert-butanol. Preferred solvent systems are also mixtures of one, two or three of the aliphatic alcohols mentioned before in any mixing ratio, whereby a mixture of isopropanol and tert-butanol in any mixing ratio between 0:100 vol-% and 100:0 vol-% is to be particularly mentioned.

A solvent or a solvent system essentially comprises a specific solvent or a mixture of specific solvents if it contains at least 50%, in particular at least 70% of said specific solvent or said mixture of specific solvents. The other components the solvent or the solvent system are further additives such as for example other organic solvents or water.

The solvent systems mentioned above may comprise, in addition to the alcohol or mixture of alcohols, between 0 and 50 vol-%, preferably between 5 and 30 vol-% of water.

Other additives, such as for example toluene, might also be beneficial for the course of the reaction. Due to his expert knowledge, these additives and their ratio in comparison to the solvent or the solvent system can be identified by a person skilled in the art.

The asymmetric catalytic hydrogenation reaction according to the present invention is advantageously carried out at temperatures between 0 and 80° C., preferably between 20 and 80° C. Below 20° C., the reaction rate might be low, which might result in long reaction times. Above 80° C., the reaction might proceed with concomitant decomposition of the hydrogenation catalyst. This might result in incomplete turnover and/or reduced enantioselectivities.

The reaction time depends on many parameters, like e.g. structure of the substrate, substrate to catalyst ratio (S/C-ratio), amount of base, temperature, hydrogen pressure, solvent, hydrogenation apparatus and the like. Typically, complete transformation is achieved within a time range of 1 hour to 7 days. A person skilled in the art is able to identify the optimum reaction time for each reaction condition.

The asymmetric catalytic hydrogenation reaction according to the present invention is advantageously carried out at hydrogen pressures between 1 and 200 bars, preferably between 10 and 80 bars. As a general rule, the higher the hydrogen pressure the higher is the reaction rate whereby an increase of the hydrogen pressure does not lead to an erosion of enantioselectivity.

The asymmetric catalytic hydrogenation reaction according to the present invention is carried out in the presence of a base in order to generate the active hydrogenation catalyst and in order to increase the turnover number.

The reaction mixture therefore comprises between 1.0 and 50, preferably between 1.01 and 10 and particularly between 1.1 and 3.0 equivalents of an inorganic or organic base (relating to the substrate of the formula 2). Suitable inorganic bases are for example hydroxides, alkoxides or carbonates of alkali metals (caesium, rubidium, potassium, sodium, lithium) or earth alkali metals (magnesium, calcium). Suitable organic bases are for example tertiary amines (e.g. triethylamine) and strong nitrogen bases (e.g. phosphazene bases, like e.g. P4-t-Bu, CAS 111324-04-0). Preferred bases are inorganic bases, such as for example the hydroxides, alkoxides or carbonates of the alkali or earth alkali metals mentioned above. Particular mention may be made of the inorganic bases KOMe, KOⁱPr, LiOH, LiOMe, LiOⁱPr, NaOH, NaOMe or NaOⁱPr, and especially KOH, KO^{t}Bu, K₂CO₃ and Cs₂CO₃. The use of the bases KO^{t}Bu and KOH is particularly preferred.

Preferably, a solution of the corresponding base in one or more of the solvents employed for the hydrogenation reaction—rather than the solid base—is added to the reaction mixture. Specific examples comprise a solution of potassium tert-butoxide in tert-butanol or a solution of potassium hydroxide in water.

The asymmetric catalytic hydrogenation reaction according to the present invention is carried out in concentrations of 0.001 to 10 M, preferably 0.01 to 10 M and especially 0.1 to 1 M solutions of the substrate of the formula 2 in the solvent. The maximum concentration is, however, determined by the solubility of the ketone of the formula 2 in the solvent mixture used for the hydrogenation reaction. A high substrate concentration is beneficial for the reaction rate and the person skilled in the art is able to identify the optimum concentration for each substrate of the formula 2 in each solvent system.

The molar ratio of the substrate of the formula 2 compared to the catalyst (S/C-ratio) depends inter alia on the structure of the ketone of the formula 2. The S/C-ratio applicable according to the present invention is between 5:1 to 100000:1, preferably between 10:1 and 50000:1 and in particular between 100:1 and 1000:1. The person skilled in the art is able to identify the optimum S/C-ratio for each substrate of the formula 2.

The sample preparation according to the present invention might be performed as described in the following examples without being limited to these procedures: Under inert atmosphere, a solution of the corresponding base and additional solvent is added to a mixture the ketone of the formula 2 and the hydrogenation pre-catalyst. The reaction solution is purged with hydrogen, hydrogen pressure is applied and the mixture is heated to the corresponding temperature. Alternatively, a suspension of the ketone of the formula 2 in degassed solvent is treated with base. Subsequently, the hydrogenation catalyst is added to the clear solution, followed by application of hydrogen pressure and heating as described above.

Another possibility is the use of pre-activated hydrogenation catalyst {prepared e.g. by heating a solution of RuCl₂[(S)-Xyl-PPhos][(S)-DAIPEN] (or another pre-catalyst) and potassium-tert-butylate (or another base) in isopropanol to 60° C. for 1 h). In both modes of sample preparation the pre-activated catalyst is added last (prior to application of hydrogen pressure).

Likewise, the isolation of the alcohol of the formula 1-a or 1-b from the reaction mixture relies on processes known to the expert. The isolation of highly pure, Ruthenium-free alcohols of the formula 1-a or 1-b can be accomplished for example applying one of the following procedures or by any other suitable method known to the expert:
Work-up of the reaction mixture: The alcohol of the formula 1-a or 1-b is obtained in the form of its phenolate salt. The neutral form of the corresponding product is obtained by addition of a suitable acid, which is known to a person skilled in the art. Both, weak and strong acids, can be used to generate the neutral form of the hydrogenation product. For example, the crude reaction mixture can be dissolved in a biphasic mixture of ammonium chloride and dichloromethane, optionally followed by addition of a mineral acid (e.g. hydrochloric acid, sulphuric acid), and extraction of the alcohol of the formula 1-a or 1-b.
Purification: The alcohol of the formula 1-a or 1-b can be purified by column chromatography or preferably, by crystallization using suitable organic solvents, like for example ketones (e.g. acetone, methyl ethyl ketone, methyl tert-butyl ketone), alcohols (e.g. methanol, ethanol, isopropanol), ethers (e.g. diethyl ether, methyl tert-butyl ether) or mixtures of these solvents. Removal of Ruthenium residues is effected by crystallization or by the use of scavenger resins. Suitable scavenger resins contain functional groups that form water-soluble ruthenium complexes, which can be removed by a subsequent extraction step.

The invention particularly relates to a process for the preparation of compounds of the formula 1-a and of the formula 1-b according to the present invention, which process is performed in the presence of a base which is selected from KOH, KO^{t}Bu, K₂CO₃ and Cs₂CO₃ and where the solvent essentially comprises isopropanol or tert-butanol or a mixture of isopropanol and tert-butanol in any mixing ratio between 0:100 vol-% and 100:0 vol-% and where the process is carried out in a homogenous solution containing the ketone of the formula 2 in concentrations between 0.1 and 1 M.

The invention particularly relates to a process of preparing a compound of the formula 1-a and of the formula 1-b according to the present invention, which process is performed in the presence of a base which is selected from KOH, KO^{t}Bu, K₂CO₃ and Cs₂CO₃ and where the solvent essentially comprises isopropanol or tert-butanol or a mixture of isopropanol and tert-butanol in any mixing ratio between 0:100 vol-% and 100:0 vol-% and where the solvent additionally comprises between 5 and 30 vol-% of water and where the process is carried out in a homogenous solution containing the ketone of the formula 2 in concentrations between 0.1 and 1 M.

The invention also relates to a compound of the formula 1-a, wherein R1, R2, R3 and Ar have the meanings as indicated in the outset prepared by a process according to the present invention.

The invention particularly relates to a compound of the formula 1-a, wherein R1, R2, R3 and Ar have the meanings as indicated in table 1a and 1b which are outlined below for the compounds of the formula 3-a and 3-b.

The invention further relates to use of RuXY[(S)-Xyl-P-Phos][(S)-DAIPEN] or RuXY[(S)-Xyl-BINAP][(S)-DAIPEN] as the hydrogenation catalyst in a process according to the present invention for the preparation of compounds of the formula 1-a wherein R1, R2, R3 and Ar have the meanings as indicated in the outset.

The invention also relates to a compound of the formula 1-b, wherein R1, R2, R3 and Ar have the meanings as indicated in the outset prepared by a process according to the present invention.

The invention particularly relates to a compound of the formula 1-b, wherein R1, R2, R3 and Ar have the meanings as indicated in table 1a and 1b which are outlined below for the compounds of the formula 3-a and 3-b.

The invention further relates to use of RuXY[(R)-Xyl-P-Phos][(R)-DAIPEN] or RuXY[(R)-Xyl-BINAP][(R)-DAIPEN] as the hydrogenation catalyst in a process according to the present invention for the preparation of compounds of the formula 1-b wherein R1, R2, R3 and Ar have the meanings as indicated in the outset.

Transformation of derivatives of the formula 1-a into pharmacologically active enantiopure (8S)-8-aryl-3,6,7,8-tetrahydro-chromeno[7,8-d]imidazoles derivatives of the formula 3-a can be accomplished by methods which proceed under S_{N}2 conditions, like for example those methods which are disclosed in WO 04/087701. For this purpose, the hydroxyl group in alpha-position to the Ar radical can be transformed into a suitable leaving group LG, e.g. by esterification with acid halides or sulfonyl chlorides. The preparation of compounds of the formula 4-a might require temporary protection of the phenolic hydroxyl group. Suitable protecting groups are described for example in T. W. Greene, P. G. M. Wuts “Protective Groups in Organic Synthesis” 3^{rd} edition, J. Wiley & Sons, New York, 1999. Alternatively, cyclization of the diols of the formula 1-a can be accomplished under Mitsunobu conditions, e.g. using diisopropyl azodicarboxylate and triphenylphosphine. In the same manner, derivatives of the formula 1-b can be transformed into enantiopure (8R)-8-aryl-3,6,7,8-tetrahydro-chromeno[7,8-d]imidazoles derivatives of the formula 3-b.

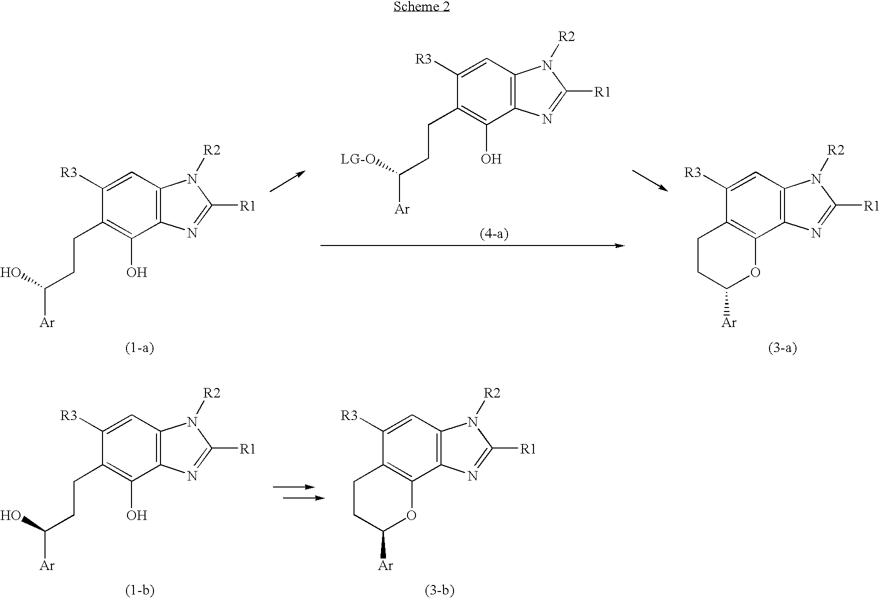

Compounds of the formula 2 are known for example from WO 04/087701, or they can be prepared in a known manner, analogously to known compounds (Scheme 3). The purity of the compounds of the formula 2 has a major impact on the reaction conditions and the outcome of the asymmetric catalytic hydrogenation.

In contrast to WO 04/087701 a further purification step is required, for example a crystallization step in the presence of a suitable organic acid, as described in an exemplary manner in the examples. A convenient method to transform compounds of the formula 2 into other compounds of the formula 2 bearing a different substituent R3 is shown in Scheme 3 and might be illustrated by the following examples: Esters of compounds of the formula 7, wherein R33 is for example a 1-4C-alkyl radical, can be transformed into acetals of the formula 8, for example by reaction with 2,2-dimethoxypropane in the presence of acids. Cleavage of the ester function, e.g. by saponification with sodium hydroxide, furnishes the corresponding carboxylic acids of the formula 9, which are then treated with a suitable coupling reagent, e.g. TBTU, followed by addition of the coupling partner, e.g. an amine, yielding derivatives of the formula 10. Alternatively, esters of the formula 8 can be reduced to the corresponding primary alcohol, e.g. using lithium aluminium hydride, and the hydroxyl group can be activated for example by conversion into a halide or a sulfonate using e.g. thionyl chloride or methanesulfonyl chloride. Interconversion of the substituent R3 can then be accomplished by nucleophilic displacement reactions using nucleophiles like e.g. alkoxides. Finally, ketones of the formula 2 are obtained by cleavage of acetals of the formula 10, e.g. in the presence of acids like hydrochloric acid.

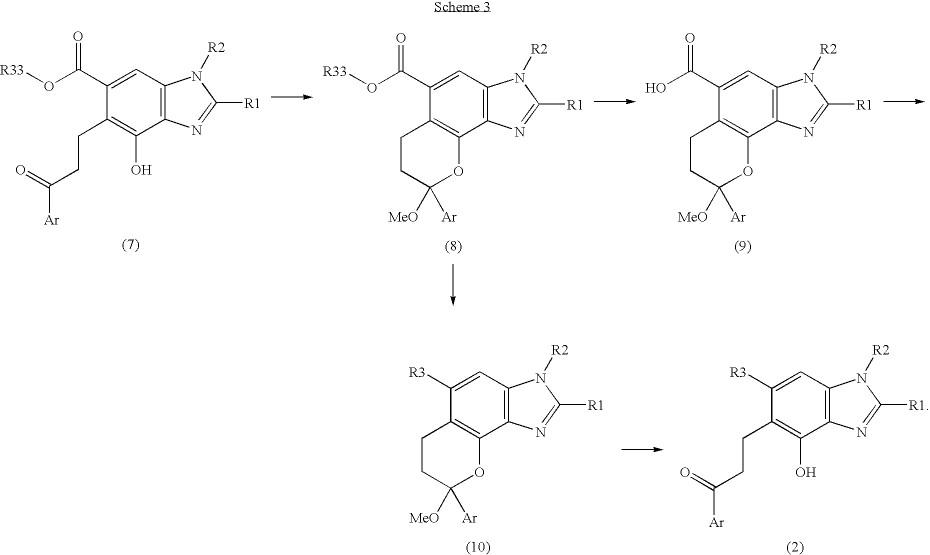

The invention further relates to the compounds of the formula 3-a and 3-b, wherein R1, R2, R3 and Ar have the meanings as indicated in the following table 1a, and the salts of these compounds.

Preferred are the compounds of the formula 3-a, wherein R1, R2, R3 and Ar have the meanings as indicated in the following table 1a and the salts of these compounds.

TABLE 1a

| R1 | R2 | R3 | Ar |
|---|---|---|---|
| —CH₃ | —CH₃ | —C(O)—N(CH₃)₂ | 2-methyl-phenyl |
| —CH₃ | —CH₃ | —C(O)—N(CH₃)₂ | 2-ethyl-phenyl |
| —CH₃ | —CH₃ | —C(O)—N(CH₃)₂ | 2-isopropyl-phenyl |
| —CH₃ | —CH₃ | —C(O)—N(CH₃)₂ | 4-fluoro-2-methyl-phenyl |
| —CH₃ | —CH₃ | —C(O)—N(CH₃)₂ | 2-trifluoromethyl-phenyl |
| —CH₃ | —CH₃ | —C(O)—N(CH₃)₂ | 2-hydroxymethyl-phenyl |
| —CH₃ | —CH₃ | —C(O)—N(CH₃)₂ | 2-chloro-phenyl |
| —CH₃ | —CH₃ | —C(O)—N(CH₃)₂ | 2-(2-hydroxyethyl)- |
| | | | phenyl |
| —CH₃ | —CH₃ | —C(O)—N(CH₃)₂ | 2-(1-hydroxyethyl)- |
| | | | phenyl |
| —CH₃ | —CH₃ | —C(O)—N(CH₃)₂ | 2-benzyloxymethyl- |
| | | | phenyl |
| —CH₃ | —CH₃ | —C(O)—N(CH₃)₂ | 2-methoxymethyl-phenyl |
| —CH₃ | —CH₃ | —C(O)—N(CH₃)₂ | 2-thienyl |
| —CH₃ | —CH₃ | —C(O)—N(CH₃)₂ | 3-methyl-2-thienyl |
| —CH₃ | —CH₃ | —C(O)—N(CH₃)₂ | 3-thienyl |
| —CH₃ | —CH₃ | —C(O)—N(CH₃)₂ | 2-methyl-3-thienyl |
| —CH₃ | —CH₃ | —C(O)—N(CH₃)₂ | 4-methyl-3-thienyl |
| —CH₃ | —CH₃ | —C(O)—N(CH₃)₂ | 1-benzothien-3-yl |
| —CH₃ | —CH₃ | —C(O)—N(CH₃)₂ | 1-naphthyl |
| —CH₃ | —CH₃ | —C(O)—N(CH₃)₂ | 2-naphthyl |
| —CH₃ | —CH₃ | —C(O)—N(CH₃)₂ | 2-N-methyl-pyrrolyl |
| —CH₃ | —CH₃ | —C(O)—N(CH₃)₂ | 4-pyridyl |
| —CH₃ | —CH₃ | —C(O)—N(H)CH₃ | 2-methyl-phenyl |
| —CH₃ | —CH₃ | —C(O)—N(H)CH₃ | 2-ethyl-phenyl |
| —CH₃ | —CH₃ | —C(O)—N(H)CH₃ | 2-isopropyl-phenyl |
| —CH₃ | —CH₃ | —C(O)—N(H)CH₃ | 4-fluoro-2-methyl-phenyl |
| —CH₃ | —CH₃ | —C(O)—N(H)CH₃ | 2-trifluoromethyl-phenyl |
| —CH₃ | —CH₃ | —C(O)—N(H)CH₃ | 2-hydroxymethyl-phenyl |
| —CH₃ | —CH₃ | —C(O)—N(H)CH₃ | 2-chloro-phenyl |
| —CH₃ | —CH₃ | —C(O)—N(H)CH₃ | 2-(2-hydroxyethyl)- |
| | | | phenyl |
| —CH₃ | —CH₃ | —C(O)—N(H)CH₃ | 2-(1-hydroxyethyl)- |
| | | | phenyl |
| —CH₃ | —CH₃ | —C(O)—N(H)CH₃ | 2-benzyloxymethyl- |
| | | | phenyl |
| —CH₃ | —CH₃ | —C(O)—N(H)CH₃ | 2-methoxymethyl-phenyl |
| —CH₃ | —CH₃ | —C(O)—N(H)CH₃ | 2-thienyl |
| —CH₃ | —CH₃ | —C(O)—N(H)CH₃ | 3-methyl-2-thienyl |
| —CH₃ | —CH₃ | —C(O)—N(H)CH₃ | 3-thienyl |
| —CH₃ | —CH₃ | —C(O)—N(H)CH₃ | 2-methyl-3-thienyl |
| —CH₃ | —CH₃ | —C(O)—N(H)CH₃ | 4-methyl-3-thienyl |
| —CH₃ | —CH₃ | —C(O)—N(H)CH₃ | 1-benzothien-3-yl |
| —CH₃ | —CH₃ | —C(O)—N(H)CH₃ | 1-naphthyl |
| —CH₃ | —CH₃ | —C(O)—N(H)CH₃ | 2-naphthyl |
| —CH₃ | —CH₃ | —C(O)—N(H)CH₃ | 2-N-methyl-pyrrolyl |
| —CH₃ | —CH₃ | —C(O)—N(H)CH₃ | 4-pyridyl |
| —CH₃ | —CH₃ | —C(O)—N(H)-cyclopropyl | 2-methyl-phenyl |
| —CH₃ | —CH₃ | —C(O)—N(H)-cyclopropyl | 2-ethyl-phenyl |
| —CH₃ | —CH₃ | —C(O)—N(H)-cyclopropyl | 2-isopropyl-phenyl |
| —CH₃ | —CH₃ | —C(O)—N(H)-cyclopropyl | 4-fluoro-2-methyl-phenyl |
| —CH₃ | —CH₃ | —C(O)—N(H)-cyclopropyl | 2-trifluoromethyl-phenyl |
| —CH₃ | —CH₃ | —C(O)—N(H)-cyclopropyl | 2-hydroxymethyl-phenyl |
| —CH₃ | —CH₃ | —C(O)—N(H)-cyclopropyl | 2-chloro-phenyl |
| —CH₃ | —CH₃ | —C(O)—N(H)-cyclopropyl | 2-(2-hydroxyethyl)- |
| | | | phenyl |
| —CH₃ | —CH₃ | —C(O)—N(H)-cyclopropyl | 2-(1-hydroxyethyl)- |
| | | | phenyl |
| —CH₃ | —CH₃ | —C(O)—N(H)-cyclopropyl | 2-benzyloxymethyl- |
| | | | phenyl |
| —CH₃ | —CH₃ | —C(O)—N(H)-cyclopropyl | 2-methoxymethyl-phenyl |
| —CH₃ | —CH₃ | —C(O)—N(H)-cyclopropyl | 2-thienyl |
| —CH₃ | —CH₃ | —C(O)—N(H)-cyclopropyl | 3-methyl-2-thienyl |
| —CH₃ | —CH₃ | —C(O)—N(H)-cyclopropyl | 3-thienyl |
| —CH₃ | —CH₃ | —C(O)—N(H)-cyclopropyl | 2-methyl-3-thienyl |
| —CH₃ | —CH₃ | —C(O)—N(H)-cyclopropyl | 4-methyl-3-thienyl |
| —CH₃ | —CH₃ | —C(O)—N(H)-cyclopropyl | 1-benzothien-3-yl |
| —CH₃ | —CH₃ | —C(O)—N(H)-cyclopropyl | 1-naphthyl |
| —CH₃ | —CH₃ | —C(O)—N(H)-cyclopropyl | 2-naphthyl |
| —CH₃ | —CH₃ | —C(O)—N(H)-cyclopropyl | 2-N-methyl-pyrrolyl |
| —CH₃ | —CH₃ | —C(O)—N(H)-cyclopropyl | 4-pyridyl |
| —CH₃ | —CH₃ | —C(O)-pyrrolidin-1-yl | 2-methyl-phenyl |
| —CH₃ | —CH₃ | —C(O)-pyrrolidin-1-yl | 2-ethyl-phenyl |
| —CH₃ | —CH₃ | —C(O)-pyrrolidin-1-yl | 2-isopropyl-phenyl |
| —CH₃ | —CH₃ | —C(O)-pyrrolidin-1-yl | 4-fluoro-2-methyl-phenyl |
| —CH₃ | —CH₃ | —C(O)-pyrrolidin-1-yl | 2-trifluoromethyl-phenyl |
| —CH₃ | —CH₃ | —C(O)-pyrrolidin-1-yl | 2-hydroxymethyl-phenyl |
| —CH₃ | —CH₃ | —C(O)-pyrrolidin-1-yl | 2-chloro-phenyl |
| —CH₃ | —CH₃ | —C(O)-pyrrolidin-1-yl | 2-(2-hydroxyethyl)- |
| | | | phenyl |
| —CH₃ | —CH₃ | —C(O)-pyrrolidin-1-yl | 2-(1-hydroxyethyl)- |
| | | | phenyl |
| —CH₃ | —CH₃ | —C(O)-pyrrolidin-1-yl | 2-benzyloxymethyl- |
| | | | phenyl |
| —CH₃ | —CH₃ | —C(O)-pyrrolidin-1-yl | 2-methoxymethyl-phenyl |
| —CH₃ | —CH₃ | —C(O)-pyrrolidin-1-yl | 2-thienyl |
| —CH₃ | —CH₃ | —C(O)-pyrrolidin-1-yl | 3-methyl-2-thienyl |
| —CH₃ | —CH₃ | —C(O)-pyrrolidin-1-yl | 3-thienyl |
| —CH₃ | —CH₃ | —C(O)-pyrrolidin-1-yl | 2-methyl-3-thienyl |
| —CH₃ | —CH₃ | —C(O)-pyrrolidin-1-yl | 4-methyl-3-thienyl |
| —CH₃ | —CH₃ | —C(O)-pyrrolidin-1-yl | 1-benzothien-3-yl |
| —CH₃ | —CH₃ | —C(O)-pyrrolidin-1-yl | 1-naphthyl |
| —CH₃ | —CH₃ | —C(O)-pyrrolidin-1-yl | 2-naphthyl |
| —CH₃ | —CH₃ | —C(O)-pyrrolidin-1-yl | 2-N-methyl-pyrrolyl |
| —CH₃ | —CH₃ | —C(O)-pyrrolidin-1-yl | 4-pyridyl |
| —CH₃ | —CH₃ | —C(O)-azetidin-1-yl | 2-methyl-phenyl |
| —CH₃ | —CH₃ | —C(O)-azetidin-1-yl | 2-ethyl-phenyl |
| —CH₃ | —CH₃ | —C(O)-azetidin-1-yl | 2-isopropyl-phenyl |
| —CH₃ | —CH₃ | —C(O)-azetidin-1-yl | 4-fluoro-2-methyl-phenyl |
| —CH₃ | —CH₃ | —C(O)-azetidin-1-yl | 2-trifluoromethyl-phenyl |
| —CH₃ | —CH₃ | —C(O)-azetidin-1-yl | 2-hydroxymethyl-phenyl |
| —CH₃ | —CH₃ | —C(O)-azetidin-1-yl | 2-chloro-phenyl |
| —CH₃ | —CH₃ | —C(O)-azetidin-1-yl | 2-(2-hydroxyethyl)- |
| | | | phenyl |
| —CH₃ | —CH₃ | —C(O)-azetidin-1-yl | 2-(1-hydroxyethyl)- |
| | | | phenyl |
| —CH₃ | —CH₃ | —C(O)-azetidin-1-yl | 2-benzyloxymethyl- |
| | | | phenyl |
| —CH₃ | —CH₃ | —C(O)-azetidin-1-yl | 2-methoxymethyl-phenyl |
| —CH₃ | —CH₃ | —C(O)-azetidin-1-yl | 2-thienyl |
| —CH₃ | —CH₃ | —C(O)-azetidin-1-yl | 3-methyl-2-thienyl |
| —CH₃ | —CH₃ | —C(O)-azetidin-1-yl | 3-thienyl |
| —CH₃ | —CH₃ | —C(O)-azetidin-1-yl | 2-methyl-3-thienyl |
| —CH₃ | —CH₃ | —C(O)-azetidin-1-yl | 4-methyl-3-thienyl |
| —CH₃ | —CH₃ | —C(O)-azetidin-1-yl | 1-benzothien-3-yl |
| —CH₃ | —CH₃ | —C(O)-azetidin-1-yl | 1-naphthyl |
| —CH₃ | —CH₃ | —C(O)-azetidin-1-yl | 2-naphthyl |
| —CH₃ | —CH₃ | —C(O)-azetidin-1-yl | 2-N-methyl-pyrrolyl |
| —CH₃ | —CH₃ | —C(O)-azetidin-1-yl | 4-pyridyl |
| —CH₃ | —CH₃ | —CH₂—O—CH₃ | 2-methyl-phenyl |
| —CH₃ | —CH₃ | —CH₂—O—CH₃ | 2-ethyl-phenyl |
| —CH₃ | —CH₃ | —CH₂—O—CH₃ | 2-isopropyl-phenyl |
| —CH₃ | —CH₃ | —CH₂—O—CH₃ | 4-fluoro-2-methyl-phenyl |
| —CH₃ | —CH₃ | —CH₂—O—CH₃ | 2-trifluoromethyl-phenyl |
| —CH₃ | —CH₃ | —CH₂—O—CH₃ | 2-hydroxymethyl-phenyl |
| —CH₃ | —CH₃ | —CH₂—O—CH₃ | 2-chloro-phenyl |
| —CH₃ | —CH₃ | —CH₂—O—CH₃ | 2-(2-hydroxyethyl)- |
| | | | phenyl |
| —CH₃ | —CH₃ | —CH₂—O—CH₃ | 2-(1-hydroxyethyl)- |
| | | | phenyl |
| —CH₃ | —CH₃ | —CH₂—O—CH₃ | 2-benzyloxymethyl- |
| | | | phenyl |
| —CH₃ | —CH₃ | —CH₂—O—CH₃ | 2-methoxymethyl-phenyl |
| —CH₃ | —CH₃ | —CH₂—O—CH₃ | 2-thienyl |
| —CH₃ | —CH₃ | —CH₂—O—CH₃ | 3-methyl-2-thienyl |
| —CH₃ | —CH₃ | —CH₂—O—CH₃ | 3-thienyl |
| —CH₃ | —CH₃ | —CH₂—O—CH₃ | 2-methyl-3-thienyl |
| —CH₃ | —CH₃ | —CH₂—O—CH₃ | 4-methyl-3-thienyl |
| —CH₃ | —CH₃ | —CH₂—O—CH₃ | 1-benzothien-3-yl |
| —CH₃ | —CH₃ | —CH₂—O—CH₃ | 1-naphthyl |
| —CH₃ | —CH₃ | —CH₂—O—CH₃ | 2-naphthyl |
| —CH₃ | —CH₃ | —CH₂—O—CH₃ | 2-N-methyl-pyrrolyl |
| —CH₃ | —CH₃ | —CH₂—O—CH₃ | 4-pyridyl |
| —CH₃ | H | —C(O)—N(CH₃)₂ | 2-methyl-phenyl |
| —CH₃ | H | —C(O)—N(CH₃)₂ | 2-ethyl-phenyl |
| —CH₃ | H | —C(O)—N(CH₃)₂ | 2-isopropyl-phenyl |
| —CH₃ | H | —C(O)—N(CH₃)₂ | 4-fluoro-2-methyl-phenyl |
| —CH₃ | H | —C(O)—N(CH₃)₂ | 2-trifluoromethyl-phenyl |
| —CH₃ | H | —C(O)—N(CH₃)₂ | 2-hydroxymethyl-phenyl |
| —CH₃ | H | —C(O)—N(CH₃)₂ | 2-chloro-phenyl |
| —CH₃ | H | —C(O)—N(CH₃)₂ | 2-(2-hydroxyethyl)- |
| | | | phenyl |
| —CH₃ | H | —C(O)—N(CH₃)₂ | 2-(1-hydroxyethyl)- |
| | | | phenyl |
| —CH₃ | H | —C(O)—N(CH₃)₂ | 2-benzyloxymethyl- |
| | | | phenyl |
| —CH₃ | H | —C(O)—N(CH₃)₂ | 2-methoxymethyl-phenyl |
| —CH₃ | H | —C(O)—N(CH₃)₂ | 2-thienyl |
| —CH₃ | H | —C(O)—N(CH₃)₂ | 3-methyl-2-thienyl |
| —CH₃ | H | —C(O)—N(CH₃)₂ | 3-thienyl |
| —CH₃ | H | —C(O)—N(CH₃)₂ | 2-methyl-3-thienyl |
| —CH₃ | H | —C(O)—N(CH₃)₂ | 4-methyl-3-thienyl |
| —CH₃ | H | —C(O)—N(CH₃)₂ | 1-benzothien-3-yl |
| —CH₃ | H | —C(O)—N(CH₃)₂ | 1-naphthyl |
| —CH₃ | H | —C(O)—N(CH₃)₂ | 2-naphthyl |
| —CH₃ | —H | —C(O)—N(CH₃)₂ | 2-N-methyl-pyrrolyl |
| —CH₃ | —H | —C(O)—N(CH₃)₂ | 4-pyridyl |
| —CH₃ | H | —C(O)—N(H)CH₃ | 2-methyl-phenyl |
| —CH₃ | H | —C(O)—N(H)CH₃ | 2-ethyl-phenyl |
| —CH₃ | H | —C(O)—N(H)CH₃ | 2-isopropyl-phenyl |
| —CH₃ | H | —C(O)—N(H)CH₃ | 4-fluoro-2-methyl-phenyl |
| —CH₃ | H | —C(O)—N(H)CH₃ | 2-trifluoromethyl-phenyl |
| —CH₃ | H | —C(O)—N(H)CH₃ | 2-hydroxymethyl-phenyl |
| —CH₃ | H | —C(O)—N(H)CH₃ | 2-chloro-phenyl |
| —CH₃ | H | —C(O)—N(H)CH₃ | 2-(2-hydroxyethyl)- |
| | | | phenyl |
| —CH₃ | H | —C(O)—N(H)CH₃ | 2-(1-hydroxyethyl)- |
| | | | phenyl |
| —CH₃ | H | —C(O)—N(H)CH₃ | 2-benzyloxymethyl- |
| | | | phenyl |
| —CH₃ | H | —C(O)—N(H)CH₃ | 2-methoxymethyl-phenyl |
| —CH₃ | H | —C(O)—N(H)CH₃ | 2-thienyl |
| —CH₃ | H | —C(O)—N(H)CH₃ | 3-methyl-2-thienyl |
| —CH₃ | H | —C(O)—N(H)CH₃ | 3-thienyl |
| —CH₃ | H | —C(O)—N(H)CH₃ | 2-methyl-3-thienyl |
| —CH₃ | H | —C(O)—N(H)CH₃ | 4-methyl-3-thienyl |
| —CH₃ | H | —C(O)—N(H)CH₃ | 1-benzothien-3-yl |
| —CH₃ | H | —C(O)—N(H)CH₃ | 1-naphthyl |
| —CH₃ | H | —C(O)—N(H)CH₃ | 2-naphthyl |
| —CH₃ | H | —C(O)—N(H)CH₃ | 2-N-methyl-pyrrolyl |
| —CH₃ | H | —C(O)—N(H)CH₃ | 4-pyridyl |
| —CH₃ | H | —C(O)—N(H)-cyclopropyl | 2-methyl-phenyl |
| —CH₃ | H | —C(O)—N(H)-cyclopropyl | 2-ethyl-phenyl |
| —CH₃ | H | —C(O)—N(H)-cyclopropyl | 2-isopropyl-phenyl |
| —CH₃ | H | —C(O)—N(H)-cyclopropyl | 4-fluoro-2-methyl-phenyl |
| —CH₃ | H | —C(O)—N(H)-cyclopropyl | 2-trifluoromethyl-phenyl |
| —CH₃ | H | —C(O)—N(H)-cyclopropyl | 2-hydroxymethyl-phenyl |
| —CH₃ | H | —C(O)—N(H)-cyclopropyl | 2-chloro-phenyl |
| —CH₃ | H | —C(O)—N(H)-cyclopropyl | 2-(2-hydroxyethyl)- |
| | | | phenyl |
| —CH₃ | H | —C(O)—N(H)-cyclopropyl | 2-(1-hydroxyethyl)- |
| | | | phenyl |
| —CH₃ | H | —C(O)—N(H)-cyclopropyl | 2-benzyloxymethyl- |
| | | | phenyl |
| —CH₃ | H | —C(O)—N(H)-cyclopropyl | 2-methoxymethyl-phenyl |
| —CH₃ | H | —C(O)—N(H)-cyclopropyl | 2-thienyl |
| —CH₃ | H | —C(O)—N(H)-cyclopropyl | 3-methyl-2-thienyl |
| —CH₃ | H | —C(O)—N(H)-cyclopropyl | 3-thienyl |
| —CH₃ | H | —C(O)—N(H)-cyclopropyl | 2-methyl-3-thienyl |
| —CH₃ | H | —C(O)—N(H)-cyclopropyl | 4-methyl-3-thienyl |
| —CH₃ | H | —C(O)—N(H)-cyclopropyl | 1-benzothien-3-yl |
| —CH₃ | H | —C(O)—N(H)-cyclopropyl | 1-naphthyl |
| —CH₃ | H | —C(O)—N(H)-cyclopropyl | 2-naphthyl |
| —CH₃ | H | —C(O)—N(H)-cyclopropyl | 2-N-methyl-pyrrolyl |
| —CH₃ | H | —C(O)—N(H)-cyclopropyl | 4-pyridyl |
| —CH₃ | H | —C(O)-pyrrolidin-1-yl | 2-methyl-phenyl |
| —CH₃ | H | —C(O)-pyrrolidin-1-yl | 2-ethyl-phenyl |
| —CH₃ | H | —C(O)-pyrrolidin-1-yl | 2-isopropyl-phenyl |
| —CH₃ | H | —C(O)-pyrrolidin-1-yl | 4-fluoro-2-methyl-phenyl |
| —CH₃ | H | —C(O)-pyrrolidin-1-yl | 2-trifluoromethyl-phenyl |
| —CH₃ | H | —C(O)-pyrrolidin-1-yl | 2-hydroxymethyl-phenyl |
| —CH₃ | H | —C(O)-pyrrolidin-1-yl | 2-chloro-phenyl |
| —CH₃ | H | —C(O)-pyrrolidin-1-yl | 2-(2-hydroxyethyl)- |
| | | | phenyl |
| —CH₃ | H | —C(O)-pyrrolidin-1-yl | 2-(1-hydroxyethyl)- |
| | | | phenyl |
| —CH₃ | H | —C(O)-pyrrolidin-1-yl | 2-benzyloxymethyl- |
| | | | phenyl |
| —CH₃ | H | —C(O)-pyrrolidin-1-yl | 2-methoxymethyl-phenyl |
| —CH₃ | H | —C(O)-pyrrolidin-1-yl | 2-thienyl |
| —CH₃ | H | —C(O)-pyrrolidin-1-yl | 3-methyl-2-thienyl |
| —CH₃ | H | —C(O)-pyrrolidin-1-yl | 3-thienyl |
| —CH₃ | H | —C(O)-pyrrolidin-1-yl | 2-methyl-3-thienyl |
| —CH₃ | H | —C(O)-pyrrolidin-1-yl | 4-methyl-3-thienyl |
| —CH₃ | H | —C(O)-pyrrolidin-1-yl | 1-benzothien-3-yl |
| —CH₃ | H | —C(O)-pyrrolidin-1-yl | 1-naphthyl |
| —CH₃ | H | —C(O)-pyrrolidin-1-yl | 2-naphthyl |
| —CH₃ | H | —C(O)-pyrrolidin-1-yl | 2-N-methyl-pyrrolyl |
| —CH₃ | H | —C(O)-pyrrolidin-1-yl | 4-pyridyl |
| —CH₃ | H | —C(O)-azetidin-1-yl | 2-methyl-phenyl |
| —CH₃ | H | —C(O)-azetidin-1-yl | 2-ethyl-phenyl |
| —CH₃ | H | —C(O)-azetidin-1-yl | 2-isopropyl-phenyl |
| —CH₃ | H | —C(O)-azetidin-1-yl | 4-fluoro-2-methyl-phenyl |
| —CH₃ | H | —C(O)-azetidin-1-yl | 2-trifluoromethyl-phenyl |
| —CH₃ | H | —C(O)-azetidin-1-yl | 2-hydroxymethyl-phenyl |
| —CH₃ | H | —C(O)-azetidin-1-yl | 2-chloro-phenyl |
| —CH₃ | H | —C(O)-azetidin-1-yl | 2-(2-hydroxyethyl)- |
| | | | phenyl |
| —CH₃ | H | —C(O)-azetidin-1-yl | 2-(1-hydroxyethyl)- |
| | | | phenyl |
| —CH₃ | H | —C(O)-azetidin-1-yl | 2-benzyloxymethyl- |
| | | | phenyl |
| —CH₃ | H | —C(O)-azetidin-1-yl | 2-methoxymethyl-phenyl |
| —CH₃ | H | —C(O)-azetidin-1-yl | 2-thienyl |
| —CH₃ | H | —C(O)-azetidin-1-yl | 3-methyl-2-thienyl |
| —CH₃ | H | —C(O)-azetidin-1-yl | 3-thienyl |
| —CH₃ | H | —C(O)-azetidin-1-yl | 2-methyl-3-thienyl |
| —CH₃ | H | —C(O)-azetidin-1-yl | 4-methyl-3-thienyl |
| —CH₃ | H | —C(O)-azetidin-1-yl | 1-benzothien-3-yl |
| —CH₃ | H | —C(O)-azetidin-1-yl | 1-naphthyl |
| —CH₃ | H | —C(O)-azetidin-1-yl | 2-naphthyl |
| —CH₃ | H | —C(O)-azetidin-1-yl | 2-N-methyl-pyrrolyl |
| —CH₃ | H | —C(O)-azetidin-1-yl | 4-pyridyl |
| —CH₃ | H | —CH₂—O—CH₃ | 2-methyl-phenyl |
| —CH₃ | H | —CH₂—O—CH₃ | 2-ethyl-phenyl |
| —CH₃ | H | —CH₂—O—CH₃ | 2-isopropyl-phenyl |
| —CH₃ | H | —CH₂—O—CH₃ | 4-fluoro-2-methyl-phenyl |
| —CH₃ | H | —CH₂—O—CH₃ | 2-trifluoromethyl-phenyl |
| —CH₃ | H | —CH₂—O—CH₃ | 2-hydroxymethyl-phenyl |
| —CH₃ | H | —CH₂—O—CH₃ | 2-chloro-phenyl |
| —CH₃ | H | —CH₂—O—CH₃ | 2-(2-hydroxyethyl)- |
| | | | phenyl |
| —CH₃ | H | —CH₂—O—CH₃ | 2-(1-hydroxyethyl)- |
| | | | phenyl |
| —CH₃ | H | —CH₂—O—CH₃ | 2-benzyloxymethyl- |
| | | | phenyl |
| —CH₃ | H | —CH₂—O—CH₃ | 2-methoxymethyl-phenyl |
| —CH₃ | H | —CH₂—O—CH₃ | 2-thienyl |
| —CH₃ | H | —CH₂—O—CH₃ | 3-methyl-2-thienyl |
| —CH₃ | H | —CH₂—O—CH₃ | 3-thienyl |
| —CH₃ | H | —CH₂—O—CH₃ | 2-methyl-3-thienyl |
| —CH₃ | H | —CH₂—O—CH₃ | 4-methyl-3-thienyl |
| —CH₃ | H | —CH₂—O—CH₃ | 1-benzothien-3-yl |
| —CH₃ | H | —CH₂—O—CH₃ | 1-naphthyl |
| —CH₃ | H | —CH₂—O—CH₃ | 2-naphthyl |
| —CH₃ | H | —CH₂—O—CH₃ | 2-N-methyl-pyrrolyl |
| —CH₃ | H | —CH₂—O—CH₃ | 4-pyridyl |

Particularly preferred are the compounds of the formula 3-a, wherein R1, R2, R3 and Ar have the meanings as indicated in the following table 1b and the salts of these compounds.

TABLE 1b

| R1 | R2 | R3 | Ar |
|---|---|---|---|
| —CH₃ | —CH₃ | —C(O)—N(CH₃)₂ | 2-methyl-phenyl |
| —CH₃ | —CH₃ | —C(O)—N(CH₃)₂ | 2-ethyl-phenyl |
| —CH₃ | —CH₃ | —C(O)—N(CH₃)₂ | 2-isopropyl-phenyl |
| —CH₃ | —CH₃ | —C(O)—N(CH₃)₂ | 4-fluoro-2-methyl-phenyl |
| —CH₃ | —CH₃ | —C(O)—N(CH₃)₂ | 2-trifluoromethyl-phenyl |
| —CH₃ | —CH₃ | —C(O)—N(CH₃)₂ | 2-hydroxymethyl-phenyl |
| —CH₃ | —CH₃ | —C(O)—N(CH₃)₂ | 2-chloro-phenyl |
| —CH₃ | —CH₃ | —C(O)—N(CH₃)₂ | 2-(2-hydroxyethyl)- |
| | | | phenyl |
| —CH₃ | —CH₃ | —C(O)—N(CH₃)₂ | 2-(1-hydroxyethyl)- |
| | | | phenyl |
| —CH₃ | —CH₃ | —C(O)—N(CH₃)₂ | 2-methoxymethyl-phenyl |
| —CH₃ | —CH₃ | —C(O)—N(CH₃)₂ | 2-thienyl |
| —CH₃ | —CH₃ | —C(O)—N(CH₃)₂ | 3-methyl-2-thienyl |
| —CH₃ | —CH₃ | —C(O)—N(CH₃)₂ | 3-thienyl |
| —CH₃ | —CH₃ | —C(O)—N(CH₃)₂ | 2-methyl-3-thienyl |
| —CH₃ | —CH₃ | —C(O)—N(CH₃)₂ | 4-methyl-3-thienyl |
| —CH₃ | —CH₃ | —C(O)—N(CH₃)₂ | 1-benzothien-3-yl |
| —CH₃ | —CH₃ | —C(O)—N(CH₃)₂ | 1-naphthyl |
| —CH₃ | —CH₃ | —C(O)—N(CH₃)₂ | 2-naphthyl |
| —CH₃ | —CH₃ | —C(O)—N(CH₃)₂ | 2-N-methyl-pyrrolyl |
| —CH₃ | —CH₃ | —C(O)—N(CH₃)₂ | 4-pyridyl |
| —CH₃ | —CH₃ | —C(O)—N(H)CH₃ | 2-methyl-phenyl |
| —CH₃ | —CH₃ | —C(O)—N(H)CH₃ | 2-ethyl-phenyl |
| —CH₃ | —CH₃ | —C(O)—N(H)CH₃ | 2-isopropyl-phenyl |
| —CH₃ | —CH₃ | —C(O)—N(H)CH₃ | 4-fluoro-2-methyl-phenyl |
| —CH₃ | —CH₃ | —C(O)—N(H)CH₃ | 2-trifluoromethyl-phenyl |
| —CH₃ | —CH₃ | —C(O)—N(H)CH₃ | 2-hydroxymethyl-phenyl |
| —CH₃ | —CH₃ | —C(O)—N(H)CH₃ | 2-chloro-phenyl |
| —CH₃ | —CH₃ | —C(O)—N(H)CH₃ | 2-(2-hydroxyethyl)- |
| | | | phenyl |
| —CH₃ | —CH₃ | —C(O)—N(H)CH₃ | 2-(1-hydroxyethyl)- |
| | | | phenyl |
| —CH₃ | —CH₃ | —C(O)—N(H)CH₃ | 2-methoxymethyl-phenyl |
| —CH₃ | —CH₃ | —C(O)—N(H)CH₃ | 2-thienyl |
| —CH₃ | —CH₃ | —C(O)—N(H)CH₃ | 3-methyl-2-thienyl |
| —CH₃ | —CH₃ | —C(O)—N(H)CH₃ | 3-thienyl |
| —CH₃ | —CH₃ | —C(O)—N(H)CH₃ | 2-methyl-3-thienyl |
| —CH₃ | —CH₃ | —C(O)—N(H)CH₃ | 4-methyl-3-thienyl |
| —CH₃ | —CH₃ | —C(O)—N(H)CH₃ | 1-benzothien-3-yl |
| —CH₃ | —CH₃ | —C(O)—N(H)CH₃ | 1-naphthyl |
| —CH₃ | —CH₃ | —C(O)—N(H)CH₃ | 2-naphthyl |
| —CH₃ | —CH₃ | —C(O)—N(H)CH₃ | 2-N-methyl-pyrrolyl |
| —CH₃ | —CH₃ | —C(O)—N(H)CH₃ | 4-pyridyl |
| —CH₃ | —CH₃ | —C(O)—N(H)-cyclopropyl | 2-methyl-phenyl |
| —CH₃ | —CH₃ | —C(O)—N(H)-cyclopropyl | 2-ethyl-phenyl |
| —CH₃ | —CH₃ | —C(O)—N(H)-cyclopropyl | 2-isopropyl-phenyl |
| —CH₃ | —CH₃ | —C(O)—N(H)-cyclopropyl | 4-fluoro-2-methyl-phenyl |
| —CH₃ | —CH₃ | —C(O)—N(H)-cyclopropyl | 2-trifluoromethyl-phenyl |
| —CH₃ | —CH₃ | —C(O)—N(H)-cyclopropyl | 2-hydroxymethyl-phenyl |
| —CH₃ | —CH₃ | —C(O)—N(H)-cyclopropyl | 2-chloro-phenyl |
| —CH₃ | —CH₃ | —C(O)—N(H)-cyclopropyl | 2-(2-hydroxyethyl)- |
| | | | phenyl |
| —CH₃ | —CH₃ | —C(O)—N(H)-cyclopropyl | 2-(1-hydroxyethyl)- |
| | | | phenyl |
| —CH₃ | —CH₃ | —C(O)—N(H)-cyclopropyl | 2-methoxymethyl-phenyl |
| —CH₃ | —CH₃ | —C(O)—N(H)-cyclopropyl | 2-thienyl |
| —CH₃ | —CH₃ | —C(O)—N(H)-cyclopropyl | 3-methyl-2-thienyl |
| —CH₃ | —CH₃ | —C(O)—N(H)-cyclopropyl | 3-thienyl |
| —CH₃ | —CH₃ | —C(O)—N(H)-cyclopropyl | 2-methyl-3-thienyl |
| —CH₃ | —CH₃ | —C(O)—N(H)-cyclopropyl | 4-methyl-3-thienyl |
| —CH₃ | —CH₃ | —C(O)—N(H)-cyclopropyl | 1-benzothien-3-yl |
| —CH₃ | —CH₃ | —C(O)—N(H)-cyclopropyl | 1-naphthyl |
| —CH₃ | —CH₃ | —C(O)—N(H)-cyclopropyl | 2-naphthyl |
| —CH₃ | —CH₃ | —C(O)—N(H)-cyclopropyl | 2-N-methyl-pyrrolyl |
| —CH₃ | —CH₃ | —C(O)—N(H)-cyclopropyl | 4-pyridyl |
| —CH₃ | —CH₃ | —C(O)-pyrrolidin-1-yl | 2-methyl-phenyl |
| —CH₃ | —CH₃ | —C(O)-pyrrolidin-1-yl | 2-ethyl-phenyl |
| —CH₃ | —CH₃ | —C(O)-pyrrolidin-1-yl | 2-isopropyl-phenyl |
| —CH₃ | —CH₃ | —C(O)-pyrrolidin-1-yl | 4-fluoro-2-methyl-phenyl |
| —CH₃ | —CH₃ | —C(O)-pyrrolidin-1-yl | 2-trifluoromethyl-phenyl |
| —CH₃ | —CH₃ | —C(O)-pyrrolidin-1-yl | 2-hydroxymethyl-phenyl |
| —CH₃ | —CH₃ | —C(O)-pyrrolidin-1-yl | 2-chloro-phenyl |
| —CH₃ | —CH₃ | —C(O)-pyrrolidin-1-yl | 2-(2-hydroxyethyl)- |
| | | | phenyl |
| —CH₃ | —CH₃ | —C(O)-pyrrolidin-1-yl | 2-(1-hydroxyethyl)- |
| | | | phenyl |
| —CH₃ | —CH₃ | —C(O)-pyrrolidin-1-yl | 2-methoxymethyl-phenyl |
| —CH₃ | —CH₃ | —C(O)-pyrrolidin-1-yl | 2-thienyl |
| —CH₃ | —CH₃ | —C(O)-pyrrolidin-1-yl | 3-methyl-2-thienyl |
| —CH₃ | —CH₃ | —C(O)-pyrrolidin-1-yl | 3-thienyl |
| —CH₃ | —CH₃ | —C(O)-pyrrolidin-1-yl | 2-methyl-3-thienyl |
| —CH₃ | —CH₃ | —C(O)-pyrrolidin-1-yl | 4-methyl-3-thienyl |
| —CH₃ | —CH₃ | —C(O)-pyrrolidin-1-yl | 1-benzothien-3-yl |
| —CH₃ | —CH₃ | —C(O)-pyrrolidin-1-yl | 1-naphthyl |
| —CH₃ | —CH₃ | —C(O)-pyrrolidin-1-yl | 2-naphthyl |
| —CH₃ | —CH₃ | —C(O)-pyrrolidin-1-yl | 2-N-methyl-pyrrolyl |
| —CH₃ | —CH₃ | —C(O)-pyrrolidin-1-yl | 4-pyridyl |
| —CH₃ | —CH₃ | —C(O)-azetidin-1-yl | 2-methyl-phenyl |
| —CH₃ | —CH₃ | —C(O)-azetidin-1-yl | 2-ethyl-phenyl |
| —CH₃ | —CH₃ | —C(O)-azetidin-1-yl | 2-isopropyl-phenyl |
| —CH₃ | —CH₃ | —C(O)-azetidin-1-yl | 4-fluoro-2-methyl-phenyl |
| —CH₃ | —CH₃ | —C(O)-azetidin-1-yl | 2-trifluoromethyl-phenyl |
| —CH₃ | —CH₃ | —C(O)-azetidin-1-yl | 2-hydroxymethyl-phenyl |
| —CH₃ | —CH₃ | —C(O)-azetidin-1-yl | 2-chloro-phenyl |
| —CH₃ | —CH₃ | —C(O)-azetidin-1-yl | 2-(2-hydroxyethyl)- |
| | | | phenyl |
| —CH₃ | —CH₃ | —C(O)-azetidin-1-yl | 2-(1-hydroxyethyl)- |
| | | | phenyl |
| —CH₃ | —CH₃ | —C(O)-azetidin-1-yl | 2-methoxymethyl-phenyl |
| —CH₃ | —CH₃ | —C(O)-azetidin-1-yl | 2-thienyl |
| —CH₃ | —CH₃ | —C(O)-azetidin-1-yl | 3-methyl-2-thienyl |
| —CH₃ | —CH₃ | —C(O)-azetidin-1-yl | 3-thienyl |
| —CH₃ | —CH₃ | —C(O)-azetidin-1-yl | 2-methyl-3-thienyl |
| —CH₃ | —CH₃ | —C(O)-azetidin-1-yl | 4-methyl-3-thienyl |
| —CH₃ | —CH₃ | —C(O)-azetidin-1-yl | 1-benzothien-3-yl |
| —CH₃ | —CH₃ | —C(O)-azetidin-1-yl | 1-naphthyl |
| —CH₃ | —CH₃ | —C(O)-azetidin-1-yl | 2-naphthyl |
| —CH₃ | —CH₃ | —C(O)-azetidin-1-yl | 2-N-methyl-pyrrolyl |
| —CH₃ | —CH₃ | —C(O)-azetidin-1-yl | 4-pyridyl |

Suitable salts of compounds of the formula 3-a and 3-b according to table 1a and 1b are—depending on the substitution—in particular all acid addition salts. Particular mention may be made of the pharmacologically acceptable salts of the inorganic and organic acids customarily used in pharmacy. Those suitable are water-soluble and water-insoluble acid addition salts with acids such as, for example, hydrochloric acid, hydrobromic acid, phosphoric acid, nitric acid, sulfuric acid, acetic acid, citric acid, D-gluconic acid, benzoic acid, 2-(4-hydroxybenzoyl)benzoic acid, butyric acid, sulfosalicylic acid, maleic acid, lauric acid, malic acid, malonic acid, fumaric acid, succinic acid, oxalic acid, tartaric acid, embonic acid, stearic acid, toluenesulfonic acid, methanesulfonic acid or 3-hydroxy-2-naphthoic acid.

Salts of the compounds of formula 3-a and 3-b according to the invention can be obtained by dissolving the free compound in a suitable solvent (for example a ketone such as acetone, methylethylketone or methylisobutylketone, an ether such as diethyl ether, tetrahydrofuran or dioxane, a chlorinated hydrocarbon such as methylene chloride or chloroform, or a low molecular weight aliphatic alcohol such as methanol, ethanol or isopropanol) which contains the desired acid or to which the desired acid is then added, if necessary upon heating. For salt preparation, the acid can be employed in an equimolar quantitative ratio or one differing therefrom, depending on whether a mono- or polybasic acid is concerned and depending on which salt is desired. The salts are obtained for example by evaporating the solvent or by precipitating upon cooling, by re-precipitating, or by precipitating with a non-solvent for the salt and separation, for example by filtration, of the salt after precipitation.

Pharmacologically unacceptable salts, which can be initially obtained, for example, as process products in the preparation of the compounds of the formula 3-a and 3-b on an industrial scale, are converted into pharmacologically acceptable salts by processes known to the person skilled in the art.

It is known to the person skilled in the art that the compounds of the formula 3-a and 3-b and their salts can, for example when they are isolated in crystalline form, comprise varying amounts of solvents. The invention therefore also embraces all solvates and, in particular, all hydrates of the compounds of the formula 3-a and 3-b listed in table 1a and 1b, and all solvates and, in particular, all hydrates of the salts of the compounds of the formula 3-a and 3-b listed in table 1a and 1b.

In particular, the invention relates to compounds of the formula 3-a according to table 1a and 1b and/or their salts being substantially-free of compounds of the formula 3-b according to table 1a and 1b and/or their salts.

“Substantially free” in the context of the invention means that the compounds of the formula 3-a and/or their salts contain less than 30% by weight of compounds of the formula 3-b and/or their salts. Preferably, “substantially free” means that compounds of the formula 3-a and/or their salts contain less than 10% by weight of compounds of the formula 3-b and/or their salts. In a more preferred embodiment, “substantially free” means that compounds of the formula 3-a and/or their salts contain less than 5% by weight of compounds of the formula 3-b and/or their salts. In the most preferred embodiment, “substantially free” means that compounds of the formula 3-a and/or their salts contain less than 2% by weight of compounds of the formula 3-b and/or their salts.

Particularly preferred are the compounds of the formula 3-a and/or their salts described by way of example in the experimental section below.

### ADVANTAGEOUS EFFECTS

The excellent gastric protective action and the gastric acid secretion-inhibiting action of the compounds of the formula 3-a and 3-b, particularly those of the formula 3-a, according to the invention can be demonstrated in investigations on animal experimental models. The compounds of the formula 3-a according to the invention investigated in the model mentioned below have been provided with numbers which correspond to the numbers of these compounds in the examples.

Testing of the Secretion-Inhibiting Action on the Perfused Rat Stomach

In Table A which follows, the influence of the compounds of the formula 3-a according to the invention on the pentagastrin-stimulated acid secretion of the perfused rat stomach after intraduodenal administration in vivo is shown.

TABLE A

| | Dose | Inhibition of |
|---|---|---|
| | (μmol/kg) | acid secretion |
| Letter | i.d. | (%) |
| A | 1 | >60 |
| C | 1 | >90 |
| D | 1 | >50 |
| E | 1 | >80 |
| F | 1 | >70 |
| G | 1 | 100 |
| H | 1 | 100 |
| I | 1 | >70 |
| L | 1 | >70 |
| N | 1 | 100 |
| O | 1 | >60 |
| P | 1 | 100 |
| Q | 1 | 100 |
| V | 1 | 100 |
| W | 1 | 100 |

Methodology

The abdomen of anesthetized rats (CD rat, female, 200-250 g; 1.5 g/kg i.m. urethane) was opened after tracheotomy by a median upper abdominal incision and a PVC catheter was fixed transorally in the esophagus and another via the pylorus such that the ends of the tubes just projected into the gastric lumen. The catheter leading from the pylorus led outward into the right abdominal wall through a side opening.

After thorough rinsing (about 50-100 ml), warm (37° C.) physiological NaCl solution was continuously passed through the stomach (0.5 ml/min, pH 6.8-6.9; Braun-Unita I). The pH (pH meter 632, glass electrode EA 147; φ=5 mm, Metrohm) and, by titration with a freshly prepared 0.01N NaOH solution to pH 7 (Dosimat 665 Metrohm), the secreted HCl were determined in the effluent in each case collected at an interval of 15 minutes.

The gastric secretion was stimulated by continuous infusion of 1 μg/kg (=1.65 ml/h) of i.v. pentagastrin (left femoral vein) about 30 min after the end of the operation (i.e. after determination of 2 preliminary as fractions). The substances to be tested were administered intraduodenally in a 2.5 ml/kg liquid volume 60 min after the start of the continuous pentagastrin infusion. The body temperature of the animals was kept at a constant 37.8-38° C. by infrared irradiation and heat pads (automatic, stepless control by means of a rectal temperature sensor).

### MODE(S) FOR CARRYING OUT THE INVENTION

The examples below serve to illustrate the invention in more detail without limiting it. Further compounds of the formula 1-a or 1-b whose preparation is not described explicitly can likewise be prepared in an analogous manner or in a manner known per se to the person skilled in the art, using customary process techniques. The abbreviation ee stands for enantiomeric excess, S/C for substrate to catalyst ratio, v for volume. For the assignment of NMR signals, the following abbreviations are used: s (singlet), d (duplet), t (triplet), q (quartet), m (multiplet), b (broad). The following units are used: ml (millilitre), l (litre), nm (nanometer), mm (millimeter), mg (milligramme), g (gramme), mmol (millimol), N (normal), M (molar), min (minute), h (hour/s), MHz (megahertz).

Furthermore the following abbreviations are used for the chemical substances indicated:
(S)-Xyl-P-Phos (S)-4,4′-bis-[bis-(3,5-dimethyl-phenyl)-phosphanyl]-2,6,2′,6′-tetramethoxy-[3,3′]bipyridinyl
(R)-Xyl-P-Phos (R)-4,4′-bis-[bis-(3,5-dimethyl-phenyl)-phosphanyl]-2,6,2′,6′-tetramethoxy-[3,3′]bipyridinyl
(S)-Xyl-BINAP (S)-(2,2′-bis(di(3,5-dimethylphenyl)phosphino)-1,1′-binaphthyl)
(R)-Xyl-BINAP (R)-(2,2′-bis(di(3,5-dimethylphenyl)phosphino)-1,1′-binaphthyl)
(S)-DAIPEN (2S)-(+)-1,1-bis(4-methoxyphenyl)-3-methyl-1,2-butanediamine
(R)-DAIPEN (2R)-(−)-1,1-bis(4-methoxyphenyl)-3-methyl-1,2-butanediamine
DIAD diisopropyl azodicarboxylate
DIPEA diisopropylethylamine
DMAP 4-(dimethylamino)pyridine
DMSO dimethylsulfoxide
THF tetrahydrofuran
DME 1,2-dimethoxyethane
DMF dimethylformamide
TBTU O-benzotriazol-1-yl-N,N,N′,N′-tetramethyluronium tetrafluoroborate

The optical purity of the compounds of the formulae 1-a, 1-b, and 3-a was determined by capillary electrophoresis (CE) and/or high pressure liquid chromatography (HPLC). The experimental conditions for the separation of the enantiomers by HPLC are given for each example in the experimental section (RT=retention time). The separation by CE was performed using the following experimental set-up (MT=migration time):

### Instrument: Agilent CE-3D

Capillary: 56/64.5 cm×50 μm barefused silica bubble (Agilent, all examples except for 24)
56/64.5 cm×75 μm barefused silica bubble (Agilent, example 24)
   Buffer: 50 mM sodium phosphate, pH 2.5 (Agilent)
   Chiral selector: 40 mM heptakis(2,3,6-tri-O-methyl)-β-cyclodextrin (all examples except for H)
40 mM heptakis(2,3-di-O-methyl)-6-sulfato-β-cyclodextrin (example H)

### Voltage: 30 kV

Temperature: 20° C. (all examples except for 8), 10° C. (example 8)
Detection: Diode array 219/226 nm

The HPLC columns used for analytical purposes are commercially available:
CHIRALPAK® AD-H: DAICEL Chemical Industries Ltd, Tokyo or Chiral Technologies-Europe SARL, Ilkirch, France
LichroCART® 250-4 ChiraDex (5 μg)®: Merck KgaA, Darmstadt, Germany

If NMR (nuclear magnetic resonance) chemical shifts are given without integration, overlay of the signal of the corresponding proton of the compound with signals of the solvent, water, or impurities was observed.

### Preparation of the catalyst RuCl₂[(S)-Xyl-P-Phos][(S)-DAIPEN]

(Benzene)dichlororuthenium dimer (CAS 37366-09-9, 1 equivalent) and (S)-Xyl-P-Phos (CAS 443347-10-2, commercially available from Strem Chemicals and Alfa Aesar, 1.03 equivalents) were placed in a Schlenk flask that was evacuated and filled with argon. Anhydrous, degassed DMF (2 ml per mmol) was added and the flask was placed in an oil bath pre-heated to 105° C. The reaction was stirred at 105° C. for 1.5 hours. (S)-DAIPEN (CAS 148369-91-9, commercially available from Strem Chemicals, 1.1 equivalents) was added and the reaction was stirred at room temperature for 3 hours. At this stage, a sample of the reaction mixture was diluted in chloroform-d and analysed by ³¹P-NMR spectroscopy. Only the two doublets of the desired complex+the small excess of free ligand were visible. The DMF was evaporated under vacuum (heating necessary) and the residue was dissolved in anhydrous degassed dichloromethane (5-10 ml per mmol) and placed on top of a silica gel column in a Schlenk filter under argon. The product was eluted with dichloromethane/methyl tert-butyl ether=1:1 (v/v). The clear yellow solution was collected in a Schlenk flask and the solvent was evaporated to give a yellow/green solid that was further dried under vacuum overnight. The isolated yield was 90% (Adaptation of a general procedure described by Noyori in Angew. Chem. 1998, 110, 1792-1796).

### Synthesis of Compounds of the Formula 1-a by Asymmetric Reduction of Prochiral Ketones of the Formula 2

### 1. (3R)-7-Hydroxy-6-(3-hydroxy-3-phenyl-propyl)-2,3-dimethyl-3H-benzolmidazole-5-carboxylic Acid Dimethylamide

In a flask filled with argon, 7-hydroxy-2,3-dimethyl-6-(3-oxo-3-phenyl-propyl)-3H-benzoimidazole-5-carboxylic acid dimethylamide (2.0 g, 5.5 mmol) was suspended in isopropanol (2.8 ml) and water (4.1 ml). Potassium tert-butylate solution (1 M in tert-butanol, 8.0 ml), tert-butanol (20 ml) and 22 mg of the hydrogenation catalyst RuCl₂[(S)-Xyl-P-Phos][(S)-DAIPEN] were added and the mixture was transferred into an autoclave and hydrogenated for 23 h at 65° C. and 80 bar H₂-pressure. After cooling to room temperature and releasing of the hydrogen pressure, the reaction mixture was concentrated in vacuo. The residue was purified by flash chromatography on silica gel (Dichloromethane/Methanol=14:1). Two batches of the title compound were obtained, which were crystallized from acetone: Batch 1: 0.5 g of a beige solid (25% yield, m.p. 261-263° C.), batch 2: 0.9 g of a beige solid (45% yield, 98% ee, m.p. 263-265° C.).

¹H-NMR (DMSO-d₆, 200 MHz): δ=1.80 (bs, 2H), 2.35 (bs), 2.50 (s), 2.68 (s, bs, 4H), 2.89 (s, 3H), 3.64 (s, 3H), 4.48 (t, 1H), 5.13 (bs, 1H), 6.70 (s, 1H), 7.25 (m_{c}, 5H), 9.85 (bs, 1H).

### Asymmetric catalytic hydrogenation with RuCl₂[(S)-Xyl-P-Phos][(S)-DAIPEN], screen of reaction conditions

Samples of 7-hydroxy-2,3-dimethyl-6-(3-oxo-3-phenyl-propyl)-3H-benzoimidazole-5-carboxylic acid dimethylamide (cf. table, 0.37 g, 1.0 mmol, entry 16: 0.48 g, 1.3 mmol) and RuCl₂[(S)-Xyl-P-Phos][(S)-DAIPEN](preparation described above) were weighed in glass liners that were then placed in an Argonaut Endeavour (eight wells pressure parallel reactor, overhead stirrers and heating block). The vessel was sealed and the wells purged by pressurising five times with nitrogen to 2 bar and releasing the pressure. The base (1 M solution of potassium tert-butylate in tert-butanol or aqueous solution of potassium hydroxide, cf. table) and the solvent (cf. table) were then injected. The wells were purged by pressurising five times with hydrogen to 25 bar (under stirring) and releasing the pressure. The reaction was then heated to the set temperature (of table) and pressurised to the set pressure of hydrogen (cf table). After the period specified in the table, the hydrogen pressure was released and the reaction mixtures were transferred to round bottomed flasks with the help of methanol (10 ml). The solvent was evaporated and the crude samples were analysed by HPLC and/or NMR (determination of conversion).

| | | Substrate | | Temp. | Press. | Approx | Conv. |
|---|---|---|---|---|---|---|---|
| Entry | S/C | conc. | Solvent and base | (° C.) | (bar) | Time | (%) |
| 1 | 500/1 | 0.1 M | 1.1 mL t-BuOK 1M | 65 | 20 | 16 | 86 |
| | | | 0.5 mL water | | | | |
| | | | 8.4 mL i-PrOH | | | | |
| 2 | 500/1 | 0.25 M | 1 mL KOH 10M | 70 | 25 | 16 | 100 |
| | | | 2.0 mL t-BuOH | | | | |
| | | | 1.0 mL i-PrOH | | | | |
| 3 | 500/1 | 0.25 M | 1.2 mL t-BuOK 1M | 70 | 25 | 16 | 100 |
| | | | 0.4 mL water | | | | |
| | | | 2.4 mL i-PrOH | | | | |
| 4 | 500/1 | 0.5 M | 1.1 mL t-BuOK 1M | 65 | 25 | 16 | >97 |
| | | | 0.2 mL water | | | | |
| | | | 0.7 mL i-PrOH | | | | |
| 5 | 750/1 | 0.25 M | 1.1 mL t-BuOK 1M | 65 | 20 | 16 | 83 |
| | | | 0.2 mL water | | | | |
| | | | 2.7 mL i-PrOH | | | | |
| 6 | 750/1 | 0.2 M | 2.0 mL t-BuOK 1M | 70 | 25 | 16 | 91 |
| | | | 0.5 mL H₂O | | | | |
| | | | 2.5 mL i-PrOH | | | | |
| 7 | 1000/1 | 0.33 M | 1.2 mL t-BuOK 1M | 75 | 25 | 16 | 51 |
| | | | 0.6 mL H₂O | | | | |
| | | | 1.2 mL i-PrOH | | | | |
| 8 | 1000/1 | 0.33 M | 1.2 mL t-BuOK 1M | 75 | 25 | 16 | 79 |
| | | | 0.3 mL H₂O | | | | |
| | | | 1.5 mL i-PrOH | | | | |
| 9 | 1000/1 | 0.5 M | 1.2 mL t-BuOK 1M | 75 | 25 | 16 | 83 |
| | | | 0.2 mL H₂O | | | | |
| | | | 0.6 mL i-PrOH | | | | |
| 10 | 1000/1 | 0.33 M | 1.2 mL t-BuOK 1M | 70 | 25 | 16 | 94 |
| | | | 0.3 mL H₂O | | | | |
| | | | 1.5 mL i-PrOH | | | | |
| 11 | 1000/1 | 0.33 M | 1.2 mL t-BuOK 1M | 65 | 25 | 16 | 75 |
| | | | 0.3 mL H₂O | | | | |
| | | | 1.5 mL i-PrOH | | | | |
| 12 | 1000/1 | 0.2 M | 1.2 mL t-BuOK 1M | 70 | 25 | 16 | 67 |
| | | | 0.25 mL H₂O (5%) | | | | |
| | | | 3.25 mL i-PrOH | | | | |
| 13 | 1000/1 | 0.33 M | 1.2 mL t-BuOK 1M | 70 | 25 | 16 | 62 |
| | | | 0.3 mL H₂O | | | | |
| | | | 1.5 mL i-PrOH | | | | |
| 14 | 1000/1 | 0.33 M | 1.5 mL t-BuOK 1M | 70 | 25 | 16 | 86 |
| | | | 0.3 mL H₂O | | | | |
| | | | 1.2 mL i-PrOH | | | | |
| 15 | 1000/1 | 0.33 M | 0.3 mL KOH 10M | 70 | 25 | 16 | 89 |
| | | | 1.2 mL t-BuOH | | | | |
| | | | 1.5 mL i-PrOH | | | | |
| 16 | 1000/1 | 0.33 M | 1.0 mL KOH 10M 2.0 mL | 70 | 25 | 16 | 92 |
| | | | t-BuOH | | | | |
| | | | 1.0 mL i-PrOH * | | | | |
| 17 | 1500/1 | 0.2 M | 2.0 mL t-BuOK 1M | 70 | 25 | 16 | 65 |
| | | | 0.5 mL H₂O | | | | |
| | | | 2.5 mL i-PrOH | | | | |

Asymmetric catalytic hydrogenation with RuCl₂[(S)-Xyl-BINAP][(S)-DAIPEN]: Two samples of 7-hydroxy-2,3-dimethyl-6-(3-oxo-3-phenyl-propyl)-3H-benzoimidazole-5-carboxylic acid dimethylamide (2×470 mg, 1.26 mmol) and RuCl₂[(S)-Xyl-BINAP][(S)-DAIPEN] (each sample: 6 mg) were weighed in glass liners that were then placed in an Argonaut Endeavour (eight wells pressure parallel reactor, overhead stirrers and heating block). The vessel was sealed and the wells purged by pressurising five times with nitrogen to 2 bar and releasing the pressure. Potassium tert-butylate (1 M solution in tert-butanol, each sample: 1.60 ml, 1.6 mmol) and isopropanol (each sample: 3.6 ml) were then injected. The wells were purged by pressurising five times with hydrogen to 25 bar (under stirring) and releasing the pressure. The reaction was then heated to 70° C. and pressurised to 25 bar hydrogen pressure. After 20 h, the hydrogen pressure was released and the reaction mixture was evaporated to dryness. The residue was dissolved in dichloromethane and washed with saturated ammonium chloride solution. The aqueous phase was extracted several times with dichloromethane. The combined organic layers were dried over sodium sulfate and concentrated in vacuo leading to a green solid (760 mg). The crude product was purified by flash chromatography on silica gel (Dichloromethane/Methanol 10:1). This afforded the pure title compound (560 mg of a yellow foam, 60% yield, 25.3% ee).

Determination of the optical purity by CE: MT [(3S)-enantiomer]=19.1 min/36.2 area-%; MT [(3R)-enantiomer]=19.6 min/60.7 area-%; 25.3% ee.

### 2. (3R)-7-Hydroxy-6-(3-hydroxy-3-phenyl-propyl)-2-methyl-3-(2-trimethylsilanyl-ethoxymethyl)-3H-benzoimidazole-5-carboxylic Acid Dimethylamide

In a flask filled with argon, 7-hydroxy-2-methyl-6-(3-oxo-3-phenyl-propyl)-3-(2-trimethylsilanyl-ethoxymethyl)-3H-benzoimidazole-5-carboxylic acid dimethylamide (example a, 6.4 g, 13.2 mmol) was dissolved in isopropanol (9.3 ml), water (2.7 ml), tert-butanol (10 ml), and potassium tert-butylate solution (1 M in tert-butanol, 14.6 ml). 33 mg of the hydrogenation catalyst RuCl₂[(S)-Xyl-P-Phos][(S)-DAIPEN] were added and the mixture was transferred into an autoclave and hydrogenated at 65° C. and 80 bar pressure for 20 h. After cooling to room temperature and releasing of the hydrogen pressure, the reaction mixture was concentrated in vacuo. The residue was purified by flash chromatography on silica gel (first column: Toluene/1,4-Dioxane, second column: Dichloromethane/Methanol=15:1) to afford 4.9 g (77% yield) of the title compound as a white solid.—An analytical sample was crystallized from diisopropyl ether/isopropanol: m.p. 142-143° C.

The enantiomeric excess was determined after transformation of the title compound into (8S)-2-methyl-8-phenyl-3,6,7,8-tetrahydro-chromeno[7,8-d]imidazole-5-carboxylic acid dimethylamide (example B, C).

¹H-NMR (DMSO-d₆, 200 MHz): δ=−0.10 (s, 9H), 0.82 (t, 2H), 1.80 (bs, 2H), 2.55 (s, bs); 2.67 (s), 2.89 (s, 3H), 3.51 (t, 2H), 4.49 (dt, 1H), 5.14 (d, 1H), 5.49 (s, 2H), 6.82 (s, 1H), 7.26 (m_{c}, 5H), 9.77 (bs, 1H).

### 3. (3R)-Azetidin-1-yl-[7-hydroxy-6-(3-hydroxy-3-phenyl-propyl)-2,3-dimethyl-3H-benzoimidazol-5-yl]-methanone

In a flask filled with argon, 3-[6-(azetidine-1-carbonyl)-4-hydroxy-1,2-dimethyl-1H-benzoimidazol-5-yl]-1-phenyl-propan-1-one (example e, 2.8 g, 7.6 mmol) was suspended in isopropanol (18.2 ml), water (3 ml), and potassium tert-butylate solution (1 M in tert-butanol, 9.1 ml). 22 mg of the hydrogenation catalyst RuCl₂[(S)-Xyl-P-Phos][(S)-DAIPEN] were added and the mixture was transferred into an autoclave and hydrogenated at 65° C. and 80 bar pressure for 2 d. After cooling to room temperature and releasing of the hydrogen pressure, the reaction mixture was concentrated in vacuo. The residue was purified by flash chromatography on silica gel (Dichloromethane/Methanol=10:1) to afford 0.93 g (32% yield; 85% ee) of the title compound as a beige solid.—m.p. 265-266° C.

Determination of the optical purity by CE: MT [(3S)-enantiomer]=23.1 min/7.6 area-%; MT [(3R)-enantiomer]=23.8 min/92.4 area-%; 84.8% ee.

¹H-NMR (DMSO-d₆, 200 MHz): δ=1.83 (m_{c}, 2H), 2.15 (m_{c}, 2H), 2.50, 2.55 (s, m_{c}), 2.77 (m_{c}, 1H), 3.66 (s, 3H), 3.80 (t, 2H), 3.93 (t, 2H), 4.48 (t, 1H), 5.21 (bs, 1H), 6.81 (s, 1H), 7.26 (m_{c}, 5H), 9.80 (bs, 1H).

### 4. (3R)-7-Hydroxy-6-(3-hydroxy-3-phenyl-propyl)-2,3-dimethyl-3H-benzoimidazole-5-carboxylic Acid Methylamide

In a flask filled with argon, 7-hydroxy-2,3-dimethyl-6-(3-oxo-3-phenyl-propyl)-3H-benzoimidazole-5-carboxylic acid methylamide (example g, 4.4 g, 12.5 mmol) was suspended in isopropanol (30 ml), water (5 ml), and potassium tert-butylate solution (1 M in tert-butanol, 15 ml). 31 mg of the hydrogenation catalyst RuCl₂[(S)-Xyl-P-Phos][(S)-DAIPEN] were added and the suspension was transferred into an autoclave and hydrogenated at 65° C. and 80 bar pressure for 3 d. After cooling to room temperature and releasing of the hydrogen pressure, the reaction mixture was concentrated in vacuo. The residue was purified by flash chromatography on silica gel (Dichloromethane/Methanol 10:1) to afford 2.3 g (52% yield, 77% ee) of the title compound as a light green solid.—m.p. 247-249° C.

Determination of the optical purity by CE: MT [(3S)-enantiomer]=24.5 min/11.7 area-%: MT [(3R)-enantiomer]=25.3 min/87.7 area-%; 76.5% ee.

¹H-NMR (DMSO-d₆, 200 MHz): δ=1.85 (m_{c}, 2H), 2.50, 2.53 (s, m_{c}), 2.71, 2.77 (d, ma, 4H), 3.66 (s, 3H), 4.46 (bt, 1H), 5.25 (bs, 1H), 6.88 (s, 1H), 7.25 (m_{c}, 5H), 8.00 (q, 1H), 9.70 (bs, 1H).

### 5. (3R)-6-[3-(2-Fluoro-phenyl)-3-hydroxy-propyl]-7-hydroxy-2,3-dimethyl-3-benzolmidazole-5-carboxylic Acid Dimethylamide

In a flask filled with argon, 6-[3-(2-fluoro-phenyl)-3-oxo-propyl]-7-hydroxy-2,3-dimethyl-3H-benzoimidazole-5-carboxylic acid dimethylamide (example k, 3.7 g, 9.6 mmol) was dissolved in isopropanol (4.9 ml), water (1.2 ml), and potassium tert-butylate solution (1 M in tert-butanol, 14.1 ml). 38.6 mg of the hydrogenation catalyst RuCl₂[(S)-Xyl-P-Phos][(S)-DAIPEN] were added and the mixture was transferred into an autoclave and hydrogenated at 65° C. and 80 bar pressure for 20 h. After cooling to room temperature and releasing of the hydrogen pressure, the reaction mixture was concentrated in vacuo. The residue was dissolved in dichloromethane (300 ml) and saturated ammonium chloride solution (50 ml). The solution was neutralized by cautiously adding 2 N HCl and the phases were separated. The aqueous phase was extracted with dichloromethane (2×50 ml). The combined organic phases were dried over magnesium sulfate and concentrated in vacuo. The residue was purified by flash chromatography on silica gel (Dichloromethane/Methanol=14:1) and crystallized from acetone to afford 1.7 g (49%) of the title compound as a light green solid.—m.p. 264-266° C.

The enantiomeric excess was determined after transformation of the title compound into (8S)-8-(2-fluoro-phenyl)-2,3-dimethyl-3,6,7,8-tetrahydro-chromeno[7,8-d]imidazole-5-carboxylic acid dimethylamide (example F).

¹H-NMR (DMSO-d₆, 200 MHz): δ=1.79 (m_{c}, 2H), 2.50 (s, bs), 2.68 (s, bs, 4H), 2.88 (s, 3H), 3.64 (s, 3H), 4.82 (bt, 1H), 5.26 (bs, 1H), 6.70 (s, 1H), 7.17 (m_{c}, 3H), 7.49 (dt, 1H), 9.80 (bs, 1H).

### 6. (3R)-6-[3-(4-Fluoro-phenyl)-3-hydroxy-propyl]-7-hydroxy-2,3-dimethyl-3H-benzoimidazole-5-carboxylic Acid Dimethylamide

In a flask filled with nitrogen, 6-[3-(4-fluoro-phenyl)-3-oxo-propyl]-7-hydroxy-2,3-dimethyl-3H-benzoimidazole-5-carboxylic acid dimethylamide (example 1, 5.1 g, 13.3 mmol) was suspended in isopropanol (9.5 ml), water (2.7 ml), potassium tert-butylate solution (1 M in tert-butanol, 14.6 ml), and tert-butanol (1.9 ml). The suspension was diluted with isopropanol (35 ml). 33 mg of the hydrogenation catalyst RuCl₂[(S)-Xyl-P-Phos][(S)-DAIPEN] were added and the suspension was transferred into an autoclave and hydrogenated at 65° C. and 80 bar pressure for 16 h. After cooling to room temperature and releasing of the hydrogen pressure, the reaction mixture was concentrated in vacuo. The residue was dissolved in dichloromethane and saturated ammonium chloride solution was added. The phases were separated. The organic phase was dried over magnesium sulfate and concentrated in vacuo in the presence of silica gel. The crude product was purified by flash chromatography on silica gel (Dichloromethane/Methanol=20:1) and crystallization from acetone to afford 1.8 g (35% yield; 88% ee) of the title compound as a white solid.—m.p. 276-277° C.

Determination of the optical purity by CE: MT [(3S)-enantiomer]=21.4 min/6.2 area-%; MT [(3R)-enantiomer]=21.8 min/93.3 area-%; 87.6% ee.

¹H-NMR (DMSO-d₆, 200 MHz): δ=1.80 (bs, 2H), 2.40, 2.49 (bs, s), 2.68 (s, bs, 4H), 2.89 (s, 3H), 3.64 (s, 3H), 4.50 (t, 1H), 5.19 (bs, 1H); 6.70 (s, 1H), 7.12 (t, 2H), 7.33 (dd, 2H), 9.90 (bs, 1H).

### 7. (3R)-7-Hydroxy-6-(3-hydroxy-3-o-tolyl-propyl)-2,3-dimethyl-3H-benzoimidazole-5-carboxylic Acid Dimethylamide

Asymmetric catalytic hydrogenation with RuCl₂[(S)-Xyl-P-Phos][(S)-DAIPEN], purification by flash chromatography: In a flask filled with argon, 7-hydroxy-2,3-dimethyl-6-(3-oxo-3-o-tolyl-propyl)-3H-benzoimidazole-5-carboxylic acid dimethylamide (example m, 6.0 g, 15.8 mmol) was dissolved in isopropanol (40 ml) and potassium tert-butylate solution (1 M in tert-butanol, 19 ml) was slowly added. 198 mg of the hydrogenation catalyst RuCl₂[(S)-Xyl-P-Phos][(S)-DAIPEN] were added and the mixture was transferred into an autoclave and hydrogenated at 70° C. and 80 bar pressure for 4 d. After cooling to room temperature and releasing of the hydrogen pressure, the reaction mixture was concentrated in vacuo. The residue was purified by flash chromatography on silica gel (Dichloromethane/Methanol=10:1) to afford 4.9 g (82% yield; 87% ee) of the title compound as a light green solid.—m.p. 139-140° C.

Determination of the optical purity by CE: MT [(3S)-enantiomer]=19.6 min/6.4 area-%; MT [(3R)-enantiomer]=20.4 min/88.8 area-%; 86.6% ee.

¹H-NMR (DMSO-d₆, 200 MHz): δ=1.79 (bs, 2H), 2.22 (s, 3H), 2.49 (s, bs), 2.70 (s, 3H), 2.93 (s, bs, 4H), 3.65 (s, 3H), 4.69 (bt, 1H), 5.03 (bs, 1H), 6.71 (s, 1H), 7.13 (m_{c}, 3H), 7.41 (m_{c}, 1H), 9.85 (bs, 1H).

Asymmetric catalytic hydrogenation with RuCl₂[(S)-Xyl-P-Phos][(S)-DAIPEN], purification by crystallization from acetone: In a flask filled with argon, 7-hydroxy-2,3-dimethyl-6-(3-oxo-3-o-tolyl-propyl)-3H-benzoimidazole-5-carboxylic acid dimethylamide (example m, 35.0 g, 92 mmol) was suspended in degassed isopropanol (340 ml) and potassium tert-butylate solution (1 M in tert-butanol, 101 ml) was slowly added. The yellow suspension was stirred at room temperature until a solution was obtained (20 min). The hydrogenation catalyst RuCl₂[(S)-Xyl-P-Phos][(S)-DAIPEN] (1.14 g, 0.92 mmol) was added and stirring was continued for several minutes. The brown solution was transferred into a 2 l autoclave with glass inlay, purged with hydrogen (3×), and hydrogenated at 70° C. and 80 bar pressure for 20 h. After cooling to room temperature and releasing of the hydrogen pressure, the reaction mixture was poured onto a stirred mixture of saturated ammonium chloride solution (400 ml) and dichloromethane (700 ml). The phases were separated and the aqueous phase was extracted with dichloromethane (2×80 ml). The combined organic phases were washed with water (400 ml), dried over sodium sulfate, and concentrated under reduced pressure. The residue (41 g of a green foam, 94.9% ee) was dissolved in hot acetone (100 ml). Upon cooling to room temperature, crystallization started. After a period of 3 h at room temperature and 2 h at 0° C., the precipitate was isolated by filtration, washed with acetone (20 ml) and diethyl ether (40 ml), and dried in vacuo. The title compound was isolated in the form of a colourless solid (26.5 g, 76% yield, 93.8% ee).—m.p. 215-217° C.

Determination of the optical purity by CE: MT [(3S)-enantiomer]=19.9 min/3.1 area-%; MT [(3R)-enantiomer]=20.7 min/96.9 area-%; 93.8% ee.

Asymmetric catalytic hydrogenation with RuCl₂[(S)-Xyl-P-Phos][(S)-DAIPEN], purification by crystallization from methyl isobutyl ketone: In a flask filled with argon, 7-hydroxy-2,3-dimethyl-6-(3-oxo-3-o-tolyl-propyl)-3H-benzoimidazole-5-carboxylic acid dimethylamide (example m, 4.0 g, 10.5 mmol) was suspended in degassed isopropanol (37 ml) and potassium tert-butylate solution (1 M in tert-butanol, 13 ml) was slowly added. The yellow suspension was stirred at room temperature until a solution was obtained (20 min). The hydrogenation catalyst RuCl₂[(S)-Xyl-P-Phos][(S)-DAIPEN] (0.13 g, 0.10 mmol) was added and stirring was continued for several minutes. The brown solution was transferred into a 100 ml autoclave, purged with hydrogen (3×), and hydrogenated at 70° C. and 80 bar pressure for 20 h. After cooling to room temperature and releasing of the hydrogen pressure, the reaction mixture was poured onto a stirred mixture of saturated ammonium chloride solution (80 ml) and dichloromethane (200 ml). The phases were separated and the aqueous phase was extracted with dichloromethane (2×30 ml). The combined organic phases were washed with water (80 ml), dried over sodium sulfate, and concentrated under reduced pressure. The residue (3.6 g of a green foam, 83.8% ee) was dissolved in warm (50° C.) methyl isobutyl ketone (20 ml). Upon cooling to room temperature, crystallization started. After a period of 40 h at room temperature, the precipitate was isolated by filtration, washed with methyl isobutyl ketone (4 ml) and diethyl ether (10 ml), and dried in vacuo. The title compound was isolated in the form of a colourless solid (3.2 g, 80% yield, 83.8% ee).—m.p. 200-202° C.

Determination of the optical purity by CE: MT [(3S)-enantiomer]=21.1 min/8.1 area-%; MT [(3R)-enantiomer]=21.9 min/91.9 area-%; 83.8% ee.

Asymmetric catalytic hydrogenation with RuCl₂[(S)-Xyl-P-Phos][(S)-DAIPEN], purification by crystallization from methyl ethyl ketone: In a flask filled with argon, 7-hydroxy-2,3-dimethyl-6-(3-oxo-3-o-tolyl-propyl)-3H-benzoimidazole-5-carboxylic acid dimethylamide (example m, 4.7 g, 12.4 mmol) was suspended in degassed isopropanol (29 ml) and potassium tert-butylate solution (1 M in tert-butanol, 21 ml) was slowly added. The yellow suspension was stirred at room temperature until a solution was obtained (20 min). The hydrogenation catalyst RuCl₂[(S)-Xyl-P-Phos][(S)-DAIPEN] (153 mg, 0.12 mmol) was added and stirring was continued for several minutes. The brown solution was transferred into a 100 ml autoclave, purged with hydrogen (3×), and hydrogenated at 70° C. and 80 bar pressure for 20 h. After cooling to room temperature and releasing of the hydrogen pressure, the reaction mixture was poured onto a stirred mixture of saturated ammonium chloride solution (80 ml) and dichloromethane (220 ml). The phases were separated and the aqueous phase was extracted with dichloromethane (2×30 ml). The combined organic phases were washed with water (50 ml), dried over sodium sulfate, and concentrated under reduced pressure. A green foam was obtained (3.5 g, 84.2% ee). A part of the crude product (2.5 g) was dissolved in hot (70° C.) methyl ethyl ketone (20 ml). The solution was stirred for 40 h at room temperature. A precipitate was formed, which was isolated by filtration, washed with hot methyl ethyl ketone (5 ml) and diethyl ether (10 ml), and dried in vacuo. The title compound (460 mg) was obtained with an optical purity of 42.8% ee. The mother liquor was concentrated and the optical purity was determined (92.4% ee). The residue was dissolved in hot isopropanol (8 ml) and a solution of oxalic acid (0.71 g, 7.9 mmol) in isopropanol (4 ml) was added. A suspension was obtained, which was diluted with isopropanol (4 ml) and stirred for 3 d at room temperature. The precipitate was isolated by filtration, washed with isopropanol (5 ml) and diethyl ether (10 ml), and dried in vacuo. The salt of the title compound with oxalic acid (2.1 g, m.p. 158° C.) was added portion-wise to a stirred mixture of sodium bicarbonate (1.8 g), water (30 ml) and dichloromethane (70 ml). The phases were separated and the aqueous phase was extracted with dichloromethane (2×10 ml). The combined organic phases were washed with water (50 ml), dried over sodium sulfate, and concentrated under reduced pressure. The residue (1.8 g of a yellow foam) was dissolved in warm (50° C.) methyl isobutyl ketone (10 ml). Upon cooling to room temperature, crystallization started. After a period of 2 h at room temperature, the precipitate was isolated by filtration, washed with methyl isobutyl ketone (2 ml) and diethyl ether (10 ml), and dried in vacuo. The title compound was isolated in the form of a colourless solid (1.3 g, 28% yield, 39% corrected yield, 94.0% ee).—m.p. 202-204° C.

Determination of the optical purity by CE: MT [(3S)-enantiomer]=21.1 min/3.0 area-%; MT [(3R)-enantiomer]=22.0 min/97.0 area-%; 94.0% ee.

Asymmetric catalytic hydrogenation with RuCl₂[(S)-Xyl-P-Phos][(S)-DAIPEN], purification by crystallization in the presence of oxalic acid: In a flask filled with argon, 7-hydroxy-2,3-dimethyl-6-(3-oxo-3-o-tolyl-propyl)-3H-benzoimidazole-5-carboxylic acid dimethylamide (example m, 5.0 g, 13.2 mmol) was suspended in degassed isopropanol (35.5 ml) and potassium tert-butylate solution (1 M in tert-butanol, 14.5 ml) was slowly added. The yellow suspension was stirred at room temperature until a solution was obtained (30 min). The hydrogenation catalyst RuCl₂[(S)-Xyl-P-Phos][(S)-DAIPEN] (165 mg, 0.13 mmol) was added and stirring was continued for several minutes. The brown solution was transferred into a 100 ml autoclave, purged with hydrogen (3×), and hydrogenated at 70° C. and 80 bar pressure for 20 h. After cooling to room temperature and releasing of the hydrogen pressure, the reaction mixture Was poured onto a stirred mixture of saturated ammonium chloride solution (100 ml) and dichloromethane (220 ml). The phases were separated and the aqueous phase was extracted with dichloromethane (2×30 ml). The combined organic phases were washed with water (100 ml), dried over sodium sulfate, and concentrated under reduced pressure. The residue (4.9 g of a green foam, 89.0% ee) was dissolved in warm (60° C.) isopropanol (15 ml) and a hot solution of oxalic acid (1.7 g, 18.9 mmol) in isopropanol (10 ml) was added. A suspension was formed, which was stirred for 2 h at room temperature and for 2 h at 0° C. The precipitate was isolated by filtration, washed with isopropanol (8 ml) and diethyl ether (15 ml), and dried in vacuo. This afforded the salt of the title compound with oxalic acid in 90% yield (5.6 g of a colourless solid, m.p. 146°-148° C.), which was added portion-wise to a stirred mixture of sodium bicarbonate (5 g), water (80 ml) and dichloromethane (100 ml). The phases were separated and the aqueous phase was extracted with dichloromethane (3×20 ml). The combined organic phases were washed with water (80 ml), dried over sodium sulfate, and concentrated under reduced pressure. The residue (4.45 g of a light brown foam) was dissolved in warm (50° C.) methyl isobutyl ketone (25 ml). Upon cooling to room temperature, crystallization started. After a period of 3 d at room temperature and 2 h at 0° C., the precipitate was isolated by filtration, washed with methyl isobutyl ketone (8 ml) and diethyl ether (30 ml), and dried in vacuo. The title compound was isolated in the form of a colourless solid (3.95 g, 78% yield, 89.4% ee).—m.p. 203-205° C.

Determination of the optical purity by CE: MT [(3S)-enantiomer]=20.1 min/5.3 area-%; MT [(3R)-enantiomer]=20.8 min/94.7 area-%; 89.4% ee.

¹H NMR data of the salt of the title compound with oxalic acid (DMSO-d₆, 200 MHz): δ=1.81 (m_{c}, 2H), 2.23 (s, 3H), 2.58 (s, bs, 4H), 2.70 (s, 3H), 2.93 (s, bs, 4H), 3.70 (s, 3H), 4.71 (m_{c}, 1H), 6.85 (s, 1H), 7.08 (m_{c}, 3H), 7.41 (m_{c}, 1H).

Asymmetric catalytic hydrogenation with RuCl₂[(S)-Xyl-P-Phos][(S)-DAIPEN], purification by crystallization in the presence of mandelic acid: In a flask filled with argon, 7-hydroxy-2,3-dimethyl-6-(3-oxo-3-o-tolyl-propyl)-3H-benzoimidazole-5-carboxylic acid dimethylamide (example m, 70.0 g, 184 mmol) was suspended in degassed isopropanol (680 ml) and potassium tert-butylate solution (1 M in tert-butanol, 203 ml) was slowly added. The yellow suspension was stirred at room temperature until a solution was obtained (30 min). The hydrogenation catalyst RuCl₂[(S)-Xyl-P-Phos][(S)-DAIPEN] (2.3 g, 1.84 mmol) was added and stirring was continued for 30 min. The brown solution was transferred into a 2 l autoclave with glass inlay, purged with hydrogen (3×), and hydrogenated at 70° C. and 80 bar pressure for 20 h. After cooling to room temperature and releasing of the hydrogen pressure, the reaction mixture was poured onto a stirred mixture of saturated ammonium chloride solution (700 ml) and dichloromethane (1300 ml). The phases were separated and the aqueous phase was extracted with dichloromethane (2×100 ml). The combined organic phases were washed with water (1 l), dried over sodium sulfate, and concentrated under reduced pressure. The residue (80 g of a green foam, 90.6% ee) was dissolved in hot acetone (500 ml) and a hot solution of (S)-mandelic acid (33.0 g, 217 mmol) in acetone (100 ml) was added. The dark solution was stirred for 10 min at 60° C. and for 17 h at room temperature. A suspension was formed, which was stirred for 2 h at 0° C. The precipitate was isolated by filtration, washed with acetone (50 ml) and diethyl ether (80 ml), and dried in vacuo. This afforded the salt of the title compound with (S)-mandelic acid in 78% yield (77.0 g of a colourless solid, m.p. 178°-180° C.), which was added portion-wise to a stirred mixture of sodium bicarbonate (60 g), water (400 ml) and dichloromethane (400 ml). The phases were separated and the aqueous phase was extracted with dichloromethane (2×80 ml). The combined organic phases were washed with water (200 ml), dried over sodium sulfate, and concentrated under reduced pressure. The residue (66.5 g of a greenish foam) was dissolved in hot acetone (150 ml). Upon cooling to room temperature, crystallization started. After a period of 17 h at room temperature and 2 h at 0° C., the precipitate was isolated by filtration, washed with acetone (30 ml) and diethyl ether (50 ml), and dried in vacuo. The title compound was isolated in the form of a colourless solid (50.0 g, 71% yield, 95.4% ee).—m.p. 207-209° C.

Determination of the optical purity by CE: MT [(3S)-enantiomer]=19.8 min/2.3 area-%; MT [(3R)-enantiomer]=20.7 min/97.7 area-%; 95.4% ee.

¹H NMR data of the salt of the title compound with (S)-mandelic acid (DMSO-d₆, 200 MHz): δ=1.79 (m_{c}, 2H), 2.22 (s, 3H), 2.51 (s, bs), 2.69 (s, 3H), 2.92 (s, bs, 4H), 3.65 (s, 3H), 4.69 (m_{c}, 1H), 5.02 (s, bs, 2H), 6.71 (s, 1H), 7.13 (m_{c}, 3H), 7.34 (m_{c}, 6H), 9.82 (bs, 1H).

Asymmetric catalytic hydrogenation with RuCl₂[(S)-Xyl-P-Phos][(S)-DAIPEN] with a S/C ratio of 250:1: In a flask filled with argon, 7-hydroxy-2,3-dimethyl-6-(3-oxo-3-o-tolyl-propyl)-3H-benzoimidazole-5-carboxylic acid dimethylamide (example m, 10.0 g, 26.3 mmol) was suspended in degassed isopropanol (20 ml) and potassium tert-butylate solution (1 M in tert-butanol, 30 ml) was slowly added. The yellow suspension was stirred at room temperature until a solution was obtained (30 min). The hydrogenation catalyst RuCl₂[(S)-Xyl-P-Phos][(S)-DAIPEN] (130 mg, 0.10 mmol) was added and stirring was continued for several minutes. The brown solution was transferred into a 100 ml autoclave, purged with hydrogen (3×), and hydrogenated at 70° C. and 80 bar pressure for 2 d. After cooling to room temperature and releasing of the hydrogen pressure, the reaction mixture was poured onto a stirred mixture of saturated ammonium chloride solution (120 ml) and dichloromethane (200 ml). The phases were separated and the aqueous phase was extracted with dichloromethane (2×50 ml). The combined organic phases were washed with water (150 ml), dried over sodium sulfate, and concentrated under reduced pressure. The residue (10 g of a green foam, 90.5% ee) was dissolved in hot acetone (40 ml) and a hot solution of (S)-mandelic acid (4.6 g, 30.2 mmol) in acetone (20 ml) was added. Upon cooling to room temperature a suspension was obtained, which was stirred for 3 h at room temperature. The precipitate was isolated by filtration, washed with acetone (15 ml) and diethyl ether (20 ml), and dried in vacuo. This afforded the salt of the title compound with (S)-mandelic acid in 66% yield (9.3 g of a slightly yellow solid, m.p. 174°-176° C.), which was added portion-wise to a stirred mixture of sodium bicarbonate (7 g), water (40 ml) and dichloromethane (100 ml). The phases were separated and the aqueous phase was extracted with dichloromethane (2×10 ml). The combined organic phases were washed with water (60 ml), dried over sodium sulfate, and concentrated under reduced pressure. The residue (7.5 g of a yellow foam) was dissolved in hot acetone (25 ml). Upon cooling to room temperature, crystallization started. After a period of 3 h at room temperature, the precipitate was isolated by filtration, washed with acetone (15 ml) and diethyl ether (20 ml), and dried in vacuo. The title compound was isolated in the form of a slightly yellow solid (5.1 g, 51% yield, 94.9% ee).—m.p. 200-202° C.

Determination of the optical purity by CE: MT [(3S)-enantiomer]=18.6 min/2.55 area-%; MT [(3R)-enantiomer]=19.3 min/97.45 area-%; 94.9% ee.

### Asymmetric catalytic hydrogenation with RuCl₂[(S)-Xyl-P-Phos][(S)-DAIPEN], screen of reaction conditions

The catalyst RuCl₂[(S)-Xyl-PPhos][(S)-DAIPEN] and 7-hydroxy-2,3-dimethyl-6-(3-oxo-3-o-tolyl-propyl)-3H-benzoimidazole-5-carboxylic acid dimethylamide were weighed in the glass liner and placed in a Parr microreactor (volume: 25 ml-300 ml) that was purged 5 times with nitrogen and 5 times with hydrogen. Potassium tert-butylate solution (1 M in tert-butanol) and isopropanol was added. The autoclave was then purged with hydrogen 5 times without stirring and 5 time with stirring. The pressure was set up to 30 bar and the mixture was heated up to 65° C. The reaction was stirred under these conditions for 17-24 h. After cooling to room temperature (presence of a yellow precipitate), the solvent was evaporated; the residue was dissolved in dichloromethane (100 ml) and washed with saturated ammonium chloride solution (100 ml). The aqueous phase was extracted several times with dichloromethane. The combined organic layers were dried over sodium sulfate and concentrated in vacuo leading to a green solid. The conversion and the enantiomeric excess were measured by HPLC.

| | | | | Temp. | Press. | | Conv. | |
|---|---|---|---|---|---|---|---|---|
| Entry | S/C | Substrate | Solvent and base | (° C.) | (bar) | Time | (%) | ee (%) |
| 1 | 100/1 | 0.38 g | 2.8 ml t-BuOK (1 M | 65 | 30 | 16 h | 100 | 93 |
| | | (1.0 mmol) | in t-BuOH), 2.2 ml | | | | | |
| | | | iPrOH (0.18 M) | | | | | |
| 2 | 100/1 | 0.82 g | 5.9 ml t-BuOK (1M in | 65 | 30 | 72 h | 100 | 95 |
| | | (2.2 mmol) | t-BuOH), 6.1 ml | | | | | |
| | | | iPrOH (0.18 M) | | | | | |
| 3 | 100/1 | 2.04 g | 14.9 ml t-BuOK (1 M | 65 | 30 | 16 h | 100 | 92 |
| | | (5.4 mmol) | in t-BuOH), 15.1 ml | | | | | |
| | | | iPrOH (0.18 M) | | | | | |
| 4 | 100/1 | 5.47 g | 39.7 ml t-BuOK (1 M | 65 | 30 | 16 h | >97 | 95 |
| | | (14.4 mmol) | in t-BuOH), 40.3 ml | | | | | |
| | | | iPrOH (0.18 M) | | | | | |
| 5 | 250/1 | 2.04 g | 15 ml t-BuOK (1M in | 65 | 30 | 16 h | 100 | 92 |
| | | (5.4 mmol) | t-BuOH), 15 ml | | | | | |
| | | | iPrOH (0.18 M) | | | | | |
| 6 | 250/1 | 0.38 g | 1.7 ml t-BuOK (1 M | 65 | 30 | 16 h | 100 | 95 |
| | | (1.0 mmol) | in t-BuOH), 3.3 ml | | | | | |
| | | | iPrOH (0.18 M) | | | | | |
| 7 | 250/1 | 0.38 g | 2.8 ml t-BuOK (1 M | 65 | 30 | 16 h | 100 | 89 |
| | | (1.0 mmol) | in t-BuOH), 2.2 ml | | | | | |
| | | | iPrOH (0.18 M) | | | | | |

HPLC analytical method: column: Merck LichroCART 250-4, Chiradex (5 μg)—eluant: methanol/water: 20/80, 1 ml/min—first eluting enantiomer: 12.8 min, second eluting enantiomer: 17.2 min, starting material: 23.0 min.

### 8. (3R)-6-[3-(2-Chloro-phenyl)-3-hydroxy-propyl]-7-hydroxy-2,3-dimethyl-3H-benzoimidazole-5-carboxylic Acid Dimethylamide

The catalyst RuCl₂[(S)-Xyl-PPhos][(S)-DAIPEN] (15 mg) and 6-[3-(2-chloro-phenyl)-3-oxo-propyl]-7-hydroxy-2,3-dimethyl-3H-benzoimidazole-5-carboxylic acid dimethylamide (example n, 479 mg, 1.25 mmol) were weighed in the glass liner and placed in a Parr microreactor (volume: 25 ml) that was purged 5 times with nitrogen and 5 times with hydrogen. Potassium tert-butylate solution (1 M in tert-butanol, 3.4 ml) and isopropanol (3 ml) was added. The autoclave was then purged with hydrogen 5 times without stirring and 5 time with stirring. The pressure was set up to 25-30 bar and the mixture was heated up to 65° C. The reaction was stirred under these conditions for 20 h. After cooling to room temperature, the solvent was evaporated. The residue was dissolved in dichloromethane and washed with saturated ammonium chloride solution. The aqueous phase was extracted several times with dichloromethane. The combined organic layers were dried over sodium sulfate and concentrated in vacuo leading to a green solid (440 mg). The conversion (>95%) and the enantiomeric excess (94% ee) were measured by HPLC. A part of the crude product (400 mg) was purified by flash chromatography on silica gel (Dichloromethane/Methanol=100:3). This afforded the pure title compound (220 mg of a foamy solid, 44% yield, 48% corrected yield, 88.4% ee).

HPLC analytical method: column: Merck LichroCART 250-4, Chiradex (5 μg)—eluant: methanol/water: 25/75, 1 ml/min—first eluting enantiomer: 19.2 min, second eluting enantiomer: 24.8 min, starting material: 27.6 min.

Determination of the optical purity by CE: MT [(3S)-enantiomer]=21.9 min/5.8 area-%; MT [(3R)-enantiomer]=23.7 min/94.2 area-%; 88.4% ee.

¹H-NMR (DMSO-d₆, 400 MHz): δ=1.69 (bs, 1H), 1.81 (bs, 1H), 2.38, 2.50 (bs, s), 2.69, 2.70 (s, bs, 4H), 2.89 (s, 3H), 3.64 (s, 3H), 4.89 (bs, 1H), 5.34 (bs, 1H), 6.70 (s, 1H), 7.24 (m_{c}, 1H), 7.35 (m_{c}, 2H), 7.58 (d, 1H), 9.78 (bs, 1H).

### 9. (3R)-7-Hydroxy-6-[3-hydroxy-3-(2-trifluoromethyl-phenyl)-propyl]-2,3-dimethyl-31 benzolmidazole-5-carboxylic Acid Dimethylamide

Samples of 7-hydroxy-2,3-dimethyl-6-[3-oxo-3-(2-trifluoromethyl-phenyl)-propyl]-3H-benzoimidazole-5-carboxylic acid dimethylamide (example o, cf. table) and RuCl₂[(S)-Xyl-P-Phos][(S)-DAIPEN] (preparation described above) were weighed in glass liners that were then placed in an Argonaut Endeavour (eight wells pressure parallel reactor, overhead stirrers and heating block). The vessel was sealed and the wells purged by pressurising five times with nitrogen to 2 bar and releasing the pressure. The base (1 M solution of potassium tert-butylate in tert-butanol) and isopropanol were then injected. The wells were purged by pressurising five times with hydrogen to 25 bar (under stirring) and releasing the pressure. The reaction was then heated to the set temperature (cf table) and pressurised to the set pressure of hydrogen (cf table). After the period specified in the table, the hydrogen pressure was released and the reaction mixtures were transferred to round bottomed flasks with the help of methanol (10 ml). The solvent was evaporated and the crude samples were analysed by HPLC (determination of conversion and optical purity).

| | | | | Temp. | Press. | | Conv. | |
|---|---|---|---|---|---|---|---|---|
| Entry | S/C | Substrate | Solvent and base | (° C.) | (bar) | Time | (%) | ee (%) |
| 1 | 100/1 | 195 mg | 0.9 ml t-BuOK (1 M | 65 | 25 | 16 h | >95 | >80 |
| | | (0.45 mmol) | in t-BuOH), 1.6 ml | | | | | |
| | | | iPrOH (0.18 M) | | | | | |
| 2-3 | 100/1 | 390 mg | 1.8 ml t-BuOK (1M in | 65 | 25 | 16 h | >95 | >80 |
| | | (0.90 mmol) | t-BuOH), 3.2 ml | | | | | |
| | | | iPrOH (0.18 M) | | | | | |

The combined samples were dissolved in dichloromethane and washed with saturated ammonium chloride solution. The aqueous phase was extracted several times with dichloromethane. The combined organic layers were dried over sodium sulfate and concentrated in vacuo furnishing a green solid (630 mg). A part of the crude product (590 mg) was purified by flash chromatography on silica gel (Dichloromethane/Methanol=50:1 to 10:1). This afforded the pure title compound (150 mg, 15% yield, 16% corrected yield, 72.6% ee).

HPLC analytical method: column: Merck LichroCART 250-4, Chiradex (5 μg)—eluant: methanol/water: 15/85, 0.8 ml/min—first eluting enantiomer: 12.8 min, second eluting enantiomer: 17.2 min, starting material: 21.0 min.

Determination of the optical purity by CE: MT [(3S)-enantiomer]=21.0 min/13.7 area-%; MT [(3R)-enantiomer]=21.4 min/86.3 area-%; 72.6% ee.

¹H-NMR (DMSO-d₈, 200 MHz): δ=1.67 (m_{c}, 1H), 1.89 (m, 1H), 2.50 (bs, s), 2.69 (s, 3H), 2.92 (bs, s, 4H), 3.64 (s, 3H), 4.85 (bs, 1H), 5.43 (bs, 1H), 6.70 (s, 1H), 7.43 (m_{c}, 1H), 7.66 (m_{c}, 2H), 7.77 (m_{c}, 1H), 9.93 (bs, 1H).

### 10. (3R)-7-Hydroxy-6-(3-hydroxy-3-naphthalen-2-yl-propyl)-2,3-dimethyl-3H-benzoimidazole-5-carboxylic Acid Dimethylamide

The catalyst RuCl₂[(S)-Xyl-PPhos][(S)-DAIPEN] (12 mg) and 7-hydroxy-2,3-dimethyl-6-(3-naphthalen-2-yl-3-oxo-propyl)-3H-benzoimidazole-5-carboxylic acid dimethylamide (example p, 400 mg, 1.0 mmol) were weighed in the glass liner and placed in a Parr microreactor (volume: 25 ml) that was purged 5 times with nitrogen and 5 times with hydrogen. Potassium tert-butylate solution (1 M in tert-butanol, 2.75 ml) and isopropanol (2.5 ml) was added. The autoclave was then purged with hydrogen 5 times without stirring and 5 time with stirring. The pressure was set up to 25-30 bar and the mixture was heated up to 65° C. The reaction was stirred under these conditions for 20 h. After cooling to room temperature, the solvent was evaporated. The residue was dissolved in dichloromethane and washed with saturated ammonium chloride solution. The aqueous phase was extracted several times with dichloromethane. The combined organic layers were dried over sodium sulfate and concentrated in vacuo leading to a green solid (340 mg). The conversion (100%) was measured by HPLC. A part of the crude product (300 mg) was purified by flash chromatography on silica gel (Dichloromethane/Methanol=15:1). This afforded the pure title compound (164 mg of a green solid, 39% yield, 45% corrected yield).—m.p. 145-147° C.

The enantiomeric excess was determined after transformation of the title compound into (8S)-2,3-dimethyl-8-naphthalen-2-yl-3,6,7,8-tetrahydro-chromeno[7,8-d]imidazole-5-carboxylic acid dimethylamide hydrochloride (example K).

¹H-NMR (DMSO-d₆, 400 MHz): δ=1.81 (bs, 1H), 2.01 (bs, 1H), 2.50 (s, bs), 2.66 (s, 3H), 2.78 (s, bs, 4H), 3.64 (s, 3H), 4.66 (bs, 1H), 5.29 (bs, 1H), 6.69 (s, 1H), 7.48 (m_{c}, 3H), 7.81 (s, 1H), 7.88 (m_{c}, 3H), 9.73 (bs, 1H).

### 11. (3R)-6-[3-(2-Ethyl-phenyl)-3-hydroxy-propyl]-7-hydroxy-2,3-dimethyl-3H-benzoimidazole-5-carboxylic Acid Dimethylamide

The catalyst RuCl₂[(S)-Xyl-PPhos][(S)-DAIPEN] (11 mg) and 6-[3-(2-ethyl-phenyl)-3-oxo-propyl]-7-hydroxy-2,3-dimethyl-3H-benzoimidazole-5-carboxylic acid dimethylamide (example q, 340 mg, 0.9 mmol) were weighed in the glass liner and placed in a Parr microreactor (volume: 25 ml) that was purged 5 times with nitrogen and 5 times with hydrogen. Potassium tert-butylate solution (1 M in tert-butanol, 2.47 ml) and isopropanol (2.53 ml) was added. The autoclave was then purged with hydrogen 5 times without stirring and 5 time with stirring: The pressure was set up to 25-30 bar and the mixture was heated up to 65° C. The reaction was stirred under these conditions for 20 h. After cooling to room temperature, the solvent was evaporated. The residue was dissolved in dichloromethane and washed with saturated ammonium chloride solution. The aqueous phase was extracted several times with dichloromethane. The combined organic layers were dried over sodium sulfate and concentrated in vacuo leading to a green solid (310 mg). The conversion (>95%) and the enantiomeric excess (85% ee) were measured by HPLC. A part of the crude product (280 mg) was purified by flash chromatography on silica gel (Dichloromethane/Methanol=100:3). This afforded the pure title compound (150 mg of an off-white solid, 42% yield, 51% corrected yield, 86.4% ee).—m.p. 135-137° C.

HPLC analytical method: column: Merck LichroCART 250-4, Chiradex (5 μg)—eluant: methanol/water: 20/80, 1 ml/min—first eluting enantiomer: 13.5 min, second eluting enantiomer: 19.4 min, starting material: 28.1 min.

Determination of the optical purity by CE: MT [(3S)-enantiomer]=20.5 min/6.8 area-%; MT [(3R)-enantiomer]=21.8 min/93.2 area-%; 86.4% ee.

¹H-NMR (DMSO-d₆, 200 MHz): δ=1.12 (t, 3H), 1.75 (bs, 2H), 2.40 (bs), 2.50 (s), 2.58 (q). 2.70, 2.83, 2.94 (s, bs, s, 7H), 3.65 (s, 3H), 4.74 (bs, 1H), 5.01 (bs, 1H), 6.71 (s, 1H), 7.13 (m_{c}, 3H), 7.41 (m_{c}, 1H), 9.75 (bs, 1H).

### 12. (3R)-7-Hydroxy-6-(3-hydroxy-3-thiophen-2-yl-propyl)-2,3-dimethyl-3H-benzoimidazole-5-carboxylic Acid Dimethylamide

Two samples of 7-hydroxy-2,3-dimethyl-6-(3-oxo-3-thiophen-2-yl-propyl)-3H-benzoimidazole-5-carboxylic acid dimethylamide (example r, 400 mg, 1.08 mmol) were weighed in glass liners and placed in an Argonaut Endeavour that was purged 5 times with nitrogen and 5 times with hydrogen. Potassium tert-butylate (180 mg, 1.61 mmol) and isopropanol (3.45 ml) were added followed by addition of a solution of pre-activated hydrogenation catalyst [2.3 ml, prepared by heating a solution of RuCl₂[(S)-Xyl-PPhos][(S)-DAIPEN] (12.6 mg) and potassium-tert-butylate (1 M solution in tert-butanol, 115 μl) in isopropanol (2.185 ml) to 60° C. for 1 h)]. The reaction vessels were purged by pressurising five times with hydrogen to 25 bar (under stirring) and releasing the pressure. The reaction was then heated to 65° C. and pressurised to 25 bar hydrogen pressure. After 20 h, the hydrogen pressure was released and the reaction mixture was evaporated to dryness. The residue was dissolved in dichloromethane and washed with saturated ammonium chloride solution. The aqueous phase was extracted several times with dichloromethane. The combined organic layers were dried over sodium sulfate and concentrated in vacuo leading to a green solid (670 mg). The crude product was purified by flash chromatography on silica gel (Dichloromethane/Methanol=25:1). This afforded the pure title compound (500 mg of a yellow powder, 62% yield, 79.9% ee).—m.p. 264-265° C.

HPLC analytical method: column: Daicel Chiralpak AD-H, 250×4.6 mm, 5 μm—eluant: n-heptane/ethanol: 80/20, flow rate: 1 ml/min, detection wavelength: 218 nm—first eluting enantiomer: 21.0 min/88.8 area-%, second eluting enantiomer: 23.1 min/9.9 area-%, 79.9% ee.

¹H-NMR (DMSO-d₆, 200 MHz): δ=1.92 (bs, 2H), 2.50 (s), 2.71 (s, bs, 4H), 2.95 (s, 3H), 3.65 (s, 3H), 4.73 (bt, 1H), 5.48 (bs, 1H), 6.72 (s, 1H), 6.95 (m_{c}, 2H), 7.37 (dd, 1H), 9.80 (bs, 1H).

### 13. (3R)-6-[3-(4-Fluoro-2-methyl-phenyl)-3-hydroxy-propyl]-7-hydroxy-2,3-dimethyl-3H-benzoimidazole-5-carboxylic Acid Dimethylamide

The catalyst RuCl₂[(S)-Xyl-PPhos][(S)-DAIPEN] (35 mg) and 6-[3-(4-fluoro-2-methyl-phenyl)-3-oxo-propyl]-7-hydroxy-2,3-dimethyl-3H-benzoimidazole-5-carboxylic acid dimethylamide (example s, 1.1 g, 2.88 mmol) were weighed in the glass liner and placed in a Parr microreactor (volume: 50 ml) that was purged 5 times with nitrogen and 5 times with hydrogen. Potassium tert-butylate solution (1 M in tert-butanol, 8.0 ml) and isopropanol (8.0 ml) was added. The autoclave was then purged with hydrogen 5 times without stirring and 5 time with stirring. The pressure was set up to 25-30 bar and the mixture was heated up to 65° C. The reaction was stirred under these conditions for 2.5 d. After cooling to room temperature, the solvent was evaporated. The residue was dissolved in dichloromethane and washed with saturated ammonium chloride solution. The aqueous phase was extracted several times with dichloromethane. The combined organic layers were dried over sodium sulfate and concentrated in vacuo leading to a green solid (1.0 g). The conversion (>95%) and the enantiomeric excess (86% ee) was measured by HPLC. A part of the crude product (950 mg) was purified by flash chromatography on silica gel (Dichloromethane/Methanol=100:3). This afforded the pure title compound (670 mg of a foamy solid, 58% yield, 61% corrected yield, 83.2% ee).

HPLC analytical method: column: Merck LichroCART 250-4, Chiradex (5 μg)—eluant: methanol/water: 20/80, 1 ml/min—first eluting enantiomer: 17.5 min, second eluting enantiomer: 23.7 min, starting material: 19 min.

Determination of the optical purity by CE: MT [(3S)-enantiomer]=22.5 min/8.4 area-%; MT [(3R)-enantiomer]=23.7 min/91.6 area-%; 83.2% ee.

¹H-NMR (DMSO-d₆, 200 MHz): δ=1.78 (bs, 2H), 2.23 (s, 3H), 2.36 (bs), 2.51 (s), 2.69 (s, 3H), 2.93 (s, bs, 4H), 3.65 (s, 3H), 4.67 (bs, 1H), 5.08 (bs, 1H), 6.71 (s, 1H), 6.96 (m_{c}, 2H), 7.42 (m_{c}, 1H), 9.82 (bs, 1H).

### 14. (3R)-[7-Hydroxy-6-(3-hydroxy-3-o-tolyl-propyl)-2,3-dimethyl-3H-benzoimidazol-5-yl]-pyrrolidin-1-yl-methanone

Two samples of 3-[4-hydroxy-1,2-dimethyl-6-(pyrrolidine-1-carbonyl)-1H-benzoimidazol-5-yl]-1-o-tolyl-propan-1-one (example x, sample A and B: 450 mg, 1.15 mmol) and RuCl₂[(S)-Xyl-P-Phos][(S)-DAIPEN] (sample A and B: 14 mg) were weighed in glass liners that were then placed in an Argonaut Endeavour (eight wells pressure parallel reactor, overhead stirrers and heating block). The vessel was sealed and the wells purged by pressurising five times with nitrogen to 2 bar and releasing the pressure. Potassium tert-butylate (1 M solution in tert-butanol, sample A: 3.1 ml, sample B: 2.3 ml) and isopropanol (sample A: 2.5 ml, sample B: 3.3 ml) were then injected. The wells were purged by pressurising five times with hydrogen to 25 bar (under stirring) and releasing the pressure. The reaction was then heated to 65° C. and pressurised to 25 bar hydrogen pressure. After 20 h, the hydrogen pressure was released and the reaction mixtures were evaporated to dryness. Each residue was dissolved in dichloromethane and washed with saturated ammonium chloride solution. The aqueous phase was extracted several times with dichloromethane. The combined organic layers were dried over sodium sulfate and concentrated in vacuo leading to a green solid. The conversion (sample A and B: >95%) and the enantiomeric excess (sample A and B: 91% ee) was measured by HPLC. The samples were combined (0.85 g). The optical purity was determined by CE (90.8% ee) and the title compound was used for the next step (example O) without further purification.

HPLC analytical method: column: Merck LichroCART 250-4, Chiradex (5 μg)—eluant: methanol/water: 20/80, 1 ml/min—first eluting enantiomer: 21.8 min, second eluting enantiomer: 30.9 min, starting material: 32.0 min.

Determination of the optical purity by CE: MT [(3S)-enantiomer]=21.0 min/4.6 area-%; MT [(3R)-enantiomer]=21.7 min/95.4 area-%; 90.8% ee.

¹H-NMR (DMSO-d₆, 400 MHz): δ=1.71 (m_{c}, 3H), 1.82 (m_{c}, 3H), 2.22 (s, 3H), 2.50 (bs), 2.60 (s, 3H), 2.78 (bs, 1H), 3.00 (bs, 2H), 3.38 (m_{c}, 2H), 3.72 (s, 3H), 4.72 (t, 1H), 5.17 (bs, 1H), 6.97 (s, 1H), 7.13 (m_{c}, 3H), 7.40 (d, 1H), 9.92 (bs, 1H).

### 15. (3R-7-Hydroxy-6-(3-hydroxy-3-o-tolyl-propyl)-2,3-dimethyl-3H-benzolmidazole-5-carboxylic Acid Methylamide

7-Hydroxy-2,3-dimethyl-6-(3-oxo-3-o-tolyl-propyl)-3H-benzoimidazole-5-carboxylic acid methylamide (example z, 524 mg, 1.44 mmol) and RuCl₂[(S)-Xyl-P-Phos][(S)-DAIPEN] (18.5 mg) were weighed in a glass liner that was then placed in an Argonaut Endeavour (eight wells pressure parallel reactor, overhead stirrers and heating block). The vessel was sealed and the well was purged by pressurising five times with nitrogen to 2 bar and releasing the pressure. Potassium hydroxide solution (10 M solution in water, 0.9 ml) and isopropanol (2.1 ml) were then injected. The well was purged by pressurising five times with hydrogen to 25 bar (under stirring) and releasing the pressure. The reaction was then heated to 65° C. and pressurised to 25 bar hydrogen pressure. After 3 d, the hydrogen pressure was released and the reaction mixture was evaporated to dryness. The conversion (>95%) and the enantiomeric excess (90% ee) was measured by HPLC. The crude product was combined with another sample which was obtained by asymmetric reduction of 608 mg (1.68 mmol) of 7-hydroxy-2,3-dimethyl-6-(3-oxo-3-o-tolyl-propyl)-3H-benzoimidazole-5-carboxylic acid methylamide under analogous conditions (>95% conversion, >90% ee). The combined samples were dissolved in dichloromethane and washed with saturated ammonium chloride solution. The aqueous phase was extracted several times with dichloromethane. The combined organic layers were dried over sodium sulfate and concentrated in vacuo furnishing a green solid (0.5 g). A part of the crude product (422 mg) was purified by flash chromatography on silica gel (Dichloromethane/Methanol=20:1). This afforded the pure title compound (288 mg of a grey solid, 25% yield, 30% corrected yield, 94.7% ee).—m.p. 238-240° C.

HPLC analytical method: column: Merck LichroCART 250-4, Chiradex (5 μg)—eluant: methanol/water: 20/80, 1 ml/min—first eluting enantiomer: 18.0 min, second eluting enantiomer: 25.0 min, starting material: 27.0 min.

Determination of the optical purity by CE: MT [(3S)-enantiomer]=20.7 min/2.6 area-%; MT [(3R)-enantiomer]=21.7 min/94.7 area-%; 94.7% ee.

¹H-NMR (DMSO-d₆, 400 MHz): δ=1.67 (m, 1H), 1.87 (m_{c}, 1H), 2.18 (s, 3H), 2.50 (s), 2.70, 2.72 (m_{c}, d, 4H), 2.91 (m_{c}, 1H), 3.66 (s, 3H), 4.66 (m_{c}, 1H), 5.23 (d, 1H), 6.89 (s, 1H), 7.07 (m_{c}, 2H), 7.14 (m_{c}, 1H), 7.43 (d, 1H), 8.05 (q, 1H), 9.69 (bs, 1H).

### 16. (3R)-Azetidin-1-yl-[7-hydroxy-6-(3-hydroxy-3-o-tolyl-propyl)-2,3-dimethyl-3H-benzoimidazol-5-yl]-methanone

Samples of 3-[6-(azetidine-1-carbonyl)-4-hydroxy-1,2-dimethyl-1H-benzoimidazol-5-yl]-1-o-tolyl-propan-1-one (example bb, cf. table) and RuCl₂[(S)-Xyl-P-Phos][(S)-DAIPEN] (preparation described above) were weighed in glass liners that were then placed in an Argonaut Endeavour (eight wells pressure parallel reactor, overhead stirrers and heating block). The vessel was sealed and the wells purged by pressurising five times with nitrogen to 2 bar and releasing the pressure. The base (10 M solution of potassium hydroxide in water) and isopropanol were then injected. The wells were purged by pressurising five times with hydrogen to 25 bar (under stirring) and releasing the pressure. The reaction was then heated to the set temperature (cf table) and pressurised to the set pressure of hydrogen (cf table). After the period specified in the table, the hydrogen pressure was released and the reaction mixtures were transferred to round bottomed flasks with the help of methanol (10 ml). The solvent was evaporated and the crude samples were analysed by HPLC (determination of conversion and optical purity).

| | | | | Temp. | Press. | | Conv. | |
|---|---|---|---|---|---|---|---|---|
| Entry | S/C | Substrate | Solvent and base | (° C.) | (bar) | Time | (%) | ee (%) |
| 1 | 100/1 | 660 mg | 1.0 ml KOH (10 M in | 65 | 25 | 3 d | >95 | 80 |
| | | (1.69 mmol) | water), 2.5 ml iPrOH | | | | | |
| | | | (0.5 M) | | | | | |
| 2 | 100/1 | 404 mg | 0.75 ml KOH(10 M in | 65 | 25 | 16 h | 100 | 80 |
| | | (1.03 mmol) | water), 1.75 ml | | | | | |
| | | | iPrOH (0.43 M) | | | | | |

The combined samples were dissolved in dichloromethane and washed with saturated ammonium chloride solution. The aqueous phase was extracted several times with dichloromethane. The combined organic layers were dried over sodium sulfate and concentrated in vacuo furnishing a green solid (0.8 g). A part of the crude product (700 mg) was purified by flash chromatography on silica gel (Dichloromethane/Methanol=100:3). This afforded the pure title compound (90 mg of a foamy solid, 8% yield, 10% corrected yield, 95.2% ee).

HPLC analytical method: column: Merck LichroCART 250-4, Chiradex (5 μg)—eluant: methanol/water: 20/80, 1 ml/min—first eluting enantiomer: 14.9 min, second eluting enantiomer: 20.4 min, starting material: 26.0 min.

Determination of the optical purity by CE: MT [(3S)-enantiomer]=20.9 min/2.4 area-%; MT [(3R)-enantiomer]=21.7 min/97.6 area-%; 95.2% ee.

¹H-NMR (DMSO-d₆, 400 MHz): δ=1.69 (m_{c}, 1H), 1.84 (m, 1H), 2.16 (m_{c}, 2H), 2.20 (s, 3H), 2.51 (s), 2.65 (m_{c}, 1H), 2.86 (m_{c}, 1H), 3.68 (s, 3H), 3.81 (m_{c}, 2H), 3.96 (m_{c}, 2H), 4.68 (bs, 1H), 5.16 (bs, 1H), 6.83 (s, 1H), 7.09 (m_{c}, 2H), 7.16 (m_{c}, 1H), 7.44 (m_{c}, 1H), 9.78 (bs, 1H).

### 17. (3R)-6-[3-(2-Benzyloxymethyl-phenyl)-3-hydroxy-propyl]-7-hydroxy-2,3-dimethyl-3H-benzoimidazole-5-carboxylic acid dimethylamide

Samples of 6-[3-(2-benzyloxy-phenyl)-3-oxo-propyl]-7-hydroxy-2,3-dimethyl-3H-benzoimidazole-5-carboxylic acid dimethylamide (example cc, cf. table) and RuCl₂[(S)-Xyl-P-Phos][(S)-DAIPEN] (preparation described above) were weighed in glass liners that were then placed in an Argonaut Endeavour (eight wells pressure parallel reactor, overhead stirrers and heating block). The vessel was sealed and the wells purged by pressurising five times with nitrogen to 2 bar and releasing the pressure. The base (1 M solution of potassium tert-butylate in tert-butanol) and isopropanol were then injected. The wells were purged by pressurising five times with hydrogen to 25 bar (under stirring) and releasing the pressure. The reaction was then heated to the set temperature (cf table) and pressurised to the set pressure of hydrogen (cf table). After the period specified in the table, the hydrogen pressure was released and the reaction mixtures were transferred to round bottomed flasks with the help of methanol (10 ml). The solvent was evaporated and the crude samples were analysed by HPLC (determination of conversion).

| | | | | Temp. | Press. | | Conv. | |
|---|---|---|---|---|---|---|---|---|
| Entry | S/C | Substrate | Solvent and base | (° C.) | (bar) | Time | (%) | ee (%) |
| 1 | 100/1 | 218 mg | 0.5 ml t-BuOk (1 M | 65 | 25 | 16 h | >95 | n.d. |
| | | (0.45 mmol) | in t-BuOH), 2.0 ml | | | | | |
| | | | iPrOH (0.18 M) | | | | | |
| 2-4 | 100/1 | 540 mg | 1.25 ml t-BuOK (1M | 65 | 25 | 16 h | >95 | n.d. |
| | | (1.13 mmol) | in t-BuOH), 4.4 ml | | | | | |
| | | | iPrOH (0.2 M) | | | | | |
| 5 | 100/1 | 540 mg | 1.25 ml t-BuOK (1M | 65 | 25 | 16 h | >85 | n.d. |
| | | (1.13 mmol) | in t-BuOH), 4.4 ml | | | | | |
| | | | iPrOH (0.2 M) | | | | | |

The combined samples were dissolved in dichloromethane and washed with saturated ammonium chloride solution. The aqueous phase was extracted several times with dichloromethane. The combined organic layers were dried over sodium sulfate and concentrated in vacuo furnishing a green solid (2.3 g). A part of the crude product (2.2 g) was purified by flash chromatography on silica gel (Dichloromethane/Methanol=20:1). This afforded the pure title compound (1.47 g of a foamy solid, 61% yield, 64% corrected yield, 96.4% ee).

Determination of the optical purity by HPLC: column: Daicel Chiralpak AD-H 250×4.6 mm, 5 μm—eluant: n-heptane/ethanol: 70/30, 1 ml/min—diode array detection at 218 nm—(3R)-enantiomer: 14.1 min/98.2 area-%, (3S)-enantiomer: 25.9 min/1.8 area-%, 96.4% ee.

¹H-NMR (DMSO-d₆, 400 MHz): δ=1.60-2.02 (bm, 2H), 2.20-2.63 (bm), 2.50 (s), 2.69 (s, 3H), 2.94 (s, 3H), 3:63 (s, 3H), 4.44 (m_{c}, 4H), 4.76 (bs, 1H), 5.12 (bs, 1H), 6.72 (s, 1H), 7.26 (m_{c}, 8H), 7.48 (m_{c}, 1H), 9.85 (bs, 1H).

### 18. (3R)-7-Hydroxy-6-[3-hydroxy-3-(2-methoxymethyl-phenyl)-propyl]-2,3-dimethyl-3H-benzoimidazole-5-carboxylic acid dimethylamide

Samples of 7-hydroxy-6-[3-(2-methoxy-phenyl)-3-oxo-propyl]-2,3-dimethyl-3H-benzoimidazole-5-carboxylic acid dimethylamide (example dd, cf. table) and RuCl₂[(S)-Xyl-P-Phos][(S)-DAIPEN] (preparation described above) were weighed in glass liners that were then placed in an Argonaut Endeavour (eight wells pressure parallel reactor, overhead stirrers and heating block). The vessel was sealed and the wells purged by pressurising five times with nitrogen to 2 bar and releasing the pressure. The base (1 M solution of potassium tert-butylate in tert-butanol) and isopropanol were then injected. The wells were purged by pressurising five times with hydrogen to 25 bar (under stirring) and releasing the pressure. The reaction was then heated to the set temperature (cf table) and pressurised to the set pressure of hydrogen (cf table). After the period specified in the table, the hydrogen pressure was released and the reaction mixtures were transferred to round bottomed flasks with the help of methanol (10 ml). The solvent was evaporated and the crude samples were analysed by HPLC (determination of conversion and optical purity).

| | | | | Temp. | Press. | | Conv. | |
|---|---|---|---|---|---|---|---|---|
| Entry | S/C | Substrate | Solvent and base | (° C.) | (bar) | Time | (%) | ee (%) |
| 1 | 100/1 | 184 mg | 0.5 ml t-BuOK (1 M | 65 | 25 | 16 h | 100 | >95 |
| | | (0.45 mmol) | in t-BuOH), 2.0 ml | | | | | |
| | | | iPrOH (0.18 M) | | | | | |
| 2-4 | 100/1 | 410 mg | 1.1 ml t-BuOK (1M in | 65 | 25 | 16 h | 100 | >95 |
| | | (1.00 mmol) | t-BuOH), 3.9 ml | | | | | |
| | | | iPrOH (0.2 M) | | | | | |

The combined samples were dissolved in dichloromethane and washed with saturated ammonium chloride solution. The aqueous phase was extracted several times with dichloromethane. The combined organic layers were dried over sodium sulfate and concentrated in vacuo furnishing a green solid (1.22 g). The crude product was purified by flash chromatography on silica gel (Dichloromethane/Methanol=20:1). This afforded the pure title compound (940 mg of a foamy solid, 66% yield, 97.4% ee).

HPLC analytical method: column: Merck LichroCART 250-4, Chiradex (5 μg)—eluant: methanol/water: 15/85, 1 ml/min—first eluting enantiomer: 9.0 min, second eluting enantiomer: 12.0 min, starting material: 15.0 min.

Determination of the optical purity by CE: MT [(3S)-enantiomer]=20.4 min/1.3 area-%; MT [(3R)-enantiomer]=20.6 min/98.7 area-%; 97.4% ee.

¹H-NMR (DMSO-d₆, 400 MHz): δ=1.81 (m_{c}, 2H), 2.48 (s, bs), 2.70 (s, 3H), 2.94 (s, bs, 4H), 3.24 (s, 3H), 3.65 (s, 3H), 4.40 (s, 2H), 4.75 (bs, 1H), 5.04 (bs, 1H), 6.71 (s, 1H), 7.22 (m_{c}, 3H), 7.47 (m, 1H), 9.76 (bs, 1H).

### 19. 7-Hydroxy-6-(3-hydroxy-3-o-tolyl-propyl)-2-methyl-3-(2-trimethylsilanyl-ethoxymethyl)-3H-benzoimidazole-5-carboxylic acid dimethylamide

Two samples of 7-hydroxy-2-methyl-6-(3-oxo-3-o-tolyl-propyl)-3-(2-trimethylsilanyl-ethoxymethyl)-3H-benzoimidazole-5-carboxylic acid dimethylamide (example ee, cf. table) and RuCl₂[(S)-Xyl-P-Phos][(S)-DAIPEN] (preparation described above) were weighed in glass liners that were then placed in an Argonaut Endeavour (eight wells pressure parallel reactor, overhead stirrers and heating block). The vessel was sealed and the wells purged by pressurising five times with nitrogen to 2 bar and releasing the pressure. The base (1 M solution of potassium tert-butylate in tert-butanol) and isopropanol were then injected. The wells were purged by pressurising five times with hydrogen to 25 bar (under stirring) and releasing the pressure. The reaction was then heated to the set temperature (cf table) and pressurised to the set pressure of hydrogen (cf table). After the period specified in the table, the hydrogen pressure was released and the reaction mixtures were transferred to round bottomed flasks with the help of methanol (10 ml). The solvent was evaporated and the crude samples were analysed by HPLC (determination of conversion and optical purity).

| | | | | Temp. | Press. | | Conv. | |
|---|---|---|---|---|---|---|---|---|
| Entry | S/C | Substrate | Solvent and base | (° C.) | (bar) | Time | (%) | ee (%) |
| 1 | 100/1 | 223 mg | 0.5 ml t-BuOK (1 M | 65 | 25 | 16 h | 100 | 92 |
| | | (0.45 mmol) | in t-BuOH), 2.0 ml | | | | | |
| | | | iPrOH (0.18 M) | | | | | |
| 2 | 100/1 | 271 mg | 0.6 ml t-BuOK (1M | 65 | 25 | 16 h | 100 | 91 |
| | | (0.54 mmol) | in t-BuOH), 2.4 ml | | | | | |
| | | | iPrOH (0.18 M) | | | | | |

The combined samples were dissolved in dichloromethane and washed with saturated ammonium chloride solution. The aqueous phase was extracted several times with dichloromethane. The combined organic layers were dried over sodium sulfate and concentrated in vacuo furnishing a green solid (390 mg). A part of the crude product (350 mg) was purified by flash chromatography on silica gel (Dichloromethane/Methanol=20:1). This afforded the pure title compound (320 mg of a foamy solid, 64% yield, 70% corrected yield, 95.8% ee).

HPLC analytical method: column: Merck LichroCART 250-4, Chiradex (5 μg)—eluant: methanol/water: 50/50, 0.8 ml/min—first eluting enantiomer: 14.5 min, second eluting enantiomer: 16.0 min, starting material: 19.0 min.

Determination of the optical purity by CE: MT [(3S)-enantiomer]=27.5 min/2.1 area-%; MT [(3R)-enantiomer]=27.9 min/97.9 area-%; 95.8% ee.

¹H-NMR (DMSO-d₆, 400 MHz): δ=−0.10 (s, 9H), 0.82 (t, 2H), 1.78 (bm, 2H), 2.21 (s, 3H), 2.55 (s), 2.69 (s, 3H), 2.92 (s, 4H), 3.50 (t, 2H), 4.69 (bs, 1H), 5.03 (bd, 1H), 5.50 (s, 2H), 6.84 (s, 1H), 7.13 (m_{c}, 3H), 7.41 (m_{c}, 1H), 9.77 (bs, 1H).

### 20. (3R)-6-(3-Benzo[b]thiophen-3-yl-3-hydroxy-propyl)-7-hydroxy-2,3-dimethyl-3H-benzoimidazole-5-carboxylic acid dimethylamide

6-(3-Benzo[b]thiophen-3-yl-3-oxo-propyl)-7-hydroxy-2,3-dimethyl-3H-benzoimidazole-5-carboxylic acid dimethylamide (example ff, 190 mg, 0.45 mmol) and RuCl₂[(S)-Xyl-P-Phos][(S)-DAIPEN] (5.5 mg) were weighed in a glass liner that was then placed in an Argonaut Endeavour (eight wells pressure parallel reactor, overhead stirrers and heating block). The vessel was sealed and the wells purged by pressurising five times with nitrogen to 2 bar and releasing the pressure. Potassium tert-butylate (1 M solution in tert-butanol, 1.00 ml, 1.0 mmol) and isopropanol (1.5 ml) were then injected. The wells were purged by pressurising five times with hydrogen to 25 bar (under stirring) and releasing the pressure. The reaction was then heated to 65° and pressurised to 25 bar hydrogen pressure. After 20 h, the hydrogen pressure was released and the reaction mixture was evaporated to dryness. The conversion (>95%) was measured by HPLC. The residue was dissolved in dichloromethane and washed with saturated ammonium chloride solution. The aqueous phase was extracted several times with dichloromethane. The combined organic layers were dried over sodium sulfate and concentrated in vacuo leading to a green solid (140 mg). A part of the crude product (120 mg) was purified by flash chromatography on silica gel (Dichloromethane/Methanol=100:3). This afforded the title compound (40 mg of a yellow-green foam, 21% yield, 24% corrected yield, 82.2% ee).

HPLC analytical method: column: Merck LichroCART 250-4, Chiradex (5 μg)—eluant: methanol/water: 50/50, 0.8 ml/min—title compound (both enantiomers): 5.0 min, starting material: 11.0 min.

HPLC analytical method: column: Daicel Chiralpak AD-H, 250×4.6 mm, 5 μm—eluant: n-heptane/ethanol: 80/20, flow rate: 1 ml/min, detection wavelength: 218 nm—first eluting enantiomer: 33.1 min/91.1 area-%, second eluting enantiomer: 39.9 min/8.9 area-%, 82.2% ee.

¹H-NMR (DMSO-d₆, 400 MHz): δ=1.80-2.26 (m_{c}, 2H), 2.51 (s, bs), 2.63 (s, 3H), 2.83 (bs, 4H), 3.65 (s, 3H), 4.89 (bs, 1H), 5.34 (d, 1H), 6.71 (s, 1H), 7.36 (m_{c}, 2H), 7.54 (s, 1H), 7.90 (bs, 1H), 7.96 (m_{c}, 1H), 9.79 (bs, 1H).

### 21. (3R)-7-Hydroxy-6-[3-hydroxy-3-(2-methyl-thiophen-3-yl)-propyl]-2,3-dimethyl-3R benzoimidazole-5-carboxylic acid dimethylamide

Samples of 7-hydroxy-2,3-dimethyl-6-[3-(2-methyl-thiophen-3-yl)-3-oxo-propyl]-3H-benzoimidazole-5-carboxylic acid dimethylamide (example gg, cf. table) and RuCl₂[(S)-Xyl-P-Phos][(S)-DAIPEN] (preparation described above) were weighed in glass liners that were then placed in an Argonaut Endeavour (eight wells pressure parallel reactor, overhead stirrers and heating block). The vessel was sealed and the wells purged by pressurising five times with nitrogen to 2 bar and releasing the pressure. The base (1 M solution of potassium tert-butylate in tert-butanol) and isopropanol were then injected. The wells were purged by pressurising five times with hydrogen to 25 bar (under stirring) and releasing the pressure. The reaction was then heated to the set temperature (cf table) and pressurised to the set pressure of hydrogen (cf table). After the period specified in the table, the hydrogen pressure was released and the reaction mixtures were transferred to round bottomed flasks with the help of methanol (10 ml). The solvent was evaporated and the crude samples were analysed by HPLC (determination of conversion).

| | | | | Temp. | Press. | | Conv. | |
|---|---|---|---|---|---|---|---|---|
| Entry | S/C | Substrate | Solvent and base | (° C.) | (bar) | Time | (%) | ee (%) |
| 1 | 100/1 | 173 mg | 0.5 ml t-BuOK (1 M | 65 | 25 | 16 h | 100 | n.d. |
| | | (0.45 mmol) | in t-BuOH), 2.0 ml | | | | | |
| | | | iPrOH (0.18 M) | | | | | |
| 2-3 | 100/1 | 410 mg | 1.18 ml t-BuOK (1M | 65 | 25 | 16 h | 100 | n.d. |
| | | (1.06 mmol) | in t-BuOH), 4.74 ml | | | | | |
| | | | iPrOH (0.18 M) | | | | | |

The combined samples were dissolved in dichloromethane and washed with saturated ammonium chloride solution. The aqueous phase was extracted several times with dichloromethane. The combined organic layers were dried over sodium sulfate and concentrated in vacuo furnishing a green solid (860 mg). A part of the crude product (820 mg) was purified by flash chromatography on silica gel (Dichloromethane/Methanol=100:3). This afforded the pure title compound (600 mg of a pale green foam, 60% yield, 63% corrected yield, 95.8% ee).

HPLC analytical method: column: Merck LichroCART 250-4, Chiradex (5 μg)—eluant: methanol/water: 20/80, 1 ml/min—title compound (both enantiomers): 7.0 min, starting material: 10.0 min.

Determination of the optical purity by CE: MT [(3S)-enantiomer]=20.0 min/2.1 area-%; MT [(3R)-enantiomer]=21.0 min/97.9 area-%; 95.8% ee.

¹H-NMR (CDCl₃, 200 MHz): δ=2.10, 2.24 (bs, bs, 5H), 2.59, 2.63 (s, bs, 4H), 2.82 (s, 3H), 3.12, 3.14 (bs, s, 4H), 3.67 (s, 3H), 4.64 (m_{c}, 1H), 6.69 (s, 1H), 6.96 (d, 1H), 7.03 (d, 1H).

### 22. (3R)-7-Hydroxy-6-(3-hydroxy-3-o-tolyl-propyl)-2,3-dimethyl-3H-benzoimidazole-5-carboxylic acid cyclopropylamide

Samples of 7-hydroxy-2,3-dimethyl-6-(3-oxo-3-o-tolyl-propyl)-3H-benzoimidazole-5-carboxylic acid cyclopropylamide (example 11, cf. table) and RuCl₂[(S)-Xyl-P-Phos][(S)-DAIPEN] (preparation described above) were weighed in glass liners that were then placed in an Argonaut Endeavour (eight wells pressure parallel reactor, overhead stirrers and heating block). The vessel was sealed and the wells purged by pressurising five times with nitrogen to 2 bar and releasing the pressure. The base (1 M solution of potassium tert-butylate in tert-butanol) and isopropanol were then injected. The wells were purged by pressurising five times with hydrogen to 25 bar (under stirring) and releasing the pressure. The reaction was then heated to the set temperature (cf table) and pressurised to the set pressure of hydrogen (cf table). After the period specified in the table, the hydrogen pressure was released and the reaction mixtures were transferred to round bottomed flasks with the help of methanol (10 ml). The solvent was evaporated and the crude samples were analysed by HPLC (determination of conversion).

| | | | | Temp. | Press. | | Conv. | |
|---|---|---|---|---|---|---|---|---|
| Entry | S/C | Substrate | Solvent and base | (° C.) | (bar) | Time | (%) | ee (%) |
| 1 | 100/1 | 175 mg | 1.25 ml t-BuOK (1 M | 65 | 25 | 16 h | 100 | >95 |
| | | (0.45 mmol) | in t-BuOH), 1.25 ml | | | | | |
| | | | iPrOH (0.18 M) | | | | | |
| 2-3 | 100/1 | 350 mg | 2.5 ml t-BuOK (1M | 65 | 25 | 16 h | 100 | >95 |
| | | (0.90 mmol) | in t-BuOH), 2.5 ml | | | | | |
| | | | iPrOH (0.18 M) | | | | | |

The combined samples were dissolved in dichloromethane and washed with saturated ammonium chloride solution. The aqueous phase was extracted several times with dichloromethane. A part of the title compound (610 mg) was isolated in the form of a colourless solid between the organic and the aqueous phase. The combined organic layers were dried over sodium sulfate and concentrated in vacuo. This afforded another 70 mg of the crude title compound. A part of the crude product (610 mg) was purified by flash chromatography on silica gel (Dichloromethane/Methanol=100:3). This afforded the pure title compound (350 mg of a colourless solid, 40% yield, 45% corrected yield, 98.4% ee).—m.p. 299-300° C.

HPLC analytical method: column: Merck LichroCART 250-4, Chiradex (5 μg)—eluant: methanol/water: 20/80, 1 ml/min—first eluting enantiomer: 11.0 min, second eluting enantiomer: 15.0 min, starting material: 18.0 min.

Determination of the optical purity by CE: MT [(3S)-enantiomer]=22.0 min/0.8 area-%; MT [(3R)-enantiomer]=23.1 min/99.2 area-%; 98.4% ee.

¹H-NMR (DMSO-d₆, 200 MHz): δ=0.54 (m_{c}, 2H), 0.65 (m_{c}, 2H), 1.75 (m_{c}, 2H), 2.21 (s, 3H), 2.50 (s), 2.80 (m_{c}, 3H), 3.66 (s, 3H), 4.65 (t, 1H), 5.19 (d, 1H), 6.85 (s, 1H), 7.10 (m, 3H), 7.43 (m_{c}, 1H), 8.17 (d, 1H), 9.63 (bs, 1H).

### 23. (3R)-5-(3-Hydroxy-3-o-tolyl-propyl)-6-methoxymethyl-1,2-dimethyl-1H-benzoimidazol-4-ol

In a flask filled with argon, 3-(4-hydroxy-6-methoxymethyl-1,2-dimethyl-1H-benzoimidazol-5-yl)-1-o-tolyl-propan-1-one (example mm, 1.4 g, 4.0 mmol) was suspended in isopropanol (40 ml) and potassium tert-butylate solution (1 M in tert-butanol, 5.6 ml) was slowly added. The suspension was stirred for 1 h at room temperature and the hydrogenation catalyst RuCl₂[(S)-Xyl-P-Phos][(S)-DAIPEN] (50 mg) was added. The mixture was transferred into an autoclave and hydrogenated at 70° C. and 80 bar pressure for 20 h. After cooling to room temperature and releasing of the hydrogen pressure, saturated ammonium chloride solution, dichloromethane, and water was added to the brown solution. The phases were separated. The organic phase was washed with water (1×) and the aqueous phase was extracted with dichloromethane (3×). The combined organic phases were dried over magnesium sulfate and concentrated to dryness. The residue was purified by column chromatography on silica gel (Dichloromethane/Methanol 50:1) to afford 1.0 g (71% yield; 98.2% ee) of the title compound as a light green foam.

HPLC analytical method: column: Daicel Chiralpak AD-H, 250×4.6 mm, 5 μm—eluant: n-hexane/isopropanol: 80/20, flow rate: 1 ml/min, detection wavelength: 218 nm—first eluting enantiomer: 16.9 min/99.1 area-%, second eluting enantiomer: 19.2 min/0.9 area-%, 98.2% ee.

¹H-NMR (DMSO-d₆, 200 MHz): δ=1.76 (m_{c}, 2H), 2.21 (s, 3H), 2.49 (s), 2.64 (m_{c}, 1H), 2.81 (m_{c}, 1H), 3.22 (s, 3H), 3.64 (s, 3H), 4.39 (s, 2H), 4.75 (bt, 1H), 5.04 (bd, 1H), 6.84 (s, 1H), 7.14 (m_{c}, 3H), 7.46 (d, 1H), 9.49 (bs, 1H).

### Synthesis of Compounds of the Formula 1-b by Asymmetric Reduction of Prochiral Ketones of the Formula 2

### 24. (3S)-7-Hydroxy-6-(3-hydroxy-3-phenyl-propyl)-2,3-dimethyl-3H-benzolmidazole-5-carboxylic Acid Dimethylamide

7-Hydroxy-2,3-dimethyl-6-(3-oxo-3-phenyl-propyl)-3H-benzoimidazole-5-carboxylic acid dimethylamide (0.37 g, 1.0 mmol) and RuCl₂[(R)-Xyl-P-Phos][(R)-DAIPEN] (1.7 mg, preparation analogous to the procedure given above) were weighed in a glass liner that was then placed in an Argonaut Endeavour (eight wells pressure parallel reactor, overhead stirrers and heating block). The vessel was sealed and the well was purged by pressurising five times with nitrogen to 2 bar and releasing the pressure. A solution of potassium tert-butylate (1 M in tert-butanol, 1.2 ml), tert-butanol (0.6 ml), and water (0.2 ml) were then injected. The wells were purged by pressurising five times with hydrogen to 25 bar (under stirring) and releasing the pressure. The reaction was then heated to 65° C. and pressurised to a hydrogen pressure of 25 bar. After a period of 16 h, the hydrogen pressure was released. Dichloromethane and aqueous saturated ammonium chloride solution was added to the crude reaction and a neutral pH was adjusted by addition of 2 N HCl. The phases were separated, the organic phase was dried over magnesium sulfate and was evaporated to dryness. The conversion and the optical purity of the crude product were determined by ¹H NMR spectroscopy (>95% conversion) and capillary electrophoresis (99% ee).

Determination of the optical purity by CE: MT [(3S)-enantiomer]=15.2 min/99.4 area-%: MT [(3R)-enantiomer]=15.7 min/0.6 area-%; 98.8% ee.

¹H-NMR (DMSO-d₆, 200 MHz): δ=1.80 (m_{c}, 2H), 2.35 (bs), 2.50 (s), 2.68 (s, bs, 4H), 2.89 (s, 3H), 3.64 (s, 3H), 4.48 (t, 1H), 5.14 (bs, 1H), 6.70 (s, 1H), 7.25 (m_{c}, 5H), 9.80 (bs, 1H).

### 25. (3S)-7-Hydroxy-6-(3-hydroxy-3-o-tolyl-propyl)-2,3-dimethyl-3H-benzoimidazole-5-carboxylic Acid Dimethylamide

Three samples of 7-hydroxy-2,3-dimethyl-6-(3-oxo-3-o-tolyl-propyl)-3H-benzoimidazole-5-carboxylic acid dimethylamide (example m, 3×400 mg, 1.05 mmol) and RuCl₂[(R)-Xyl-PPhos][(R)-DAIPEN] (each sample: 13 mg) were weighed in a glass liner that was then placed in an Argonaut Endeavour (eight wells pressure parallel reactor, overhead stirrers and heating block). The vessel was sealed and the wells purged by pressurising five times with nitrogen to 2 bar and releasing the pressure. Potassium tert-butylate (1 M solution in tert-butanol, each sample: 1.15 ml, 1.2 mmol) and isopropanol (each sample: 4.7 ml) were then injected. The wells were purged by pressurising five times with hydrogen to 25 bar (under stirring) and releasing the pressure. The reaction was then heated to 65′ and pressurised to 25 bar hydrogen pressure. After 20 h, the hydrogen pressure was released and the reaction mixture was evaporated to dryness. Conversion (1^{st} and 2^{nd} sample: 100%, 3^{rd} sample: 60%) and enantioselectivity (1^{st} sample: 93% ee, 2^{nd} sample: 92% ee, 3^{rd} sample: 90% ee) were determined by HPLC. Each residue was dissolved in dichloromethane and washed with saturated ammonium chloride solution. The aqueous phase was extracted several times with dichloromethane. The combined organic layers of each reaction were dried over sodium sulfate and concentrated in vacuo affording green solids. The crude product obtained from the 3^{rd} sample was re-hydrogenated using the same reaction conditions. The work-up of the reaction mixture was performed as described above. The crude products of all reactions were combined (1.1 g) and purified by column chromatography on silica gel (Ethyl acetate/Methanol=20:1). Evaporation of the corresponding fractions afforded a green foam (950 mg), which was dissolved in hot acetone (2 ml). Upon cooling to room temperature a suspension was obtained, which was stirred for 1 h at room temperature. The precipitate was isolated by filtration, washed with acetone (1 ml) and diethyl ether (5 ml), and dried in vacuo. This afforded the pure title compound (680 mg of a colourless solid, 57% yield, 88.4% ee).—m.p. 203-205° C.

HPLC analytical method: column: Merck LichroCART 250-4, Chiradex (5 μg)—eluant: methanol/water: 20/80, 1 ml/min—first eluting enantiomer: 12.8 min, second eluting enantiomer: 17.2 min, starting material: 23.0 min.

Determination of the optical purity by CE: MT [(3S)-enantiomer]=21.7 min/94.2 area-%; MT [(3R)-enantiomer]=22.9 min/5.8 area-%; 88.4% ee.

¹H-NMR (DMSO-d₆, 200 MHz): δ=1.79 (bs, 2H), 2.22 (s, 3H), 2.49 (s, bs), 2.70 (s, 3H), 2.93 (s, bs, 4H), 3.65 (s, 3H), 4.69 (bt, 1H), 5.03 (bs, 1H), 6.71 (s, 1H), 7.13 (m_{c}, 3H), 7.41 (m_{c}, 1H), 9.85 (bs, 1H).

Conversion of Compounds of the Formula 1-a into Tricyclic Benzimidazoles of the Formula 3-a

### A. (8S)-2,3-Dimethyl-8-phenyl-3,6,7,8-tetrahydro-chromeno[7,8-d]imidazole-5-carboxylic Acid Dimethylamide

To a suspension of (3R-7-hydroxy-6-(3-hydroxy-3-phenyl-propyl)-2,3-dimethyl-3H-benzoimidazole-5-carboxylic acid dimethylamide (example 1, 1.3 g, 3.5 mmol) and triphenylphosphine (2.7 g, 10.2 mmol) in tetrahydrofuran (60 ml), DIAD (2.1 ml, 10.5 mmol) was added and the mixture was stirred for 15 min. at room temperature. The reaction was concentrated in vacuo, the residue was treated with saturated ammonium chloride solution (100 ml) and was extracted with ethyl acetate (2×100 ml). The combined organic phases were washed with saturated ammonium chloride solution (20 ml) and water (20 ml), dried over magnesium sulfate, and concentrated in vacuo. The crude product was purified by flash chromatography on silica gel (Ethyl acetate/Methanol=9:1) to afford 1.03 g of the title compound. Crystallization from diisopropyl ether (20 ml) furnished the pure title compound (0.95 g of a white solid, 77% yield; 96% ee).—m.p. 226-227° C.

[α]²⁰_{D}=−32° (c=0.57, methanol)

Determination of the optical purity by CE: MT [(8S)-enantiomer]=19.7 min/97.9 area-%: MT [(8R)-enantiomer]=21.0 min/2.1 area-%; 95.8% ee.

¹H-NMR (DMSO-d₆, 200 MHz): δ=2.13 (m_{c}, 2H), 2.47 (s), 2.57 (m_{c}), 2.77, 2.80 (s, m_{c}, 4H), 3.00 (s, 3H), 3.68 (s, 3H), 5.22 (dd, 1H), 6.91 (s, 1H), 7.42 (m_{c}, 5H).

### B. (8S)-2-Methyl-8-phenyl-3-(2-trimethylsilanyl-ethoxymethyl)-3,6,7,8-tetrahydro-chromeno[7,8-d]imidazole-5-carboxylic Acid Dimethylamide

To a solution of (3R)-7-hydroxy-6-(3-hydroxy-3-phenyl-propyl)-2-methyl-3-(2-trimethylsilanyl-ethoxymethyl)-3H-benzoimidazole-5-carboxylic acid dimethylamide (example 2, 3.3 g, 6.8 mmol) in tetrahydrofuran (100 ml) were added triphenylphosphine (5.2 g, 19.7 mmol) and DIAD (4.0 ml, 20.5 mmol) and the mixture was stirred for 90 min at room temperature. The reaction was concentrated in vacuo. The residue was treated with saturated ammonium chloride solution and was extracted twice with ethyl acetate. The organic phase was washed with saturated ammonium chloride solution and water, dried over magnesium sulfate, and concentrated in vacuo. The residue was purified by flash chromatography on silica gel (Toluene Ethyl acetate=1:4) to afford a mixture of the title compound with triphenylphosphine oxide (3.3 g of a beige solid).

¹H-NMR (DMSO-d6, 200 MHz): δ=−0.1 (s, 9H), 0.83 (t, 2H), 2.13 (m_{c}, 2H), 2.50 (s, bs), 2.76, 2.80 (s, bs, 4H), 3.01 (s, 3H), 3.51 (t, 2H), 5.23 (dd, 1H), 5.54 (s, 2H), 7.05 (s, 1H), 7.31-7.70 (m).

### C. (8S)-2-Methyl-8-phenyl-3,6,7,8-tetrahydro-chromeno[7,8-d]imidazole-5-carboxylic Acid Dimethylamide

A solution of (8S)-2-methyl-8-phenyl-3-(2-trimethylsilanyl-ethoxymethyl)-3,6,7,8-tetrahydro-chromeno[7,8-d]imidazole-5-carboxylic acid dimethylamide (3.3 g, product of example B) in dichloromethane (40 ml) was cooled to 0° C. and boron trifluoride diethyl etherate (3.6 ml, 28.5 mmol) was added drop-wise. The reaction mixture was warmed to room temperature, stirred for 19 h, and concentrated in vacuo. The residue was purified by flash chromatography on silica gel (Dichloromethane/Methanol=20:1). A beige solid was isolated (1.0 g, free base of the title compound), which was dissolved in acetone (10 ml) and treated with oxalic acid (0.27 g, 3.0 mmol). The suspension was stirred for 18 h at room temperature and the precipitate was isolated by filtration and dried in vacuo. This afforded the title compound in 24% overall yield (0.70 g of a colourless solid, 94% ee).—m.p. 215-216° C.

[α]²⁰_{D}=−18° (c=0.58, methanol)

Determination of the optical purity by CE: MT [(8S)-enantiomer]=21.1 min/96.9 area-%; MT [(8R)-enantiomer]=22.5 min/3.1 area-%; 93.8% ee.

¹H-NMR (DMSO-d₆, 200 MHz): δ=2.18 (m_{c}, 2H), 2.51 (s), 2.57 (m_{c}), 2.77, 2.80 (s, m_{c}, 4H), 3.01 (s, 3H), 5.29 (dd, 1H), 6.95 (s, 1H), 7.46 (m_{c}, 5H).

### D. (8S)-Azetidin-1-yl-(2,3-dimethyl-8-phenyl-3,6,7,8-tetrahydro-chromeno[7,8-d]imidazol-5-yl)-methanone

To a suspension of (3R)-azetidin-1-yl-[7-hydroxy-6-(3-hydroxy-3-phenyl-propyl)-2,3-dimethyl-3H-benzoimidazol-5-yl]-methanone (example 3, 0.8 g, 2.2 mmol) and triphenylphosphine (1.6 g, 6.3 mmol) in tetrahydrofuran (40 ml) was added DIAD (1.3 ml, 6.5 mmol) and the mixture was stirred overnight at room temperature. The precipitate was filtered and dried in vacuo at 40° C. to afford 0.6 g (76% yield; 82% ee) of the title compound as a white solid.—m.p. 241-242° C.

Determination of the optical purity by CE: MT [(8S)-enantiomer]=21.3 min 90.9 area-%: MT [(8R)-enantiomer]=23.2 min/9.1 area-%; 81.8% ee.

¹H-NMR (DMSO-d₆, 200 MHz): δ=2.15 (m_{c}, 4H), 2.47 (s), 2.74 (m_{c}, 1H), 2.96 (m_{c}, 1H), 3.69 (s, 3H), 3.94 (m_{c}, 4H), 5.21 (dd, 1H), 7.03 (s, 1H), 7.40 (m_{c}, 5H).

### E. (8S)-2,3-Dimethyl-8-phenyl-3,6,7,8-tetrahydro-chromeno[7,8-d]imidazole-5-carboxylic Acid Methylamide

To a suspension of (3R)-7-hydroxy-6-(3-hydroxy-3-phenyl-propyl)-2,3-dimethyl-3H-benzoimidazole-5-carboxylic acid methylamide (example 4, 1.0 g, 2.8 mmol) and triphenylphosphine (2.1 g, 8.1 mmol) in tetrahydrofuran (50 ml) was added DIAD (1.6 ml, 8.4 mmol) and the suspension was stirred overnight at room temperature. The reaction was concentrated in vacuo and the residue was purified by flash chromatography on silica gel (Dichloromethane/Methanol=10:1) to afford 0.79 g (75% yield; 73% ee) of the title compound as a white solid.—m.p. 290-291° C.

[α]²⁰_{D}=−13° (c=0.56, methanol)

Determination of the optical purity by CE: MT [(8S)-enantiomer]=22.0 min/86.6 area-%; MT [(8R)-enantiomer]=24.4 min/13.4 area-%; 73.2% ee.

¹H-NMR (DMSO-d₆, 200 MHz): δ=2.02 (m_{c}, 1H), 2.22 (m_{c}, 1H), 2.48 (s), 2.76, 2.80 (d, m_{c}, 4H), 3.05 (m_{c}, 1H), 3.69 (s, 3H), 5.19 (dd, 1H), 7.11 (s, 1H), 7.42 (m_{c}, 5H), 8.08 (q, 1H).

### F. (8S)-8-(2-Fluoro-phenyl)-2,3-dimethyl-3,6,7,8-tetrahydro-chromeno[7,8-d]imidazole-5-carboxylic Acid Dimethylamide

A solution of (3R)-6-[3-(2-fluoro-phenyl)-3-hydroxy-propyl]-7-hydroxy-2,3-dimethyl-3H-benzoimidazole-5-carboxylic acid dimethylamide (example 5, 1.6 g, 4.1 mmol) and triphenylphosphine (3.1 g, 12 mmol) in tetrahydrofuran (100 ml) was treated with DIAD (2.5 g, 12 mmol) and the mixture was stirred for 1 h at room temperature. The reaction was concentrated in vacuo, the residue was treated with saturated ammonium chloride solution (100 ml) and extracted with ethyl acetate (3×100 ml). The combined organic phases were dried over magnesium sulfate and concentrated in vacuo. The residue was purified by flash chromatography on silica gel (Dichloromethane/Methanol=14:1) and crystallized from acetone to afford 0.8 g (62% yield, 96% ee) of the title compound as a white solid.—m.p. 199-201° C.

[α]²⁰_{D}=−50° (c=0.49, methanol)

Determination of the optical purity by HPLC: column: Daicel Chiralpak AD-H 250×4.6 mm, 5 μm—eluant: n-heptane/ethanol: 80/20+0.1% diethylamine, 1 ml/min—diode array detection at 230 nm—(8R)-enantiomer: 12.4 min/1.9 area-%, (8S)-enantiomer: 13.6 min/96.4 area-%, 96.1% ee

¹H-NMR (DMSO-d₆, 200 MHz): δ=2.15 (m_{c}, 2H), 2.47, 2.55 (s, m_{c}), 2.79, 2.83 (s, m_{c}, 4H), 3.01 (s, 3H), 3.68 (s, 3H), 5.44 (dd, 1H), 6.94 (s, 1H), 7.27 (t, 2H), 7.44 (m_{c}, 1H), 7.56 (t, 1H).

### G. (8S)-8-(4-Fluoro-phenyl)-2,3-dimethyl-3,6,7,8-tetrahydro-chromeno[7,8-d]imidazole-5-carboxylic Acid Dimethylamide Hydrochloride

To a suspension of (3R)-6-[3-(4-fluoro-phenyl)-3-hydroxy-propyl]-7-hydroxy-2,3-dimethyl-3H-benzoimidazole-5-carboxylic acid dimethylamide (example 6, 1.8 g, 4.7 mmol) in tetrahydrofuran (60 ml), triphenylphosphine (3.6 g, 13.5 mmol) and DIAD (2.9 ml, 14.5 mmol) were added and the mixture was stirred for 3 h at room temperature. The reaction was concentrated in vacuo in the presence of silica gel and the residue was purified by flash chromatography on silica gel (Dichloromethane/Methanol=100:0 to 87:13) and crystallized from a mixture of acetone and a saturated solution of HCl in diethyl ether to afford two batches of the title compound (batch 1: 0.81 g of a colourless solid, 43% yield, batch 2: 0.36 g of a colourless solid, 19% yield, 82% ee, m.p. 290-292° C.). A batch of the free base of the title compound was obtained by purification of the mother liquor (flash chromatography on silica gel, Dichloromethane/Methanol=20:1): 0.50 g of a colourless solid (30% yield).

Determination of the optical purity by CE: MT [(8S)-enantiomer]=20.1 min/90.1 area-%: MT [(8R)-enantiomer]=21.0 min/9.0 area-%; 81.8% ee.

¹H-NMR (DMSO-d₆, 200 MHz): δ=2.13 (m_{c}, 1H), 2.33 (m_{c}, 1H), 2.75, 2.77, 2.79 (m_{c}, 2 s, 8H), 3.04 (s, 3H), 3.89 (s, 3H), 5.42 (dd, 1H), 7.28 (t, 2H), 7.41 (s, 1H), 7.62 (dd, 2H).

### H. (8S)-2,3-Dimethyl-8-o-tolyl-3,6,7,8-tetrahydro-chromeno[7,8-d]imidazole-5-carboxylic Acid Dimethylamido

Method A: To a solution of (3R)-7-hydroxy-6-(3-hydroxy-3-o-tolyl-propyl)-2,3-dimethyl-3H-benzoimidazole-5-carboxylic acid dimethylamide (example 7, 4.8 g, 12.6 mmol) in tetrahydrofuran (60 ml) was added triphenylphosphine (6.1 g, 36.5 mmol) and DIAD (7.7 ml, 39 mmol) and the mixture was stirred for 16.75 h at room temperature. The reaction was concentrated in vacuo in the presence of silica gel and the crude product was purified by flash chromatography (first column, Dichloromethane/Methanol=20:1, second column: Dichloromethane/Methanol=100:0 to 88:12, third column: Toluene/1,4-Dioxane=1:1) to afford 2.3 g (50% yield, 87% ee) of the title compound as a beige foam.

[α]²⁰_{D}=−14° (c=0.50, methanol)

Determination of the optical purity by CE: MT [(8R)-enantiomer]=38.9 min 6.3 area-%; MT [(8S)-enantiomer]=46.9 min/93.7 area-%; 87.4% ee.

¹H-NMR (DMSO-d₆, 200 MHz): δ=1.99 (m, 1H), 2.22 (m_{c}, 1H), 2.38 (s, 3H), 2.47 (s), 2.65 (m, 1H), 2.81, 2.85 (s, m_{c}, 4H), 3.02 (s, 3H), 3.68 (s, 3H), 5.32 (dd, 1H), 6.92 (s, 1H), 7.24 (m_{c}, 3H), 7.47 (m_{c}, 1H).

Method B: To a suspension of (3R)-7-hydroxy-6-(3-hydroxy-3-o-tolyl-propyl)-2,3-dimethyl-3H-benzoimidazole-5-carboxylic acid dimethylamide (example 7, 26.0 g, 68.1 mmol) and triphenylphosphine (35.0 g, 133 mmol) in dry tetrahydrofuran (600 ml), DIAD (27.7 ml, 27.0 g, 134 mmol) was added over a period of 10 min. A yellow solution was obtained, which was stirred for 5 min at room temperature and concentrated under reduced pressure. The residue was purified by column chromatography on silica gel (Dichloromethane/Methanol=100:2). Evaporation of the corresponding fractions afforded the title compound in 84% corrected yield (23.0 g of a colourless foam containing 10 weight-% of ethyl acetate, 95.6% ee).—After intense drying in vacuo, an amorphous solid was obtained: m.p. 126-128° C.

HPLC analytical method: column: Daicel Chiralpak AD-H, 250×4.6 mm, 5 μm—eluant: n-heptane/ethanol: 80/20, flow rate: 1 ml/min, detection wavelength: 218 nm—first eluting enantiomer: 10.2 min/2.2 area-%, second eluting enantiomer: 13.7 min/97.8 area-%, 95.6% ee.

### Ha. (8S)-2,3-Dimethyl-8-o-tolyl-3,6,7,8-tetrahydro-chromeno[7,8-d]imidazole-5-carboxylic acid dimethylamide; salt with hydrochloric acid

(8S)-2,3-Dimethyl-8-o-tolyl-3,6,7,8-tetrahydro-chromeno[7,8-d]imidazole-5-carboxylic acid dimethylamide (1.00 g, 2.75 mmol) was dissolved in a 0.1 N solution of hydrochloric acid in isopropanol (30 ml, 3.0 mmol) The solvent was evaporated and a colourless foam was isolated (1.1 g). A part of the foam (500 mg) was treated with diethyl ether (6 ml) and the resulting suspension was stirred for 30 min at room temperature. The precipitate was isolated by filtration, washed with diethyl ether (4 ml), and dried in vacuo. This afforded 470 mg of a colourless solid (99% corrected yield, m.p. 145° C.).

¹H-NMR (DMSO-d₆, 200 MHz): δ=2.08 (m_{c}, 1H), 2.32 (m_{c}, 1H), 2.42 (s, 3H), 2.71, 2.78, 2.83 (m_{c}, 2 s, 7H), 3.01, 3.06 (m_{c}, s, 4H), 3.90 (s, 3H), 5.54 (dd, 1H), 7.30 (m_{c}, 3H), 7.42 (s, 1H), 7.52 (m_{c}, 1H).

### Hb. (8S)-2,3-Dimethyl-8-o-tolyl-3,6,7,8-tetrahydro-chromeno[7,8-d]imidazole-5-carboxylic acid dimethylamide; salt with maleic acid

(8S)-2,3-Dimethyl-8-o-tolyl-3,6,7,8-tetrahydro-chromeno[7,8-d]imidazole-5-carboxylic acid dimethylamide (1.00 g, 2.75 mmol) was dissolved in hot acetone (2 ml) and a solution of maleic acid (0.35 g, 3.01 mmol) in acetone (5 ml) was added at a temperature of 60° C. The solution was allowed to cool to room temperature. The solvent was evaporated and a colourless foam was isolated (1.4 g). A part of the foam (500 mg) was treated with diethyl ether (6 ml) and the resulting suspension was stirred for 30 min at room temperature. The precipitate was isolated by filtration, washed with diethyl ether (5 ml), and dried in vacuo. This afforded 450 mg of a colourless solid (95% corrected yield, m.p. 110-112

¹H-NMR (DMSO-d₆, 200 MHz): δ=2.08 (m_{c}, 1H), 2.28 (m_{c}, 1H), 2.40 (s, 3H), 2.63, 2.70 (s, m_{c}, 4H), 2.82 (s, 3H), 2.98, 3.04 (m_{c}, s, 4H), 3.81 (s, 3H), 5.46 (dd, 1H), 6.14 (s, 2H), 7.24 (s, 1H), 7.29 (m_{c}, 3H), 7.49 (m_{c}, 1H).

### Hc. (8S)-2,3-Dimethyl-8-o-tolyl-3,6,7,8-tetrahydro-chromeno[7,8-d]imidazole-5-carboxylic acid dimethylamide; salt with fumaric acid

(8S)-2,3-Dimethyl-8-o-tolyl-3,6,7,8-tetrahydro-chromeno[7,8-d]imidazole-5-carboxylic acid dimethylamide (1.00 g, 2.75 mmol) was dissolved in hot acetone (2 ml) and a solution of fumaric acid (0.35 g, 3.01 mmol) in acetone (3 ml) was added at a temperature of 60 AC. The solution was allowed to cool to room temperature. The solvent was evaporated and a colourless foam was isolated (1.4 g). A part of the foam (500 mg) was treated with diethyl ether (5 ml) and the resulting suspension was stirred for 15 min at room temperature. The precipitate was isolated by filtration, washed with diethyl ether (3 ml), and dried in vacuo. This afforded 460 mg of a colourless solid (98% corrected yield, m.p. 118-120° C.).

¹H-NMR (DMSO-d₆, 200 MHz): δ=2.02 (m_{c}, 1H), 2.22 (m_{c}, 1H), 2.38 (s, 3H), 2.47 (s, 3H), 2.64 (m_{c}, 1H), 2.81, 2.87 (s, m_{c}, 4H), 3.02 (s, 3H), 3.68 (s, 3H), 5.33 (dd, 1H), 6.63 (s, 2H), 6.93 (s, 1H), 7.26 (m_{c}, 3H), 7.47 (m_{c}, 1H).

### Hd. (8S)-2,3-Dimethyl-8-o-tolyl-3,6,7,8-tetrahydro-chromeno[7,8-d]imidazole-5-carboxylic acid dimethylamide; salt with oxalic acid

(8S)-2,3-Dimethyl-8-o-tolyl-3,6,7,8-tetrahydro-chromeno[7,8-d]imidazole-5-carboxylic acid dimethylamide (1.00 g, 2.75 mmol) was dissolved in hot acetone (2 ml) and a solution of oxalic acid (0.27 g, 3.00 mmol) in acetone (1 ml) was added at a temperature of 60° C. The solution was allowed to cool to room temperature. The solvent was evaporated and a colourless foam was isolated (1.3 g). A part of the foam (500 mg) was treated with diethyl ether (3 ml) and acetone (0.3 ml) and the resulting suspension was stirred for 30 min at room temperature. The precipitate was isolated by filtration, washed with diethyl ether (2 ml), and dried in vacuo. This afforded 480 mg of a colourless solid (99% corrected yield, m.p. 112° C.).

¹H-NMR (DMSO-d₆, 200 MHz): δ=2.02 (m_{c}, 1H), 2.25 (m_{c}, 1H), 2.38 (s, 3H), 2.53, 2.63 (s, m_{c}), 2.81, 2.88 (s, m_{c}, 4H), 3.03 (s, 3H), 3.72 (s, 3H), 5.37 (dd, 1H), 7.05 (s, 1H), 7.27 (m_{c}, 3H), 7.47 (m_{c}, 1H).

### He. (8S)-2,3-Dimethyl-8-o-tolyl-3,6,7,8-tetrahydro-chromeno[7,8-d]imidazole-5-carboxylic acid dimethylamide; salt with citric acid

(8S)-2,3-Dimethyl-8-o-tolyl-3,6,7,8-tetrahydro-chromeno[7,8-d]imidazole-5-carboxylic acid dimethylamide (1.00 g, 2.75 mmol) was dissolved in hot acetone (2 ml) and a solution of citric acid (0.58 g, 3.02 mmol) in acetone (4 ml) was added at a temperature of 60° C. The solution was allowed to cool to room temperature. The solvent was evaporated and a colourless foam was isolated (1.6 g). A part of the foam (500 mg) was treated with diethyl ether (5 ml) and the resulting suspension was stirred for 30 min at room temperature. The precipitate was isolated by filtration, washed with diethyl ether (3 ml), and dried in vacuo. This afforded 250 mg of a colourless solid (52% corrected yield, m.p. 105° C.).

¹H-NMR (DMSO-d₆, 200 MHz): δ=1.98 (m_{c}, 1H), 2.22 (m_{c}, 1H), 2.38 (s, 3H), 2.49 (s), 2.70, 2.80, 2.81 (dd, m_{c}, s, 9H), 3.02 (s, 3H), 3.70 (s, 3H), 5.34 (dd, 1H), 6.97 (s, 1H), 7.27 (m_{c}, 3H), 7.47 (m_{c}; 1H).

### Hf. (8S)-2,3-Dimethyl-8-o-tolyl-3,6,7,8-tetrahydro-chromeno[7,8-d]imidazole-5-carboxylic acid dimethylamide; salt with methanesulfonic acid

(8S)-2,3-Dimethyl-8-o-tolyl-3,6,7,8-tetrahydro-chromeno[7,8-d]imidazole-5-carboxylic acid dimethylamide (1.00 g, 2.75 mmol) was dissolved in hot acetone (2 ml) and a solution of methanesulfonic acid (0.29 g, 3.02 mmol) in acetone (0.5 ml) was added at a temperature of 60° C. The solution was allowed to cool to room temperature. The solvent was evaporated and a colourless foam was isolated (1.3 g). A part of the foam (500 mg) was treated with diethyl ether (5 ml) and acetone (0.5 ml) and the resulting suspension was stirred for 15 min at room temperature. The precipitate was isolated by filtration, washed with diethyl ether (3 ml), and dried in vacuo. This afforded 450 mg of a colourless solid (93% corrected yield, m.p. 114-116° C.).

¹H-NMR (DMSO-d₆, 200 MHz): δ=2.12 (m_{c}, 1H), 2.33, 2.37 (m_{c}, s, 7H), 2.42 (s, 3H), 2.49 (s), 2.75, 2.76, 2.83 (m_{c}, 2 s, 7H), 3.00, 3.06 (m_{c}, s, 4H), 3.90 (s, 3H), 5.55 (dd, 1H), 7.31 (m_{c}, 3H), 7.43 (s, 1H), 7.52 (m_{c}, 1H).

### I. (8S)-8-(2-Chloro-phenyl)-2,3-dimethyl-3,6,7,8-tetrahydro-chromeno[7,8-d]imidazol-5-carboxylic Acid Dimethylamide

To a solution of (3R)-6-[3-(2-chloro-phenyl)-3-hydroxy-propyl]-7-hydroxy-2,3-dimethyl-3H-benzoimidazole-5-carboxylic acid dimethylamide (example 8, 200 mg, 0.50 mmol) in tetrahydrofuran (10 ml) was added triphenylphosphine (260 mg, 0.99 mmol) and DIAD (197 μl, 202 mg, 1.00 mmol) and the green solution was stirred for 5 min at room temperature. The reaction mixture was concentrated in vacuo and the crude product was purified by flash chromatography (Dichloromethane/Methanol=100:3) to afford 175 mg (91% yield, 89.6% ee) of the title compound as a colourless foam.

[α]²⁰_{D}=−93° (c=0.55, MeOH)

HPLC analytical method: column: Daicel Chiralpak AD-H, 250×4.6 mm, 5 μm—eluant: n-heptane/ethanol: 90/10, flow rate: 1 ml/min, detection wavelength: 218 nm—first eluting enantiomer: 28.5 min/5.2 area-%, second eluting enantiomer: 31.3 min/94.8 area-%, 89.6% ee.

¹H-NMR (DMSO-d₆, 200 MHz): δ=1.99 (m_{c}, 1H), 2.30 (m_{c}, 1H), 2.52 (s), 2.64 (bs, 1H), 2.80, 2.88 (s, m_{c}, 4H), 3.01 (s; 3H), 3.69 (s, 3H), 5.47 (dd, 1H), 6.96 (s, 1H), 7.46 (m_{c}, 3H), 7.63 (m_{c}, 1H).

### J. (8S-2,3-Dimethyl-8-(2-trifluoromethyl-phenyl)-3,6,7,8-tetrahydro-chromeno[7,8-d]imidazole-5-carboxylic Acid Dimethylamide

To a solution of (3R)-7-hydroxy-6-[3-hydroxy-3-(2-trifluoromethyl-phenyl)-propyl]-2,3-dimethyl-3H-benzoimidazole-5-carboxylic acid dimethylamide (example 9, 110 mg, 0.25 mmol) in tetrahydrofuran (10 ml) was added triphenylphosphine (115 mg, 0.44 mmol) and DIAD (88 μl, 90 mg, 0.44 mmol) and the mixture was stirred for 30 min at room temperature. Another portion of DIAD (20 μl, 21 mg, 0.10 mmol) was added and stirring was continued for 30 min. The reaction was concentrated in vacuo and the crude product was purified by column chromatography on silica gel (Dichloromethane/Methanol 40:1). This afforded 20 mg (19% yield, 85.3% ee) of the title compound as a brown wax.

HPLC analytical method: column: Daicel Chiralpak AD-H, 250×4.6 mm, 5 μm—eluant: n-heptane/ethanol: 80/20, flow rate: 1 ml/min, detection wavelength: 218 nm—first eluting enantiomer: 7.3 min/6.6 area-%, second eluting enantiomer: 8.6 min/83.1 area-%, 85.3% ee.

¹H-NMR (DMSO-d₆, 400 MHz): δ=2.07 (m_{c}, 1H), 2.20 (m_{c}, 1H), 2.46 (s, 3H), 2.67 (m_{c}, 1H), 2.82, 2.88 (s, m_{c}, 4H), 3.02 (s, 3H), 3.69 (s, 3H), 5.34 (d, 1H), 6.98 (s, 1H), 7.60 (m_{c}), 7.88 (m_{c}).

### K. (8S)-2,3-Dimethyl-8-naphthalen-2-yl-3,6,7,8-tetrahydro-chromeno[7,8-d]imidazole-5-carboxylic Acid Dimethylamide Hydrochloride

To a solution of (3R)-7-hydroxy-6-(3-hydroxy-3-naphthalen-2-yl-propyl)-2,3-dimethyl-3H-benzoimidazole-5-carboxylic acid dimethylamide (example 10, 150 mg, 0.36 mmol) in tetrahydrofuran (10 ml) was added triphenylphosphine (277 mg, 1.04 mmol) and DIAD (221 μl, 1.12 mmol) and the mixture was stirred for 4.25 h at room temperature. The reaction was concentrated in vacuo and the crude product was purified by flash chromatography on silica gel (Dichloromethane/Methanol=20:1). The residue obtained on evaporation of the corresponding fractions was dissolved in acetone and a 2 M solution of hydrochloric acid in diethyl ether was added. The precipitate was isolated by filtration and was dried in vacuo. This afforded 101 mg (64% yield, 38.7% ee) of the title compound as a beige solid.—m.p. 262-264° C.

[α]²⁰_{D}=−34° (c=0.49, chloroform)

HPLC analytical method: column: Daicel Chiralpak AD-H, 250×4.6 mm, 5 μm—eluant: n-heptane/ethanol: 80/20+0.1% diethylamine, flow rate: 1 ml/min, detection wavelength: 230 nm—first eluting enantiomer: 25.3 min/30.4 area-%, second eluting enantiomer: 31.2 min/68.7 area-%, 38.7% ee.

¹H-NMR (DMSO-d₆, 200 MHz): δ=2.25 (m_{c}, 1H), 2.45 (m_{c}), 2.72, 2.78, 2.81 (m_{c}, s, s, 7H), 3.01, 3.05 (m_{c}, s, 4H), 3.90 (s, 3H), 5.59 (dd, 1H), 7.43 (s, 1H), 7.56 (m_{c}, 2H), 7.70 (m_{c}, 1H), 7.99 (m_{c}, 3H), 8.14 (s, 1H).

### L. (8S)-(2-Ethyl-phenyl)-2,3-dimethyl-3,6,7,8-tetrahydro-chromeno[7,8-d]imidazole-5-carboxylic Acid Dimethylamide

To a solution of (3R)-6-[3-(2-ethyl-phenyl)-3-hydroxy-propyl]-7-hydroxy-2,3-dimethyl-3H-benzoimidazole-5-carboxylic acid dimethylamide (example 11, 130 mg, 0.33 mmol) in tetrahydrofuran (10 ml) was added triphenylphosphine (172 mg, 0.66 mmol) and DIAD (127 μl, 130 mg, 0.64 mmol) and the green solution was stirred for 5 min at room temperature. The reaction mixture was concentrated in vacuo and the crude product was purified by flash chromatography (Dichloromethane/Methanol=100:3) to afford 90 mg (73% yield, 83.4% ee) of the title compound as a colourless foam.

HPLC analytical method: column: Daicel Chiralpak AD-H, 250×4.6 mm, 5 μm—eluant: n-heptane/ethanol: 80/20, flow rate: 1 ml/min, detection wavelength: 218 nm—first eluting enantiomer: 8.3 min 8.3 area-%, second eluting enantiomer: 9.5 min/91.7 area-%, 83.4% ee.

¹H-NMR (DMSO-d₆, 200 MHz): δ=1.21 (t, 3H), 1.90-2.30 (m_{c}, 2H), 2.46 (s, 3H), 2.55-2.95, 2.73, 2.82 (m_{c}, dq, s, 7H), 3.02 (s, 3H), 3.67 (s, 3H), 5.34 (dd, 1H), 6.92 (s, 1H), 7.29 (m_{c}, 3H), 7.48 (m_{c}, 1H).

### M. (8S)-2,3-Dimethyl-8-thiophen-2-yl-3,6,7,8-tetrahydro-chromeno[7,8-d]imidazole-5-carboxylic Acid Dimethylamide

To a solution of (3R)-7-hydroxy-6-(3-hydroxy-3-thiophen-2-yl-propyl)-2,3-dimethyl-3H-benzoimidazole-5-carboxylic acid dimethylamide (example 12, 450 mg, 1.20 mmol) in tetrahydrofuran (30 ml) was added triphenylphosphine (600 mg, 2.25 mmol) and DIAD (470 μl, 483 mg, 2.38 mmol) and the red-brown solution was stirred for 45 min at room temperature. The solvent was evaporated in the presence of silica gel and the residue was loaded on top of a column filled with silica gel. The title compound (260 mg of a colourless foam, 61% yield, 77.2% ee) was eluted with a mixture of Dichloromethane and Methanol [30:1 (v/v)].

HPLC analytical method: column: Daicel Chiralpak AD-H, 250×4.6 mm, 5 μm—eluant: n-hexane/isopropanol: 80/20, flow rate: 1 ml min, detection wavelength: 218 nm—first eluting enantiomer: 12.3 min/81.5 area-%, second eluting enantiomer: 16.9 min/10.5 area-%, 77.2% ee.

¹H-NMR (DMSO-d₆, 400 MHz): δ=2.16 (m_{c}, 1H), 2.35 (m_{c}, 1H), 2.47 (s, 3H), 2.65 (m_{c}, 1H), 2.76, 2.79 (s, m_{c}, 4H), 3.01 (s, 3H), 3.67 (s, 3H), 5.50 (dd, 1H), 6.92 (s, 1H), 7.07 (m_{c}, 1H), 7.20 (m_{c}, 1H), 7.55 (m_{c}, 1H).

### N. (8S)-8-(4-Fluoro-2-methyl-phenyl)-2,3-dimethyl-3,6,7,8-tetrahydro-chromeno[7,8-d]imidazole-5-carboxylic Acid Dimethylamide

To a solution of (3R)-6-[3-(4-fluoro-2-methyl-phenyl)-3-hydroxy-propyl]-7-hydroxy-2,3-dimethyl-3H-benzoimidazole-5-carboxylic acid dimethylamide (example 13, 630 mg, 1.58 mmol) in tetrahydrofuran (20 ml) was added triphenylphosphine (415 mg, 1.58 mmol) and DIAD (633 μl, 650 mg, 3.20 mmol) and the green solution was stirred for 30 min at room temperature. The reaction mixture was concentrated in vacuo and the crude product was purified by flash chromatography (Dichloromethane/Methanol=100:3) to afford 290 mg (48% yield, 83.0% ee) of the title compound as a colourless foam.

[α]²⁰_{D}=−15° (c=0.41, MeOH)

HPLC analytical method: column: Daicel Chiralpak AD-H, 250×4.6 mm, 5 μm—eluant: n-heptane/ethanol: 80/20, flow rate: 1 ml/min, detection wavelength: 218 nm—first eluting enantiomer: 12.4 min/8.5 area-%, second eluting enantiomer: 17.5 min/91.5 area-%, 83.0% ee.

¹H-NMR (DMSO-d₆, 200 MHz): δ=1.99 (m_{c}, 1H), 2.21 (m_{c}, 1H), 2.40 (s, 3H), 2.47 (s, 3H), 2.57-2.74 (m, 1H), 2.74-2.96, 2.80 (m, s, 4H), 3.02 (s, 3H), 3.68 (s, 3H), 5.30 (dd, 1H), 6.93 (s, 1H), 7.09 (m_{c}, 2H), 7.49 (m_{c}, 1H).

### O. (8S)-(2,3-Dimethyl-8-o-tolyl-3,6,7,8-tetrahydro-chromeno[7,8-d]imidazol-5-yl)-pyrrolidin-1-yl-methanone

To a solution of (3R)-[7-hydroxy-6-(3-hydroxy-3-o-tolyl-propyl)-2,3-dimethyl-3H-benzoimidazol-5-yl]-pyrrolidin-1-yl-methanone (example 14, 750 mg, 1.8 mmol) in tetrahydrofuran (50 ml) was added triphenylphosphine (1.40 g, 5.3 mmol) and DIAD (1.10 ml, 1.13 g, 5.6 mmol) and the mixture was stirred for 5 h at room temperature. The reaction was concentrated in vacuo and the crude product was purified by flash chromatography on silica gel (Dichloromethane/Methanol=20:1). The residue obtained on evaporation of the corresponding fractions was dissolved in acetone and a 2 M solution of hydrochloric acid in diethyl ether was added. The solution was stirred for 17 h at room temperature and was concentrated in vacuo. A beige solid was isolated, which was dissolved in acetone. After addition of diethyl ether, a precipitate was formed, which was isolated by filtration and dried in vacuo. This afforded 401 mg of a beige solid (hydrochloride salt of the title compound, 51% yield).

Note: The title compound can be purified further by preparative HPLC using a GROM Saphire C8 column, 125×20 mm, 65 Å pore diameter, 5 μm particle size.—beige solid, m.p. 127-128° C., 79.8% ee

[α]²⁰_{D}=−19° (c=0.50, chloroform)

HPLC analytical method: column: Daicel Chiralpak AD-H, 250×4.6 mm, 5 μm—eluant: n-heptane/ethanol: 80/20, flow rate: 1 ml/min, detection wavelength: 218 nm—first eluting enantiomer: 12.7 min/10.1 area-%, second eluting enantiomer: 20.0 min/89.9 area-%, 79.8% ee.

¹H-NMR (DMSO-d₆, 400 MHz): δ=1.92 (m_{c}, 5H), 2.21 (m_{c}, 1H), 2.38 (s, 3H), 2.47 (s, 3H), 2.66 (m_{c}, 1H), 2.93 (m_{c}, 1H), 3.07 (m_{c}, 1H), 3.21 (m, 1H), 3.48 (m_{c}, 2H), 3.68 (s, 3H), 5.32 (dd, 1H), 6.98 (s, 1H), 7.26 (m_{c}, 3H), 7.47 (m_{c}, 1H).

### P. (8S)-2,3-Dimethyl-8-o-tolyl-3,6,7,8-tetrahydro-chromeno[7,8-d]imidazole-5-carboxylic Acid Methylamide

To a suspension of (3R)-7-hydroxy-6-(3-hydroxy-3-o-tolyl-propyl)-2,3-dimethyl-3H-benzoimidazole-5-carboxylic acid methylamide (example 15, 270 mg, 0.73 mmol) in tetrahydrofuran (10 ml) was added triphenylphosphine (564 mg, 2.12 mmol) and DIAD (446 μl, 2.26 mmol) and the obtained solution was stirred at room temperature. In the course of 18 h a precipitate was formed, which was isolated by filtration. The title compound was obtained in 35% yield (89 mg of a colourless solid, 97.5% ee).—m.p. 259-260° C.

[α]²⁰_{D}=−4°(c=0.40, chloroform)

HPLC analytical method: column: Daicel Chiralpak AD-H, 250×4.6 mm, 5 μm—eluant: n-heptane/ethanol: 80/20+0.1% diethylamine, flow rate: 1 ml/min, detection wavelength: 218 nm—first eluting enantiomer: 6.0 min/1.3 area-%, second eluting enantiomer: 7.9 min/98.7 area-%, 97.5% ee.

¹H-NMR (DMSO-d₆, 400 MHz): δ=1.94 (m_{c}, 1H), 2.22 (m_{c}, 1H), 2.38 (s, 3H), 2.47 (s, 3H), 2.77, 2.83 (d, m_{c}, 4H), 3.12 (m_{c}, 1H), 3.69 (s, 3H), 5.29 (dd, 1H), 7.12 (s, 1H), 7.27 (m_{c}, 3H), 7.45 (m_{c}, 1H), 8.09 (q, 1H).

### Q. (8S)-Azetidin-1-yl-(2,3-dimethyl-8-o-tolyl-3,6,7,8-tetrahydro-chromeno[7,8-d]imidazol-5-yl)-methanone

To a solution of (3R)-azetidin-1-yl-[7-hydroxy-6-(3-hydroxy-3-o-tolyl-propyl)-2,3-dimethyl-3H-benzoimidazol-5-yl]-methanone (example 16, 65 mg, 0.17 mmol) in tetrahydrofuran (5 ml) was added triphenylphosphine (105 mg, 0.40 mmol) and DIAD (79 μl, 81 mg, 0.40 mmol) and the green solution was stirred for 5 min at room temperature. The reaction mixture was concentrated in vacuo and the crude product was purified by flash chromatography (Dichloromethane/Methanol=100:3) to afford 55 mg (86% yield, 94.8% ee) of the title compound as a colourless foam.

HPLC analytical method: column: Daicel Chiralpak AD-H, 250×4.6 mm, 5 μm—eluant: n-heptane/ethanol: 80/20, flow rate: 1 ml min, detection wavelength: 218 nm—first eluting enantiomer: 13.0 min/2.6 area-%, second eluting enantiomer: 21.7 min/97.4 area-%, 94.8% ee.

¹H-NMR (DMSO-d₆, 200 MHz): δ=1.95 (m_{c}, 1H), 2.22 (m_{c}, 3H), 2.38 (s, 3H), 2.47 (s, 3H), 2.82 (m_{c}, 1H), 3.04 (m_{c}, 1H), 3.69 (s, 3H), 3.88 (m_{c}, 1H), 4.05 (m_{c}, 3H), 5.31 (dd, 1H), 7.05 (s, 1H), 7.26 (m_{c}, 3H), 7.47 (m_{c}, 1H).

### R. (8S)-8-(2-Benzyloxymethyl-phenyl)-2,3-dimethyl-3,6,7,8-tetrahydro-chromeno[7,8-d]imidazole-5-carboxylic acid dimethylamide

To a solution of (3R-6-[3-(2-benzyloxymethyl-phenyl)-3-hydroxy-propyl]-7-hydroxy-2,3-dimethyl-3H-benzoimidazole-5-carboxylic acid dimethylamide (example 17, 50 mg, 0.0 mmol) in tetrahydrofuran (4 ml) was added triphenylphosphine (50 mg, 0.19 mmol) and DIAD (40 μl, 41 mg, 0.20 mmol) and the solution was stirred for 30 min at room temperature. Another portion of DIAD (20 μl, 21 mg, 0.10 mmol) was added and stirring was continued for 1 h. The reaction mixture was concentrated in vacuo in the presence of silica gel and the residue was loaded on top of a column filled with silica gel. The title compound (25 mg of a colourless foam, 53% yield, 95.9% ee) was eluted with mixtures of dichloromethane and methanol (50:1 then 30:1).

HPLC analytical method: column: Daicel Chiralpak AD-H, 250×4.6 mm, 5 μm—eluant: n-hexane/isopropanol 80/20, flow rate: 1 ml/min, detection wavelength: 218 nm—first eluting enantiomer: 11.4 min/96.1 area-%, second eluting enantiomer: 13.6 min/2.0 area-%, 95.9% ee.

¹H-NMR (DMSO-d₆, 400 MHz): δ=1.98 (m_{c}, 1H), 2.21 (m_{c}, 1H), 2.46 (s, 3H), 2.55 (m_{c}), 2.80 (s, bs, 4H), 3.02 (s, 3H), 3.68 (s, 3H), 4.55 (s, 2H), 4.63 (d, 1H), 4.72 (d, 1H), 5.36 (d, 1H), 6.93 (s, 1H), 7.36 (m_{c}, 8H), 7.56 (d, 1H).

### S. (8S)-8-(2-Methoxymethyl-phenyl)-2,3-dimethyl-3,6,7,8-tetrahydro-chromeno[7,8-d]imidazole-5-carboxylic acid dimethylamide

To a solution of (3R)-7-hydroxy-6-[3-hydroxy-3-(2-methoxymethyl-phenyl)-propyl]-2,3-dimethyl-3H-benzoimidazole-5-carboxylic acid dimethylamide (example 18, 900 mg, 2.19 mmol) in tetrahydrofuran (40 ml) was added triphenylphosphine (1.14 g, 4.3 mmol) and DIAD (885 μl, 903 mg, 4.46 mmol) and the brown solution was stirred for 10 min at room temperature. The reaction mixture was concentrated in vacuo and the crude product was purified by flash chromatography (Dichloromethane/Methanol=20:1). Evaporation of the corresponding fractions and treatment of the residue (700 mg) with diethyl ether afforded a light brown foam, which was slurried in diisopropyl ether. The title compound (430 mg of a colourless solid, 50% yield, 97.9% ee) was isolated by filtration.—m.p. 200° C.

[α]²⁰_{D}=−9° (c=0.51, CH₂Cl₂/MeOH=1:1)

HPLC analytical method: column: Daicel Chiralpak AD-H, 250×4.6 mm, 5 μm—eluant: n-hexane/isopropanol: 90/10, flow rate: 1 ml/min, detection wavelength: 218 nm—first eluting enantiomer: 46.1 min/1.0 area-%, second eluting enantiomer: 48.8 min/97.6 area-%, 97.9% ee.

¹H-NMR (DMSO-d₆, 200 MHz): δ=1.99 (m_{c}, 1H), 2.23 (m_{c}, 1H), 2.46 (s, 3H), 2.64 (m_{c}, 1H), 2.81, 2.85 (s, m_{c}, 4H), 3.02 (s, 3H), 3.30 (s), 3.68 (s, 3H), 4.56 (dd, 2H), 5.36 (dd, 1H), 6.93 (s, 1H), 7.38 (m_{c}, 3H), 7.65 (m_{c}, 1H).

### T. (8S)-2-Methyl-8-o-tolyl-3-(2-trimethylsilanyl-ethoxymethyl)-3,6,7,8-tetrahydro-chromeno[7,8-d]imidazole-5-carboxylic acid dimethylamide

To a solution of (3R)-7-hydroxy-6-(3-hydroxy-3-o-tolyl-propyl)-2-methyl-3-(2-trimethylsilanyl-ethoxymethyl)-3H-benzoimidazole-5-carboxylic acid dimethylamide (example 19, 300 mg, 0.60 mmol) in tetrahydrofuran (10 ml) was added triphenylphosphine (300 mg, 1.14 mmol) and DIAD (240 μl, 245 mg, 1.21 mmol) and the solution was stirred for 10 min at room temperature. The reaction mixture was concentrated in vacuo and the crude product was purified by flash chromatography (first column: Dichloromethane/Methanol=40:1, second column: Petrol ether/Ethyl acetate=1:1 to 1:3). Evaporation of the corresponding fractions afforded 430 mg of a mixture of the title compound (30 weight-%, 45% corrected yield) and triphenylphosphine oxide (70 weight-%):

¹H-NMR (DMSO-d₆, 400 MHz): δ=−0.08 (s, 9H), 0.83 (t, 2H), 1.99 (m_{c}), 2.24 (m_{c}, 1H), 2.39 (s, 3H), 2.50 (s), 2.67 (bs, 1H), 2.80 (s, 3H), 2.90 (bs, 1H), 3.02 (s, 3H), 3.52 (t, 2H), 5.35 (dd, 1H), 5.54 (s, 2H), 7.06 (s, 1H), 7.27 (m_{c}, 3H), 7.48 (m_{c}, 1H), triphenylphospine oxide: 7.59 (m_{c}).

### U. (8S)-2-Methyl-8-o-tolyl-3,6,7,8-tetrahydro-chromeno[7,8-d]imidazole-5-carboxylic acid dimethylamide

At a temperature of 0° C., borontrifluoride etherate (290 μl, 325 mg, 2.3 mmol) was added drop-wise to a solution of (8S)-2-methyl-8-o-tolyl-3-(2-trimethylsilanyl-ethoxymethyl)-3,6,7,8-tetrahydro-chromeno[7,8-d]imidazole-5-carboxylic acid dimethylamide (example T, 400 mg, 30 weight-%, 0.25 mmol) in dichloromethane (10 ml). The reaction mixture was stirred for 3 h at room temperature and the solvent was evaporated in the presence of silica gel. The residue was loaded on top of a column filled with silica gel and the title compound was eluted with dichloromethane/methanol=50:1. Evaporation of the corresponding fractions afforded the title compound (110 mg, quant. yield, 96.1% ee).—m.p. 219° C.

[α]²⁰_{D}=−9° (c=0.44, CH₂Cl₂/MeOH=1:1)

HPLC analytical method: column: Daicel Chiralpak AD-H, 250×4.6 mm; 5 μm—eluant: n-hexane/isopropanol: 90/10, flow rate: 1 ml/min detection wavelength: 218 nm—first eluting enantiomer: 21.9 min/97.7 area-%, second eluting enantiomer: 32.5 min/1.9 area-%, 96.1% ee.

¹H-NMR (DMSO-d₆, 200 MHz): δ=2.04 (m_{c}, 1H), 2.26 (m_{c}, 1H), 2.40 (s, 3H), 2.53, 2.63 (s, m_{c}), 2.81 (s, 3H), 2.90, 3.02 (m_{c}, s, 4H), 5.43 (dd, 1H), 6.99 (s, 1H), 7.28 (m_{c}, 3H), 7.50 (m_{c}, 1H).

### V. (8S)-2,3-Dimethyl-8-(2-methyl-thiophen-3-yl)-3,6,7,8-tetrahydro-chromeno[7,8-d]imidazole-5-carboxylic acid dimethylamide

To a suspension of (3R)-7-hydroxy-6-[3-hydroxy-3-(2-methyl-thiophen-3-yl)-propyl]-2,3-dimethyl-3H-benzoimidazole-5-carboxylic acid dimethylamide (example 21, 0.54 g, 1.4 mmol) in tetrahydrofuran (20 ml) was added triphenylphosphine (0.70 g, 2.7 mmol) and DIAD (564 mg, 2.8 mmol) and the solution was stirred for 10 min at room temperature. The reaction mixture was concentrated in vacuo and the crude product (2.5 g of a brown oil) was purified by column chromatography (Ethyl acetate, then Ethyl acetate/Methanol=9:1). Evaporation of the corresponding fractions afforded a yellow solid, which was slurried in diethyl ether (5 ml). After a period of 10 minutes at room temperature, the precipitate was isolated by filtration, washed with diethyl ether (2 ml), and dried in vacuo. The title compound was isolated in 39% yield (200 mg of a colourless solid).—m.p. 233° C.

¹H-NMR (DMSO-d₆, 200 MHz): δ=2.20 (m_{c}, 2H), 2.47 (s, 3H), 2.57 (s, 3H), 2.88 (s, bs, 5H), 3.15 (s, 3H), 3.67 (s, 3H), 5.27 (dd, 1H), 6.76 (s, 1H), 7.03 (m_{c}, 2H).

### W. (8S)-2,3-Dimethyl-8-o-tolyl-3,6,7,8-tetrahydro-chromeno[7,8-d]imidazole-5-carboxylic acid cyclopropylamide

To a suspension of (3R)-7-hydroxy-6-(3-hydroxy-3-o-tolyl-propyl)-2,3-dimethyl-3H-benzoimidazole-5-carboxylic acid cyclopropylamide (example 22, 0.32 g, 0.81 mmol) in tetrahydrofuran (20 ml) was added triphenylphosphine (0.41 g, 1.6 mmol) and DIAD (328 mg, 1.62 mmol) and the solution was stirred for 10 min at room temperature. The reaction mixture was concentrated in vacuo and the crude product (1.6 g of a brown solid) was purified by column chromatography on silica gel (Ethyl acetate, then Ethyl acetate/Methanol=9:1). Evaporation of the corresponding fractions afforded a yellow solid (0.26 g), which was slurried in diethyl ether (3 ml). After a period of 10 minutes at room temperature, the precipitate was isolated by filtration, washed with diethyl ether (2 ml), and dried in vacuo. The title compound was isolated in 66% yield (200 mg of a colourless solid, 97.5% ee).—m.p. 289° C.

HPLC analytical method: column: Daicel Chiralpak AD-H, 250×4.6 mm, 5 μm—eluant: n-heptane/ethanol: 80/20, flow rate: 1 ml/min, detection wavelength: 218 nm—first eluting enantiomer: 6.0 min/1.2 area-%, second eluting enantiomer: 7.9 min/97.5 area-%, 97.5% ee.

¹H-NMR (CDCl₃, 200 MHz): δ=0.64 (m_{c}, 2H), 0.90 (m_{c}, 2H), 2.08 (m_{c}, 1H), 2.26 (m_{c}, 1H), 2.37 (s, 3H), 2.56 (s, 3H), 2.98 (m_{c}, 2H), 3.22 (m_{c}, 1H), 3.68 (s, 3H), 5.39 (dd, 1H), 6.02 (bs, 1H), 6.94 (s, 1H), 7.19 (m_{c}, 3H), 7.56 (m_{c}, 1H).

### X. 5-Methoxymethyl-2,3-dimethyl-8-o-tolyl-3,6,7,8-tetrahydro-chromeno[7,8-d]imidazole

To a solution of (3R)-5-(3-hydroxy-3-o-tolyl-propyl)-6-methoxymethyl-1,2-dimethyl-1H-benzoimidazol-4-ol (example 23, 800 mg, 2.26 mmol) in tetrahydrofuran (40 ml) was added triphenylphosphine (1.20 g, 4.6 mmol) and DIAD (900 μl, 918 mg, 4.5 mmol) and the solution was stirred for 30 min at room temperature. The reaction mixture was concentrated in vacuo in the presence of silica gel. A column filled with silica gel was charged with the residue and a mixture of the title compound with triphenylphosphine oxide was eluted with ethyl acetate. After further purification by column chromatography on silica gel (Ethyl acetate/Petrol ether=3:2, then Ethyl acetate) the title compound was obtained in the form of a colourless solid (600 mg containing 11 mol-% of triphenylphosphine oxide, 79% yield, 95.4% ee).

HPLC analytical method: column: Daicel Chiralpak AD-H, 250×4.6 mm, 5 μm—eluant: n-hexane/isopropanol: 95/5, flow rate: 1 ml/min, detection wavelength: 218 nm—first eluting enantiomer: 30.0 min/2.3 area-%, second eluting enantiomer: 37.7 min/97.7 area-%, 95.4% ee.

¹H-NMR (DMSO, 200 MHz): δ=1.99 (m_{c}, 1H), 2.26 (m_{c}, 1H), 2.38 (s, 3H), 2.45 (s, 3H), 2.94 (m_{c}, 2H), 3.33 (s, 3H), 3.67 (s, 3H), 4.48 (s, 2H), 5.24 (dd, 1H), 7.02 (s, 1H), 7.27 (m_{c}, 3H), 7.49 (m_{c}, 1 H), 7.62 (m_{c}, triphenylphosphine oxide).

### Synthesis of Prochiral Ketones of the Formula 2

### a. 7-Hydroxy-2-methyl-6-(3-oxo-3-phenyl-propyl)-3-(2-trimethylsilanyl-ethoxymethyl)-3H-benzoimidazole-5-carboxylic Acid Dimethylamide

A solution of 7-hydroxy-2-methyl-3-(2-trimethylsilanyl-ethoxymethyl)-3H-benzoimidazole-5-carboxylic acid dimethylamide (example b, 10.0 g, 28.6 mmol) in dichloromethane (600 ml) was treated with N,N-dimethyl-methyleneiminium iodide (6.4 g, 34.3 mmol) and the reaction was stirred for 3 h at room temperature. The reaction mixture was poured into saturated sodium hydrogencarbonate solution and extracted twice with dichloromethane. The organic layers were dried over magnesium sulfate and concentrated in vacuo. The residue (11.3 g, 98%) was suspended in toluene (250 ml) and 1-(1-phenyl-vinyl)-pyrrolidine (CAS 3433-56-5, 7.2 g, 41.6 mmol) was added. The suspension was refluxed for 3 h and after cooling to room temperature, the solvent was evaporated in vacuo. The residue was purified by flash chromatography on silica gel (Ethyl acetate/Petroleum ether=5:1) to afford 10.6 g of a beige solid which was dissolved in acetone and treated with fumaric acid. The precipitate was filtered, dissolved in dichloromethane-methanol and the solution was neutralized with 1 M NaOH solution. The layers were separated, the organic layer was dried over magnesium sulfate and concentrated in vacuo. This afforded 9.7 g (73% yield) of the title compound as a brown solid.—m.p. 192-194° C.

### b. 7-Hydroxy-2-methyl-3-(2-trimethylsilanyl-ethoxymethyl)-3H-benzolmidazole-5-carboxylic Acid Dimethylamide

A solution of 7-benzyloxy-2-methyl-3-(2-trimethylsilanyl-ethoxymethyl)-3H-benzoimidazole-5-carboxylic acid dimethylamide (example c, 13.7 g, 31.1 mmol) in ethanol (1.2 l) was hydrogenated over 10% Pd/C (1.4 g) in an autoclave (5 bar H₂) for 16 h at room temperature. The catalyst was filtered off and the filtrate was concentrated in vacuo. The residue was crystallized from diisopropyl ether to afford 10.1 g (93% yield) of the title compound as a white solid.—m.p. 154-156° C.

### c. 7-Benzyloxy-2-methyl-3-(2-trimethylsilanyl-ethoxymethyl)-3H-benzoimidazole-5-carboxylic Acid Dimethylamide

4-Benzyloxy-6-bromo-2-methyl-1-(2-trimethylsilanyl-ethoxymethyl)-1H-benzoimidazole (example d, 19.5 g, 43.6 mmol), triphenylphosphine (4.6 g, 17.9 mmol), palladium(II) acetate (1.5 g, 6.5 mmol) and dimethylamine solution (2 M in THF, 218 ml, 436 mmol) were transferred to an autoclave and carbonylated (6 bar CO) for 60 h at 120° C. The catalyst was filtered off and the filtrate was concentrated in vacuo. The residue was purified by flash chromatography on silica gel (Toluene/Dioxane=2:1) to afford 13.8 g (72% yield) of the title compound as a white solid.—m.p. 118-120° C.

### d. 4-Benzyloxy-6-bromo-2-methyl-(2-trimethylsilanyl-ethoxymethyl)-1-H-benzoimidazole

To a suspension of 4-benzyloxy-6-bromo-2-methyl-1H-benzoimidazole (36.5 g, 115 mmol) and triethylamine (17.7 ml, 138 mmol) in a dimethylformamide-dichloromethane mixture (10:1) was added drop-wise (2-chloromethoxy-ethyl)-trimethylsilane (24.5 ml, 138 mmol) and the suspension was stirred for 5 h at room temperature. The reaction was poured into water and extracted with dichloromethane (3×). The combined organic layers were dried over magnesium sulfate and concentrated in vacuo. The residue was purified by flash chromatography on silica gel (Toluene/Dioxane=9:1) to afford 19.5 g (39% yield) of the title compound as a white solid.—m.p. 94-95° C.

### e. 3-[6-(Azetidine-1-carbonyl)-4-hydroxy-1,2-dimethyl-1H-benzoimidazol-5-yl]-1-phenyl-propan-1-one

A suspension of azetidin-1-yl-(8-methoxy-2,3-dimethyl-8-phenyl-3,6,7,8-tetrahydro-chromeno[7,8-d]imidazol-5-yl)-methanone (example f, 4.7 g, 12 mmol) in THF (75 ml) was treated with 1 N hydrochloric acid (30 ml) and the mixture was heated to 50° C. for 2 h. After cooling to room temperature, the reaction mixture was cautiously poured into water (100 ml) and neutralized with 2 M NaOH. The precipitate was filtered and dried in vacuo to afford 2.9 g (65% yield) of the title compound as a white solid.—m.p. 242-243° C.

### f. Azetidin-1-yl-(8-methoxy-2,3-dimethyl-8-phenyl-3,6,7,8-tetrahydro-chromeno[7,8-d]imidazol-5-yl)-methanone

A solution of 8-methoxy-2,3-dimethyl-8-phenyl-3,6,7,8-tetrahydro-chromeno[7,8-d]imidazole-5-carboxylic acid (example 1, 6 g, 17 mmol) in dimethylformamide (60 ml) was treated with TBTU (6.5 g, 20.4 mmol), DIPEA (7.3 ml, 42.6 mmol) and was stirred for 1 h at room temperature. Azetidine (2 ml. 29 mmol) was added and the reaction mixture was stirred for 2 h at room temperature. The mixture was poured into water (400 ml), the precipitate was filtered and dried in vacuo to afford 4.8 g (72% yield) of the title compound as a beige solid.—m.p. 147-150° C.

### g. 7-Hydroxy-2,3-dimethyl-6-(3-oxo-3-phenyl-propyl)-3H-benzoimidazole-5-carboxylic Acid Methylamide

A suspension of 8-methoxy-2,3-dimethyl-8-phenyl-3,6,7,8-tetrahydro-chromeno[7,8-d]imidazole-5-carboxylic acid methylamide (example h, 5.1 g, 14 mmol) in THF (75 ml) was treated with 1 N hydrochloric acid (30 ml) and the mixture was heated to 50° C. for 3 h. After cooling to 5° C., the reaction mixture was cautiously poured into water (100 ml) and neutralized with 2 M NaOH. The precipitate was filtered and dried in vacuo to afford 4.4 g (90% yield) of the title compound as a white solid.—m.p. 284-285° C.

### h. 8-Methoxy-2,3-dimethyl-8-phenyl-3,6,7,8-tetrahydro-chromeno[7,8-d]imidazole-5-carboxylic Acid Methylamide

A solution of 8-methoxy-2,3-dimethyl-8-phenyl-3,6,7,8-tetrahydro-chromeno[7,8-d]imidazole-5-carboxylic acid (example 1, 6.0 g, 17 mmol) in dimethylformamide (60 ml) was treated with TBTU (6.5 g, 20.4 mmol), DIPEA (7.3 ml, 42.6 mmol) and was stirred for 1 h at room temperature. Methylamine solution (2 M in THF, 11.5 ml, 29 mmol) was added and the reaction mixture was stirred for 2 h at room temperature. The mixture was poured into water (400 ml), the precipitate was filtered and dried in vacuo to afford 5.2 g (84% yield) of the title compound as a white solid.—m.p. 237-239° C.

### i. 8-Methoxy-2,3-dimethyl-8-phenyl-3,6,7,8-tetrahydro-chromeno[7,8-d]imidazole-5-carboxylic Acid

To a suspension of 8-methoxy-2,3-dimethyl-8-phenyl-3,6,7,8-tetrahydro-chromeno[7,8-d]imidazole-5-carboxylic acid ethyl ester (example j, 13.1 g, 34.4 mmol) in methanol (290 ml) was added a 2 M solution of potassium hydroxide in water (35 ml) and the mixture was heated to 55° C. for 16 h. After cooling to room temperature, the solvent was removed in vacuo and the residue was suspended in water (300 ml). The suspension was adjusted to pH 5-6 by adding 2M aq. HCl solution and the solution was stirred for 1 h at room temperature. The precipitate was isolated by filtration and dried in vacuo to afford 12 g (99% yield) of the title compound as a beige solid.—m.p. 288-289° C.

### j. 8-Methoxy-2,3-dimethyl-8-phenyl-3,6,7,8-tetrahydro-chromeno[7,8-d]imidazole-5-carboxylic Acid Ethyl Ester

2,2-Dimethoxypropane (70 ml, 570 mmol) was added to a solution of 7-hydroxy-2,3-dimethyl-6-(3-oxo-3-phenyl-propyl)-3H-benzoimidazole-5-carboxylic acid ethyl ester (13.9 g, 37.9 mmol) in dichloromethane (170 ml). After slow addition of methanesulfonic acid (3.2 ml, 49.3 mmol), the obtained red solution was refluxed for 16 h. After cooling to room temperature, the reaction mixture was poured into a mixture of 170 ml saturated sodium hydrogencarbonate solution (170 ml) and dichloromethane (140 ml). The phases were separated and the aqueous layer was extracted with dichloromethane (2×50 ml). The collected organic layers were dried over magnesium sulfate and concentrated in vacuo. The residue was crystallized from diisopropyl ether to afford 13.2 g (92% yield) of the title compound as a beige solid.—m.p. 176-178° C.

### k. 6-[3-(2-Fluoro-phenyl)-3-oxo-propyl]-7-hydroxy-2,3-dimethyl-3H-benzoimidazole-5-carboxylic Acid Dimethylamide

6-Dimethylaminomethyl-7-hydroxy-2,3-dimethyl-3H-benzoimidazole-5-carboxylic acid dimethylamide (8.5 g, 29.2 mmol) was suspended in toluene (130 ml) and the suspension was heated to 50° C. A solution of 1-[1-(2-fluorophenyl)-vinyl]-pyrrolidine (CAS 237436-15-6, 8.3 g, 43.9 mmol) in 20 ml toluene was slowly added and the mixture was heated to 100° for 1 h. After cooling to room temperature, the solvent was evaporated in vacuo. The residue was dissolved in methanol (100 ml) and treated with fumaric acid. After stirring for 1 h, the precipitate was filtered and dissolved in dichloromethane-water (1:1). The solution was adjusted to pH 8 by adding 2 N aqueous NaOH solution. The layers were separated, the organic layer was dried over magnesium sulfate and concentrated in vacuo. The residue was crystallized from diethyl ether to afford 3.8 g (36% yield) of the title compound as a white solid.—m.p. 218-221° C.

### l. 6-[3-(4-Fluoro-phenyl)-3-oxo-propyl]-7-hydroxy-2,3-dimethyl-3H-benzoimidazole-5-carboxylic Acid Dimethylamide

6-Dimethylaminomethyl-7-hydroxy-2,3-dimethyl-3H-benzoimidazole-5-carboxylic acid dimethylamide (8.9 g, 30.6 mmol) was suspended in toluene (200 ml) and treated with 1-[1-(4-fluorophenyl)-vinyl]-pyrrolidine (CAS 237436-54-3, 8.8 g, 46 mmol). The reaction mixture was refluxed for 6 h. After cooling to room temperature, the solvent was evaporated in vacuo. The residue was purified by flash chromatography on silica gel (Dichloromethane/Methanol=20:1) and crystallized from acetone to afford 5.2 g (44% yield) of the title compound as a white solid.—m.p. 248-249° C.

### m. 7-Hydroxy-2,3-dimethyl-6-(3-oxo-3-o-tolyl-propyl)-3H-benzoimidazole-5-carboxylic Acid Dimethylamide

6-Dimethylaminomethyl-7-hydroxy-2,3-dimethyl-3H-benzoimidazole-5-carboxylic acid dimethylamide (11.3 g, 32.8 mmol) was suspended in toluene (350 ml) and treated with 1-[1-(2-methylphenyl)-vinyl]-pyrrolidine (CAS 156004-72-7, 11.4 g, 60.8 mmol). The reaction mixture was refluxed for 4 h. After cooling to room temperature, the solvent was evaporated in vacuo. The residue was purified by flash chromatography on silica gel (Dichloromethane/Methanol=10:1) and then dissolved in acetone (70 ml). Fumaric acid (3 g) was added and the solution was stirred overnight at room temperature. The precipitate was isolated by filtration, dissolved in dichloromethane and washed with aqueous saturated sodium hydrogen carbonate solution. The phases were separated, the organic layer was dried over magnesium sulfate and concentrated in vacuo. This afforded 6.7 g (55% yield) of the title compound as a brown foam.

¹H-NMR (DMSO-d₆, 200 MHz): δ=2.42 (s, 3H), 2.48 (s), 2.77, 2.80, 3.00, 3.05 (s, bm_{c}, s, bm_{c}, 10H), 3.67 (s, 3H), 6.78 (s, 1H), 7.30 (m_{c}, 2H), 7.42 (m_{c}, 1H), 7.70 (m_{c}, 1H), 10.00 (bs, 1H).

### n. 6-[3-(2-Chloro-phenyl)-3-oxo-propyl]-7-hydroxy-2,3-dimethyl-3H-benzoimidazole-5-carboxylic Acid Dimethylamide

A suspension of 6-dimethylaminomethyl-7-hydroxy-2,3-dimethyl-3H-benzoimidazole-5-carboxylic acid dimethylamide (2.5 g, 8.6 mmol) in 1,2-dimethoxyethane (80 ml) was heated to 80° C. and 1-[1-(2-chlorophenyl)-vinyl]-pyrrolidine (CAS 237436-24-7, 2.9 g, 14.0 mmol) was added over a period of 15 min. The reaction mixture was kept at 80° C. for 3.5 h. After cooling to room temperature, the solvent was evaporated in vacuo. The residue was dissolved in acetone, fumaric acid (1.0 g) was added, and the solution was stirred overnight at room temperature. The precipitate was isolated by filtration and was washed with hot isopropanol. The salt of the title compound with fumaric acid was dissolved in dichloromethane and aqueous saturated sodium hydrogen carbonate solution. The phases were separated, the organic layer was dried over magnesium sulfate and concentrated in vacuo. The residue was purified by flash chromatography on silica gel (Dichloromethane/Methanol=20:1). This afforded 1.55 g (45% yield) of the title compound as a colourless foam.

¹H-NMR (DMSO-d₆, 200 MHz): δ=2.50 (s), 2.76, 2.81 (s, m_{c}, 5H), 2.98, 3.08 (s, m_{c}, 5H), 3.66 (s, 3H), 6.77 (s, 1H), 7.51 (m_{c}, 4H), 9.99 (bs, 1H).

### o. 7-Hydroxy-2,3-dimethyl-6-[3-oxo-3-(2-trifluoromethyl-phenyl)-propyl]-3H-benzoimidazole-5-carboxylic Acid Dimethylamide

A suspension of 6-dimethylaminomethyl-7-hydroxy-2,3-dimethyl-3H-benzoimidazole-5-carboxylic acid dimethylamide (4.3 g, 14.8 mmol) and 1-[1-(2-trifluoromethyl-phenyl)-vinyl]-pyrrolidine (CAS 237436-26-9, 5.7 g, 23.6 mmol) in toluene (200 ml) was heated to reflux for 3 h. After cooling to room temperature, the solvent was evaporated in vacuo. The residue was dissolved in acetone, fumaric acid (1.7 g) was added, and the solution was stirred overnight at room temperature. No precipitate was formed. The solution was concentrated in vacuo and the residue was purified by flash chromatography on silica gel (Dichloromethane/Methanol=20:1) and subsequent crystallization from isopropanol. This afforded the title compound in 54% yield (3.49 g of a beige solid).—m.p. 204-206° C.

### p. 7-Hydroxy-2,3-dimethyl-6-(3-naphthalen-2-yl-3-oxo-propyl)-3H-benzoimidazole-5-carboxylic Acid Dimethylamido

A suspension of 6-dimethylaminomethyl-7-hydroxy-2,3-dimethyl-3H-benzoimidazole-5-carboxylic acid dimethylamide (2.6 g, 9.0 mmol) in toluene (80 ml) was heated to 40° C. A solution of 1-(1-naphthalen-2-yl-vinyl)-pyrrolidine (CAS 156004-71-6, 3.45 g, 15.4 mmol) in toluene (40 ml) was added and the reaction mixture was heated to 93° C. After a period of 2.5 h, the solution was cooled to room temperature and concentrated in vacuo. The residue was dissolved in acetone (100 ml), fumaric acid (1.2 g) was added, and the solution was stirred overnight at room temperature. The precipitate was isolated by filtration and was washed with acetone (2×20 ml). The salt of the title compound with fumaric acid was dissolved in dichloromethane (100 ml) and aqueous ammonia was added until a pH-value of 9 was reached. The phases were separated and the aqueous phase was extracted with dichloromethane (3×50 ml). The combined organic phases were dried over magnesium sulfate and concentrated in vacuo. The residue was purified by flash chromatography on silica gel (Dichloromethane/Methanol=15:1) and crystallization from acetone. This afforded 0.48 g (13% yield) of the title compound as a colourless solid.—m.p. 221-225° C.

### q. 6-[3-(2-Ethyl-phenyl)-3-oxo-propyl]-7-hydroxy-2,3-dimethyl-3H-benzolmidazole-5-carboxylic Acid Dimethylamide

A suspension of 6-dimethylaminomethyl-7-hydroxy-2,3-dimethyl-3H-benzoimidazole-5-carboxylic acid dimethylamide (3.0 g, 10.3 mmol) in 1,2-dimethoxyethane (100 ml) was heated to 80° C. and a solution of 1-[1-(2-ethylphenyl)-vinyl]-pyrrolidine (synthesis described below, 3.3 g, 16.4 mmol) in DME was added over a period of 15 min. The reaction mixture was kept at 80° C. for 3 h. After cooling to room temperature, the solvent was evaporated in vacuo. The residue was dissolved in acetone, fumaric acid (1.2 g) was added, and the solution was stirred for 3 d at room temperature.

(a) The precipitate was isolated by filtration and was washed with hot isopropanol. The salt of the title compound with fumaric acid was dissolved in dichloromethane and aqueous saturated sodium hydrogen carbonate solution. The phases were separated, the organic layer was dried over magnesium sulfate and concentrated in vacuo. This afforded 1.1 g (27% yield) of the title compound as a colourless foam.

(b) The mother liquor was concentrated and the residue was purified by flash chromatography on silica gel (Toluene/1,4-Dioxane=1:1) and subsequent crystallization in the presence of citric acid (solvent: acetone). The salt of the title compound with citric acid was dissolved in dichloromethane and aqueous saturated sodium hydrogen carbonate solution. The phases were separated, the organic layer was dried over magnesium sulfate and concentrated in vacuo. This afforded another 0.58 g (14% yield) of the title compound as a beige solid.

Note: The title compound can be purified further by preparative HPLC: column: GROM Saphire C8 125×20 mm, 65 Å pore diameter, 5 μm particle size, solvent gradient: ammonium formiate buffer (pH 3.75)/acetonitrile=97:3 (v/v) to 5:95 (v/v), flow rate: 30 ml/min, run time: 16 min.—m.p. 159°-160° C.

¹H-NMR (DMSO-d₆, 200 MHz): δ=1.14 (t, 3H), 2.50, 2.52 (bs, s), 2.75, 2.75, 2.77 (q, bs, s 6H), 2.99, 3.04 (s, bs, 5H), 3.67 (s, 3H), 6.78 (s, 1H), 7.30 (m_{c}, 2H), 7.44 (m_{c}, 1H), 7.62 (d, 1H), 9.98 (bs, 1H).

### r. 7-Hydroxy-2,3-dimethyl-6-(3-oxo-3-thiophen-2-yl-propyl)-3H-benzoimidazole-5-carboxylic Acid Dimethylamide

A suspension of 6-dimethylaminomethyl-7-hydroxy-2,3-dimethyl-3H-benzoimidazole-5-carboxylic acid dimethylamide (2.5 g, 8.6 mmol) in 1,2-dimethoxyethane (80 ml) was heated to 80° C. and a solution of 1-(1-thiophen-2-yl-vinyl)-pyrrolidine (synthesis described below, 2.5 g, 13.9 mmol) in DME was added over a period of 15 min. The reaction mixture was kept at 80° C. for 3 h. After cooling to room temperature, the solvent was evaporated in vacuo. The crude product was purified by flash chromatography on silica gel (Dichloromethane/Methanol=20:1) and subsequent crystallization from isopropanol (2×). This afforded the title compound in 12% yield (391 mg of a beige solid). The mother liquor was concentrated and the residue was purified by flash chromatography on silica gel (Toluene/1,4-Dioxane=1:1) and subsequent crystallization in the presence of citric acid. The salt of the title compound with citric acid was dissolved in dichloromethane and aqueous saturated sodium hydrogen carbonate solution. The phases were separated, the organic layer was dried over magnesium sulfate and concentrated in vacuo. This afforded another 324 mg (10% yield) of the title compound in the form of a brown foam.

Note: The title compound can be purified further by preparative HPLC: column: GROM Saphire C8 125×20 mm, 65 Å pore diameter, 5 μm particle size, solvent gradient: ammonium formiate buffer (pH 3.75)/acetonitrile=97:3 (v/v) to 5:95 (v/v), flow rate: 30 ml/min, run time: 16 min.—m.p. 233-235° C.

¹H-NMR (DMSO-dc, 200 MHz): δ=2.50, 2.53 (bs, s), 2.78 (bs, s, 4H), 3.01, 3.09 (s, bs, 5H), 3.68 (s, 3H), 6.80 (s, 1H), 7.25 (m_{c}, 1H), 7.93 (m_{c}, 1H), 7.99 (m_{c}, 1H), 10.10 (bs, 1H).

### s. 6-[3-(4-Fluoro-2-methyl-phenyl)-3-oxo-propyl]-7-hydroxy-2,3-dimethyl-3H-benzoimidazole-5-carboxylic Acid Dimethylamide

A suspension of 6-dimethylaminomethyl-7-hydroxy-2,3-dimethyl-3H-benzoimidazole-5-carboxylic acid dimethylamide (2.50 g, 8.6 mmol) in 1,2-dimethoxyethane (80 ml) was heated to 80° C. and a solution of 1-[1-(4-fluoro-2-methylphenyl)-vinyl]-pyrrolidine (synthesis described below, 2.8 g, 13.6 mmol) in DME was added over a period of 15 min. The reaction mixture was kept at 80° C. for 3.5 h. After cooling to room temperature, the solvent was evaporated in vacuo. The residue was dissolved in acetone, fumaric acid (1.0 g) was added, and the solution was stirred for 17 h at room temperature. The precipitate was isolated by filtration and washed with hot isopropanol. The salt of the title compound with fumaric acid was dissolved in dichloromethane and aqueous saturated sodium hydrogen carbonate solution. The phases were separated, the organic layer was dried over magnesium sulfate and concentrated in vacuo. The residue was washed with diisopropyl ether and purified by flash chromatography on silica gel (Dichloromethane/Methanol=20:1). Evaporation of the corresponding fractions afforded a colourless solid, which was washed with a mixture of isopropanol and diisopropyl ether. The title compound was obtained in 44% yield (1.49 g of a colourless solid).

¹H-NMR (DMSO-d₆, 200 MHz): δ=2.45 (s, 3H), 2.48 (s, bs), 2.75, 2.77 (bs, s, 4H), 3.00, 3.05 (s, bs, 5H), 3.67 (s, 3H), 6.78 (s, 1H), 7.14 (m_{c}, 2H), 7.83 (m_{c}, 1H), 9.98 (bs, 1H).

### t. 7-Hydroxy-2,3-dimethyl-6-(3-oxo-3-o-tolyl-propyl)-3H-benzolmidazole-5-carboxylic acid ethyl ester

A suspension of 6-dimethylaminomethyl-7-hydroxy-2,3-dimethyl-3H-benzoimidazole-5-carboxylic acid ethyl ester (45.0 g, 0.15 mol) in DME (600 ml) was heated to 85° and a solution of 1-[1-(2-methylphenyl)-vinyl]-pyrrolidine (46.0 g, 0.25 mol) in DME (100 ml) was added drop-wise. The red reaction mixture was stirred for 4 h at 85° C. and was concentrated under reduced pressure in the presence of silica gel. A column filled with 1 kg of silica gel was charged with the solid residue and the title compound was eluted with a mixture of Dichloromethane and Methanol [30:1 (v/v)]. Evaporation of the corresponding fractions furnished two batches of the title compound, which were slurried in hot isopropanol. Filtration of the first batch afforded 16.0 g of a colourless solid (pure title compound, 27% yield, m.p. 183° C.). The precipitate isolated by filtration of the second batch was treated with another portion of hot isopropanol. After filtration, 14.7 g of a pink solid was obtained (mixture of the title compound and 7-hydroxy-2,3-dimethyl-3H-benzoimidazole-5-carboxylic acid ethyl ester, (molar ratio 85:15, 23% corrected yield).

### u. 8-Methoxy-2,3-dimethyl-8-o-tolyl-3,6,7,8-tetrahydro-chromeno[7,8-d]imidazole-5-carboxylic acid ethyl ester

2,2-Dimethoxypropane (135.7 ml, 1097 mmol) was added to a solution of 7-hydroxy-2,3-dimethyl-6-(3-oxo-3-o-tolyl-propyl)-3H-benzoimidazole-5-carboxylic acid ethyl ester (example t, 28.0 g, 73.6 mmol) in dichloromethane (350 ml). After slow addition of methanesulfonic acid (6.2 ml, 95.5 mmol), the mixture was refluxed for 3 d. After cooling to room temperature, the reaction mixture was poured onto saturated sodium hydrogencarbonate solution. The biphasic mixture was stirred for 10 min. The phases were separated and the aqueous layer was extracted with dichloromethane (2×). The collected organic layers were dried over magnesium sulfate and concentrated in vacuo. The residue was crystallized from diisopropyl ether to afford 27.8 g (96% yield) of the title compound as a beige solid.—m.p. 198° C.

### v. 8-Methoxy-2,3-dimethyl-8-o-tolyl-3,6,7,8-tetrahydro-chromeno[7,8-d]imidazole-5-carboxylic acid

To a suspension of 8-methoxy-2,3-dimethyl-8-o-tolyl-3,6,7,8-tetrahydro-chromeno[7,8-d]imidazole-5-carboxylic acid ethyl ester (example u, 27.7 g, 70.2 mmol) in methanol (300 ml) and 1,4-dioxane (300 ml) was added a 2 M solution of sodium hydroxide in water (140 ml) and the mixture was heated to reflux for 2 h. After cooling to room temperature, the reaction mixture was poured into water and a pH value of 5-6 was adjusted by addition of concentrated hydrochloric acid. A precipitate was formed, which was isolated by filtration and dried in vacuo to afford 22.45 g (87% yield) of the title compound as a beige solid. The mother liquor was concentrated and the pH value was re-adjusted to 5-6. Isolation of the precipitate afforded another batch of the title compound (3.2 g of a beige solid, 12% yield)—m.p. 304-309° C.

### w. (8-Methoxy-2,3-dimethyl-8-o-tolyl-3,6,7,8-tetrahydro-chromeno[7,8-d]imidazol-5-yl)-pyrrolidin-1-yl-methanone

A suspension of 8-methoxy-2,3-dimethyl-8-o-tolyl-3,6,7,8-tetrahydro-chromeno[7,8-d]imidazole-5-carboxylic acid (example v, 2.00 g, 5.5 mmol) in DMF (50 ml) was treated with TBTU (2.1 g, 6.5 mmol) and DIPEA (2.40 ml, 1.78 g, 12.8 mmol). The reaction mixture was stirred for 30 min at room temperature and pyrrolidine (767 μl, 660 mg, 9.3 mmol) was added. Stirring was continued for 17 h at room temperature and the reaction was poured onto saturated ammonium chloride solution. After a period of 1 h, the precipitate was isolated by filtration and dried in vacuo (40° C.). The title compound was isolated in the form of a beige solid (1.57 g, 69% yield).—m.p. 202-204° C.

### x. 3-[4-Hydroxy-1,2-dimethyl-6-(pyrrolidine-1-carbonyl)-1H-benzoimidazol-5-yl]-1-o-tolyl-propan-1-one

Hydrochloric acid (8.40 ml of a 1 M solution in water, 8.4 mmol) was added to a suspension of (8-methoxy-2,3-dimethyl-8-o-tolyl-3,6,7,8-tetrahydro-chromeno[7,8-d]imidazol-5-yl)-pyrrolidin-1-yl-methanone (example w, 1.40 g, 3.3 mmol) in THF (40 ml). A solution was obtained, which was stirred for 1 d at 50° C. The reaction mixture was allowed to come to room temperature, poured onto ice water and neutralized by addition of aqueous sodium hydroxide solution (2 N). The precipitate was isolated by filtration and dried in vacuo (40° C.). This afforded 1.0 g of the title compound (colourless solid, 74% yield). The mother liquor was extracted with dichloromethane. The combined organic phases were dried over magnesium sulfate and concentrated in vacuo. The residue was purified by flash chromatography on silica gel (Dichloromethane/Methanol=15:1). Evaporation of the corresponding fractions furnished another 104 mg of the title compound (8% yield).—m.p. 216-217° C.

### y. 8-Methoxy-2,3-dimethyl-8-o-tolyl-3,6,7,8-tetrahydro-chromeno[7,8-d]imidazole-5-carboxylic acid methylamide

A suspension of 8-methoxy-2,3-dimethyl-8-o-tolyl-3,6,7,8-tetrahydro-chromeno[7,8-d]imidazole-5-carboxylic acid (example v, 2.00 g, 5.5 mmol) in DMF (50 ml) was treated with DIPEA (2.45 ml, 1.85 g, 14.3 mmol) and TBTU (2.2 g, 6.9 mmol). The yellow solution was stirred for 20 mill at room temperature and methylamine (7.1 ml of a 2 M solution in THF, 14.2 mmol) was added. Stirring was continued for 5 h at room temperature. The reaction mixture was poured onto saturated ammonium chloride solution (250 ml) and extracted with dichloromethane (3×200 ml). The combined organic phases were dried over magnesium sulfate and evaporated to dryness. The yellow-brown solid residue was crystallized from acetone (20 ml). This afforded the title compound in 82% yield (1.7 g of colourless crystals). The mother liquor was purified by flash chromatography on silica gel (Dichloromethane/Methanol=9:1) and subsequent crystallization from acetone. This afforded another 0.16 g of the title compound (8% yield)—m.p. 257-261° C.

### z. 7-Hydroxy-2,3-dimethyl-6-(3-oxo-3-o-tolyl-propyl)-3H-benzoimidazole-5-carboxylic Acid Methylamide

Hydrochloric acid (18.0 ml of a 1 M solution in water, 18 mmol) was added to a suspension of 8-methoxy-2,3-dimethyl-8-o-tolyl-3,6,7,8-tetrahydro-chromeno[7,8-d]imidazole-5-carboxylic acid methylamide (example y, 1.70 g, 4.5 mmol) in THF (40 ml). A solution was obtained, which was stirred at 50° C. After a period of 3 h, more hydrochloric acid (10.0 ml of a 1 M solution in water, 10 mmol) was added and stirring was continued for 3 h at 50° C. The reaction mixture was allowed to come to room temperature, poured onto ice water (100 ml), neutralized by addition of aqueous sodium hydroxide solution (2 N), and extracted with dichloromethane (3×150 ml). The combined organic phases were dried over magnesium sulfate and concentrated in vacuo. The solid residue was crystallized from a mixture of diethyl ether (100 ml) and acetone (10 ml). This afforded the title compound in 79% yield (1.3 g of beige crystals).—m.p. 224-227° C.

### aa. Azetidin-1-yl-(8-methoxy-2,3-dimethyl-8-o-tolyl-3,6,7,8-tetrahydro-chromeno[7,8-d]imidazol-5-yl)-methanone

A suspension of 8-methoxy-2,3-dimethyl-8-o-tolyl-3,6,7,8-tetrahydro-chromeno[7,8-d]imidazole-5-carboxylic acid (example v, 2.00 g, 5.5 mmol) in DMF (50 ml) was treated with DIPEA (2.45 ml, 1.85 g, 14.3 mmol) and TBTU (2.2 g, 6.9 mmol). The yellow solution was stirred for 20 min at room temperature and azetidine (700 μl, 592 mg, 10.4 mmol) was added. Stirring was continued for 1 d at room temperature. The reaction mixture was poured onto saturated ammonium chloride solution (200 ml) and extracted with dichloromethane (3×200 ml). The combined organic phases were dried over magnesium sulfate and evaporated to dryness. A yellow oil was obtained, which was purified by flash chromatography on silica gel (Dichloromethane/Methanol=9:1) and subsequent crystallization from diethyl ether (50 ml). This afforded the title compound in 72% yield (1.6 g of beige crystals).—m.p. 209-211° C.

### bb. 3-[6-(Azetidine-1-carbonyl)-4-hydroxy-1,2-dimethyl-1H-benzoimidazol-5-yl]-1-o-tolyl-propan-1-one

Hydrochloric acid (18.0 ml of a 1 M solution in water, 18 mmol) was added to a suspension of azetidin-1-yl-(8-methoxy-2,3-dimethyl-8-o-tolyl-3,6,7,8-tetrahydro-chromeno[7,8-o]imidazol-5-yl)-methanone (example aa, 1.60 g, 3.9 mmol) in THF (46 ml). A yellow solution was obtained, which was stirred at 50° C. After a period of 3 h, more hydrochloric acid (10.0 ml of a 1 M solution in water, 10 mmol) was added and stirring was continued for 3 h at 50° C. The reaction mixture was allowed to come to room temperature, poured onto ice water (100 ml), neutralized by addition of aqueous sodium hydroxide solution (2 N), and extracted with dichloromethane (3×150 ml). The combined organic phases were dried over magnesium sulfate and concentrated in vacuo. The solid residue was crystallized from a mixture of diethyl ether (100 ml) and acetone (10 ml). This afforded the title compound in 78% yield (1.20 g of colourless crystals).—m.p. 229-232° C.

### cc. 6-[3-(2-Benzyloxy-phenyl)-3-oxo-propyl]-7-hydroxy-2,3-dimethyl-3H-benzoimidazole-5-carboxylic acid dimethylamide

A suspension of 6-dimethylaminomethyl-7-hydroxy-2,3-dimethyl-3H-benzoimidazole-5-carboxylic acid dimethylamide (9.20 g, 31.7 mmol) in 1,2-dimethoxyethane (300 ml) was heated to 85° C. and a solution of 1-[1-(2-benzyloxymethyl-phenyl)-vinyl]-pyrrolidine (synthesis described below, 15.0 g, 51 mmol) in DME (15 ml) was added slowly. The reaction mixture was kept at 85° C. for 3 h and was stirred at room temperature for 17 h. The solvent was evaporated in vacuo. The residue was dissolved in isopropanol (300 ml). A precipitate was formed, which was isolated by filtration and washed with isopropanol. The title compound was purified further by flash chromatography on silica gel (Dichloromethane/Methanol=20:1). Evaporation of the corresponding fractions furnished a beige solid (5.3 g, 34% yield).—m.p. 193-194° C.

### dd. 7-Hydroxy-6-[3-(2-methoxy-phenyl)-3-oxo-propyl]-2,3-dimethyl-3H-benzoimidazole-5-carboxylic acid dimethylamido

A suspension of 6-dimethylaminomethyl-7-hydroxy-2,3-dimethyl-3H-benzoimidazole-5-carboxylic acid dimethylamide (5.40 g, 18.6 mmol) in 1,2-dimethoxyethane (100 ml) was heated to 85 Cc and a solution of 1-[1-(2-methoxymethyl-phenyl)-vinyl]-pyrrolidine (synthesis described below, 6.5 g, 31 mmol) in DME (20 ml) was added slowly. The reaction mixture was kept at 85° C. for 3 h and was stirred at room temperature for 17 h. The solvent was evaporated in vacuo. The residue was dissolved in a mixture of dichloromethane and methanol, silica gel was added, and the suspension was evaporated to dryness. A column packed with 200 g of silica gel was loaded with the solid and the title compound was eluted with a mixture of dichloromethane and methanol (20:1). Evaporation of the corresponding fractions furnished a brown solid, which was treated with hot isopropanol (150 ml). The precipitate was isolated by filtration, washed with isopropanol, and dried. This afforded 3.0 g of the title compound (colourless solid, 39% yield).—m.p. 165° C.

### ee. 7-Hydroxy-2-methyl-6-(3-oxo-3-o-tolyl-propyl)-3-(2-trimethylsilanyl-ethoxymethyl)-3H-benzoimidazole-5-carboxylic acid dimethylamide

A solution of 7-hydroxy-2-methyl-3-(2-trimethylsilanyl-ethoxymethyl)-3H-benzoimidazole-5-carboxylic acid dimethylamide (example b, 2.4 g, 6.9 mmol) in dichloromethane (40 ml) was treated with N,N-dimethyl-methyleneiminium iodide (1.8 g, 9.8 mmol) and the reaction was stirred for 7 h at room temperature. The reaction mixture was poured into saturated sodium hydrogencarbonate solution, stirred for 30 min at room temperature, and extracted with dichloromethane (3×). The organic layers were dried over magnesium sulfate and concentrated in vacuo. A suspension of the residue (2.77 g of a beige solid, 6.8 mmol, 99% yield) in 1,2-dimethoxyethane (100 ml) was heated to 85° C. and a solution of 1-[1-(2-methylphenyl)-vinyl]-pyrrolidine (CAS 156004-72-7, 2.0 g, 10.7 mmol) was slowly added. The suspension was heated at 85° C. for 3 h. The reaction mixture was cooled to room temperature and the solvent was evaporated in vacuo. A solution of the crude product and fumaric acid (0.79 g, 6.8 mmol) in acetone was stirred for 17 h at room temperature. No precipitate was formed. The solution was concentrated and a mixture of dichloromethane and saturated sodium hydrogencarbonate solution was added. The organic phase was dried over sodium sulfate, silica gel was added, and the solvent was evaporated. A column filled with silica gel was loaded with the residue and the title compound was eluted with toluene/1,4-dioxane=2:1. Evaporation of the corresponding fractions afforded a beige solid, which was recrystallized from isopropanol. The title compound was obtained in 20% yield (0.67 g of a colourless solid).—m.p. 163-164° C.

### ff. 6-(3-Benzo[b]thiophen-3-yl-3-oxo-propyl)-7-hydroxy-2,3-dimethyl-3H-benzoimidazole-5-carboxylic acid dimethylamide

The crude title compound was prepared from 6-dimethylaminomethyl-7-hydroxy-2,3-dimethyl-3H-benzoimidazole-5-carboxylic acid dimethylamide (5.30 g, 18.3 mmol) and 1-(1-benzo[b]thiophen-3-yl-vinyl)-pyrrolidine (synthesis described below, 4.8 g, 21 mmol) and was purified by flash chromatography on silica gel (first column: Toluene/1,4-Dioxane=4:1, second column: Dichloromethane/Methanol 100:0 to 88:12). Evaporation of the corresponding fractions furnished a brown solid, which was dissolved in acetone. Fumaric acid was added and the precipitate was isolated by filtration. The salt of the title compound with fumaric acid was dissolved in dichloromethane and aqueous saturated sodium hydrogen carbonate solution. The phases were separated, the organic layer was dried over magnesium sulfate and concentrated in vacuo. The residue was purified by flash chromatography on silica gel (Dichloromethane/Methanol=20:1). This afforded 709 mg of the title compound (brown solid, 9% yield, 12% corrected yield).—m.p. 150-151° C.

### gg. 7-Hydroxy-2,3-dimethyl-6-[3-(2-methyl-thiophen-3-yl)-3-oxo-propyl]-3 benzolmidazole-5-carboxylic acid dimethylamide

A suspension of 6-dimethylaminomethyl-7-hydroxy-2,3-dimethyl-3H-benzoimidazole-5-carboxylic acid dimethylamide (4.0 g, 13.8 mmol) in 1,2-dimethoxyethane (100 ml) was heated to 85 Cc and a solution of 1-[1-(2-methyl-thiophen-3-yl)-vinyl]-pyrrolidine (synthesis described below, 4.0 g, 20.7 mmol) in DME was added slowly. The reaction mixture was kept at 85° C. for 3 h. The solvent was evaporated in vacuo. The residue was purified by flash chromatography on silica gel (Toluene/1,4-Dioxane=4:1, then Dichloromethane/Methanol 10:1). Evaporation of the corresponding fractions furnished a brown solid, which was slurried in hot isopropanol (2×). The title compound was isolated by filtration and was further purified by column chromatography on silica gel (Dichloromethane/Methanol=20:1). This afforded 1.3 g of a beige solid (24% yield).

¹H-NMR (DMSO-d₆, 400 MHz): δ=2.53 (s), 2.67 (s, 3H), 2.77 (s, 3H), 3.01 (s, bs, 6H), 3.67 (s, 3H), 6.78 (s, 1H), 7.33 (d, 1H), 7.49 (d, 1H), 10.00 (bs, 1H).

### hh. 8-Methoxy-2,3-dimethyl-8-o-tolyl-3,6,7,8-tetrahydro-chromeno[7,8-h]imidazole-5-carboxylic acid cyclopropylamide

N-(3-Dimethylaminopropyl)-N′-ethylcarbodiimide hydrochloride (6.67 g, 34.8 mmol), DMAP (0.03 g, 0.25 mmol), and cyclopropylamine (1.98 g, 34.7 mmol) were added to a suspension of 8-methoxy-2,3-dimethyl-8-o-tolyl-3,6,7,8-tetrahydro-chromeno[7,8-d]imidazole-5-carboxylic acid (example v, 7.0 g, 17.4 mmol) in dichloromethane (200 ml). The reaction mixture was stirred for 20 h at room temperature and was then treated with sodium bicarbonate solution (100 ml). The phases were separated and the aqueous phase was extracted with dichloromethane (2×30 ml). The combined organic phases were dried over sodium sulfate and evaporated to dryness. The brown residue (8.3 g) was purified by column chromatography on silica gel (Ethyl acetate/Methanol=9:1). The title compound was isolated in 95% yield (6.7 g of a colourless solid).—m.p. 271° C.

¹H-NMR (DMSO-d₆, 200 MHz): δ=0.64 (m_{c}, 2H), 0.89 (m_{c}, 2H), 1.95 (m_{c}, 1H), 2.44 (m_{c}, 1H), 2.53 (s, 3H), 2.62 (s, 3H), 2.90 (m_{c}, 2H), 3.20, 3.23 (s, m_{c}, 4H), 3.69 (s, 3H), 6.01 (bs, 1H), 7.01 (s, 1H), 7.21 (m_{c}, 3H), 7.87 (m_{c}, 1H).

### ii. 7-Hydroxy-2,3-dimethyl-6-(3-oxo-3-o-tolyl-propyl)-3H-benzolmidazole-5-carboxylic acid cyclopropylamide

A suspension of 8-methoxy-2,3-dimethyl-8-o-tolyl-3,6,7,8-tetrahydro-chromeno[7,8-d]imidazole-5-carboxylic acid cyclopropylamide (example hh, 6.6 g, 16.3 mmol) in THF (150 ml) was treated with 2 N hydrochloric acid (50 ml). The reaction mixture was stirred for 2 h at room temperature and for 2 h at 50° C. Sodium bicarbonate solution (100 ml) and dichloromethane (200 ml) was added and a pH-value of 8 was adjusted by addition of sodium hydroxide solution. The mixture was diluted with dichloromethane (200 ml) and water (200 ml). The phases were separated and the aqueous phase was extracted with dichloromethane (2×200 ml). The combined organic phases were washed with water (2×200 ml) and the solvent was evaporated in vacuo. This afforded the title compound in 88% yield (5.6 g of a colourless solid).—m.p. 297-299° C.

### jj. (8-Methoxy-2,3-dimethyl-8-o-tolyl-3,6,7,8-tetrahydro-chromeno[7,8-d]imidazol-5-yl)-methanol

To a suspension of 8-methoxy-2,3-dimethyl-8-o-tolyl-3,6,7,8-tetrahydro-chromeno[7,8-d]imidazole-5-carboxylic acid (example v, 6.0 g, 15.2 mmol), lithium aluminium hydride (1.5 g, 39.5 mmol) was added portion-wise. The reaction mixture was stirred for 1.5 h at room temperature and another portion of lithium aluminium hydride (800 mg, 21.1 mmol) was added. After a period of 30 min, the reaction mixture was carefully poured into a mixture of saturated ammonium chloride solution and dichloromethane. The biphasic mixture was stirred for 30 min, the phases were separated and the aqueous phase was extracted with dichloromethane (3×). The combined organic phases were washed with water (2×), dried over magnesium sulfate, and concentrated under reduced pressure. The title compound (4.79 g of a colourless solid, 90% yield) was directly used for the next step.

¹H-NMR (DMSO-d₆, 200 MHz): δ=1.88 (m_{c}, 1H), 2.40, 2.49, 2.50 (m_{c}, s, s), 2.70 (m_{c}, 1H), 2.87 (m_{c}, 1H), 2.99 (s, 3H), 3.69 (s, 3H), 4.57 (m_{c}, 2H), 5.04 (t, 1H), 7.10 (m_{c}, 1H), 7.30 (m_{c}, 3H), 7.73 (m_{c}, 1H).

### kk. 5-Chloromethyl-8-methoxy-2,3-dimethyl-8-o-tolyl-3,6,7,8-tetrahydro-chromeno[7,8-d]imidazole

A suspension of (8-methoxy-2,3-dimethyl-8-o-tolyl-3,6,7,8-tetrahydro-chromeno[7,8-d]imidazol-5-yl)-methanol (example jj, 4.7 g, 13.3 mmol) in dichloromethane (150 ml) was cooled to 0° C. and thionyl chloride (1.20 ml, 1.96 g, 16.5 mmol) was added drop-wise. The yellow solution was stirred for 1.5 h at 0° C. Saturated sodium bicarbonate solution was added and stirring was continued for 10 min. The organic phase was separated, extracted with saturated ammonium chloride solution and water, and dried over magnesium sulfate. The solvent was evaporated in vacuo and a light-brown solid was isolated (5.4 g, quant. yield), which was used for the next step without further purification.

¹H-NMR (DMSO-d₆, 200 MHz): δ=1.93 (m_{c}, 1H), 2.48, 2.50 (m_{c}, s), 2.90, 3.02, 3.13 (m_{c}, s, m_{c}, 5H), 3.77 (s, 3H), 4.93 (2 d, 2H), 7.34 (m_{c}, 4H), 7.75 (m_{c}, 1H).

### ll. 8-Methoxy-5-methoxymethyl-2,3-dimethyl-8-o-tolyl-3,6,7,8-tetrahydro-chromeno[7,8-d]imidazole

5-Chloromethyl-8-methoxy-2,3-dimethyl-8-o-tolyl-3,6,7,8-tetrahydro-chromeno[7,8-d]imidazole (example kk, 5.0 g, 13.5 mmol) was dissolved in methanol (50 ml) and a solution of sodium methylate (30 weight-% in methanol, 2.0 g, 11.1 mmol) was added. The reaction mixture was stirred for 30 min at 50° C. and for 1 h at room temperature. The white suspension was concentrated under reduced pressure, diluted with saturated ammonium chloride solution, and extracted with dichloromethane. The organic phase was washed with water and the aqueous phase was extracted with dichloromethane (2×). The combined organic phases were dried over sodium sulfate and the solvent was evaporated in vacuo. This afforded 4.7 g (95% yield) of a yellow foam, which was directly used for the next step.

¹H-NMR (DMSO-d₆, 200 MHz): δ=1.91 (m_{c}, 1H), 2.46, 2.49 (m_{c}, s), 2.62 (s, 3H), 2.76, 2.91, 3.02 (m_{c}, m_{c}, s, 5H), 3.36 (s, 3H), 3.78 (s, 3H), 4.52 (2 d, 2H), 7.28 (m, 4H), 7.75 (m_{c}, 1H).

### mm. 3-(4-Hydroxy-6-methoxymethyl-1,2-dimethyl-1H-benzoimidazol-5-yl)-1-o-tolyl-propan-1-one

A suspension of 8-methoxy-5-methoxymethyl-2,3-dimethyl-8-o-tolyl-3,6,7,8-tetrahydro-chromeno[7,8-d]imidazole (example ll, 4.6 g, 12.6 mmol) in THF (100 ml) was treated with 2 N hydrochloric acid (35 ml). The yellow solution was stirred for 1 h at room temperature and for 1 h at 50° C. and was concentrated under reduced pressure until most of the THF had been removed. The reaction mixture was diluted with ice water and neutralised by addition of 2 N sodium hydroxide solution. A light-brown precipitate was formed, which was isolated by filtration and dried in vacuo. The title compound was further purified by column chromatography on silica gel (Dichloromethane/Methanol=20:1). This afforded 3.1 g of an off-white solid (70% yield).—m.p. 163° C.

¹H-NMR (DMSO-d₆, 200 MHz): δ=2.41 (s, 3H), 2.49 (s), 2.96 (m_{c}, 2H), 3.07 (m_{c}, 2H), 3.26 (s, 3H), 3.66 (s, 3H), 4.48 (s, 2H), 6.89 (s, 1H), 7.35 (m_{c}, 3H), 7.72 (m_{c}, 1H), 9.77 (bs, 1H).

Synthesis of 1-[1-(aryl)-vinyl]-pyrrolidines

The enamines (1-[1-(2-fluorophenyl)-vinyl]-pyrrolidine, 1-[1-(4-fluorophenyl)-vinyl]-pyrrolidine, 1-[1-(2-methylphenyl)-vinyl]-pyrrolidine, 1-[1-(2-chlorophenyl)-vinyl]-pyrrolidine, 1-[1-(2-trifluoromethyl-phenyl)-vinyl]-pyrrolidine, 1-(1-naphthalen-2-yl-vinyl)-pyrrolidine) are known compounds and were synthesized following procedures analogous to those described in Synthesis 2004, 4, 521-524.

### 1-[1-(2-Ethylphenyl)vinyl]-pyrrolidine

(a) 1-(2-Ethyl-phenyl)-ethanone: In a flame-dried flask filled with nitrogen, ethyl magnesium bromide (1 M solution in THF, 62.1 ml) was added at −50° C. to a suspension of manganese(II) chloride (7.8 g, 62.0 mmol) and lithium chloride (5.3 g, 125.0 mmol) in dry THF (140 ml). A solution of 1-(2-chloro-phenyl)-ethanone (6.7 ml, 51.5 mmol) in THF (50 ml) was added over a period of 15 min and the reaction mixture was stirred at −50° C. for 4 hours. The reaction was quenched by addition of 2 N HCl (50 ml) and was extracted with dichloromethane (3×). The combined organic phases were washed with saturated sodium bicarbonate solution and with water, dried over magnesium sulfate, and concentrated under reduced pressure. The residue was purified by vacuum distillation. The title compound (6.1 g, 80% yield) was obtained in the form of a colourless liquid.

(b) 1-[1-(2-Ethylphenyl)-vinyl]-pyrrolidine: In a flame-dried flask filled with nitrogen, a solution of 1-(2-ethyl-phenyl)-ethanone (6.1 g, 41.1 mmol) in n-hexane (70 ml) was treated with pyrrolidine (20.4 ml, 247 mmol). Titanium tetrachloride (2.7 ml, 24.7 mmol) was added at 0° C. and the reaction mixture was stirred at room temperature for 15 h. The suspension was filtered. Evaporation of the filtrate furnished the title compound (6.3 g of a yellow oil, 74% yield).

¹H-NMR (DMSO-d₆, 400 MHz): δ=1.13 (t, 3H), 1.78 (m_{c}, 4H), 2.61 (q, 2H), 2.89 (m_{c}, 4H), 3.40 (s, 1H), 3.69 (s, 1H), 7.09-7.48 (m, 4H).

### 1-(1-Thiophen-2-yl-vinyl)-pyrrolidine

In a flame-dried flask filled with nitrogen, a solution of 2-acetylthiophene (3.7 ml, 34.4 mmol) in n-hexane (60 ml) was treated with pyrrolidine (17.1 ml, 207 mmol). Titanium tetrachloride (2.3 ml, 20.7 mmol) was added at 0° C. and the reaction mixture was stirred at room temperature for 15 h. The suspension was filtered. Evaporation of the filtrate furnished the title compound (4.2 g of a brown oil, 68% yield).

¹H-NMR (DMSO-d₆, 200 MHz): δ=1.84 (m_{c}, 4H), 3.02 (m_{c}, 4H), 3.84 (s, 1H), 3.95 (s, 1H), 7.00 (m_{c}, 2H), 7.48 (m_{c}, 1H).

### 1-[1-(4-Fluoro-2-methylphenyl)-vinyl]-pyrrolidine

In a flame-dried flask filled with nitrogen, a solution of 4-fluoro-2-methylacetophenone (5.2 g, 34.2 mmol) in n-hexane (60 ml) was treated with pyrrolidine (17.1 ml, 207 mmol). Titanium tetrachloride (2.3 ml, 20.7 mmol) was added at 0° C. and the reaction mixture was stirred at room temperature for 15.5 h. The suspension was filtered. Evaporation of the filtrate furnished the title compound (6.4 g of a yellow oil, 91% yield).

¹H-NMR (DMSO-d₆, 200 MHz): δ=1.80 (m_{c}, 4H), 2.24 (s, 3H), 2.85 (m_{c}, 4H), 3.40 (s, 1H), 3.68 (s, 1H), 7.05 (m_{c}, 3H).

### 1-[1-(2-Benzyloxymethyl-phenyl)-vinyl]-pyrrolidine

(a) 2-(2-Methyl-[1,3]dioxolan-2-yl)-benzoic acid ethyl ester: Triethyl orthoformate (68.4 g, 0.46 mol), 1,2-ethanediol (104.2 g, 1.68 mol), and p-toluenesulfonic acid monohydrate (0.8 g, 4.2 mmol) was added to a solution of 2-acetyl-benzoic acid ethyl ester (80.7 g, 0.42 mol) in THF (120 ml). The reaction mixture was heated at 40° for 17 h and was poured onto a solution of sodium hydrogencarbonate (370 mg) in water (160 ml). The biphasic mixture was stirred for 15 min, the phases were separated, and the aqueous phase was extracted with ethyl acetate (150 ml). The combined organic phases were dried over sodium sulfate, and concentrated under reduced pressure. The residue (121 g of a yellow liquid) was purified by flash chromatography on silica gel (Petrolether/Ethyl acetate=9:1). Evaporation of the corresponding fractions afforded the title compound (74.8 g of an off-white solid, 75% yield).

¹H-NMR (DMSO-d₆, 400 MHz): δ=1.26 (t, 3H), 1.69 (s, 3H), 3.48 (m_{c}, 2H), 3.91 (m_{c}, 2H), 4.24 (q, 2H), 7.30-7.43 (m_{c}, 2H), 7.48 (m_{c}, 2H).

(b) [2-(2-Methyl-[1,3]dioxolan-2-yl)-phenyl]-methanol: At a temperature of 0° C., lithium aluminium hydride (3.3 g, 87 mmol) was added portion-wise to a solution of 2-(2-methyl-[1,3]dioxolan-2-yl)-benzoic acid ethyl ester (27.4 g, 116 mmol) in dry THF (150 ml). The grey suspension was stirred for 30 min at room temperature. The reaction mixture was carefully quenched with a mixture of ice (100 g) and saturated ammonium chloride solution (100 ml). Dichloromethane (300 ml) was added and the biphasic mixture was stirred for 10 min. The phases were separated and the aqueous phase was extracted with dichloromethane (2×200 ml). The combined organic phases were washed with water (150 ml), dried over sodium sulfate, and concentrated under reduced pressure. The reduction of 47.3 g (200 mmol) of 2-(2-methyl-[1,3]dioxolan-2-yl)-benzoic acid ethyl ester with lithium aluminium hydride (5.7 g, 150 mmol) was performed under analogous conditions. The crude products (21.7 g from the first experiment, 38.7 g from the second experiment) were combined and purified by flash chromatography on silica gel (Petrolether/Ethyl acetate=7:3). The title compound was isolated in 88% yield (60.6 g of a colourless oil containing 11 weight-% of ethyl acetate).

¹H-NMR (CDCl₃, 200 MHz): δ=1.25 (t, 3H, EtOAc), 1.71 (s, 3H), 2.03 (s, 3H, EtOAc), 3.82 (m_{c}, 2H), 4.01 (m_{c}, 2H), 4.11 (q, 2H, EtOAc), 4.76 (s, 2H), 7.33 (m_{c}, 3H), 7.56 (m_{c}, 1H).

(c) 2-(2-Benzyloxymethyl-phenyl)-2-methyl-[1,3]dioxolane: Portions of sodium hydride (60 weight-% in oil, total amount: 9.9 g, 248 mmol) were added to a solution of [2-(2-methyl-[1,3]dioxolan-2-yl)-phenyl]-methanol (48.0 g, 247 mmol) in dry DMF (450 ml). The solution was stirred for 1 h at room temperature and tetrabutylammonium iodide (9.2 g, 249 mmol) and benzylbromide (42.3 g, 247 mmol) were added. Stirring was continued for 2 h at room temperature. The reaction mixture was quenched with saturated ammonium chloride solution (400 ml) and dichloromethane (500 ml). The phases were separated and the aqueous phase was extracted with dichloromethane (200 ml). The combined organic phases were washed with water (2×200 ml), dried over sodium sulfate, and concentrated. The residue (80 g of a brown oil) was purified by flash chromatography on silica gel (Petrolether, then Petrolether/Ethyl acetate=9:1). The title compound was isolated in 57% yield (40.1 g of a yellow oil).

¹H-NMR (CDCl₃, 200 MHz): δ=1.67 (s, 3H), 3.71 (m_{c}, 2H), 4.01 (m_{c}, 2H), 4.51 (s, 2H); 4.84 (s, 2H), 7.32 (m_{c}, 7H), 7.59 (m_{c}, 2H).

(d) 1-(2-Benzyloxymethyl-phenyl)-ethanone: Hydrochloric acid (250 ml of a 2 N solution) was added to a solution of 2-(2-benzyloxymethyl-phenyl)-2-methyl-[1,3]dioxolane (40.1 g, 141 mmol) in THF (500 ml). The reaction mixture was heated at 50° C. for 2 h and was poured onto a mixture of water (400 ml) and dichloromethane (300 ml). A pH value of 8 was adjusted by addition of 6 N sodium hydroxide solution. The phases were separated and the aqueous phase was extracted with dichloromethane (200 ml). The combined organic phases were dried over sodium sulfate and concentrated in vacuo. The crude product (33 g of a yellow liquid) was purified by flash chromatography on silica gel (Petrolether/Ethyl acetate=9:1). Evaporation of the corresponding fractions furnished the title compound (31.9 g of a yellow liquid, 94% yield).

¹H-NMR (DMSO-d₆, 200 MHz): δ=2.58 (s, 3H), 4.63 (s, 2H), 4.89 (s, 2H), 7.34 (m_{c}, 6H), 7.51 (m_{c}, 1H), 7.74 (m_{c}, 2H).

(e) 1-[1-(2-Benzyloxymethyl-phenyl)-vinyl]-pyrrolidine: In a flame-dried flask filled with argon, a solution of 1-(2-benzyloxymethyl-phenyl)-ethanone (31.9 g, 133 mmol) in n-hexane (450 ml) was treated with pyrrolidine (47.1 g, 663 mmol). A solution of titanium tetrachloride (12.6 g, 663 mmol) in n-hexane (60 ml) was added at 0° C. and the reaction mixture was stirred at room temperature for 17 h. The suspension was filtered and the precipitate was washed with n-hexane (3×200 ml). Evaporation of the filtrate furnished the title compound (36 g of an orange oil, 93% yield).

¹H-NMR (CDCl₃, 200 MHz): δ=1.78 (m_{c}, 4H), 2.90 (m_{c}, 4H), 3.59 (s, 1H), 3.75 (s, 1H), 4.56 (s, 2H), 4.63 (s, 2H), 7.31 (m_{c}, 8H), 7.55 (d, 1H).

### 1-[1-(2-Methoxymethyl-phenyl)-vinyl]-pyrrolidine

(a) 2-(2-Methoxymethyl-phenyl)-2-methyl-[1,3]dioxolane: Sodium hydride (60 weight-% in oil, 2.3 g, 57.5 mmol) was added portion-wise to a solution of [2-(2-methyl-[1,3]dioxolan-2-yl)-phenyl]-methanol (10.0 g, 51.5 mmol) in THF (100 ml). After drop-wise addition of methyl iodide (3.5 ml, 8.0 g, 56.0 mmol) the reaction mixture was stirred for 1.5 h at room temperature and quenched by addition of ammonia solution (25 weight-% in water, 2 ml), water, and ethyl acetate. The phases were separated and the aqueous phase was extracted with ethyl acetate (2×). The combined organic phases were dried over magnesium sulfate and concentrated under reduced pressure. The residue (12 g of a yellow oil) was purified by flash chromatography on silica gel (Petrol ether/Ethyl acetate=9:1). The title compound was isolated in 67% yield (7.1 g of a colourless oil).

¹H-NMR (DMSO-d₆, 200 MHz): δ=1.58 (s, 3H), 3.34 (s, 3H), 3.62 (m_{c}, 2H), 3.97 (m_{c}, 2H), 4.63 (s, 2H), 7.30 (m_{c}, 2H), 7.47 (m_{c}, 2H).

(b) 1-(2-Methoxymethyl-phenyl)-ethanone: A solution of 2-(2-methoxymethyl-phenyl)-2-methyl-[1,3]dioxolane (7.0 g, 33.7 mmol) in THF (100 ml) and 2 N hydrochloric acid (50 ml) was stirred for 1.5 h at 50° C. The reaction mixture was diluted with dichloromethane and water and a pH-value of 7-8 was adjusted by addition of 6 N sodium hydroxide solution. The organic phase was washed with water and the aqueous phase was extracted with dichloromethane. The combined organic phases were dried over magnesium sulfate and concentrated under reduced pressure. The title compound was isolated in the form of a yellow oil (5.5 g, 99% yield) and was used for the next step without further purification.

¹H-NMR (DMSO-d₆, 200 MHz): δ=2.55 (s, 3H), 3.33 (s, 3H), 4.64 (s, 2H), 7.43 (m_{c}, 3H), 7.84 (m_{c}, 1H).

(c) 1-[1-(2-Methoxymethyl-phenyl)-vinyl]-pyrrolidine: In a flame-dried flask filled with argon, a solution of 1-(2-methoxyoxymethyl-phenyl)-ethanone (5.4 g, 32.9 mmol) in n-hexane (80 ml) was treated with pyrrolidine (16.3 ml, 14.1 g, 198 mmol). A solution of titanium tetrachloride (2.2 ml, 3.8 g, 20.0 mmol) in n-hexane (2 ml) was added at 0° C. and the reaction mixture was stirred at room temperature for 17 h. The suspension was filtered and the precipitate was washed with n-hexane. Evaporation of the filtrate furnished the title compound (6.5 g of a yellow oil, 90% yield).

¹H-NMR (DMSO-d₆, 200 MHz): δ=1.79 (m_{c}, 4H), 2.85 (m_{c}, 4H), 3.28 (s, 3H), 3.45 (s, 1H), 3.72 (s, 1H), 4.42 (s, 2H), 7.29 (m_{c}, 4H).

### 1-(1-Benzo[b]thiophen-3-yl-vinyl)-pyrrolidine

In a flame-dried flask filled with nitrogen, a solution of 1-benzo[b]thiophen-3-yl-ethanone (5.0 g, 28.4 mmol) in toluene (10 ml) was diluted with n-hexane (50 ml). At a temperature of 0° C., pyrrolidine (14.0 ml, 12.0 g, 169 mmol) and a solution of titanium tetrachloride (1.9 ml, 3.3 g, 17 mmol) in n-hexane was added. The reaction mixture was stirred at room temperature for 17 h. The suspension was filtered and the precipitate was washed with n-hexane. Evaporation of the filtrate furnished the title compound (4.8 g of a yellow-brown oil, 74% yield).

¹H-NMR (DMSO-d₆, 200 MHz): δ=1.82 (m_{c}, 4H), 2.94 (m_{c}, 4H), 3.82 (s, 1H), 3.88 (s, 1H), 7.40 (m_{c}, 2H), 7.64 (s, 1H), 7.83 (m_{c}, 1H), 7.99 (m_{c}, 1H).

### 1-[1-(2-Methyl-thiophen-3-yl)-vinyl]-pyrrolidine

(a) 3-Bromo-2-methyl-thiophene: 3-Bromothiophene (25.0 g, 153 mmol) was dissolved in THF (180 ml) and a solution of lithium diisopropylamide (2 M in THF/heptane/ethylbenzene, 88.0 ml, 176 mmol) was added at a temperature of −78° C. Stirring was continued for 3 h at −78° C. The reaction mixture was warmed to −30° C. and a solution of methyl iodide (14.3 ml, 32.6 g, 230 mmol) in THF (15 ml) was added. The reaction mixture was allowed to warm to room temperature and stirring was continued for 18.5 h. The reaction was quenched with water and extracted with diethyl ether (3×). The organic phases were dried over magnesium sulfate and concentrated under reduced pressure. The residue was purified by vacuum distillation (27.8 g of a yellowish oil containing 80 weight-% of the title compound and 20 weight-% of ethylbenzene, 126 mol, 82% yield).

¹H-NMR (DMSO-d₆, 200 MHz): δ=2.36 (s, 3H), 7.01 (d, 1H), 7.44 (d, 1H), ethylbenzene: 1.18 (t, 3H), 2.60 (q, 2H), 7.22 (m, 5H)

(b) 1-(2-Methyl-thiophen-3-yl)-ethanone via 3-(1-Butoxy-vinyl)-2-methyl-thiophene: In an autoclave, a solution of 3-bromo-2-methyl-thiophene (mixture obtained in step a, 27.8 g, 126 mmol), n-butyl vinyl ether (49.6 ml, 38.4 g, 383 mmol), palladium acetate (1.70 g, 7.6 mmol), 1,3-bis(diphenylphosphino)propane (7.60 g, 18.4 mmol), and potassium carbonate (22.9 g, 166 mmol) in a degassed mixture of DMF (330 ml) and water (40 ml) was heated for 3 d at 100° C. The reaction mixture was cooled to room temperature. Hydrochloric acid (5%) was added and stirring was continued for 2.75 h at room temperature. The reaction mixture was neutralized by addition of potassium hydroxide solution (10%) and extracted with dichloromethane (3×). The combined organic phases were dried over magnesium sulfate. The solvent was evaporated in the presence of silica gel and the residue was loaded on top of a column filled with silica gel. The title compound (9.7 g of an orange oil, 55% yield) was eluted with a mixture of petrol ether and ethyl acetate [20:1 (v/v)].

¹H-NMR (DMSO-d₆, 200 MHz): δ=2.47 (s, 3H), 2.65 (s, 3H), 7.32 (d, 1H), 7.48 (d, 1H).

(c) 1-[1-(2-Methyl-thiophen-3-yl)-vinyl]-pyrrolidine: In a flame-dried flask Filled with nitrogen, a solution of 1-(2-methyl-thiophen-3-yl)-ethanone (4.2 g, 30 mmol) in n-hexane (70 ml) was treated with pyrrolidine (15.0 ml, 12.9 g, 181 mmol). A solution of titanium tetrachloride (2.0 ml, 3.4 g, 18 mmol) in n-hexane was added at 0° C. and the reaction mixture was stirred at room temperature for 17.5 h. The suspension was filtered and the precipitate was washed with n-hexane. Evaporation of the filtrate furnished the title compound (4.0 g of a yellow-brown oil, 69% yield).

¹H-NMR (DMSO-d₆, 200 MHz): δ=1.80 (m_{c}, 4H), 2.38 (s, 3H), 2.90 (m_{c}, 4H), 3.54 (s, 1H), 3.77 (s, 1H), 6.85 (d, 1H), 7.23 (d, 1H).

### INDUSTRIAL APPLICABILITY

The compounds of the formula 1-a and of the formula 1-b are valuable intermediates for the preparation of enantiomerically pure 8-aryl-3,6,7,8-tetrahydro-chromeno[7,8-d]imidazoles derivatives of the formula 3-a or 3-b respectively.

The compounds of the formula 3-a and 3-b and their pharmaceutically acceptable salts (=active compounds according to the invention), preferably those compounds of the formula 3-a and their pharmaceutically acceptable salts, have valuable pharmacological properties which make them commercially utilizable. In particular, they exhibit marked inhibition of gastric acid secretion and an excellent gastric and intestinal protective or curative action in warm-blooded animals, in particular humans. In this connection, the active compounds according to the invention are distinguished by a high selectivity of action, a fast onset of action, an advantageous duration of action, efficient control of the duration of action by the dosage, a particularly good antisecretory efficacy, the absence of significant side effects and a large therapeutic range. Compared to compounds known from the prior art, the active compounds according to the present invention are particularly distinguished by an excellent efficacy with regard to inhibition of gastric acid secretion and/or by a low potential to cause side effects for example due to a low affinity to one or more other enzymes whose inhibition is related to these side effects and/or by a low potential of drug-drug interactions.

“Gastric and intestinal protection or cure” in this connection is understood to include, according to general knowledge, the prevention, the treatment and the maintenance treatment of gastrointestinal diseases, in particular of gastrointestinal inflammatory diseases and lesions (such as, for example, reflux esophagitis, gastritis, hyperacidic or drug-related functional dyspepsia, and peptic ulcer disease [including peptic ulcer bleeding, gastric ulcer, duodenal ulcer]), which can be caused, for example, by microorganisms (e.g. Helicobacter pylori), bacterial toxins, drugs (e.g. certain antiinflammatories and antirheumatics, such as NSAIDs and COX-inhibitors), chemicals (e.g. ethanol), gastric acid or stress situations.

The term “gastrointestinal diseases” is understood to include, according to general knowledge,

A) gastroesophageal reflux disease (GERD), the symptoms of which include, but are not limited to, heartburn and/or acid regurgitation and/or non-acid regurgitation.
B) other extra-esophageal manifestations of GERD that include, but are not limited to, acid-related asthma, bronchitis, laryngitis and sleep disorders.
C) other diseases that can be connected to undiagnosed reflux and/or aspiration include, but are not limited to, airway disorders such as asthma, bronchitis, COPD (chronic obstructive pulmonary disease).
D) Helicobacter pylori infection whose eradication is playing a key role in the treatment of gastrointestinal diseases.
E) Furthermore, “gastrointestinal diseases” comprise other gastrointestinal conditions that might be related to acid secretion, such as Zollinger-Ellison syndrome, acute upper gastrointestinal bleeding, nausea, vomiting due to chemotherapy or post-operative conditions, stress ulceration, IBD (inflammatory bowel disease) and particularly IBS (irritable bowel syndrome).

In their excellent properties, the active compounds according to the invention surprisingly prove to be clearly superior to the compounds known from the prior art in various models in which the antiulcerogenic and the antisecretory properties are determined. On account of these properties, the active compounds according to the invention are outstandingly suitable for use in human and veterinary medicine, where they are used, in particular, for the treatment and/or prophylaxis of disorders of the stomach and/or intestine and/or upper digestive tract, particularly of the abovementioned diseases.

A further subject of the invention are therefore the active compounds according to the invention for use in the treatment and/or prophylaxis of the abovementioned diseases.

The invention likewise includes the use of the active compounds according to the invention for the production of medicaments which are employed for the treatment and/or prophylaxis of the abovementioned diseases.

The invention furthermore includes the use of the active compounds according to the invention for the treatment and/or prophylaxis of the abovementioned diseases.

A further subject of the invention are medicaments which comprise one or more active compounds according to the invention.

As medicaments, the active compounds according to the invention are either employed as such, or preferably in combination with suitable pharmaceutical excipients in the form of tablets, coated tablets (e.g. film-coated tablets), multi unit particulate system tablets, capsules, suppositories, granules, powders (e.g. lyophilized compounds), pellets, patches (e.g. as TTS [transdermal therapeutic system]), emulsions, suspensions or solutions. The content of the active compound is advantageously being between 0.1 and 95 wt % (weight percent in the final dosage form), preferably between 1 and 60 wt %. By means of the appropriate selection of the excipients, it is possible to obtain a pharmaceutical administration form adapted to the active compound and/or to the desired onset and/or duration of action (e.g. a sustained release form or a delayed release form).

The active compounds according to the invention can be administered orally, parenterally (e.g. intravenously), rectally or percutaneously. Oral or intravenous administration is preferred.

The excipients or combinations of excipients which are suitable for the desired pharmaceutical formulations are known to the person skilled in the art on the basis of his/her expert knowledge and are composed of one or more accessory ingredients. In addition to solvents, antioxidants, stabilizers, surfactants, complexing agents (e.g. cyclodextrins), the following excipients may be mentioned as examples: For oral administration, gelling agents, antifoams, plasticizer, adsorbent agents, wetting agents, colorants, flavorings, sweeteners and/or tabletting excipients (e.g. carriers, fillers, binders, disintegrating agents, lubricants, coating agents); for intravenous administration, dispersants, emulsifiers, preservatives, solubilizers, buffer substances and/or isotonic adjusting substances. For percutaneous administration, the person skilled in the art may choose as excipients, for example: solvents, gelling agents, polymers, permeation promoters, adhesives, matrix substances and/or wetting agents.

In general, it has been proven advantageous in human medicine to administer the active compound(s) in the case of oral administration in a daily dose (given continuously or on-demand) of approximately 0.01 to approximately 20, preferably 0.02 to 5, in particular 0.02 to 1.5, mg/kg of body weight, if appropriate in the form of several, preferably 1 to 2, individual doses to achieve the desired result. In the case of a parenteral treatment, similar or (in particular in the case of the intravenous administration of the active compounds), as a rule, lower doses can be used. Furthermore, the frequency of administration can be adapted to intermittent, weekly, monthly, even more infrequent (e.g. implant) dosing. The establishment of the optimal dose and manner of administration of the active compounds necessary in each case can easily be carried out by any person skilled in the art on the basis of his/her expert knowledge.

The medicaments may conveniently be presented in unit dosage form and may be prepared by any of the methods well known in the art of pharmaceutical science. All methods include the step of bringing the active compounds according to the invention into association with the excipients or a combination of excipients. In general the formulations are prepared by uniformly and intimately bringing into association the active compounds according to the invention with liquid excipients or finely divided solid excipients or both and then, if necessary, formulating the product into the desired medicament.

The active compounds according to the invention or their pharmaceutical preparations can also be used in combination with one or more pharmacologically active constituents from other groups of drugs [combination partner(s)]. “Combination” is understood to be the supply of both the active compound(s) according to the invention and the combination partner(s) for separate, sequential, simultaneous or chronologically staggered use. A combination is usually designed with the aim of increasing the principal action in an additive or super-additive sense and/or of eliminating or decreasing the side effects of the combination partner(s), or with the aim to obtain a more rapid onset of action and a fast symptom relief. By choosing the appropriate pharmaceutical formulation of the drugs contained in the combination, the drug release profile of the components can be exactly adapted to the desired effect, e.g. the release of one compound and its onset of action is chronologically previous to the release of the other compound.

A combination can be, for example, a composition containing all active compounds (for example a fixed combination) or a kit-of-parts comprising separate preparations of all active compounds.

A “fixed combination” is defined as a combination wherein a first active ingredient and a second active ingredient are present together in one unit dosage or in a single entity. One example of a “fixed combination” is a pharmaceutical composition wherein the said first active ingredient and the said second active ingredient are present in admixture of simultaneous administration, such as in a formulation. Another example of a “fixed combination” is a pharmaceutical composition wherein the said first active ingredient and the said second active ingredient are present in one unit without being in admixture.

A “kit-of-parts” is defined as a combination wherein the said first active ingredient and the said second active ingredient are present in more than one unit. One example of a “kit-of-parts” is a combination wherein the said first active ingredient and the said second active ingredient are present separately. The components of the kit-of-parts may be administered separately, sequentially, simultaneously or chronologically staggered.

“Other groups of drugs” are understood to include, for example: tranquilizers (for example from the group of the benzodiazepines, like diazepam), spasmolytics (for example butylscopolaminium bromide [Buscopan®]), anticholinergics (for example atropine sulfate, pirenzepine, tolterodine), pain perception reducing or normalizing agents (for example, paracetamol, tetracaine or procaine or especially oxetacain), and, if appropriate, also enzymes, vitamins, trace elements or amino acids.

To be emphasized in this connection is in particular the combination of the active compounds according to the invention with pharmaceuticals which buffer or neutralize gastric acid (such as, for example, magaldrat, aluminium hydroxide, magnesium carbonate, magnesium hydroxide or other antacids), or especially with pharmaceuticals which inhibit or reduce acid secretion, such as, for example:

(I) histamine-H2 blockers [e.g. cimetidine, ranitidine], or
(II) proton pump inhibitors [e.g. omeprazole, esomeprazole, pantoprazole, lansoprazole, rabeprazole, tenatoprazole, ilaprazole, leminoprazole, all including their salts and enantiomers] or
(III) other potassium-competitive acid blockers [e.g. soraprazan and its stereoisomers, linaprazan, revaprazan, all including their salts]), or
(IV) so-called peripheral anticholinergics (e.g. pirenzepine), with gastrin antagonists such as CCK2 antagonists (cholestocystokinin 2 receptor antagonists).

An important combination to be mentioned is the combination with antibacterially active substances, and especially substances with a bactericidal effect, or combinations thereof. These combination partner(s) are especially useful for the control of Helicobacter pylori infection whose eradication is playing a key role in the treatment of gastrointestinal diseases. As suitable antibacterially active combination partner(s) may be mentioned, for example:

(A) cephalosporins, such as, for example, cifuroximaxetil
(B) penicillines, such as, for example, amoxicillin, ampicillin
(C) tetracyclines, such as, for example, tetracyline itself, doxycycline
(D) β-lactamase inhibitors, such as, for example, clavulanic acid
(E) macrolide antibiotics, such as, for example, erythromycin, clarithromycin, azithromycin
(F) rifamycines, such as, for example, rifamycine itself
(G) glycoside antibiotics, such as, for example, gentamicin, streptomycin
(H) gyrase inhibitors, such as, for example, ciprofloxaxin, gatifloxacin, moxifloxacin
(I) oxazolidines, such as, for example, linezolid
(J) nitrofuranes or nitroimidazoles, such as, for example, metronidazole, tinidazole, nitrofurantoin
(K) bismuth salts, such as, for example, bismuth subcitrat
(L) other antibacterially active substances
and combinations of substances selected from (A) to (L), for example clarithromycin+metronidazole. Preferred is the use of two combination partners. Preferred is the use of two combination partners selected from amoxicillin, clarithromycin and metronidazole. A preferred example is the use of amoxicillin and clarithromycin.

In view of their excellent activity regarding gastric and intestinal protection or cure, the active compounds according to the invention are especially suited for a free or fixed combination with drugs, which are known to cause “drug-induced dyspepsia” or are known to have a certain ulcerogenic potency, such as, for example, acetylsalicylic acid, certain antiinflammatories and antirheumatics, such as NSAIDs (non-steroidal antiinflammatory drugs, e.g. etofenamate, diclofenac, indometacin, ibuprofen, piroxicam, naproxen, meloxicam), oral steroids, bisphosphonates (e.g. alendronate), or even NO-releasing NSAIDs, COX-2 inhibitors (e.g. celecoxib, lumiracoxib).

In addition, the active compounds according to the invention are suited for a free or fixed combination with motility-modifying or -regulating drugs (e.g. gastroprokinetics like mosapride, tegaserod, itopride, metoclopramid), and especially with pharmaceuticals which reduce or normalize the incidence of transient lower esophageal sphincter relaxation (TLESR), such as, for example, GABA-B agonists (e.g. baclofen, (2R)-3-amino-2-fluoropropylphosphinic acid) or allosteric GABA-B agonists (e.g. 3,5-bis(1,1-dimethylethyl)-4-hydroxy-β,β-dimethylbenzenepropanol), GABA-B re-uptake inhibitors (e.g. tiagabine), metabotropic glutamate receptor type 5 (mGluR5) antagonists (e.g. 2-methyl-6-(phenylethynyl)pyridine hydrochloride), CB2 (cannabinoid receptor) agonists (e.g. [(3R)-2,3-dihydro-5-methyl-3-(4-morpholinyl-methyl)pyrrolo[1,2,3,de]-1,4-benzoxazin-6-yl]-1-naphthalenyl-methanone mesylate). Pharmaceuticals used for the treatment of IBS or IBD are also suitable combination partner(s), such as, for example: 5-HT4 receptor agonists like mosapride, tegaserod; 5-HT3 receptor antagonists like alosetron, cilansetron; NK2 antagonists like saredutant, nepadutant; κ-opiate agonists like fedotozine.

Suitable combination partner(s) also comprise airway therapeutica, for example for the treatment of acid-related asthma and bronchitis. In some cases, the use of a hypnotic aid (such as, for example, Zolpidem [Bikalm®]) as combination partner(s) may be rational, for example for the treatment of GERD-induced sleep disorders.

1. A process of preparing a compound of the formula 1-a comprising a catalytic hydrogenation of a compound of the formula 2 in the presence of a hydrogenation catalyst which is selected from the group consisting of RuXY[(S)-Xyl-P-Phos][(S)-DAIPEN] and RuXY[(S)-Xyl-BINAP][(S)-DAIPEN], 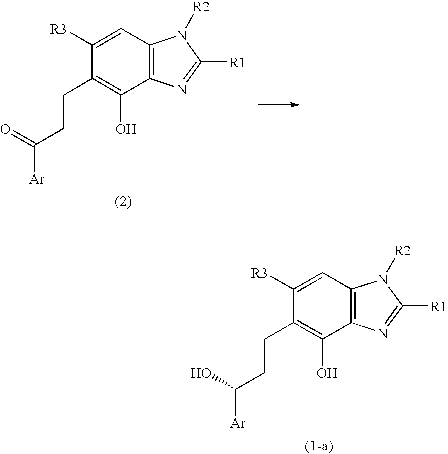
where
X and Y are the same or different substituents selected from the group consisting of hydrogen, halogen, BH₄ and carboxylate,
and in which
R1 is hydrogen, halogen, hydroxyl, 1-4C-alkyl, 3-7C-cycloalkyl, 3-7C-cycloalkyl-1-4C-alkyl, 1-4C-alkoxy, 1-4C-alkoxy-1-4C-alkyl, 1-4C-alkoxycarbonyl, 2-4C-alkenyl, 2-4C-alkynyl, fluoro-1-4C-alkyl, hydroxy-1-4C-alkyl or mono- or di-1-4C-alkylamino,
R2 is hydrogen, 1-4C-alkyl, 1-4C-alkoxy, 1-4C-alkoxy-1-4C-alkyl, aryl, 3-7C-cycloalkyl, 3-7C-cycloalkyl-1-4C-alkyl, 1-4C-alkoxycarbonyl, mono- or di-1-4C-alkylamino-1-4C-alkylcarbonyl, hydroxy-1-4C-alkyl, fluoro-2-4C-alkyl, 1-4C-alkoxy-1-4C-alkoxy-1-4C-alkyl, silyl substituted 1-4C-alkoxy-1-4Calkyl, 1-4C-alkylcarbonyl, or aryl-CH₂-oxycarbonyl.
R3 is hydrogen, halogen, fluoro-1-4C-alkyl, carboxyl, 1-4C-alkoxycarbonyl, hydroxy-1-4C-alkyl, 1-4C-alkoxy-1-4C-alkyl, 1-4C-alkoxy-1-4C-alkoxy-1-4C-alkyl, fluoro-1-4C-alkoxy-1-4C-alkyl, 1-4C-alkoxy-1-4C-alkoxy, 1-4C-alkylcarbonylamino, 1-4C-alkylcarbonyl-N-1-4C-alkylamino, 1-4C-alkoxy-1-4C-alkylcarbonylamino or the group —CO—NR31R32,
where
R31 is hydrogen, hydroxyl, 1-7C-alkyl, 3-7C-cycloalkyl, hydroxy-1-4C-alkyl or 1-4C-alkoxy-1-4C-alkyl and
R32 is hydrogen, 1-7C-alkyl, 3-7C-cycloalkyl, hydroxy-1-4C-alkyl or 1-4C-alkoxy-1-4C-alkyl,
or where
R31 and R32 together, including the nitrogen atom to which both are bonded, are a pyrrolidino, hydroxy-pyrrolidino, aziridino, azetidino, piperidino, piperazino, N-1-4C-alkylpiperazino or morpholino group,
Ar is a mono- or bicyclic aromatic residue, substituted by R4, R5, R6 and R7, which is selected from the group consisting of phenyl, naphthyl, pyrrolyl, pyrazolyl, imidazolyl, 1,2,3-triazolyl, indolyl, benzimidazolyl, furyl, benzofuryl, thienyl, benzothienyl, thiazolyl, isoxazolyl, pyridinyl, pyrimidinyl, chinolinyl and isochinolinyl,
wherein
R4 is hydrogen, 1-4C-alkyl, hydroxy-1-4C-alkyl, 1-4C-alkoxy, 24C-alkenyloxy, carboxy, 1-4C-alkoxycarbonyl, carboxy-1-4C-alkyl, 1-4C-alkoxycarbonyl-1-4C-alkyl, 1-4C-alkoxy-1-4C-alkyl, aryloxy-1-4C-alkyl, halogen, hydroxy, aryl, aryl-1-4C-alkyl, aryl-oxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
R5 is hydrogen, 1-4C-alkyl, 1-4C-alkoxy, 1-4C-alkoxycarbonyl, halogen, trifluoromethyl or hydroxy,
R6 is hydrogen, 1-4C-alkyl or halogen and
R7 is hydrogen, 1-4C-alkyl or halogen,
and wherein
aryl is phenyl or substituted phenyl with one, two or three same or different substituents selected from the group consisting of 1-4C-alkyl, 1-4C-alkoxy, carboxy, 1-4C-alkoxycarbonyl, halogen, trifluoromethyl, nitro, trifluoromethoxy, hydroxy and cyano.

2. A process of preparing a compound of the formula 1-b comprising a catalytic hydrogenation of a compound of the formula 2 in the presence of a hydrogenation catalyst which is selected from the group consisting of RuXY[(R)-Xyl-P-Phos][(R)-DAIPEN] and RuXY[(R)-Xyl-BINAP][(R)-DAIPEN], 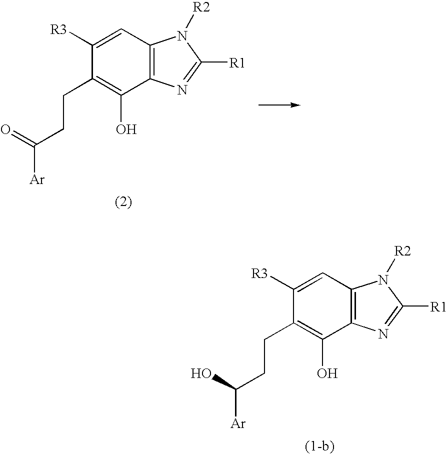
where
X and Y are the same or different substituents selected from the group consisting of hydrogen, halogen, BH₄ and carboxylate
and in which
R1 is hydrogen, halogen, hydroxyl, 1-4C-alkyl, 3-7C-cycloalkyl, 3-7C-cycloalkyl-1-4C-alkyl, 1-4C-alkoxy, 1-4C-alkoxy-1-4C-alkyl, 1-4C-alkoxycarbonyl, 2-4C-alkenyl, 2-4C-alkynyl, fluoro-1-4C-alkyl, hydroxy-1-4C-alkyl or mono- or di-1-4C-alkylamino,
R2 is hydrogen, 1-4C-alkyl, 1-4C-alkoxy, 1-4C-alkoxy-1-4C-alkyl, aryl, 3-7C-cycloalkyl, 3-7C-cycloalkyl-1-4C-alkyl, 1-4C-alkoxycarbonyl, mono- or di-1-4C-alkylamino-1-4C-alkylcarbonyl, hydroxy-1-4C-alkyl, fluoro-2-4C-alkyl, 1-4C-alkoxy-1-4C-alkoxy-1-4C-alkyl, silyl substituted 1-4C-alkoxy-1-4Calkyl, 1-4C-alkylcarbonyl, or aryl-CH₂-oxycarbonyl.
R3 is hydrogen, halogen, fluoro-1-4C-alkyl, carboxyl, 1-4C-alkoxycarbonyl, hydroxy-1-4C-alkyl, 1-4C-alkoxy-1-4C-alkyl, 1-4C-alkoxy-1-4C-alkoxy-1-4C-alkyl, fluoro-1-4C-alkoxy-1-4C-alkyl, 1-4C-alkoxy-1-4C-alkoxy, 1-4C-alkylcarbonylamino, 1-4C-alkylcarbonyl-N-1-4C-alkylamino, 1-4C-alkoxy-1-4C-alkylcarbonylamino or the group —CO—NR31R32,
where
R31 is hydrogen, hydroxyl, 1-7C-alkyl, 3-7C-cycloalkyl, hydroxy-1-4C-alkyl or 1-4C-alkoxy-1-4C-alkyl and
R32 is hydrogen, 1-7C-alkyl, 3-7C-cycloalkyl, hydroxy-1-4C-alkyl or 1-4C-alkoxy-1-4C-alkyl,
or where
R31 and R32 together, including the nitrogen atom to which both are bonded, are a pyrrolidino, hydroxy-pyrrolidino, aziridino, azetidino, piperidino, piperazino, N-1-4C-alkylpiperazino or morpholino group,
Ar is a mono- or bicyclic aromatic residue, substituted by R4, R5, R6 and R7, which is selected from the group consisting of phenyl, naphthyl, pyrrolyl, pyrazolyl, imidazolyl, 1,2,3-triazolyl, indolyl, benzimidazolyl, furyl, benzofuryl, thienyl, benzothienyl, thiazolyl, isoxazolyl, pyridinyl, pyrimidinyl, chinolinyl and isochinolinyl,
wherein
R4 is hydrogen, 1-4C-alkyl, hydroxy-1-4C-alkyl, 1-4C-alkoxy, 2-4C-alkenyloxy, carboxy, 1-4C-alkoxycarbonyl, carboxy-1-4C-alkyl, 1-4C-alkoxycarbonyl-1-4C-alkyl, 1-4C-alkoxy-1-4C-alkyl, aryloxy-1-4C-alkyl, halogen, hydroxy, aryl, aryl-1-4C-alkyl, aryl-oxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
R5 is hydrogen, 1-4C-alkyl, 1-4C-alkoxy, 1-4C-alkoxycarbonyl, halogen, trifluoromethyl or hydroxy,
R6 is hydrogen, 1-4C-alkyl or halogen and
R7 is hydrogen, 1-4C-alkyl or halogen,
and wherein
aryl is phenyl or substituted phenyl with one, two or three same or different substituents selected from the group consisting of 1-4C-alkyl, 1-4C-alkoxy, carboxy, 1-4C-alkoxycarbonyl, halogen, trifluoromethyl, nitro, trifluoromethoxy, hydroxy and cyano.

3. The process as claimed in claim 1, using RuXY[(S)-Xyl-P-Phos][(S)-DAIPEN] as the hydrogenation catalyst, where
X and Y are the same or different substituents selected from the group consisting of hydrogen, halogen, BH₄ and carboxylate
and in which
R1 is 1-4C-alkyl,
R2 is hydrogen, 1-4C-alkyl or silyl substituted 1-4C-alkoxy-1-4Calkyl,
R3 is 1-4C-alkoxy-1-4C-alkyl or the group —CO—NR31R32,
where
R31 is hydrogen, 1-7C-alkyl or 3-7C-cycloalkyl and
R32 is hydrogen or 1-7C-alkyl,
or where
R31 and R32 together, including the nitrogen atom to which both are bonded, are a pyrrolidino, or azetidino group,
Ar is a phenyl, naphthyl, thienyl or benzothienyl substituted by R4, R5, R6 and R7,
wherein
R4 is hydrogen, 1-4C-alkyl, halogen, 1-4C-alkoxy-1-4C-alkyl, aryloxy-1-4C-alkyl or trifluoromethyl,
R5 is hydrogen or halogen,
R6 is hydrogen and
R7 is hydrogen.

4. The process as claimed in claim 1, using RuXY[(S)-Xyl-P-Phos][(S)-DAIPEN] as the hydrogenation catalyst, where X and Y are each a chlorine radical,
and in which
R1 is 1-4C-alkyl,
R2 is 1-4C-alkyl or silyl substituted 1-4C-alkoxy-1-4Calkyl,
R3 is the group —CO—NR31R32,
where
R31 is hydrogen or 1-7C-alkyl,
R32 is hydrogen or 1-7C-alkyl,
or where
R31 and R32 together, including the nitrogen atom to which both are bonded, are a azetidino group,
Ar is a phenyl substituted by R4
wherein
R4 is hydrogen, 1-4C-alkyl or halogen.

5. The process as claimed in claim 1, which is performed in the presence of a base.

6. The process as claimed in claim 5, wherein the base is selected from the group consisting of KOH, KO^{t}Bu, K₂CO₃ and Cs₂CO₃.

7. The process as claimed in claim 1, in claim 6, which is carried out in a solvent, where the solvent comprises isopropanol or tert-butanol or a mixture of isopropanol and tert-butanol in any mixing ratio between 0:100 vol-% and 100:0 vol-%.

8. The process as claimed in claim 7, where the solvent additionally comprises between 5 and 30 vol-% of water.

9. The process as claimed in claim 1, which is performed in the presence of a base which is selected from the group consisting of KOH, KO^{t}Bu, K₂CO₃ and Cs₂CO₃ and where the solvent comprises isopropanol or tert-butanol or a mixture of isopropanol and tert-butanol in any mixing ratio between 0:100 vol-% and 100:0 vol-% and where the process is carried out in a homogenous solution containing a ketone of the formula 2 in concentrations between 0.1 and 1 M.

10. The process as claimed in claim 1, which is performed in the presence of a base which is selected from the group consisting of KOH, KO^{t}Bu, K₂CO₃ and Cs₂CO₃, and which is carried out in a solvent, where the solvent comprises isopropanol or tert-butanol or a mixture of isopropanol and tert-butanol in any mixing ratio between 0:100 vol-% and 100:0 vol-%. and where the solvent additionally comprises between 5 and 30 vol-% of water and where the process is carried out in a homogenous solution containing a ketone of the formula 2 in concentrations between 0.1 and 1 M.

11. A compound of the formula 1-a, 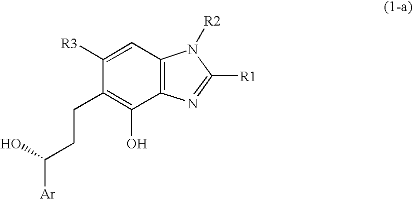
wherein R1, R2, R3 and Ar have the meanings as indicated in the following table:

| R1 | R2 | R3 | Ar |
|---|---|---|---|
| —CH₃ | —CH₃ | —C(O)—N(CH₃)₂ | 2-methyl-phenyl |
| —CH₃ | —CH₃ | —C(O)—N(CH₃)₂ | 2-ethyl-phenyl |
| —CH₃ | —CH₃ | —C(O)—N(CH₃)₂ | 2-isopropyl-phenyl |
| —CH₃ | —CH₃ | —C(O)—N(CH₃)₂ | 4-fluoro-2-methyl-phenyl |
| —CH₃ | —CH₃ | —C(O)—N(CH₃)₂ | 2-trifluoromethyl-phenyl |
| —CH₃ | —CH₃ | —C(O)—N(CH₃)₂ | 2-hydroxymethyl-phenyl |
| —CH₃ | —CH₃ | —C(O)—N(CH₃)₂ | 2-chloro-phenyl |
| —CH₃ | —CH₃ | —C(O)—N(CH₃)₂ | 2-(2-hydroxyethyl)- |
| | | | phenyl |
| —CH₃ | —CH₃ | —C(O)—N(CH₃)₂ | 2-(1-hydroxyethyl)- |
| | | | phenyl |
| —CH₃ | —CH₃ | —C(O)—N(CH₃)₂ | 2-benzyloxymethyl- |
| | | | phenyl |
| —CH₃ | —CH₃ | —C(O)—N(CH₃)₂ | 2-methoxymethyl-phenyl |
| —CH₃ | —CH₃ | —C(O)—N(CH₃)₂ | 2-thienyl |
| —CH₃ | —CH₃ | —C(O)—N(CH₃)₂ | 3-methyl-2-thienyl |
| —CH₃ | —CH₃ | —C(O)—N(CH₃)₂ | 3-thienyl |
| —CH₃ | —CH₃ | —C(O)—N(CH₃)₂ | 2-methyl-3-thienyl |
| —CH₃ | —CH₃ | —C(O)—N(CH₃)₂ | 4-methyl-3-thienyl |
| —CH₃ | —CH₃ | —C(O)—N(CH₃)₂ | 1-benzothien-3-yl |
| —CH₃ | —CH₃ | —C(O)—N(CH₃)₂ | 1-naphthyl |
| —CH₃ | —CH₃ | —C(O)—N(CH₃)₂ | 2-naphthyl |
| —CH₃ | —CH₃ | —C(O)—N(CH₃)₂ | 2-N-methyl-pyrrolyl |
| —CH₃ | —CH₃ | —C(O)—N(CH₃)₂ | 4-pyridyl |
| —CH₃ | —CH₃ | —C(O)—N(H)CH₃ | 2-methyl-phenyl |
| —CH₃ | —CH₃ | —C(O)—N(H)CH₃ | 2-ethyl-phenyl |
| —CH₃ | —CH₃ | —C(O)—N(H)CH₃ | 2-isopropyl-phenyl |
| —CH₃ | —CH₃ | —C(O)—N(H)CH₃ | 4-fluoro-2-methyl-phenyl |
| —CH₃ | —CH₃ | —C(O)—N(H)CH₃ | 2-trifluoromethyl-phenyl |
| —CH₃ | —CH₃ | —C(O)—N(H)CH₃ | 2-hydroxymethyl-phenyl |
| —CH₃ | —CH₃ | —C(O)—N(H)CH₃ | 2-chloro-phenyl |
| —CH₃ | —CH₃ | —C(O)—N(H)CH₃ | 2-(2-hydroxyethyl)- |
| | | | phenyl |
| —CH₃ | —CH₃ | —C(O)—N(H)CH₃ | 2-(1-hydroxyethyl)- |
| | | | phenyl |
| —CH₃ | —CH₃ | —C(O)—N(H)CH₃ | 2-benzyloxymethyl- |
| | | | phenyl |
| —CH₃ | —CH₃ | —C(O)—N(H)CH₃ | 2-methoxymethyl-phenyl |
| —CH₃ | —CH₃ | —C(O)—N(H)CH₃ | 2-thienyl |
| —CH₃ | —CH₃ | —C(O)—N(H)CH₃ | 3-methyl-2-thienyl |
| —CH₃ | —CH₃ | —C(O)—N(H)CH₃ | 3-thienyl |
| —CH₃ | —CH₃ | —C(O)—N(H)CH₃ | 2-methyl-3-thienyl |
| —CH₃ | —CH₃ | —C(O)—N(H)CH₃ | 4-methyl-3-thienyl |
| —CH₃ | —CH₃ | —C(O)—N(H)CH₃ | 1-benzothien-3-yl |
| —CH₃ | —CH₃ | —C(O)—N(H)CH₃ | 1-naphthyl |
| —CH₃ | —CH₃ | —C(O)—N(H)CH₃ | 2-naphthyl |
| —CH₃ | —CH₃ | —C(O)—N(H)CH₃ | 2-N-methyl-pyrrolyl |
| —CH₃ | —CH₃ | —C(O)—N(H)CH₃ | 4-pyridyl |
| —CH₃ | —CH₃ | —C(O)—N(H)-cyclopropyl | 2-methyl-phenyl |
| —CH₃ | —CH₃ | —C(O)—N(H)-cyclopropyl | 2-ethyl-phenyl |
| —CH₃ | —CH₃ | —C(O)—N(H)-cyclopropyl | 2-isopropyl-phenyl |
| —CH₃ | —CH₃ | —C(O)—N(H)-cyclopropyl | 4-fluoro-2-methyl-phenyl |
| —CH₃ | —CH₃ | —C(O)—N(H)-cyclopropyl | 2-trifluoromethyl-phenyl |
| —CH₃ | —CH₃ | —C(O)—N(H)-cyclopropyl | 2-hydroxymethyl-phenyl |
| —CH₃ | —CH₃ | —C(O)—N(H)-cyclopropyl | 2-chloro-phenyl |
| —CH₃ | —CH₃ | —C(O)—N(H)-cyclopropyl | 2-(2-hydroxyethyl)- |
| | | | phenyl |
| —CH₃ | —CH₃ | —C(O)—N(H)-cyclopropyl | 2-(1-hydroxyethyl)- |
| | | | phenyl |
| —CH₃ | —CH₃ | —C(O)—N(H)-cyclopropyl | 2-benzyloxymethyl- |
| | | | phenyl |
| —CH₃ | —CH₃ | —C(O)—N(H)-cyclopropyl | 2-methoxymethyl-phenyl |
| —CH₃ | —CH₃ | —C(O)—N(H)-cyclopropyl | 2-thienyl |
| —CH₃ | —CH₃ | —C(O)—N(H)-cyclopropyl | 3-methyl-2-thienyl |
| —CH₃ | —CH₃ | —C(O)—N(H)-cyclopropyl | 3-thienyl |
| —CH₃ | —CH₃ | —C(O)—N(H)-cyclopropyl | 2-methyl-3-thienyl |
| —CH₃ | —CH₃ | —C(O)—N(H)-cyclopropyl | 4-methyl-3-thienyl |
| —CH₃ | —CH₃ | —C(O)—N(H)-cyclopropyl | 1-benzothien-3-yl |
| —CH₃ | —CH₃ | —C(O)—N(H)-cyclopropyl | 1-naphthyl |
| —CH₃ | —CH₃ | —C(O)—N(H)-cyclopropyl | 2-naphthyl |
| —CH₃ | —CH₃ | —C(O)—N(H)-cyclopropyl | 2-N-methyl-pyrrolyl |
| —CH₃ | —CH₃ | —C(O)—N(H)-cyclopropyl | 4-pyridyl |
| —CH₃ | —CH₃ | —C(O)-pyrrolidin-1-yl | 2-methyl-phenyl |
| —CH₃ | —CH₃ | —C(O)-pyrrolidin-1-yl | 2-ethyl-phenyl |
| —CH₃ | —CH₃ | —C(O)-pyrrolidin-1-yl | 2-isopropyl-phenyl |
| —CH₃ | —CH₃ | —C(O)-pyrrolidin-1-yl | 4-fluoro-2-methyl-phenyl |
| —CH₃ | —CH₃ | —C(O)-pyrrolidin-1-yl | 2-trifluoromethyl-phenyl |
| —CH₃ | —CH₃ | —C(O)-pyrrolidin-1-yl | 2-hydroxymethyl-phenyl |
| —CH₃ | —CH₃ | —C(O)-pyrrolidin-1-yl | 2-chloro-phenyl |
| —CH₃ | —CH₃ | —C(O)-pyrrolidin-1-yl | 2-(2-hydroxyethyl)- |
| | | | phenyl |
| —CH₃ | —CH₃ | —C(O)-pyrrolidin-1-yl | 2-(1-hydroxyethyl)- |
| | | | phenyl |
| —CH₃ | —CH₃ | —C(O)-pyrrolidin-1-yl | 2-benzyloxymethyl- |
| | | | phenyl |
| —CH₃ | —CH₃ | —C(O)-pyrrolidin-1-yl | 2-methoxymethyl-phenyl |
| —CH₃ | —CH₃ | —C(O)-pyrrolidin-1-yl | 2-thienyl |
| —CH₃ | —CH₃ | —C(O)-pyrrolidin-1-yl | 3-methyl-2-thienyl |
| —CH₃ | —CH₃ | —C(O)-pyrrolidin-1-yl | 3-thienyl |
| —CH₃ | —CH₃ | —C(O)-pyrrolidin-1-yl | 2-methyl-3-thienyl |
| —CH₃ | —CH₃ | —C(O)-pyrrolidin-1-yl | 4-methyl-3-thienyl |
| —CH₃ | —CH₃ | —C(O)-pyrrolidin-1-yl | 1-benzothien-3-yl |
| —CH₃ | —CH₃ | —C(O)-pyrrolidin-1-yl | 1-naphthyl |
| —CH₃ | —CH₃ | —C(O)-pyrrolidin-1-yl | 2-naphthyl |
| —CH₃ | —CH₃ | —C(O)-pyrrolidin-1-yl | 2-N-methyl-pyrrolyl |
| —CH₃ | —CH₃ | —C(O)-pyrrolidin-1-yl | 4-pyridyl |
| —CH₃ | —CH₃ | —C(O)-azetidin-1-yl | 2-methyl-phenyl |
| —CH₃ | —CH₃ | —C(O)-azetidin-1-yl | 2-ethyl-phenyl |
| —CH₃ | —CH₃ | —C(O)-azetidin-1-yl | 2-isopropyl-phenyl |
| —CH₃ | —CH₃ | —C(O)-azetidin-1-yl | 4-fluoro-2-methyl-phenyl |
| —CH₃ | —CH₃ | —C(O)-azetidin-1-yl | 2-trifluoromethyl-phenyl |
| —CH₃ | —CH₃ | —C(O)-azetidin-1-yl | 2-hydroxymethyl-phenyl |
| —CH₃ | —CH₃ | —C(O)-azetidin-1-yl | 2-chloro-phenyl |
| —CH₃ | —CH₃ | —C(O)-azetidin-1-yl | 2-(2-hydroxyethyl)- |
| | | | phenyl |
| —CH₃ | —CH₃ | —C(O)-azetidin-1-yl | 2-(1-hydroxyethyl)- |
| | | | phenyl |
| —CH₃ | —CH₃ | —C(O)-azetidin-1-yl | 2-benzyloxymethyl- |
| | | | phenyl |
| —CH₃ | —CH₃ | —C(O)-azetidin-1-yl | 2-methoxymethyl-phenyl |
| —CH₃ | —CH₃ | —C(O)-azetidin-1-yl | 2-thienyl |
| —CH₃ | —CH₃ | —C(O)-azetidin-1-yl | 3-methyl-2-thienyl |
| —CH₃ | —CH₃ | —C(O)-azetidin-1-yl | 3-thienyl |
| —CH₃ | —CH₃ | —C(O)-azetidin-1-yl | 2-methyl-3-thienyl |
| —CH₃ | —CH₃ | —C(O)-azetidin-1-yl | 4-methyl-3-thienyl |
| —CH₃ | —CH₃ | —C(O)-azetidin-1-yl | 1-benzothien-3-yl |
| —CH₃ | —CH₃ | —C(O)-azetidin-1-yl | 1-naphthyl |
| —CH₃ | —CH₃ | —C(O)-azetidin-1-yl | 2-naphthyl |
| —CH₃ | —CH₃ | —C(O)-azetidin-1-yl | 2-N-methyl-pyrrolyl |
| —CH₃ | —CH₃ | —C(O)-azetidin-1-yl | 4-pyridyl |
| —CH₃ | —CH₃ | —CH₂—O—CH₃ | 2-methyl-phenyl |
| —CH₃ | —CH₃ | —CH₂—O—CH₃ | 2-ethyl-phenyl |
| —CH₃ | —CH₃ | —CH₂—O—CH₃ | 2-isopropyl-phenyl |
| —CH₃ | —CH₃ | —CH₂—O—CH₃ | 4-fluoro-2-methyl-phenyl |
| —CH₃ | —CH₃ | —CH₂—O—CH₃ | 2-trifluoromethyl-phenyl |
| —CH₃ | —CH₃ | —CH₂—O—CH₃ | 2-hydroxymethyl-phenyl |
| —CH₃ | —CH₃ | —CH₂—O—CH₃ | 2-chloro-phenyl |
| —CH₃ | —CH₃ | —CH₂—O—CH₃ | 2-(2-hydroxyethyl)- |
| | | | phenyl |
| —CH₃ | —CH₃ | —CH₂—O—CH₃ | 2-(1-hydroxyethyl)- |
| | | | phenyl |
| —CH₃ | —CH₃ | —CH₂—O—CH₃ | 2-benzyloxymethyl- |
| | | | phenyl |
| —CH₃ | —CH₃ | —CH₂—O—CH₃ | 2-methoxymethyl-phenyl |
| —CH₃ | —CH₃ | —CH₂—O—CH₃ | 2-thienyl |
| —CH₃ | —CH₃ | —CH₂—O—CH₃ | 3-methyl-2-thienyl |
| —CH₃ | —CH₃ | —CH₂—O—CH₃ | 3-thienyl |
| —CH₃ | —CH₃ | —CH₂—O—CH₃ | 2-methyl-3-thienyl |
| —CH₃ | —CH₃ | —CH₂—O—CH₃ | 4-methyl-3-thienyl |
| —CH₃ | —CH₃ | —CH₂—O—CH₃ | 1-benzothien-3-yl |
| —CH₃ | —CH₃ | —CH₂—O—CH₃ | 1-naphthyl |
| —CH₃ | —CH₃ | —CH₂—O—CH₃ | 2-naphthyl |
| —CH₃ | —CH₃ | —CH₂—O—CH₃ | 2-N-methyl-pyrrolyl |
| —CH₃ | —CH₃ | —CH₂—O—CH₃ | 4-pyridyl |
| —CH₃ | H | —C(O)—N(CH₃)₂ | 2-methyl-phenyl |
| —CH₃ | H | —C(O)—N(CH₃)₂ | 2-ethyl-phenyl |
| —CH₃ | H | —C(O)—N(CH₃)₂ | 2-isopropyl-phenyl |
| —CH₃ | H | —C(O)—N(CH₃)₂ | 4-fluoro-2-methyl-phenyl |
| —CH₃ | H | —C(O)—N(CH₃)₂ | 2-trifluoromethyl-phenyl |
| —CH₃ | H | —C(O)—N(CH₃)₂ | 2-hydroxymethyl-phenyl |
| —CH₃ | H | —C(O)—N(CH₃)₂ | 2-chloro-phenyl |
| —CH₃ | H | —C(O)—N(CH₃)₂ | 2-(2-hydroxyethyl)- |
| | | | phenyl |
| —CH₃ | H | —C(O)—N(CH₃)₂ | 2-(1-hydroxyethyl)- |
| | | | phenyl |
| —CH₃ | H | —C(O)—N(CH₃)₂ | 2-benzyloxymethyl- |
| | | | phenyl |
| —CH₃ | H | —C(O)—N(CH₃)₂ | 2-methoxymethyl-phenyl |
| —CH₃ | H | —C(O)—N(CH₃)₂ | 2-thienyl |
| —CH₃ | H | —C(O)—N(CH₃)₂ | 3-methyl-2-thienyl |
| —CH₃ | H | —C(O)—N(CH₃)₂ | 3-thienyl |
| —CH₃ | H | —C(O)—N(CH₃)₂ | 2-methyl-3-thienyl |
| —CH₃ | H | —C(O)—N(CH₃)₂ | 4-methyl-3-thienyl |
| —CH₃ | H | —C(O)—N(CH₃)₂ | 1-benzothien-3-yl |
| —CH₃ | H | —C(O)—N(CH₃)₂ | 1-naphthyl |
| —CH₃ | H | —C(O)—N(CH₃)₂ | 2-naphthyl |
| —CH₃ | —H | —C(O)—N(CH₃)₂ | 2-N-methyl-pyrrolyl |
| —CH₃ | —H | —C(O)—N(CH₃)₂ | 4-pyridyl |
| —CH₃ | H | —C(O)—N(H)CH₃ | 2-methyl-phenyl |
| —CH₃ | H | —C(O)—N(H)CH₃ | 2-ethyl-phenyl |
| —CH₃ | H | —C(O)—N(H)CH₃ | 2-isopropyl-phenyl |
| —CH₃ | H | —C(O)—N(H)CH₃ | 4-fluoro-2-methyl-phenyl |
| —CH₃ | H | —C(O)—N(H)CH₃ | 2-trifluoromethyl-phenyl |
| —CH₃ | H | —C(O)—N(H)CH₃ | 2-hydroxymethyl-phenyl |
| —CH₃ | H | —C(O)—N(H)CH₃ | 2-chloro-phenyl |
| —CH₃ | H | —C(O)—N(H)CH₃ | 2-(2-hydroxyethyl)- |
| | | | phenyl |
| —CH₃ | H | —C(O)—N(H)CH₃ | 2-(1-hydroxyethyl)- |
| | | | phenyl |
| —CH₃ | H | —C(O)—N(H)CH₃ | 2-benzyloxymethyl- |
| | | | phenyl |
| —CH₃ | H | —C(O)—N(H)CH₃ | 2-methoxymethyl-phenyl |
| —CH₃ | H | —C(O)—N(H)CH₃ | 2-thienyl |
| —CH₃ | H | —C(O)—N(H)CH₃ | 3-methyl-2-thienyl |
| —CH₃ | H | —C(O)—N(H)CH₃ | 3-thienyl |
| —CH₃ | H | —C(O)—N(H)CH₃ | 2-methyl-3-thienyl |
| —CH₃ | H | —C(O)—N(H)CH₃ | 4-methyl-3-thienyl |
| —CH₃ | H | —C(O)—N(H)CH₃ | 1-benzothien-3-yl |
| —CH₃ | H | —C(O)—N(H)CH₃ | 1-naphthyl |
| —CH₃ | H | —C(O)—N(H)CH₃ | 2-naphthyl |
| —CH₃ | H | —C(O)—N(H)CH₃ | 2-N-methyl-pyrrolyl |
| —CH₃ | H | —C(O)—N(H)CH₃ | 4-pyridyl |
| —CH₃ | H | —C(O)—N(H)-cyclopropyl | 2-methyl-phenyl |
| —CH₃ | H | —C(O)—N(H)-cyclopropyl | 2-ethyl-phenyl |
| —CH₃ | H | —C(O)—N(H)-cyclopropyl | 2-isopropyl-phenyl |
| —CH₃ | H | —C(O)—N(H)-cyclopropyl | 4-fluoro-2-methyl-phenyl |
| —CH₃ | H | —C(O)—N(H)-cyclopropyl | 2-trifluoromethyl-phenyl |
| —CH₃ | H | —C(O)—N(H)-cyclopropyl | 2-hydroxymethyl-phenyl |
| —CH₃ | H | —C(O)—N(H)-cyclopropyl | 2-chloro-phenyl |
| —CH₃ | H | —C(O)—N(H)-cyclopropyl | 2-(2-hydroxyethyl)- |
| | | | phenyl |
| —CH₃ | H | —C(O)—N(H)-cyclopropyl | 2-(1-hydroxyethyl)- |
| | | | phenyl |
| —CH₃ | H | —C(O)—N(H)-cyclopropyl | 2-benzyloxymethyl- |
| | | | phenyl |
| —CH₃ | H | —C(O)—N(H)-cyclopropyl | 2-methoxymethyl-phenyl |
| —CH₃ | H | —C(O)—N(H)-cyclopropyl | 2-thienyl |
| —CH₃ | H | —C(O)—N(H)-cyclopropyl | 3-methyl-2-thienyl |
| —CH₃ | H | —C(O)—N(H)-cyclopropyl | 3-thienyl |
| —CH₃ | H | —C(O)—N(H)-cyclopropyl | 2-methyl-3-thienyl |
| —CH₃ | H | —C(O)—N(H)-cyclopropyl | 4-methyl-3-thienyl |
| —CH₃ | H | —C(O)—N(H)-cyclopropyl | 1-benzothien-3-yl |
| —CH₃ | H | —C(O)—N(H)-cyclopropyl | 1-naphthyl |
| —CH₃ | H | —C(O)—N(H)-cyclopropyl | 2-naphthyl |
| —CH₃ | H | —C(O)—N(H)-cyclopropyl | 2-N-methyl-pyrrolyl |
| —CH₃ | H | —C(O)—N(H)-cyclopropyl | 4-pyridyl |
| —CH₃ | H | —C(O)-pyrrolidin-1-yl | 2-methyl-phenyl |
| —CH₃ | H | —C(O)-pyrrolidin-1-yl | 2-ethyl-phenyl |
| —CH₃ | H | —C(O)-pyrrolidin-1-yl | 2-isopropyl-phenyl |
| —CH₃ | H | —C(O)-pyrrolidin-1-yl | 4-fluoro-2-methyl-phenyl |
| —CH₃ | H | —C(O)-pyrrolidin-1-yl | 2-trifluoromethyl-phenyl |
| —CH₃ | H | —C(O)-pyrrolidin-1-yl | 2-hydroxymethyl-phenyl |
| —CH₃ | H | —C(O)-pyrrolidin-1-yl | 2-chloro-phenyl |
| —CH₃ | H | —C(O)-pyrrolidin-1-yl | 2-(2-hydroxyethyl)- |
| | | | phenyl |
| —CH₃ | H | —C(O)-pyrrolidin-1-yl | 2-(1-hydroxyethyl)- |
| | | | phenyl |
| —CH₃ | H | —C(O)-pyrrolidin-1-yl | 2-benzyloxymethyl- |
| | | | phenyl |
| —CH₃ | H | —C(O)-pyrrolidin-1-yl | 2-methoxymethyl-phenyl |
| —CH₃ | H | —C(O)-pyrrolidin-1-yl | 2-thienyl |
| —CH₃ | H | —C(O)-pyrrolidin-1-yl | 3-methyl-2-thienyl |
| —CH₃ | H | —C(O)-pyrrolidin-1-yl | 3-thienyl |
| —CH₃ | H | —C(O)-pyrrolidin-1-yl | 2-methyl-3-thienyl |
| —CH₃ | H | —C(O)-pyrrolidin-1-yl | 4-methyl-3-thienyl |
| —CH₃ | H | —C(O)-pyrrolidin-1-yl | 1-benzothien-3-yl |
| —CH₃ | H | —C(O)-pyrrolidin-1-yl | 1-naphthyl |
| —CH₃ | H | —C(O)-pyrrolidin-1-yl | 2-naphthyl |
| —CH₃ | H | —C(O)-pyrrolidin-1-yl | 2-N-methyl-pyrrolyl |
| —CH₃ | H | —C(O)-pyrrolidin-1-yl | 4-pyridyl |
| —CH₃ | H | —C(O)-azetidin-1-yl | 2-methyl-phenyl |
| —CH₃ | H | —C(O)-azetidin-1-yl | 2-ethyl-phenyl |
| —CH₃ | H | —C(O)-azetidin-1-yl | 2-isopropyl-phenyl |
| —CH₃ | H | —C(O)-azetidin-1-yl | 4-fluoro-2-methyl-phenyl |
| —CH₃ | H | —C(O)-azetidin-1-yl | 2-trifluoromethyl-phenyl |
| —CH₃ | H | —C(O)-azetidin-1-yl | 2-hydroxymethyl-phenyl |
| —CH₃ | H | —C(O)-azetidin-1-yl | 2-chloro-phenyl |
| —CH₃ | H | —C(O)-azetidin-1-yl | 2-(2-hydroxyethyl)- |
| | | | phenyl |
| —CH₃ | H | —C(O)-azetidin-1-yl | 2-(1-hydroxyethyl)- |
| | | | phenyl |
| —CH₃ | H | —C(O)-azetidin-1-yl | 2-benzyloxymethyl- |
| | | | phenyl |
| —CH₃ | H | —C(O)-azetidin-1-yl | 2-methoxymethyl-phenyl |
| —CH₃ | H | —C(O)-azetidin-1-yl | 2-thienyl |
| —CH₃ | H | —C(O)-azetidin-1-yl | 3-methyl-2-thienyl |
| —CH₃ | H | —C(O)-azetidin-1-yl | 3-thienyl |
| —CH₃ | H | —C(O)-azetidin-1-yl | 2-methyl-3-thienyl |
| —CH₃ | H | —C(O)-azetidin-1-yl | 4-methyl-3-thienyl |
| —CH₃ | H | —C(O)-azetidin-1-yl | 1-benzothien-3-yl |
| —CH₃ | H | —C(O)-azetidin-1-yl | 1-naphthyl |
| —CH₃ | H | —C(O)-azetidin-1-yl | 2-naphthyl |
| —CH₃ | H | —C(O)-azetidin-1-yl | 2-N-methyl-pyrrolyl |
| —CH₃ | H | —C(O)-azetidin-1-yl | 4-pyridyl |
| —CH₃ | H | —CH₂—O—CH₃ | 2-methyl-phenyl |
| —CH₃ | H | —CH₂—O—CH₃ | 2-ethyl-phenyl |
| —CH₃ | H | —CH₂—O—CH₃ | 2-isopropyl-phenyl |
| —CH₃ | H | —CH₂—O—CH₃ | 4-fluoro-2-methyl-phenyl |
| —CH₃ | H | —CH₂—O—CH₃ | 2-trifluoromethyl-phenyl |
| —CH₃ | H | —CH₂—O—CH₃ | 2-hydroxymethyl-phenyl |
| —CH₃ | H | —CH₂—O—CH₃ | 2-chloro-phenyl |
| —CH₃ | H | —CH₂—O—CH₃ | 2-(2-hydroxyethyl)- |
| | | | phenyl |
| —CH₃ | H | —CH₂—O—CH₃ | 2-(1-hydroxyethyl)- |
| | | | phenyl |
| —CH₃ | H | —CH₂—O—CH₃ | 2-benzyloxymethyl- |
| | | | phenyl |
| —CH₃ | H | —CH₂—O—CH₃ | 2-methoxymethyl-phenyl |
| —CH₃ | H | —CH₂—O—CH₃ | 2-thienyl |
| —CH₃ | H | —CH₂—O—CH₃ | 3-methyl-2-thienyl |
| —CH₃ | H | —CH₂—O—CH₃ | 3-thienyl |
| —CH₃ | H | —CH₂—O—CH₃ | 2-methyl-3-thienyl |
| —CH₃ | H | —CH₂—O—CH₃ | 4-methyl-3-thienyl |
| —CH₃ | H | —CH₂—O—CH₃ | 1-benzothien-3-yl |
| —CH₃ | H | —CH₂—O—CH₃ | 1-naphthyl |
| —CH₃ | H | —CH₂—O—CH₃ | 2-naphthyl |
| —CH₃ | H | —CH₂—O—CH₃ | 2-N-methyl-pyrrolyl |
| —CH₃ | H | —CH₂—O—CH₃ | 4-pyridyl |

12. (canceled)

13. A compound of the formula 3-a 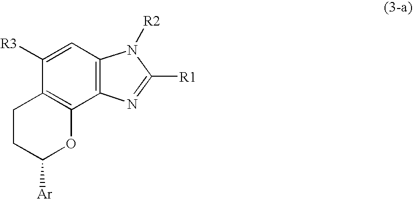
in which the substituents R1, R2, R3, and Ar have the meanings given in the following table,

| R1 | R2 | R3 | Ar |
|---|---|---|---|
| —CH₃ | —CH₃ | —C(O)—N(CH₃)₂ | 2-methyl-phenyl |
| —CH₃ | —CH₃ | —C(O)—N(CH₃)₂ | 2-ethyl-phenyl |
| —CH₃ | —CH₃ | —C(O)—N(CH₃)₂ | 2-isopropyl-phenyl |
| —CH₃ | —CH₃ | —C(O)—N(CH₃)₂ | 4-fluoro-2-methyl-phenyl |
| —CH₃ | —CH₃ | —C(O)—N(CH₃)₂ | 2-trifluoromethyl-phenyl |
| —CH₃ | —CH₃ | —C(O)—N(CH₃)₂ | 2-hydroxymethyl-phenyl |
| —CH₃ | —CH₃ | —C(O)—N(CH₃)₂ | 2-chloro-phenyl |
| —CH₃ | —CH₃ | —C(O)—N(CH₃)₂ | 2-(2-hydroxyethyl)- |
| | | | phenyl |
| —CH₃ | —CH₃ | —C(O)—N(CH₃)₂ | 2-(1-hydroxyethyl)- |
| | | | phenyl |
| —CH₃ | —CH₃ | —C(O)—N(CH₃)₂ | 2-benzyloxymethyl- |
| | | | phenyl |
| —CH₃ | —CH₃ | —C(O)—N(CH₃)₂ | 2-methoxymethyl-phenyl |
| —CH₃ | —CH₃ | —C(O)—N(CH₃)₂ | 2-thienyl |
| —CH₃ | —CH₃ | —C(O)—N(CH₃)₂ | 3-methyl-2-thienyl |
| —CH₃ | —CH₃ | —C(O)—N(CH₃)₂ | 3-thienyl |
| —CH₃ | —CH₃ | —C(O)—N(CH₃)₂ | 2-methyl-3-thienyl |
| —CH₃ | —CH₃ | —C(O)—N(CH₃)₂ | 4-methyl-3-thienyl |
| —CH₃ | —CH₃ | —C(O)—N(CH₃)₂ | 1-benzothien-3-yl |
| —CH₃ | —CH₃ | —C(O)—N(CH₃)₂ | 1-naphthyl |
| —CH₃ | —CH₃ | —C(O)—N(CH₃)₂ | 2-naphthyl |
| —CH₃ | —CH₃ | —C(O)—N(CH₃)₂ | 2-N-methyl-pyrrolyl |
| —CH₃ | —CH₃ | —C(O)—N(CH₃)₂ | 4-pyridyl |
| —CH₃ | —CH₃ | —C(O)—N(H)CH₃ | 2-methyl-phenyl |
| —CH₃ | —CH₃ | —C(O)—N(H)CH₃ | 2-ethyl-phenyl |
| —CH₃ | —CH₃ | —C(O)—N(H)CH₃ | 2-isopropyl-phenyl |
| —CH₃ | —CH₃ | —C(O)—N(H)CH₃ | 4-fluoro-2-methyl-phenyl |
| —CH₃ | —CH₃ | —C(O)—N(H)CH₃ | 2-trifluoromethyl-phenyl |
| —CH₃ | —CH₃ | —C(O)—N(H)CH₃ | 2-hydroxymethyl-phenyl |
| —CH₃ | —CH₃ | —C(O)—N(H)CH₃ | 2-chloro-phenyl |
| —CH₃ | —CH₃ | —C(O)—N(H)CH₃ | 2-(2-hydroxyethyl)- |
| | | | phenyl |
| —CH₃ | —CH₃ | —C(O)—N(H)CH₃ | 2-(1-hydroxyethyl)- |
| | | | phenyl |
| —CH₃ | —CH₃ | —C(O)—N(H)CH₃ | 2-benzyloxymethyl- |
| | | | phenyl |
| —CH₃ | —CH₃ | —C(O)—N(H)CH₃ | 2-methoxymethyl-phenyl |
| —CH₃ | —CH₃ | —C(O)—N(H)CH₃ | 2-thienyl |
| —CH₃ | —CH₃ | —C(O)—N(H)CH₃ | 3-methyl-2-thienyl |
| —CH₃ | —CH₃ | —C(O)—N(H)CH₃ | 3-thienyl |
| —CH₃ | —CH₃ | —C(O)—N(H)CH₃ | 2-methyl-3-thienyl |
| —CH₃ | —CH₃ | —C(O)—N(H)CH₃ | 4-methyl-3-thienyl |
| —CH₃ | —CH₃ | —C(O)—N(H)CH₃ | 1-benzothien-3-yl |
| —CH₃ | —CH₃ | —C(O)—N(H)CH₃ | 1-naphthyl |
| —CH₃ | —CH₃ | —C(O)—N(H)CH₃ | 2-naphthyl |
| —CH₃ | —CH₃ | —C(O)—N(H)CH₃ | 2-N-methyl-pyrrolyl |
| —CH₃ | —CH₃ | —C(O)—N(H)CH₃ | 4-pyridyl |
| —CH₃ | —CH₃ | —C(O)—N(H)-cyclopropyl | 2-methyl-phenyl |
| —CH₃ | —CH₃ | —C(O)—N(H)-cyclopropyl | 2-ethyl-phenyl |
| —CH₃ | —CH₃ | —C(O)—N(H)-cyclopropyl | 2-isopropyl-phenyl |
| —CH₃ | —CH₃ | —C(O)—N(H)-cyclopropyl | 4-fluoro-2-methyl-phenyl |
| —CH₃ | —CH₃ | —C(O)—N(H)-cyclopropyl | 2-trifluoromethyl-phenyl |
| —CH₃ | —CH₃ | —C(O)—N(H)-cyclopropyl | 2-hydroxymethyl-phenyl |
| —CH₃ | —CH₃ | —C(O)—N(H)-cyclopropyl | 2-chloro-phenyl |
| —CH₃ | —CH₃ | —C(O)—N(H)-cyclopropyl | 2-(2-hydroxyethyl)- |
| | | | phenyl |
| —CH₃ | —CH₃ | —C(O)—N(H)-cyclopropyl | 2-(1-hydroxyethyl)- |
| | | | phenyl |
| —CH₃ | —CH₃ | —C(O)—N(H)-cyclopropyl | 2-benzyloxymethyl- |
| | | | phenyl |
| —CH₃ | —CH₃ | —C(O)—N(H)-cyclopropyl | 2-methoxymethyl-phenyl |
| —CH₃ | —CH₃ | —C(O)—N(H)-cyclopropyl | 2-thienyl |
| —CH₃ | —CH₃ | —C(O)—N(H)-cyclopropyl | 3-methyl-2-thienyl |
| —CH₃ | —CH₃ | —C(O)—N(H)-cyclopropyl | 3-thienyl |
| —CH₃ | —CH₃ | —C(O)—N(H)-cyclopropyl | 2-methyl-3-thienyl |
| —CH₃ | —CH₃ | —C(O)—N(H)-cyclopropyl | 4-methyl-3-thienyl |
| —CH₃ | —CH₃ | —C(O)—N(H)-cyclopropyl | 1-benzothien-3-yl |
| —CH₃ | —CH₃ | —C(O)—N(H)-cyclopropyl | 1-naphthyl |
| —CH₃ | —CH₃ | —C(O)—N(H)-cyclopropyl | 2-naphthyl |
| —CH₃ | —CH₃ | —C(O)—N(H)-cyclopropyl | 2-N-methyl-pyrrolyl |
| —CH₃ | —CH₃ | —C(O)—N(H)-cyclopropyl | 4-pyridyl |
| —CH₃ | —CH₃ | —C(O)-pyrrolidin-1-yl | 2-methyl-phenyl |
| —CH₃ | —CH₃ | —C(O)-pyrrolidin-1-yl | 2-ethyl-phenyl |
| —CH₃ | —CH₃ | —C(O)-pyrrolidin-1-yl | 2-isopropyl-phenyl |
| —CH₃ | —CH₃ | —C(O)-pyrrolidin-1-yl | 4-fluoro-2-methyl-phenyl |
| —CH₃ | —CH₃ | —C(O)-pyrrolidin-1-yl | 2-trifluoromethyl-phenyl |
| —CH₃ | —CH₃ | —C(O)-pyrrolidin-1-yl | 2-hydroxymethyl-phenyl |
| —CH₃ | —CH₃ | —C(O)-pyrrolidin-1-yl | 2-chloro-phenyl |
| —CH₃ | —CH₃ | —C(O)-pyrrolidin-1-yl | 2-(2-hydroxyethyl)- |
| | | | phenyl |
| —CH₃ | —CH₃ | —C(O)-pyrrolidin-1-yl | 2-(1-hydroxyethyl)- |
| | | | phenyl |
| —CH₃ | —CH₃ | —C(O)-pyrrolidin-1-yl | 2-benzyloxymethyl- |
| | | | phenyl |
| —CH₃ | —CH₃ | —C(O)-pyrrolidin-1-yl | 2-methoxymethyl-phenyl |
| —CH₃ | —CH₃ | —C(O)-pyrrolidin-1-yl | 2-thienyl |
| —CH₃ | —CH₃ | —C(O)-pyrrolidin-1-yl | 3-methyl-2-thienyl |
| —CH₃ | —CH₃ | —C(O)-pyrrolidin-1-yl | 3-thienyl |
| —CH₃ | —CH₃ | —C(O)-pyrrolidin-1-yl | 2-methyl-3-thienyl |
| —CH₃ | —CH₃ | —C(O)-pyrrolidin-1-yl | 4-methyl-3-thienyl |
| —CH₃ | —CH₃ | —C(O)-pyrrolidin-1-yl | 1-benzothien-3-yl |
| —CH₃ | —CH₃ | —C(O)-pyrrolidin-1-yl | 1-naphthyl |
| —CH₃ | —CH₃ | —C(O)-pyrrolidin-1-yl | 2-naphthyl |
| —CH₃ | —CH₃ | —C(O)-pyrrolidin-1-yl | 2-N-methyl-pyrrolyl |
| —CH₃ | —CH₃ | —C(O)-pyrrolidin-1-yl | 4-pyridyl |
| —CH₃ | —CH₃ | —C(O)-azetidin-1-yl | 2-methyl-phenyl |
| —CH₃ | —CH₃ | —C(O)-azetidin-1-yl | 2-ethyl-phenyl |
| —CH₃ | —CH₃ | —C(O)-azetidin-1-yl | 2-isopropyl-phenyl |
| —CH₃ | —CH₃ | —C(O)-azetidin-1-yl | 4-fluoro-2-methyl-phenyl |
| —CH₃ | —CH₃ | —C(O)-azetidin-1-yl | 2-trifluoromethyl-phenyl |
| —CH₃ | —CH₃ | —C(O)-azetidin-1-yl | 2-hydroxymethyl-phenyl |
| —CH₃ | —CH₃ | —C(O)-azetidin-1-yl | 2-chloro-phenyl |
| —CH₃ | —CH₃ | —C(O)-azetidin-1-yl | 2-(2-hydroxyethyl)- |
| | | | phenyl |
| —CH₃ | —CH₃ | —C(O)-azetidin-1-yl | 2-(1-hydroxyethyl)- |
| | | | phenyl |
| —CH₃ | —CH₃ | —C(O)-azetidin-1-yl | 2-benzyloxymethyl- |
| | | | phenyl |
| —CH₃ | —CH₃ | —C(O)-azetidin-1-yl | 2-methoxymethyl-phenyl |
| —CH₃ | —CH₃ | —C(O)-azetidin-1-yl | 2-thienyl |
| —CH₃ | —CH₃ | —C(O)-azetidin-1-yl | 3-methyl-2-thienyl |
| —CH₃ | —CH₃ | —C(O)-azetidin-1-yl | 3-thienyl |
| —CH₃ | —CH₃ | —C(O)-azetidin-1-yl | 2-methyl-3-thienyl |
| —CH₃ | —CH₃ | —C(O)-azetidin-1-yl | 4-methyl-3-thienyl |
| —CH₃ | —CH₃ | —C(O)-azetidin-1-yl | 1-benzothien-3-yl |
| —CH₃ | —CH₃ | —C(O)-azetidin-1-yl | 1-naphthyl |
| —CH₃ | —CH₃ | —C(O)-azetidin-1-yl | 2-naphthyl |
| —CH₃ | —CH₃ | —C(O)-azetidin-1-yl | 2-N-methyl-pyrrolyl |
| —CH₃ | —CH₃ | —C(O)-azetidin-1-yl | 4-pyridyl |
| —CH₃ | —CH₃ | —CH₂—O—CH₃ | 2-methyl-phenyl |
| —CH₃ | —CH₃ | —CH₂—O—CH₃ | 2-ethyl-phenyl |
| —CH₃ | —CH₃ | —CH₂—O—CH₃ | 2-isopropyl-phenyl |
| —CH₃ | —CH₃ | —CH₂—O—CH₃ | 4-fluoro-2-methyl-phenyl |
| —CH₃ | —CH₃ | —CH₂—O—CH₃ | 2-trifluoromethyl-phenyl |
| —CH₃ | —CH₃ | —CH₂—O—CH₃ | 2-hydroxymethyl-phenyl |
| —CH₃ | —CH₃ | —CH₂—O—CH₃ | 2-chloro-phenyl |
| —CH₃ | —CH₃ | —CH₂—O—CH₃ | 2-(2-hydroxyethyl)- |
| | | | phenyl |
| —CH₃ | —CH₃ | —CH₂—O—CH₃ | 2-(1-hydroxyethyl)- |
| | | | phenyl |
| —CH₃ | —CH₃ | —CH₂—O—CH₃ | 2-benzyloxymethyl- |
| | | | phenyl |
| —CH₃ | —CH₃ | —CH₂—O—CH₃ | 2-methoxymethyl-phenyl |
| —CH₃ | —CH₃ | —CH₂—O—CH₃ | 2-thienyl |
| —CH₃ | —CH₃ | —CH₂—O—CH₃ | 3-methyl-2-thienyl |
| —CH₃ | —CH₃ | —CH₂—O—CH₃ | 3-thienyl |
| —CH₃ | —CH₃ | —CH₂—O—CH₃ | 2-methyl-3-thienyl |
| —CH₃ | —CH₃ | —CH₂—O—CH₃ | 4-methyl-3-thienyl |
| —CH₃ | —CH₃ | —CH₂—O—CH₃ | 1-benzothien-3-yl |
| —CH₃ | —CH₃ | —CH₂—O—CH₃ | 1-naphthyl |
| —CH₃ | —CH₃ | —CH₂—O—CH₃ | 2-naphthyl |
| —CH₃ | —CH₃ | —CH₂—O—CH₃ | 2-N-methyl-pyrrolyl |
| —CH₃ | —CH₃ | —CH₂—O—CH₃ | 4-pyridyl |
| —CH₃ | H | —C(O)—N(CH₃)₂ | 2-methyl-phenyl |
| —CH₃ | H | —C(O)—N(CH₃)₂ | 2-ethyl-phenyl |
| —CH₃ | H | —C(O)—N(CH₃)₂ | 2-isopropyl-phenyl |
| —CH₃ | H | —C(O)—N(CH₃)₂ | 4-fluoro-2-methyl-phenyl |
| —CH₃ | H | —C(O)—N(CH₃)₂ | 2-trifluoromethyl-phenyl |
| —CH₃ | H | —C(O)—N(CH₃)₂ | 2-hydroxymethyl-phenyl |
| —CH₃ | H | —C(O)—N(CH₃)₂ | 2-chloro-phenyl |
| —CH₃ | H | —C(O)—N(CH₃)₂ | 2-(2-hydroxyethyl)- |
| | | | phenyl |
| —CH₃ | H | —C(O)—N(CH₃)₂ | 2-(1-hydroxyethyl)- |
| | | | phenyl |
| —CH₃ | H | —C(O)—N(CH₃)₂ | 2-benzyloxymethyl- |
| | | | phenyl |
| —CH₃ | H | —C(O)—N(CH₃)₂ | 2-methoxymethyl-phenyl |
| —CH₃ | H | —C(O)—N(CH₃)₂ | 2-thienyl |
| —CH₃ | H | —C(O)—N(CH₃)₂ | 3-methyl-2-thienyl |
| —CH₃ | H | —C(O)—N(CH₃)₂ | 3-thienyl |
| —CH₃ | H | —C(O)—N(CH₃)₂ | 2-methyl-3-thienyl |
| —CH₃ | H | —C(O)—N(CH₃)₂ | 4-methyl-3-thienyl |
| —CH₃ | H | —C(O)—N(CH₃)₂ | 1-benzothien-3-yl |
| —CH₃ | H | —C(O)—N(CH₃)₂ | 1-naphthyl |
| —CH₃ | H | —C(O)—N(CH₃)₂ | 2-naphthyl |
| —CH₃ | H | —C(O)—N(CH₃)₂ | 2-N-methyl-pyrrolyl |
| —CH₃ | H | —C(O)—N(CH₃)₂ | 4-pyridyl |
| —CH₃ | H | —C(O)—N(H)CH₃ | 2-methyl-phenyl |
| —CH₃ | H | —C(O)—N(H)CH₃ | 2-ethyl-phenyl |
| —CH₃ | H | —C(O)—N(H)CH₃ | 2-isopropyl-phenyl |
| —CH₃ | H | —C(O)—N(H)CH₃ | 4-fluoro-2-methyl-phenyl |
| —CH₃ | H | —C(O)—N(H)CH₃ | 2-trifluoromethyl-phenyl |
| —CH₃ | H | —C(O)—N(H)CH₃ | 2-hydroxymethyl-phenyl |
| —CH₃ | H | —C(O)—N(H)CH₃ | 2-chloro-phenyl |
| —CH₃ | H | —C(O)—N(H)CH₃ | 2-(2-hydroxyethyl)- |
| | | | phenyl |
| —CH₃ | H | —C(O)—N(H)CH₃ | 2-(1-hydroxyethyl)- |
| | | | phenyl |
| —CH₃ | H | —C(O)—N(H)CH₃ | 2-benzyloxymethyl- |
| | | | phenyl |
| —CH₃ | H | —C(O)—N(H)CH₃ | 2-methoxymethyl-phenyl |
| —CH₃ | H | —C(O)—N(H)CH₃ | 2-thienyl |
| —CH₃ | H | —C(O)—N(H)CH₃ | 3-methyl-2-thienyl |
| —CH₃ | H | —C(O)—N(H)CH₃ | 3-thienyl |
| —CH₃ | H | —C(O)—N(H)CH₃ | 2-methyl-3-thienyl |
| —CH₃ | H | —C(O)—N(H)CH₃ | 4-methyl-3-thienyl |
| —CH₃ | H | —C(O)—N(H)CH₃ | 1-benzothien-3-yl |
| —CH₃ | H | —C(O)—N(H)CH₃ | 1-naphthyl |
| —CH₃ | H | —C(O)—N(H)CH₃ | 2-naphthyl |
| —CH₃ | H | —C(O)—N(H)CH₃ | 2-N-methyl-pyrrolyl |
| —CH₃ | H | —C(O)—N(H)CH₃ | 4-pyridyl |
| —CH₃ | H | —C(O)—N(H)-cyclopropyl | 2-methyl-phenyl |
| —CH₃ | H | —C(O)—N(H)-cyclopropyl | 2-ethyl-phenyl |
| —CH₃ | H | —C(O)—N(H)-cyclopropyl | 2-isopropyl-phenyl |
| —CH₃ | H | —C(O)—N(H)-cyclopropyl | 4-fluoro-2-methyl-phenyl |
| —CH₃ | H | —C(O)—N(H)-cyclopropyl | 2-trifluoromethyl-phenyl |
| —CH₃ | H | —C(O)—N(H)-cyclopropyl | 2-hydroxymethyl-phenyl |
| —CH₃ | H | —C(O)—N(H)-cyclopropyl | 2-chloro-phenyl |
| —CH₃ | H | —C(O)—N(H)-cyclopropyl | 2-(2-hydroxyethyl)- |
| | | | phenyl |
| —CH₃ | H | —C(O)—N(H)-cyclopropyl | 2-(1-hydroxyethyl)- |
| | | | phenyl |
| —CH₃ | H | —C(O)—N(H)-cyclopropyl | 2-benzyloxymethyl- |
| | | | phenyl |
| —CH₃ | H | —C(O)—N(H)-cyclopropyl | 2-methoxymethyl-phenyl |
| —CH₃ | H | —C(O)—N(H)-cyclopropyl | 2-thienyl |
| —CH₃ | H | —C(O)—N(H)-cyclopropyl | 3-methyl-2-thienyl |
| —CH₃ | H | —C(O)—N(H)-cyclopropyl | 3-thienyl |
| —CH₃ | H | —C(O)—N(H)-cyclopropyl | 2-methyl-3-thienyl |
| —CH₃ | H | —C(O)—N(H)-cyclopropyl | 4-methyl-3-thienyl |
| —CH₃ | H | —C(O)—N(H)-cyclopropyl | 1-benzothien-3-yl |
| —CH₃ | H | —C(O)—N(H)-cyclopropyl | 1-naphthyl |
| —CH₃ | H | —C(O)—N(H)-cyclopropyl | 2-naphthyl |
| —CH₃ | H | —C(O)—N(H)-cyclopropyl | 2-N-methyl-pyrrolyl |
| —CH₃ | H | —C(O)—N(H)-cyclopropyl | 4-pyridyl |
| —CH₃ | H | —C(O)-pyrrolidin-1-yl | 2-methyl-phenyl |
| —CH₃ | H | —C(O)-pyrrolidin-1-yl | 2-ethyl-phenyl |
| —CH₃ | H | —C(O)-pyrrolidin-1-yl | 2-isopropyl-phenyl |
| —CH₃ | H | —C(O)-pyrrolidin-1-yl | 4-fluoro-2-methyl-phenyl |
| —CH₃ | H | —C(O)-pyrrolidin-1-yl | 2-trifluoromethyl-phenyl |
| —CH₃ | H | —C(O)-pyrrolidin-1-yl | 2-hydroxymethyl-phenyl |
| —CH₃ | H | —C(O)-pyrrolidin-1-yl | 2-chloro-phenyl |
| —CH₃ | H | —C(O)-pyrrolidin-1-yl | 2-(2-hydroxyethyl)- |
| | | | phenyl |
| —CH₃ | H | —C(O)-pyrrolidin-1-yl | 2-(1-hydroxyethyl)- |
| | | | phenyl |
| —CH₃ | H | —C(O)-pyrrolidin-1-yl | 2-benzyloxymethyl- |
| | | | phenyl |
| —CH₃ | H | —C(O)-pyrrolidin-1-yl | 2-methoxymethyl-phenyl |
| —CH₃ | H | —C(O)-pyrrolidin-1-yl | 2-thienyl |
| —CH₃ | H | —C(O)-pyrrolidin-1-yl | 3-methyl-2-thienyl |
| —CH₃ | H | —C(O)-pyrrolidin-1-yl | 3-thienyl |
| —CH₃ | H | —C(O)-pyrrolidin-1-yl | 2-methyl-3-thienyl |
| —CH₃ | H | —C(O)-pyrrolidin-1-yl | 4-methyl-3-thienyl |
| —CH₃ | H | —C(O)-pyrrolidin-1-yl | 1-benzothien-3-yl |
| —CH₃ | H | —C(O)-pyrrolidin-1-yl | 1-naphthyl |
| —CH₃ | H | —C(O)-pyrrolidin-1-yl | 2-naphthyl |
| —CH₃ | H | —C(O)-pyrrolidin-1-yl | 2-N-methyl-pyrrolyl |
| —CH₃ | H | —C(O)-pyrrolidin-1-yl | 4-pyridyl |
| —CH₃ | H | —C(O)-azetidin-1-yl | 2-methyl-phenyl |
| —CH₃ | H | —C(O)-azetidin-1-yl | 2-ethyl-phenyl |
| —CH₃ | H | —C(O)-azetidin-1-yl | 2-isopropyl-phenyl |
| —CH₃ | H | —C(O)-azetidin-1-yl | 4-fluoro-2-methyl-phenyl |
| —CH₃ | H | —C(O)-azetidin-1-yl | 2-trifluoromethyl-phenyl |
| —CH₃ | H | —C(O)-azetidin-1-yl | 2-hydroxymethyl-phenyl |
| —CH₃ | H | —C(O)-azetidin-1-yl | 2-chloro-phenyl |
| —CH₃ | H | —C(O)-azetidin-1-yl | 2-(2-hydroxyethyl)- |
| | | | phenyl |
| —CH₃ | H | —C(O)-azetidin-1-yl | 2-(1-hydroxyethyl)- |
| | | | phenyl |
| —CH₃ | H | —C(O)-azetidin-1-yl | 2-benzyloxymethyl- |
| | | | phenyl |
| —CH₃ | H | —C(O)-azetidin-1-yl | 2-methoxymethyl-phenyl |
| —CH₃ | H | —C(O)-azetidin-1-yl | 2-thienyl |
| —CH₃ | H | —C(O)-azetidin-1-yl | 3-methyl-2-thienyl |
| —CH₃ | H | —C(O)-azetidin-1-yl | 3-thienyl |
| —CH₃ | H | —C(O)-azetidin-1-yl | 2-methyl-3-thienyl |
| —CH₃ | H | —C(O)-azetidin-1-yl | 4-methyl-3-thienyl |
| —CH₃ | H | —C(O)-azetidin-1-yl | 1-benzothien-3-yl |
| —CH₃ | H | —C(O)-azetidin-1-yl | 1-naphthyl |
| —CH₃ | H | —C(O)-azetidin-1-yl | 2-naphthyl |
| —CH₃ | H | —C(O)-azetidin-1-yl | 2-N-methyl-pyrrolyl |
| —CH₃ | H | —C(O)-azetidin-1-yl | 4-pyridyl |
| —CH₃ | H | —CH₂—O—CH₃ | 2-methyl-phenyl |
| —CH₃ | H | —CH₂—O—CH₃ | 2-ethyl-phenyl |
| —CH₃ | H | —CH₂—O—CH₃ | 2-isopropyl-phenyl |
| —CH₃ | H | —CH₂—O—CH₃ | 4-fluoro-2-methyl-phenyl |
| —CH₃ | H | —CH₂—O—CH₃ | 2-trifluoromethyl-phenyl |
| —CH₃ | H | —CH₂—O—CH₃ | 2-hydroxymethyl-phenyl |
| —CH₃ | H | —CH₂—O—CH₃ | 2-chloro-phenyl |
| —CH₃ | H | —CH₂—O—CH₃ | 2-(2-hydroxyethyl)- |
| | | | phenyl |
| —CH₃ | H | —CH₂—O—CH₃ | 2-(1-hydroxyethyl)- |
| | | | phenyl |
| —CH₃ | H | —CH₂—O—CH₃ | 2-benzyloxymethyl- |
| | | | phenyl |
| —CH₃ | H | —CH₂—O—CH₃ | 2-methoxymethyl-phenyl |
| —CH₃ | H | —CH₂—O—CH₃ | 2-thienyl |
| —CH₃ | H | —CH₂—O—CH₃ | 3-methyl-2-thienyl |
| —CH₃ | H | —CH₂—O—CH₃ | 3-thienyl |
| —CH₃ | H | —CH₂—O—CH₃ | 2-methyl-3-thienyl |
| —CH₃ | H | —CH₂—O—CH₃ | 4-methyl-3-thienyl |
| —CH₃ | H | —CH₂—O—CH₃ | 1-benzothien-3-yl |
| —CH₃ | H | —CH₂—O—CH₃ | 1-naphthyl |
| —CH₃ | H | —CH₂—O—CH₃ | 2-naphthyl |
| —CH₃ | H | —CH₂—O—CH₃ | 2-N-methyl-pyrrolyl |
| —CH₃ | H | —CH₂—O—CH₃ | 4-pyridyl |

or a salt thereof.

14. A compound of the formula 3-a 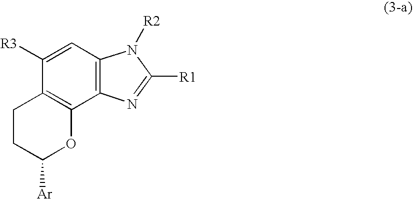
which is selected from the group consisting of
(8S)-2-Methyl-8-phenyl-3-(2-trimethylsilanyl-ethoxymethyl)-3,6,7,8-tetrahydro-chromeno[7,8-d]imidazole-5-carboxylic Acid Dimethylamide,
(8S)-8-(2-Fluoro-phenyl)-2,3-dimethyl-3,6,7,8-tetrahydro-chromeno[7,8-d]imidazole-5-carboxylic Acid Dimethylamide,
(8S)-8-(4-Fluoro-phenyl)-2,3-dimethyl-3,6,7,8-tetrahydro-chromeno[7,8-d]imidazole-5-carboxylic Acid Dimethylamide,
(8S)-2,3-Dimethyl-8-o-tolyl-3,6,7,8-tetrahydro-chromeno[7,8-d]imidazole-5-carboxylic Acid Dimethylamide,
(8S)-8-(2-Chloro-phenyl)-2,3-dimethyl-3,6,7,8-tetrahydro-chromeno[7,8-d]imidazole-5-carboxylic Acid Dimethylamide,
(8S)-2,3-Dimethyl-8-(2-trifluoromethyl-phenyl)-3,6,7,8-tetrahydro-chromeno[7,8-d]imidazole-5-carboxylic Acid Dimethylamide,
(8S)-2,3-Dimethyl-8-naphthalen-2-yl-3,6,7,8-tetrahydro-chromeno[7,8-d]imidazole-5-carboxylic Acid Dimethylamide,
(8S)-(2-Ethyl-phenyl)-2,3-dimethyl-3,6,7,8-tetrahydro-chromeno[7,8-d]imidazole-5-carboxylic Acid Dimethylamide,
(8S)2,3-Dimethyl-8-thiophen-2-yl-3,6,7,8-tetrahydro-chromeno[7,8-d]imidazole-5-carboxylic Acid Dimethylamide,
(8S)-8-(4-Fluoro-2-methyl-phenyl)-2,3-dimethyl-3,6,7,8-tetrahydro-chromeno[7,8-d]imidazole-5-carboxylic Acid Dimethylamide,
(8S)-(2,3-Dimethyl-8-o-tolyl-3,6,7,8-tetrahydro-chromeno[7,8-d]imidazol-5-yl)-pyrrolidin-1-yl-methanone,
(8S)-2,3-Dimethyl-8-o-tolyl-3,6,7,8-tetrahydro-chromeno[7,8-d]imidazole-5-carboxylic Acid Methylamide,
(8S)-Azetidin-1-yl-((S)-2,3-dimethyl-8-o-tolyl-3,6,7,8-tetrahydro-chromeno[7,8-d]imidazol-5-yl)-methanone,
(8S)-8-(2-Benzyloxymethyl-phenyl)-2,3-dimethyl-3,6,7,8-tetrahydro-chromeno[7,8-d]imidazole-5-carboxylic acid dimethylamide,
(8S)-8-(2-Methoxymethyl-phenyl)-2,3-dimethyl-3,6,7,8-tetrahydro-chromeno[7,8-d]imidazole-5-carboxylic acid dimethylamide,
(8S)-2-Methyl-8-o-tolyl-3-(2-trimethylsilanyl-ethoxymethyl)-3,6,7,8-tetrahydro-chromeno[7,8-d]imidazole-5-carboxylic acid dimethylamide,
(8S)-2-Methyl-8-o-tolyl-3,6,7,8-tetrahydro-chromeno[7,8-d]imidazole-5-carboxylic acid dimethylamide,
(8S)-2,3-Dimethyl-8-(2-methyl-thiophen-3-yl)-3,6,7,8-tetrahydro-chromeno[7,8-d]imidazole-5-carboxylic acid dimethylamide,
(8S)-2,3-Dimethyl-8-o-tolyl-3,6,7,8-tetrahydro-chromeno[7,8-d]imidazole-5-carboxylic acid cyclopropylamide,
5-Methoxymethyl-2,3-dimethyl-8-o-tolyl-3,6,7,8-tetrahydro-chromeno[7,8-d]imidazole,
and salts thereof.

15. A pharmaceutical composition comprising a compound as claimed in claim 13 and/or a pharmacologically acceptable salt thereof together with a pharmaceutically acceptable auxiliary and/or excipient.

16. (canceled)

17. (canceled)

18. A pharmaceutical composition comprising a compound as claimed in claim 14 and/or a pharmacologically acceptable salt thereof together with a pharmaceutically acceptable auxiliary and/or excipient.

19. A method of treating or preventing a gastrointestinal disorder in a patient comprising administering to a patient in need thereof a compound as claimed in claim 13 or a pharmaceutically acceptable salt thereof.

20. A method of treating or preventing a gastrointestinal disorder in a patient comprising administering to a patient in need thereof a compound as claimed in claim 14 or a pharmaceutically acceptable salt thereof.
